SEQUENCE LISTING (12 sequences)

SEQ ID NO: 1 - DNA, 1281 bp, Homo sapiens
  human SMAD family member 7 (SMAD7)
atgttcaggaccaaacgatctgcgctcgtccggcgtctctggaggagccgtgcgcccggcggcgaggacgaggaggagggcgcagggggaggtggaggaggaggcgagctgcggggagaaggggcgacggacagccgagcgcatggggccggtggcggcggcccgggcagggctggatgctgcctgggcaaggcggtgcgaggtgccaaaggtcaccaccatccccacccgccagccgcgggcgccggcgcggccgggggcgccgaggcggatctgaaggcgctcacgcactcggtgctcaagaaactgaaggagcggcagctggagctgctgctccaggccgtggagtcccgcggcgggacgcgcaccgcgtgcctcctgctgcccggccgcctggactgcaggctgggcccgggggcgcccgccggcgcgcagcctgcgcagccgccctcgtcctactcgctccccctcctgctgtgcaaagtgttcaggtggccggatctcaggcattcctcggaagtcaagaggctgtgttgctgtgaatcttacgggaagatcaaccccgagctggtgtgctgcaacccccatcaccttagccgactctgcgaactagagtctcccccccctccttactccagatacccgatggattttctcaaaccaactgcagactgtccagatgctgtgccttcctccgctgaaacagggggaacgaattatctggcccctggggggctttcagattcccaacttcttctggagcctggggatcggtcacactggtgcgtggtggcatactgggaggagaagacgagagtggggaggctctactgtgtccaggagccctctctggatatcttctatgatctacctcaggggaatggcttttgcctcggacagctcaattcggacaacaagagtcagctggtgcagaaggtgcggagcaaaatcggctgcggcatccagctgacgcgggaggtggatggtgtgtgggtgtacaaccgcagcagttaccccatcttcatcaagtccgccacactggacaacccggactccaggacgctgttggtacacaaggtgttccccggtttctccatcaaggctttcgactacgagaaggcgtacagcctgcagcggcccaatgaccacgagtttatgcagcagccgtggacgggctttaccgtgcagatcagctttgtgaagggctggggccagtgctacacccgccagttcatcagcagctgcccgtgctggctagaggtcatcttcaacagccggtag

SEQ ID NO: 2 - DNA, 21 bp, Artificial Sequence
  anti-SMAD7 oligonucleotide or a chemically modified anti-SMAD7 oligonucleotide
  misc_feature (3)..(3): n represents a nucleotide comprising a nitrogenous base selected from the group consisting of cytosine, 5-methylcytosine and 2′-O-methylcytosine
  misc_feature (4)..(4): n represents a nucleotide comprising a nitrogenous base selected from the group consisting of guanine, 5-methylguanine, or 2′-O-methylguanine
  misc_feature (16)..(16): n represents a nucleotide comprising a nitrogenous base selected from the group consisting of cytosine, 5-methylcytosine and 2′-O-methylcytosine
  misc_feature (17)..(17): n represents a nucleotide comprising a nitrogenous base selected from the group consisting of guanine, 5-methylguanine, or 2′-O-methylguanine
gtnnccccttctcccnncagc

SEQ ID NO: 3 - DNA, 21 bp, Artificial Sequence
  anti-SMAD7 oligonucleotide or a chemically modified anti-SMAD7 oligonucleotide
  misc_feature (3)..(3): represents 5-methyl-2′-deoxycytidine (5-Me-dC)
  misc_feature (16)..(16): represents 5-methyl-2′-deoxycytidine (5-Me-dC)
gtngccccttctcccngcagc

SEQ ID NO: 4 - DNA, 20 bp, Artificial Sequence
  anti-SMAD7 oligonucleotide or a chemically modified anti-SMAD7 oligonucleotide
gtcgccccttctccccgcag

SEQ ID NO: 5 - DNA, 20 bp, Artificial Sequence
  anti-SMAD7 oligonucleotide or a chemically modified anti-SMAD7 oligonucleotide
  misc_feature (3)..(3): represents 5-methyl-2′-deoxycytidine (5-Me-dC)
  misc_feature (16)..(16): represents 5-methyl-2′-deoxycytidine (5-Me-dC)
gtngccccttctcccngcag

SEQ ID NO: 6 - DNA, 18 bp, Artificial Sequence
  anti-SMAD7 oligonucleotide or a chemically modified anti-SMAD7 oligonucleotide
gtttggtcctgaacatgc

SEQ ID NO: 7 - DNA, 16 bp, Artificial Sequence
  anti-SMAD7 oligonucleotide or a chemically modified anti-SMAD7 oligonucleotide
gtttggtcctgaacat

SEQ ID NO: 8 - DNA, 17 bp, Artificial Sequence
  anti-SMAD7 oligonucleotide or a chemically modified anti-SMAD7 oligonucleotide
gtttggtcctgaacatg

SEQ ID NO: 9 - DNA, 18 bp, Artificial Sequence
  anti-SMAD7 oligonucleotide or a chemically modified anti-SMAD7 oligonucleotide
agcaccgagtgcgtgagc

SEQ ID NO: 10 - DNA, 18 bp, Artificial Sequence
  anti-SMAD7 oligonucleotide or a chemically modified anti-SMAD7 oligonucleotide
cgaacatgacctccgcac

SEQ ID NO: 11 - DNA, 17 bp, Artificial Sequence
  anti-SMAD7 oligonucleotide or a chemically modified anti-SMAD7 oligonucleotide
gatcgtttggtcctgaa

SEQ ID NO: 12 - DNA, 17 bp, Artificial Sequence
  anti-SMAD7 oligonucleotide or a chemically modified anti-SMAD7 oligonucleotide
atcgtttggtcctgaac

(19) United States
(12) Patent Application Publication
(10) Pub. No.: US 2020/0392491 A1
(43) Pub. Date: Dec. 17, 2020

(54) Method of Preparing Oligonucleotide Compounds

(71) Applicant: Nogra Pharma Limited, Dublin (IE)

(72) Inventors: Anthony Frank, Reading, MA (US); Kevin Klopfer, Flemington, NJ (US); Donald Joseph Gallagher, Jr., Hopkinton, MA (US); Laleh Jafarpour, Milford, MA (US)

(21) Appl. No.: 16/634,078

(22) PCT Filed: Jul. 26, 2018
(86) PCT No.: PCT/US18/43867

§ 371 Date: Jan. 24, 2020

Related U.S. Application Data
(60) Provisional application No. 62/538,504, filed on Jul. 28, 2017.

(51) Int. Cl. C12N 15/113

(52) U.S. Cl. CPC C12N 15/113; C12N 2330/30; C12N 2310/315; C12N 2310/11

(57) ABSTRACT

The present invention relates to compositions comprising oligonucleotides, specifically SMAD7 antisense oligonucleotides, and methods for preparing the same.

### CROSS REFERENCE TO RELATED APPLICATIONS

This application claims the benefit of priority from U.S. Provisional Application No. 62/538,504, filed Jul. 28, 2017. The foregoing related application, in its entirety, is incorporated herein by reference.

### 1. INTRODUCTION

This application relates to compositions comprising oligonucleotides, specifically Smad7 antisense oligonucleotides, and methods for preparing the same.

### 2. BACKGROUND

Oligonucleotides may be used in a variety of biological and biochemical applications. For example, oligonucleotides may be used in drug discovery and development, in diagnostic tests, as research agents, such as primers or probes in Polymerase Chain Reaction (PCR), as antisense agents in target validation, as competitive inhibitors of transcription factors, as ribozymes, as aptamers, as stimulators of the immune system, or as therapeutic biological agents to treat disease. Accordingly, the increase demand and use of oligonucleotides has led to an increasing demand for rapid, inexpensive and efficient methods for their synthesis, on increasing larger, multi-kilogram scale, and having GMP quality.

Mongersen (formerly GED-0301) is a 21-base, single-strand phosphorothioate oligonucleotide that hybridizes to the human SMAD7 messenger RNA (mRNA). Monteleone et al. 2015, NEJM 372:1104-1113. In a phase 2 clinical study, participants with Crohn's disease who received mongersen had significantly higher rates of remission and clinical response than those who received placebo. Monteleone et al. 2015, NEJM 372:1104-1113. Mongersen is a 21-base oligonucleotide with the sequence 5′-GTC GCC CCT TCT CCC CGC AGC-3′. The phosphorothioate chemistry consists of replacement of a nonbonding oxygen with a sulfur atom in each of the internucleotide linkages. The cytosine residues at nucleotide positions 3 and 16 are modified by 5-methylation.

Current methods of preparing naturally occurring oligonucleotides, and modified oligonucleotides, such as phosphorothioate and phosphorodithioate oligonucleotides, include solution and solid-phase synthesis procedures. For example, in solid-phase synthesis, the procedures rely on sequential addition of nucleotides to one end of a growing oligonucleotide chain. There remains a need for the preparation of large, multi-kilogram quantities, having GMP quality, of single-strand phosphorothioate oligonucleotides, such as Mongersen, which are often necessary for biological studies, pre-clinical and clinical trials and commercial production, and a need for methods of synthesis for preparing the same, that are rapid, inexpensive, and efficient, and that can produce the final target oligonucleotide on a large, multi-kilogram scale, and having GMP or CGMP quality.

### 3. SUMMARY

In one aspect, provided herein is a single batch composition of an oligonucleotide comprising at least 700 mmol of the oligonucleotide and at most 25 wt. % water; wherein the oligonucleotide has a nucleic acid sequence:

SEQ ID NO: 3:
(5′-GTX GCC CCT TCT CCC XGC AGC-3′),

wherein X represents 5-methyl-2′-deoxycytidine.

In another aspect, provided herein is a single batch composition of an oligonucleotide comprising at least 2 g/mmol of an at least 700 mmol synthesis scale of the oligonucleotide and at most 25 wt. % water; wherein the oligonucleotide has a nucleic acid sequence:

SEQ ID NO: 3:
(5′-GTX GCC CCT TCT CCC XGC AGC-3′),

wherein X represents 5-methyl-2′-deoxycytidine.

In another aspect, provided herein is a single batch composition of an oligonucleotide comprising at least 2 kg of the oligonucleotide and at most 25 wt. % water; wherein the oligonucleotide has a nucleic acid sequence:

SEQ ID NO: 3:
(5′-GTX GCC CCT TCT CCC XGC AGC-3′),

wherein X represents 5-methyl-2′-deoxycytidine.

In another aspect, provided herein is a single batch composition of an oligonucleotide comprising at least 50 mol % of the oligonucleotide output from at least one 700 mmol or greater oligonucleotide synthesis column and at most 25 wt. % water; wherein the oligonucleotide has a nucleic acid sequence:

SEQ ID NO: 3:
(5′-GTX GCC CCT TCT CCC XGC AGC-3′),

wherein X represents 5-methyl-2′-deoxycytidine.

In another aspect, provided herein is a substantially pure oligonucleotide composition of an oligonucleotide wherein the 5′-hydroxyl group of the 5′-terminal nucleoside is protected; wherein the oligonucleotide has a nucleic acid sequence:

SEQ ID NO: 3:
(5′-GTX GCC CCT TCT CCC XGC AGC-3′),

wherein X represents 5-methyl-2′-deoxycytidine.

In another aspect, provided herein is at least 700 mmol of an oligonucleotide composition comprising an oligonucleotide having at most 25 wt. % water and a nucleic acid sequence:

SEQ ID NO: 3:
(5′-GTX GCC CCT TCT CCC XGC AGC-3′),

wherein:
1) X represents 5-methyl-2′-deoxycytidine; and
2) the oligonucleotide is prepared according to a method comprising:
   a) providing a linker attached to a solid support wherein the linker comprises a protected hydroxyl group;
   b) deprotecting the protected hydroxyl group of the linker thereby creating a deprotected hydroxyl group;
   c) independently providing a nucleoside phosphoramidite, wherein the nucleoside phosphoramidite comprises a protected hydroxyl group and a protected phosphoramidite;
   d) independently coupling the nucleoside phosphoramidite to the deprotected hydroxyl group of the linker, or to the deprotected hydroxyl group of the nucleoside from the previous iteration of the reaction cycle, thereby creating a phosphite triester linked nucleoside;
   e) independently thiolating the protected phosphite triester linkage thereby creating a protected phosphorothioate linkage;
   f) optionally, independently capping unreacted deprotected hydroxyl groups;
   g) optionally, independently deprotecting the protected hydroxyl group of the nucleoside;
   h) repeating the providing, coupling, thiolating, capping, and deprotecting steps (steps c) through g) a predetermined number of times to provide a solid support-bound oligonucleotide;
   i) deprotecting the protected phosphorothioate linkages;
   j) cleaving the oligonucleotide from the solid support;
   k) eluting the oligonucleotide from the solid support;
   l) purifying the oligonucleotide eluate using an ion exchange chromatography column; and
   m) concentrating the solution of the oligonucleotide compound with thin film evaporation.

In certain embodiments of the single batch composition, the substantially pure oligonucleotide composition, the oligonucleotide or oligonucleotide composition prepared according to the methods of preparing, or the method of preparing, disclosed herein, at least one of the internucleotide linkages of the oligonucleotide is an O,O-linked phosphorothioate.

In certain embodiments of the single batch composition, the substantially pure oligonucleotide composition, the oligonucleotide or oligonucleotide composition prepared according to the methods of preparing, or the method of preparing, disclosed herein, all of the internucleotide linkages of the oligonucleotide are O,O-linked phosphorothioates.

In certain embodiments of the single batch composition, the substantially pure oligonucleotide composition, or of the method of preparing, disclosed herein, the purifying step 1) comprises:
1) loading the oligonucleotide eluate from eluting step k) onto the ion exchange chromatography column;
2) deprotecting the protected hydroxyl group from the terminal nucleoside; and
3) eluting the oligonucleotide from the ion exchange chromatography column using a salt gradient.

In certain embodiments of the single batch composition, the substantially pure oligonucleotide composition, or of the method of preparing, disclosed herein, the purifying step 1) comprises:
1) loading the oligonucleotide eluate from eluting step k) onto the ion exchange chromatography column;
2) deprotecting the protected hydroxyl group from the terminal nucleoside;
3) eluting the oligonucleotide from the ion exchange chromatography column using a salt gradient; and
4) desalting the oligonucleotide eluate from the ion exchange column via ultrafiltration and/or diafiltration.

In certain embodiments of the single batch composition, the substantially pure oligonucleotide composition, or of the method of preparing, disclosed herein, the concentrating step m) comprises concentrating the desalted solution of the oligonucleotide compound with thin film evaporation.

In certain embodiments of the single batch composition, the substantially pure oligonucleotide composition, or of the method of preparing, disclosed herein, the protected 5′-hydroxyl group of the terminal nucleoside of the loaded oligonucleotide eluate is deprotected with a protic acid.

In certain embodiments of the single batch composition, the substantially pure oligonucleotide composition, or of the method of preparing, disclosed herein, the 5′-hydroxyl protected group is deprotected with acetic acid, such as 80% aqueous acetic acid.

In certain embodiments of the single batch composition, the substantially pure oligonucleotide composition, or of the method of preparing, disclosed herein, the fully deprotected oligonucleotide is desalted via an ultrafiltration and/or diafiltration process.

In certain embodiments of the single batch composition, the substantially pure oligonucleotide composition, or of the method of preparing, disclosed herein, the fully deprotected oligonucleotide eluate is concentrated with thin film evaporation.

In certain embodiments of the single batch composition, the substantially pure oligonucleotide composition, or of the method of preparing, disclosed herein, the concentrated fully deprotected oligonucleotide resulting from thin film evaporation is a liquid composition and not subjected to a freeze drying process.

In certain embodiments of the single batch composition, the substantially pure oligonucleotide composition, or of the method of preparing, disclosed herein, the concentrated fully deprotected oligonucleotide resulting from thin film evaporation is further concentrated via a freeze drying process.

In certain embodiments of the single batch composition, the substantially pure oligonucleotide composition, or of the method of preparing, disclosed herein, the method steps are performed in the order in which they are recited.

In another aspect, provided herein is a pharmaceutical composition comprising at least a portion of a single batch composition or a substantially pure oligonucleotide composition as disclosed herein, or an oligonucleotide composition prepared according to the methods of preparing as disclosed herein, with a pharmaceutically acceptable adjuvant and/or excipient.

In certain embodiments of the pharmaceutical composition, the pharmaceutical composition is an oral dosage form, such as a tablet or a coated tablet.

In certain embodiments of the pharmaceutical composition, the pharmaceutical composition comprises in the range of between 10-500 mg of the oligonucleotide having a nucleic acid sequence:

SEQ ID NO: 3:
(5′-GTX GCC CCT TCT CCC XGC AGC-3′),

wherein X represents 5-methyl-2′-deoxycytidine.

In certain embodiments of the pharmaceutical composition, the pharmaceutical composition comprises about 40 mg or about 160 mg of the oligonucleotide having a nucleic acid sequence:

SEQ ID NO: 3:
(5′-GTX GCC CCT TCT CCC XGC AGC-3′),

wherein X represents 5-methyl-2′-deoxycytidine.

In another aspect, provided herein is a method of preparing a pharmaceutical composition, comprising formulating at least a portion of a single batch composition or a substantially pure oligonucleotide composition as disclosed herein, or an oligonucleotide composition prepared according to the methods of preparing as disclosed herein, with a pharmaceutically acceptable adjuvant and/or excipient.

In another aspect, provided herein is a method of preparing a series of pharmaceutical compositions, comprising partitioning a single batch composition or a substantially pure oligonucleotide composition as disclosed herein, or an oligonucleotide composition prepared according to the methods of preparing as disclosed herein, into a series of portions, amounts, or doses, suitable for oral dosage, and combining each portion, amount, or dose, suitable for oral dosage, of the series of portions amounts, or doses, suitable for oral dosage, with a pharmaceutically acceptable adjuvant and/or excipient.

In certain embodiments of the method of preparing the series of pharmaceutical compositions, the series of pharmaceutical compositions is at least 100 pharmaceutical compositions.

In certain embodiments of the method of preparing the series of pharmaceutical compositions, the series of pharmaceutical compositions is between 100-1,000,000 pharmaceutical compositions.

In certain embodiments of the method of preparing the series of pharmaceutical compositions, each portion, amount, or dose, suitable for oral dosage, of the series of portions, amounts, or doses, suitable for oral dosage, comprises in the range of between 10-500 mg of the oligonucleotide having a nucleic acid sequence:

SEQ ID NO: 3:
(5′-GTX GCC CCT TCT CCC XGC AGC-3′),

wherein X represents 5-methyl-2′-deoxycytidine.

In certain embodiments of the method of preparing the series of pharmaceutical compositions, each portion, amount, or dose, suitable for oral dosage, of the series of portions, amounts, or doses, suitable for oral dosage, comprises about 40 mg or about 160 mg of the oligonucleotide having a nucleic acid sequence:

SEQ ID NO: 3:
(5′-GTX GCC CCT TCT CCC XGC AGC-3′),

wherein X represents 5-methyl-2′-deoxycytidine.

In certain embodiments of the method of preparing the series of pharmaceutical compositions, the series of pharmaceutical compositions is a series of tablets.

In certain embodiments of the method of preparing the series of pharmaceutical compositions, the series of pharmaceutical compositions is a series of coated tablets.

In another aspect, provided herein is a pharmaceutical composition batch, comprising at least a portion of a single batch composition or a substantially pure oligonucleotide composition as disclosed herein, or an oligonucleotide composition prepared according to the methods of preparing as disclosed herein, and a pharmaceutically acceptable adjuvant and/or excipient.

In certain embodiments of the pharmaceutical composition batch, the pharmaceutical composition batch comprises at least 10 wt. %, at least 20 wt. %, at least 30 wt. %, at least 40 wt. %, at least 50 wt. %, at least 60 wt. %, at least 70 wt. %, at least 80 wt. %, at least 90 wt. %, at least 95 wt. %, or 100 wt. %, of the single batch composition, the substantially pure oligonucleotide composition, or the oligonucleotide composition prepared according to the methods of preparing as disclosed herein.

In certain embodiments of the pharmaceutical composition batch, the pharmaceutical composition batch comprises at least one oral dosage form, such as tablet or a coated tablet.

In certain embodiments of the pharmaceutical composition batch, the pharmaceutical composition batch comprises a series of oral dosage forms, such as a series of tablets or a series of coated tablets.

In certain embodiments of the single batch composition, the pharmaceutical composition batch, the method of preparing the single batch composition, the method of preparing the pharmaceutical composition, or the method of preparing the series of pharmaceutical compositions, as disclosed herein, the single batch composition, the pharmaceutical composition batch, the method of preparing the single batch composition, the method of preparing the pharmaceutical composition, or the method of preparing the series of pharmaceutical compositions, complies with the terms batch or lot as defined under 21 CFR 210.3(2) and 21 CFR 210.3(10), respectively.

In another aspect, provided herein is a method of treating or managing inflammatory bowel disease (IBD) in a patient having IBD, comprising administering a pharmaceutical composition as disclosed herein.

In certain embodiments of the method of treating or managing inflammatory bowel disease (IBD) in a patient having IBD, the inflammatory bowel disease (IBD) is Crohn's disease (CD).

In certain embodiments of the method of treating or managing inflammatory bowel disease (IBD) in a patient having IBD, the inflammatory bowel disease (IBD) is ulcerative colitis (UC).

### 4. BRIEF DESCRIPTION OF THE FIGURES

The following FIGURES exemplify aspects of the disclosed process but do not limit the scope of the process or of the examples disclosed herein.

FIG. 1. A schematic representing certain embodiments of preparing a single batch oligonucleotide composition, as used herein.

### 5. DETAILED DESCRIPTION

The terms “anti-SMAD7 ODN” and “anti-SMAD7 oligonucleotide”, as used herein, are understood to refer to an oligonucleotide (ODN) comprising a nucleic acid sequence that is complementary to a nucleic acid sequence in an mRNA molecule transcribed from the SMAD7 gene. More specifically, such an oligonucleotide can be complementary to the nucleic acid sequence in the coding region of such an mRNA. In certain embodiments, the anti-SMAD7 ODN can reduce the expression of SMAD7 when introduced into a cell (e.g., an immune cell, such as PBMC, pDC, or B-cell). In certain embodiments, an anti-SMAD7 ODN can reduce expression of an mRNA transcribed from the gene. In certain embodiments, an anti-SMAD7 ODN can reduce expression of a protein encoded by the gene. In certain embodiments, an anti-SMAD7 ODN can reduce secretion of a protein encoded by the gene from the cell into which the anti-SMAD7 ODN was introduced.

The terms “oligonucleotide” and “ODN” as used herein, are understood to refer to nucleic acids comprising a nucleic acid sequence that is at least 4, 5, 6, 7, 8, 9, 10, 11, 12, 13, 14, 15, 16, 17, 18, 19, 20, 21, 22, 23, 24, 25, 26, 27, 28, 29, or at least 30 nucleotides long; that is at most 4, 5, 6, 7, 8, 9, 10, 11, 12, 13, 14, 15, 16, 17, 18, 19, 20, 21, 22, 23, 24, 25, 26, 27, 28, 29, or at most 30 nucleotides long; or that is between 10 and 25, 15 and 30, 15 and 25, or 20 and 30 nucleotides long; or that is 18, 19, 20, 21, 22, 23, 24, 25, or 26 nucleotides long. In certain embodiments, the internucleoside linkage between two adjoining nucleosides of an ODN may be a phosphate linkage, e.g., a monophosphate linkage, or may be a phosphorothioate linkage. In certain embodiments, an ODN may contain one or more phosphate internucleoside linkages and one or more phosphorothioate internucleoside linkages. For example, in certain embodiments, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleosides of an ODN are linked by phosphate linkages, e.g., monophosphate linkages. For example, in certain embodiments, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleosides of an ODN are linked by phosphorothioate linkages. In certain embodiments, the oligo (or sugar) moiety of the ODN is a naturally occurring sugar, such as furanose ring system (e.g., ribose or 2′-deoxyribose ring systems). For example, in certain embodiments, the ODN is a deoxyribonucleotide (i.e., the oligo (or sugar) moiety of the ODN is a deoxyribose). In certain embodiments, the ODN is a ribonucleotide (i.e., the oligo (or sugar) moiety of the ODN is a ribose). For example, in certain embodiments, the oligo (or sugar) moiety of the nucleoside(s) utilized to prepare the desired ODN is deoxyribose. In certain embodiments, the oligo (or sugar) moiety of the nucleoside(s) utilized to prepare the desired ODN is ribose. In certain embodiments, one or more bases of an ODN are chemically modified, for example, methylated (e.g., 5-methyl-cytosine, 6-O-methyl-guanine, or 7-methyl-guanine). Additional exemplary chemical modifications of an ODN are described, e.g., in Section 5.2.

The term “SMAD7” (also known as CRCS3, FLJ16482, MADH7, MADH8, MAD (mothers against decapentaplegic, Drosophila) homolog 7, MAD homolog 8, SMAD, mothers against DPP homolog 7, mothers against DPP homolog 8) as used herein, is understood to mean the human protein or any of the mRNA transcripts encoded by the gene identified by Entrez GeneID No. 4092 and allelic variants thereof.

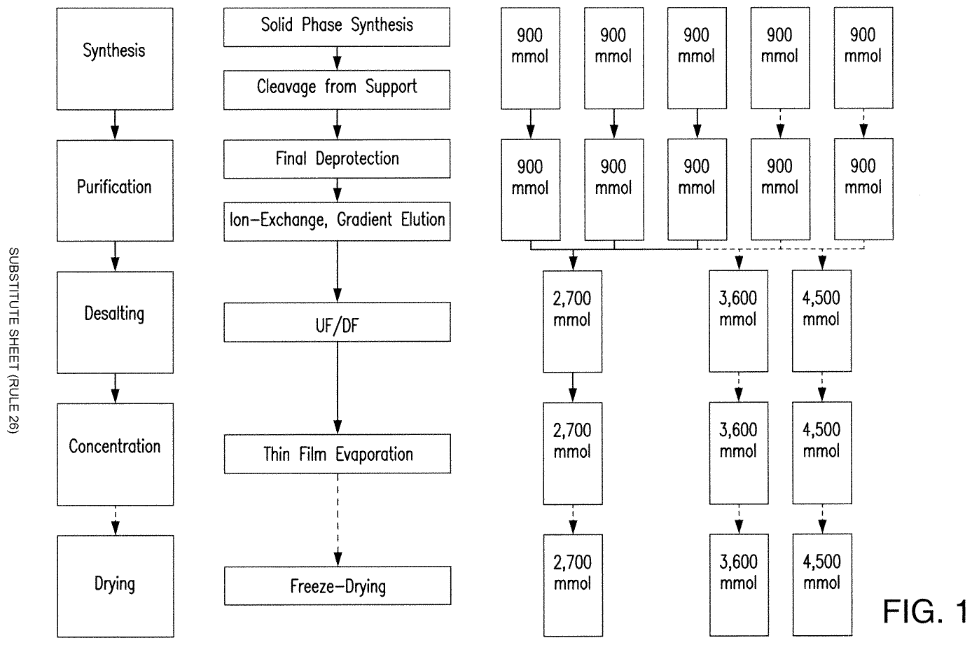

The terms “single batch composition” and “single batch oligonucleotide composition”, as used herein, are understood to refer to an oligonucleotide composition derived from at least one oligonucleotide synthesis column having a solid support (e.g., a solid support with a linker attached thereto) with a loading capacity of a specified quantity, or at least one synthesis run on the oligonucleotide synthesis column having the solid support with a loading capacity of a specified quantity. For example, in certain embodiments, the single batch composition (or single batch oligonucleotide composition) refers to an oligonucleotide composition derived from a plurality of oligonucleotide synthesis column(s) having a solid support (e.g., a solid support with a linker attached thereto) with a loading capacity of a specified quantity, or a plurality of synthesis run(s) on the oligonucleotide synthesis column having the solid support with a loading capacity of a specified quantity. For example, in certain embodiments, the single batch composition (or single batch oligonucleotide composition) refers to an oligonucleotide composition derived from at most 1, 2, 3, 4, 5, 6, 7, 8, 9, 10, 15, 20, 25, 30, 35, 40, 45, 50, 55, or at most 60 oligonucleotide synthesis columns having a solid support with a loading capacity of a specified quantity or synthesis runs on an oligonucleotide synthesis column(s) (e.g., run on parallel columns or subsequent to each other) with a loading capacity of a specified quantity, for example, no more than between 1-60, 1-55, 1-50, 1-45, 1-40, 1-35, 1-30, 1-25, 1-20, 1-15, 1-10, 1-9, 1-8, 1-7, 1-6, 1-5, 1-4, 1-3, 1-2, 3-10, 3-8, 3-5, 4-6, 5-10, 10-60, 10-50, 10-40, 10-30, 10-25, 10-20, 10-15, 20-50, 30-60, or 40-55 oligonucleotide synthesis columns having a solid support with a loading capacity of a specified quantity or synthesis runs on an oligonucleotide synthesis column(s) (e.g., run on parallel columns or subsequent to each other) with a loading capacity of a specified quantity. For example, in certain embodiments, the single batch composition (or single batch oligonucleotide composition) refers to an oligonucleotide composition derived from at least one or a plurality of oligonucleotide synthesis column(s) having a solid support (e.g., a solid support with a linker attached thereto) with a loading capacity of a specified quantity, or at least one or a plurality of synthesis run(s) on the oligonucleotide synthesis column having the solid support with a loading capacity of a specified quantity; wherein the oligonucleotide synthesis output from the at least one, or the plurality of, oligonucleotide synthesis column(s) (or synthesis run(s)) having the solid support with a loading capacity of the specified quantity, may: (1) provide the single batch composition (or single batch oligonucleotide composition) as a liquid oligonucleotide composition or a solid oligonucleotide composition; or (2) be pooled or combined (such that the resulting pooled/combined oligonucleotide composition has uniform character and quality, e.g., the physical and chemical properties are distributed throughout said resulting pooled/combined oligonucleotide composition) to provide the single batch composition (or single batch oligonucleotide composition) as a liquid oligonucleotide composition or a solid oligonucleotide composition. For example, in certain embodiments, the single batch composition (or single batch oligonucleotide composition) refers to an oligonucleotide composition derived from at least one or a plurality of oligonucleotide synthesis column(s) having a solid support (e.g., a solid support with a linker attached thereto) with a loading capacity of a specified quantity, or at least one or a plurality of synthesis run(s) on the oligonucleotide synthesis column having the solid support with a loading capacity of a specified quantity; wherein the oligonucleotide synthesis output from the at least one, or the plurality of, oligonucleotide synthesis column(s) (or synthesis run(s)) having the solid support with a loading capacity of the specified quantity, may: (1) be processed in a single iteration or in a plurality of parallel iterations (not necessarily conducted simultaneously) by at least one further process(s), comprising: an oligonucleotide chemical transformation process (e.g., an oligonucleotide deprotection or cleavage from solid support), an oligonucleotide purification process (e.g., ion-exchange chromatography), an oligonucleotide desalting process (e.g., ultrafiltration and/or diafiltration), a liquid composition concentration process (e.g., thin film evaporation), or a drying process (e.g., freeze drying), or combinations thereof; (2) be pooled or combined (such that the resulting pooled/combined oligonucleotide composition has uniform character and quality, e.g., the physical and chemical properties are distributed throughout said resulting pooled/combined oligonucleotide composition): (a) before being processed in a single iteration or in a plurality of parallel iterations (not necessarily conducted simultaneously) by the at least one further process(s) (or combinations thereof); or (b) followed by partitioning the resulting pooled/combined oligonucleotide composition into smaller portions before being processed in a plurality of parallel iterations (not necessarily conducted simultaneously) by the at least one further process(s) (or combinations thereof); or (3) be partitioned into smaller portions before being processed in a plurality of parallel iterations (not necessarily conducted simultaneously) by the at least one further process(s) (or combinations thereof). For example, in certain embodiments, the single batch composition (or single batch oligonucleotide composition) refers to an oligonucleotide composition derived from at least one or a plurality of oligonucleotide synthesis column(s) having a solid support (e.g., a solid support with a linker attached thereto) with a loading capacity of a specified quantity, or at least one or a plurality of synthesis run(s) on the oligonucleotide synthesis column having the solid support with a loading capacity of a specified quantity; wherein the oligonucleotide synthesis output from the at least one, or the plurality of, oligonucleotide synthesis column(s) (or synthesis run(s)) having the solid support with a loading capacity of the specified quantity, may be processed in a single iteration or in a plurality of parallel iterations (not necessarily conducted simultaneously) by at least one further process(s), comprising: an oligonucleotide chemical transformation process (e.g., an oligonucleotide deprotection or cleavage from solid support), an oligonucleotide purification process (e.g., ion-exchange chromatography), an oligonucleotide desalting process (e.g., ultrafiltration and/or diafiltration), a liquid composition concentration process (e.g., thin film evaporation), or a drying process (e.g., freeze drying), or combinations thereof; wherein: (1) the oligonucleotide synthesis output may: (a) be pooled or combined (such that the resulting pooled/combined oligonucleotide composition has uniform character and quality, e.g., the physical and chemical properties are distributed throughout said resulting pooled/combined oligonucleotide composition): (i) before being processed in a single iteration or in a plurality of parallel iterations (not necessarily conducted simultaneously) by the at least one further process(s) (or combinations thereof); or (ii) followed by partitioning the resulting pooled/combined oligonucleotide composition into smaller portions before being processed in a plurality of parallel iterations (not necessarily conducted simultaneously) by the at least one further process(s) (or combinations thereof); or (b) be partitioned into smaller portions before being processed in a plurality of parallel iterations (not necessarily conducted simultaneously) by the at least one further process(s) (or combinations thereof); and/or (2) the output resulting from any one, multiple, or each, of the at least one further process(s) may: (a) provide the single batch composition (or single batch oligonucleotide composition) as a liquid oligonucleotide composition or a solid oligonucleotide composition; (b) be pooled or combined (such that the resulting pooled/combined oligonucleotide composition has uniform character and quality, e.g., the physical and chemical properties are distributed throughout said resulting pooled/combined oligonucleotide composition): (i) to provide the single batch composition (or single batch oligonucleotide composition) as a liquid oligonucleotide composition or a solid oligonucleotide composition; (ii) before being processed in a single iteration or in a plurality of parallel iterations (not necessarily conducted simultaneously) by a second, next, or downstream (see, e.g., FIG. 1) at least one further process(s) (or combinations thereof); or (iii) followed by partitioning the resulting pooled/combined oligonucleotide composition into smaller portions before being processed in a plurality of parallel iterations (not necessarily conducted simultaneously) by a second, next, or downstream (see, e.g., FIG. 1) at least one further process(s) (or combinations thereof); or (c) be partitioned into smaller portions before being processed in a plurality of parallel iterations (not necessarily conducted simultaneously) by a second, next, or downstream (see, e.g., FIG. 1) at least one further process(s) (or combinations thereof). For example, in certain embodiments, the pooling or combining process, or the resulting oligonucleotide composition (e.g., the single batch composition or single batch oligonucleotide composition) derived from the pooling or combining process, of the output(s) of a particular process (such as the oligonucleotide synthesis process, chemical transformation process, purification process, desalting process, liquid concentration process, and/or drying process, or combinations thereof) is compliant with the CGMP requirements under Title 21 of the United States Code of Federal Regulations, Sections 210 and 211 (“21 CFR 210” and “21 CFR 211”, respectively), which are herein incorporated by reference in their entirety. For example, in certain embodiments, the single batch composition (or single batch oligonucleotide composition) and/or the resulting oligonucleotide composition derived from the pooling or combining process, of the output(s) of a particular process (such as the oligonucleotide synthesis process, chemical transformation process, purification process, desalting process, liquid concentration process, and/or drying process, or combinations thereof) may comply with the term “batch”, as defined under 21 CFR 210.3(2) (“‘Batch’ means a specific quantity of a drug or other material that is intended to have uniform character and quality, within specified limits, and is produced according to a single manufacturing order during the same cycle of manufacture”). For example, in certain embodiments, the single batch composition (or single batch oligonucleotide composition) and/or the resulting oligonucleotide composition derived from the pooling or combining process, of the output(s) of a particular process (such as the oligonucleotide synthesis process, chemical transformation process, purification process, desalting process, liquid concentration process, and/or drying process, or combinations thereof) may comply with the term “lot”, as defined under 21 CFR 210.3(10) (“‘Lot’ means a batch, or a specific identified portion of a batch, having uniform character and quality within specified limits; or, in the case of a drug product produced by continuous process, it is a specific identified amount produced in a unit of time or quantity in a manner that assures its having uniform character and quality within specified limits”). In certain embodiments, the specified quantity may be at least 100 mmol, such as, at least 150 mmol, at least 200 mmol, at least 250 mmol, at least 300 mmol, at least 500 mmol, at least 600 mmol, at least 700 mmol, at least 800 mmol, at least 900 mmol, at least 1,000 mmol, at least 1,200 mmol, at least 1,400 mmol, at least 1,600 mmol, at least 1,800 mmol, at least 2,000 mmol, at least 2,500 mmol, at least 3,000 mmol, at least 3,500 mmol, at least 4,000 mmol, or at least 5,000 mmol, for example, the specified quantity may be in the range of between 300-5,400 mmol, such as in the range of between 300-4,500 mmol, 300-4,000 mmol, 300-3,600 mmol, 300-3,000 mmol, 600-3,000 mmol, 700-3,600 mmol, 700-2,700 mmol, 700-2,500 mmol, 700-2,400 mmol, 700-2,000 mmol, 700-1,900 mmol, 700-1,800 mmol, 700-1,700 mmol, 700-1,600 mmol, 700-1,500 mmol, 700-1,400 mmol, 700-1,300 mmol, 700-1,200 mmol, 700-1,100 mmol, 700-1,000 mmol, 700-900 mmol, 800-1,200 mmol, 800-1,000 mmol, 900-1,600 mmol, 900-1,800 mmol, 900-2,400 mmol, 900-2,700 mmol, 900-3,600 mmol, 1,800-2,700 mmol, 1,800-3,600 mmol, 1,000-3,000 mmol, 1,000-2,500 mmol, 1,000-2,000 mmol, 1,000-1,500 mmol, 1,250-1,750 mmol, 1,500-3,000 mmol, 1,500-2,500 mmol, 1,500-2,000 mmol, 1,600-2,400 mmol, 2,000-3,000 mmol, 2,500-3,000 mmol, 2,700-3,600 mmol, 2,700-4,500 mmol, 3,000-4,000 mmol, 3,600-4,500 mmol, or 3,600-5,400 mmol. In certain embodiments, the amount of oligonucleotide synthesized by the at least one, or the plurality of, oligonucleotide synthesis column(s) (or synthesis run(s)) having the solid support with a loading capacity of the specified quantity, and/or contained in the resulting oligonucleotide synthesis output from said at least one, or said plurality of, oligonucleotide synthesis column(s) (or synthesis run(s)), may be a specified quantity of the oligonucleotide. In certain embodiments, one or more of the further processes, or each of the further processes, may be performed with an oligonucleotide composition comprising a specified quantity of the oligonucleotide. In certain embodiments, the amount of oligonucleotide processed by a further process downstream from the synthesis process (e.g., an oligonucleotide chemical transformation process (e.g., an oligonucleotide deprotection or cleavage from solid support), an oligonucleotide purification process (e.g., ion-exchange chromatography), an oligonucleotide desalting process (e.g., ultrafiltration and/or diafiltration), a liquid composition concentration process (e.g., thin film evaporation), and/or a drying process (e.g., freeze drying)), may be a specified quantity of the oligonucleotide. For example, the specified quantity of the oligonucleotide processed by a further process downstream from the synthesis process may be processed in a single iteration, may be partitioned and then processed in a plurality of parallel iterations (not necessarily conducted simultaneously), or may be processed in one or more, or each, of a plurality of parallel iterations (not necessarily conducted simultaneously). In certain embodiments, one or more of the further processes, or each of the further processes, may be performed in a single iteration, such as at a scale involving a specified quantity of the oligonucleotide. In certain embodiments, one or more of the further processes, or each of the further processes, may be performed in a plurality of iterations, such as at a scale involving a specified quantity of the oligonucleotide, for example, in a plurality of a parallel iterations (not necessarily conducted simultaneously), or may be performed in a plurality of iterations conducted serially utilizing the same equipment (e.g., synthesis column, ion-exchange column), or combinations thereof. In certain embodiments, the plurality of parallel iterations (not necessarily conducted simultaneously) may be no more than 2, 3, 4, 5, 6, 7, 8, 9, 10, 15, 20, 25, 30, 35, 40, 45, 50, 55, or at most 60 parallel iterations, for example, no more than between 2-60, 2-55, 2-50, 2-45, 2-40, 2-35, 2-30, 2-25, 2-20, 2-15, 2-10, 2-9, 2-8, 2-7, 2-6, 2-5, 2-4, 2-3, 3-10, 3-8, 3-5, 4-6, 5-10, 10-60, 10-50, 10-40, 10-30, 10-25, 10-20, 10-15, 20-50, 30-60, or 40-55 parallel iterations. In certain embodiments, the oligonucleotide synthesis output or the output resulting from the further process (such as the chemically transformed oligonucleotide output, the purified oligonucleotide output, the desalted oligonucleotide output, the concentrated oligonucleotide liquid composition output, and/or the dried oligonucleotide composition output), may comprise a specified quantity of the oligonucleotide. In certain embodiments, the at least one output, the pooled or combined output, or the partitioned output, that results from the oligonucleotide synthesis process or one or more of the further processes, may be (or comprise) a specified quantity of the oligonucleotide. In certain embodiments, the further process(s) may be conducted downstream from the synthesis process in the following sequence: an oligonucleotide chemical transformation process (e.g., an oligonucleotide deprotection or cleavage from solid support), an oligonucleotide purification process (e.g., ion-exchange chromatography), an oligonucleotide desalting process (e.g., ultrafiltration and/or diafiltration), a liquid composition concentration process (e.g., thin film evaporation), and a drying process (e.g., freeze drying). In certain embodiments, the further process(s) may be conducted downstream from the synthesis process in the following sequence: an oligonucleotide chemical transformation process (e.g., an oligonucleotide deprotection or cleavage from solid support), an oligonucleotide purification process (e.g., ion-exchange chromatography), and an oligonucleotide desalting process (e.g., ultrafiltration and/or diafiltration); wherein the sequence may be followed by a liquid composition concentration process (e.g., thin film evaporation) and/or a drying process (e.g., freeze drying); or wherein the sequence may be exclusive of a liquid composition concentration process (e.g., thin film evaporation) and/or a drying process (e.g., freeze drying). In certain embodiments, the single batch composition (or single batch oligonucleotide composition) may be a liquid oligonucleotide composition (e.g., a purified, liquid oligonucleotide composition, a desalted and purified, liquid oligonucleotide composition, or a concentrated oligonucleotide liquid composition). In certain embodiments, the method utilized to prepare the single batch composition (or single batch oligonucleotide composition) is exclusive of a drying process, such as a freeze drying process. In certain embodiments, the method utilized to prepare the single batch composition (or single batch oligonucleotide composition) is exclusive of a liquid composition concentration process, such as a thin film evaporation process, and a drying process, such as a freeze drying process. In certain embodiments, the single batch composition (or single batch oligonucleotide composition) may be a solid oligonucleotide composition. In certain embodiments, the method utilized to prepare the single batch composition (or single batch oligonucleotide composition) is inclusive of a drying process, such as a freeze drying process.

The present application provides methods of preparing an oligonucleotide compound, for example, an oligonucleotide, such as a phosphorothioate-linked oligonucleotide, in a multi-step process that includes a solid phase oligonucleotide synthesis involving an iterative synthetic cycle of coupling, thiolation (or oxidation), (optionally) capping, and (optionally) deprotecting, cleavage of the crude protected oligonucleotide from the solid support, loading the crude protected oligonucleotide onto a preparative anion exchange chromatographic column, followed by deprotection and then purification by anion exchange chromatography, desalting via ultrafiltration/diafiltration, and concentration of the oligonucleotide compound solution, such as concentration via thin film evaporation, to yield the oligonucleotide compound.

More specifically, the iterative synthetic process utilizes phosphoramidite chemistry, a trityl protecting group, such as the 5′-(4,4′-dimethoxytrityl) (DMT) protecting group, and a solid support, for example, controlled pore glass or a cross-linked polystyrene solid support, such as a cross-linked polystyrene solid support with a linker attached thereto, to assemble the oligonucleotide sequence, wherein the iterative synthesis involves cycles of coupling, thiolation (or oxidation), (optionally) capping, and (optionally) 5′-deprotecting. The coupling reaction utilizing phosphoramidite chemistry includes activation of a selected deoxyribo-amidite, reaction with a free 5′-hydroxyl group of a support-immobilized protected nucleotide or oligonucleotide. After a predetermined number of iterative synthesis cycles, the protecting groups on the phosphate-linkages or phosphorothioate-linkages, such as a cyanoethyl protecting group, are removed, and then the resulting crude oligonucleotide is cleaved from the solid support by base treatment which also deprotects the base protecting groups on the crude oligonucleotide. The resulting crude oligonucleotide, which still has a 5′-hydroxyl protecting group on the terminal oligonucleoside, is loaded onto a preparative anion exchange chromatographic column and the terminal 5′-hydroxyl protecting group is deprotected with a protic acid. The deprotected oligonucleotide is purified by anion exchange chromatography, desalted via ultrafiltration/diafiltration, and then concentrated to remove the excess water, such as concentrated via thin film evaporation, to yield the oligonucleotide compound.

In certain embodiments, the method of preparing an oligonucleotide may comprise or consist of the following steps:
a) providing a linker attached to a solid support wherein the linker comprises a protected hydroxyl group;
b) deprotecting the protected hydroxyl group of the linker thereby creating a deprotected hydroxyl group;
c) independently providing a nucleoside phosphoramidite, wherein the nucleoside phosphoramidite comprises a protected hydroxyl group and a protected phosphoramidite;
d) independently coupling the nucleoside phosphoramidite to the deprotected hydroxyl group of the linker, or to the deprotected hydroxyl group of the nucleoside from the previous iteration of the reaction cycle, thereby creating a phosphite triester linked nucleoside;
e) independently thiolating the protected phosphite triester linkage thereby creating a protected phosphorothioate linkage;
f) optionally, independently capping unreacted deprotected hydroxyl groups;
g) optionally, independently deprotecting the protected hydroxyl group of the nucleoside;
h) repeating the providing, coupling, thiolating, capping, and deprotecting steps (steps c) through g)) a predetermined number of times to provide a solid support-bound oligonucleotide;
i) deprotecting the protected phosphorothioate linkages;
j) cleaving the oligonucleotide from the solid support;
k) eluting the oligonucleotide from the solid support;
l) purifying the oligonucleotide eluate using an ion exchange chromatography column; and
m) concentrating the solution of the oligonucleotide compound, such as concentrating with thin film evaporation.
   In certain embodiments, the method of preparing an oligonucleotide includes the step of independently capping the unreacted deprotected hydroxyl groups and/or the step of independently deprotecting the protected hydroxyl group of the nucleoside. In certain embodiments, the optional step of capping the unreacted deprotected hydroxyl groups and the optional step of deprotecting the protected hydroxyl group of the nucleoside are performed in each iteration of the reaction cycle, exclusive of the last iteration (i.e., not performed in the last iteration), according to the method of preparing an oligonucleotide described herein.

In certain embodiments, the method of preparing an oligonucleotide may comprise or consist of the following steps:
a) providing a linker attached to a solid support wherein the linker comprises a protected hydroxyl group;
b) deprotecting the protected hydroxyl group of the linker thereby creating a deprotected hydroxyl group;
c) independently providing a nucleoside phosphoramidite, wherein the nucleoside phosphoramidite comprises a protected hydroxyl group and a protected phosphoramidite;
d) independently coupling the nucleoside phosphoramidite to the deprotected hydroxyl group of the linker, or to the deprotected hydroxyl group of the nucleoside from the previous iteration of the reaction cycle, thereby creating a phosphite triester linked nucleoside;
e) independently thiolating the protected phosphite triester linkage thereby creating a protected phosphorothioate linkage;
f) optionally, independently capping unreacted deprotected hydroxyl groups;
g) optionally, independently deprotecting the protected hydroxyl group of the nucleoside;
h) repeating the providing, coupling, thiolating, capping, and deprotecting steps (steps c) through g)) a predetermined number of times to provide a solid support-bound oligonucleotide;
i) deprotecting the protected phosphorothioate linkages;
j) cleaving the oligonucleotide from the solid support;
k) eluting the oligonucleotide from the solid support;
l) purifying the oligonucleotide eluate using an ion exchange chromatography column;
   1) loading the oligonucleotide eluate from eluting step k) onto the ion exchange chromatography column;
   2) deprotecting the protected hydroxyl group from the terminal nucleoside; and
   3) eluting the oligonucleotide from the ion exchange chromatography column using a salt gradient; and
m) concentrating the solution of the oligonucleotide compound, such as concentrating with thin film evaporation.
   In certain embodiments, the method of preparing an oligonucleotide includes the step of independently capping the unreacted deprotected hydroxyl groups and/or the step of independently deprotecting the protected hydroxyl group of the nucleoside. In certain embodiments, the optional step of capping the unreacted deprotected hydroxyl groups and the optional step of deprotecting the protected hydroxyl group of the nucleoside are performed in each iteration of the reaction cycle, exclusive of the last iteration (i.e., not performed in the last iteration), according to the method of preparing an oligonucleotide described herein.

In certain embodiments, the method of preparing an oligonucleotide may comprise or consist of the following steps:
a) providing a linker attached to a solid support wherein the linker comprises a protected hydroxyl group;
b) deprotecting the protected hydroxyl group of the linker thereby creating a deprotected hydroxyl group;
c) independently providing a nucleoside phosphoramidite, wherein the nucleoside phosphoramidite comprises a protected hydroxyl group and a protected phosphoramidite;
d) independently coupling the nucleoside phosphoramidite to the deprotected hydroxyl group of the linker, or to the deprotected hydroxyl group of the nucleoside from the previous iteration of the reaction cycle, thereby creating a phosphite triester linked nucleoside;
e) independently thiolating the protected phosphite triester linkage thereby creating a protected phosphorothioate linkage;
f) optionally, independently capping unreacted deprotected hydroxyl groups;
g) optionally, independently deprotecting the protected hydroxyl group of the nucleoside;
h) repeating the providing, coupling, thiolating, capping, and deprotecting steps (steps c) through g)) a predetermined number of times to provide a solid support-bound oligonucleotide;
i) deprotecting the protected phosphorothioate linkages;
j) cleaving the oligonucleotide from the solid support;
k) eluting the oligonucleotide from the solid support;
l) purifying the oligonucleotide eluate using an ion exchange chromatography column;
   1) loading the oligonucleotide eluate from eluting step k) onto the ion exchange chromatography column;
   2) deprotecting the protected hydroxyl group from the terminal nucleoside;
   3) eluting the oligonucleotide from the ion exchange chromatography column using a salt gradient; and
   4) desalting the oligonucleotide eluate from the ion exchange column via ultrafiltration and/or diafiltration; and
m) concentrating the desalted solution of the oligonucleotide compound, such as concentrating with thin film evaporation.
   In certain embodiments, the method of preparing an oligonucleotide includes the step of independently capping the unreacted deprotected hydroxyl groups and/or the step of independently deprotecting the protected hydroxyl group of the nucleoside. In certain embodiments, the optional step of capping the unreacted deprotected hydroxyl groups and the optional step of deprotecting the protected hydroxyl group of the nucleoside are performed in each iteration of the reaction cycle, exclusive of the last iteration (i.e., not performed in the last iteration), according to the method of preparing an oligonucleotide described herein.

In certain embodiments, the method of preparing an oligonucleotide may comprise or consist of the following steps:
a) providing a linker attached to a solid support wherein the linker comprises a protected hydroxyl group;
b) deprotecting the protected hydroxyl group of the linker thereby creating a deprotected hydroxyl group;
c) independently providing a nucleoside phosphoramidite, wherein the nucleoside phosphoramidite comprises a protected hydroxyl group and a protected phosphoramidite;
d) independently coupling the nucleoside phosphoramidite to the deprotected hydroxyl group of the linker, or to the deprotected hydroxyl group of the nucleoside from the previous iteration of the reaction cycle, thereby creating a phosphite triester linked nucleoside;
e) independently thiolating the protected phosphite triester linkage thereby creating a protected phosphorothioate linkage, or optionally, independently oxidizing the protected phosphite triester linkage thereby creating a protected phosphate linkage;
f) optionally, independently capping unreacted deprotected hydroxyl groups;
g) optionally, independently deprotecting the protected hydroxyl group of the nucleoside;
h) repeating the providing, coupling, thiolating or optional oxidizing, capping, and deprotecting steps (steps c) through g)) a predetermined number of times to provide a solid support-bound oligonucleotide;
i) deprotecting the protected phosphorothioate linkages and optional protected phosphate linkages;
j) cleaving the oligonucleotide from the solid support;
k) eluting the oligonucleotide from the solid support;
l) purifying the oligonucleotide eluate using an ion exchange chromatography column; and
m) concentrating the solution of the oligonucleotide compound, such as concentrating with thin film evaporation.
   In certain embodiments, the method of preparing an oligonucleotide includes the step of independently capping the unreacted deprotected hydroxyl groups and/or the step of independently deprotecting the protected hydroxyl group of the nucleoside. In certain embodiments, the optional step of capping the unreacted deprotected hydroxyl groups and the optional step of deprotecting the protected hydroxyl group of the nucleoside are performed in each iteration of the reaction cycle, exclusive of the last iteration (i.e., not performed in the last iteration), according to the method of preparing an oligonucleotide described herein.

In certain embodiments, the method of preparing an oligonucleotide may comprise or consist of the following steps:
a) providing a linker attached to a solid support wherein the linker comprises a protected hydroxyl group;
b) deprotecting the protected hydroxyl group of the linker thereby creating a deprotected hydroxyl group;
c) independently providing a nucleoside phosphoramidite, wherein the nucleoside phosphoramidite comprises a protected hydroxyl group and a protected phosphoramidite;
d) independently coupling the nucleoside phosphoramidite to the deprotected hydroxyl group of the linker, or to the deprotected hydroxyl group of the nucleoside from the previous iteration of the reaction cycle, thereby creating a phosphite triester linked nucleoside;
e) independently thiolating the protected phosphite triester linkage thereby creating a protected phosphorothioate linkage, or optionally, independently oxidizing the protected phosphite triester linkage thereby creating a protected phosphate linkage;
f) optionally, independently capping unreacted deprotected hydroxyl groups;
g) optionally, independently deprotecting the protected hydroxyl group of the nucleoside;
h) repeating the providing, coupling, thiolating or optional oxidizing, capping, and deprotecting steps (steps c) through g)) a predetermined number of times to provide a solid support-bound oligonucleotide;
i) deprotecting the protected phosphorothioate linkages and optional protected phosphate linkages;
j) cleaving the oligonucleotide from the solid support;
k) eluting the oligonucleotide from the solid support;
l) purifying the oligonucleotide eluate using an ion exchange chromatography column;
   1) loading the oligonucleotide eluate from eluting step k) onto the ion exchange chromatography column;
   2) deprotecting the protected hydroxyl group from the terminal nucleoside; and
   3) eluting the oligonucleotide from the ion exchange chromatography column using a salt gradient; and
m) concentrating the solution of the oligonucleotide compound, such as concentrating with thin film evaporation.
   In certain embodiments, the method of preparing an oligonucleotide includes the step of independently capping the unreacted deprotected hydroxyl groups and/or the step of independently deprotecting the protected hydroxyl group of the nucleoside. In certain embodiments, the optional step of capping the unreacted deprotected hydroxyl groups and the optional step of deprotecting the protected hydroxyl group of the nucleoside are performed in each iteration of the reaction cycle, exclusive of the last iteration (i.e., not performed in the last iteration), according to the method of preparing an oligonucleotide described herein.

In certain embodiments, the method of preparing an oligonucleotide may comprise or consist of the following steps:
a) providing a linker attached to a solid support wherein the linker comprises a protected hydroxyl group;
b) deprotecting the protected hydroxyl group of the linker thereby creating a deprotected hydroxyl group;
c) independently providing a nucleoside phosphoramidite, wherein the nucleoside phosphoramidite comprises a protected hydroxyl group and a protected phosphoramidite;
d) independently coupling the nucleoside phosphoramidite to the deprotected hydroxyl group of the linker, or to the deprotected hydroxyl group of the nucleoside from the previous iteration of the reaction cycle, thereby creating a phosphite triester linked nucleoside;
e) independently thiolating the protected phosphite triester linkage thereby creating a protected phosphorothioate linkage, or optionally, independently oxidizing the protected phosphite triester linkage thereby creating a protected phosphate linkage;
f) optionally, independently capping unreacted deprotected hydroxyl groups;
g) optionally, independently deprotecting the protected hydroxyl group of the nucleoside;
h) repeating the providing, coupling, thiolating or optional oxidizing, capping, and deprotecting steps (steps c) through g)) a predetermined number of times to provide a solid support-bound oligonucleotide;
i) deprotecting the protected phosphorothioate linkages and optional protected phosphate linkages;
j) cleaving the oligonucleotide from the solid support;
k) eluting the oligonucleotide from the solid support;
l) purifying the oligonucleotide eluate using an ion exchange chromatography column;
   1) loading the oligonucleotide eluate from eluting step k) onto the ion exchange chromatography column;
   2) deprotecting the protected hydroxyl group from the terminal nucleoside;
   3) eluting the oligonucleotide from the ion exchange chromatography column using a salt gradient; and
   4) desalting the oligonucleotide eluate from the ion exchange column via ultrafiltration and/or diafiltration; and
m) concentrating the desalted solution of the oligonucleotide compound, such as concentrating with thin film evaporation.
   In certain embodiments, the method of preparing an oligonucleotide includes the step of independently capping the unreacted deprotected hydroxyl groups and/or the step of independently deprotecting the protected hydroxyl group of the nucleoside. In certain embodiments, the optional step of capping the unreacted deprotected hydroxyl groups and the optional step of deprotecting the protected hydroxyl group of the nucleoside are performed in each iteration of the reaction cycle, exclusive of the last iteration (i.e., not performed in the last iteration), according to the method of preparing an oligonucleotide described herein.

In certain embodiments, the method of preparing an oligonucleotide may comprise or consist of the following steps: a) providing a linker attached to a solid support wherein the linker comprises a protected hydroxyl group; b) independently deprotecting the protected hydroxyl group of the linker thereby creating a deprotected hydroxyl group; c) independently coupling a nucleoside phosphoramidite to the deprotected hydroxyl group of the linker, or to the deprotected hydroxyl group of the nucleoside from the previous iteration of the reaction cycle, thereby creating a phosphite triester linked nucleoside; d) independently thiolating the protected phosphite triester linkage thereby creating a protected phosphorothioate linkage (or optionally, independently oxidizing the protected phosphite triester linkage thereby creating a protected phosphate linkage); e) optionally, independently capping unreacted deprotected hydroxyl groups; f) optionally, independently deprotecting the protected hydroxyl group of the nucleoside; g) repeating the coupling, thiolating (or optional oxidizing), capping, and deprotecting steps (steps c) through f)) a predetermined number of times to provide a solid support-bound oligonucleotide; h) deprotecting the protected phosphorothioate linkages; i) cleaving the oligonucleotide from the solid support; j) eluting the oligonucleotide from the solid support; k) purifying the oligonucleotide eluate using an ion exchange chromatography column; and l) concentrating the solution of the oligonucleotide compound from the ion exchange column, such as concentrating with thin film evaporation. In certain embodiments, the method of preparing an oligonucleotide includes the step of independently capping the unreacted deprotected hydroxyl groups and/or the step of independently deprotecting the protected hydroxyl group of the nucleoside. In certain embodiments, the optional step of capping the unreacted deprotected hydroxyl groups and the optional step of deprotecting the protected hydroxyl group of the nucleoside are performed in each iteration of the reaction cycle, exclusive of the last iteration (i.e., not performed in the last iteration), according to the method of preparing an oligonucleotide described herein.

In certain embodiments, the method of preparing an oligonucleotide may comprise or consist of the following steps: a) providing a linker attached to a solid support wherein the linker comprises a protected hydroxyl group; b) deprotecting the protected hydroxyl group of the linker thereby creating a deprotected hydroxyl group; c) independently coupling a nucleoside phosphoramidite to the deprotected hydroxyl group of the linker, or to the deprotected hydroxyl group of the nucleoside from the previous iteration of the reaction cycle, thereby creating a phosphite triester linked nucleoside; d) independently thiolating the protected phosphite triester linkage thereby creating a protected phosphorothioate linkage (or optionally, independently oxidizing the protected phosphite triester linkage thereby creating a protected phosphate linkage); e) optionally, independently capping unreacted deprotected hydroxyl groups; f) optionally, independently deprotecting the protected hydroxyl group of the nucleoside; g) repeating the coupling, thiolating (or optional oxidizing), capping, and deprotecting steps (steps c) through f)) a predetermined number of times to provide a solid support-bound oligonucleotide; h) deprotecting the protected phosphorothioate linkages (and optional protected phosphate linkages); i) cleaving the oligonucleotide from the solid support; j) eluting the oligonucleotide from the solid support; k) purifying the oligonucleotide eluate using an ion exchange chromatography column; 1) loading the oligonucleotide eluate from eluting step j) onto the ion exchange chromatography column; 2) deprotecting the protected hydroxyl group from the terminal nucleoside; and 3) eluting the oligonucleotide from the ion exchange chromatography column using a salt gradient; and l) concentrating the solution of the oligonucleotide compound from the ion exchange column, such as concentrating with thin film evaporation. In certain embodiments, the purifying step k) of the method of preparing an oligonucleotide may comprise 1) loading the oligonucleotide eluate from eluting step j) onto the ion exchange chromatography column; 2) deprotecting the protected hydroxyl group from the terminal nucleoside; 3) eluting the oligonucleotide from the ion exchange chromatography column using a salt gradient; and 4) desalting the oligonucleotide eluate from the ion exchange column via ultrafiltration and/or diafiltration. In certain embodiments, the method of preparing an oligonucleotide includes the step of independently capping the unreacted deprotected hydroxyl groups and/or the step of independently deprotecting the protected hydroxyl group of the nucleoside. In certain embodiments, the optional step of capping the unreacted deprotected hydroxyl groups and the optional step of deprotecting the protected hydroxyl group of the nucleoside are performed in each iteration of the reaction cycle, exclusive of the last iteration (i.e., not performed in the last iteration), according to the method of preparing an oligonucleotide described herein.

In certain embodiments, the method of preparing an oligonucleotide may comprise or consist of the following steps: a) deprotecting a protected hydroxyl group of a linker attached to a solid support thereby creating a deprotected hydroxyl group; b) independently coupling a nucleoside phosphoramidite to the deprotected hydroxyl group of the linker, or to the deprotected hydroxyl group of the nucleoside from the previous iteration of the reaction cycle, thereby creating a phosphite triester linked nucleoside; c) independently thiolating the protected phosphite triester linkage thereby creating a protected phosphorothioate linkage (or optionally, independently oxidizing the protected phosphite triester linkage thereby creating a protected phosphate linkage); d) optionally, independently capping unreacted deprotected hydroxyl groups; e) optionally, independently deprotecting the protected hydroxyl group of the nucleoside; f) repeating the, coupling, thiolating (or optional oxidizing), capping, and deprotecting steps (steps b) through e)) a predetermined number of times to provide a solid support-bound oligonucleotide; g) deprotecting the protected phosphorothioate linkages (and optional protected phosphate linkages); h) cleaving the oligonucleotide from the solid support; i) eluting the oligonucleotide from the solid support; j) purifying the oligonucleotide eluate using an ion exchange chromatography column; and k) concentrating the solution of the oligonucleotide compound from the ion exchange column, such as concentrating with thin film evaporation. In certain embodiments, the method of preparing an oligonucleotide includes the step of independently capping the unreacted deprotected hydroxyl groups and/or the step of independently deprotecting the protected hydroxyl group of the nucleoside. In certain embodiments, the optional step of capping the unreacted deprotected hydroxyl groups and the optional step of deprotecting the protected hydroxyl group of the nucleoside are performed in each iteration of the reaction cycle, exclusive of the last iteration (i.e., not performed in the last iteration), according to the method of preparing an oligonucleotide described herein.

In certain embodiments, the method of preparing an oligonucleotide may comprise or consist of the following steps: a) deprotecting a protected hydroxyl group of a linker attached to a solid support thereby creating a deprotected hydroxyl group; b) independently coupling a nucleoside phosphoramidite to the deprotected hydroxyl group of the linker, or to the deprotected hydroxyl group of the nucleoside from the previous iteration of the reaction cycle, thereby creating a phosphite triester linked nucleoside; c) independently thiolating the protected phosphite triester linkage thereby creating a protected phosphorothioate linkage (or optionally, independently oxidizing the protected phosphite triester linkage thereby creating a protected phosphate linkage); d) optionally, independently capping unreacted deprotected hydroxyl groups; e) optionally, independently deprotecting the protected hydroxyl group of the nucleoside; f) repeating the coupling, thiolating (or optional oxidizing), capping, and deprotecting steps (steps b) through e)) a predetermined number of times to provide a solid support-bound oligonucleotide; g) deprotecting the protected phosphorothioate linkages (and optional protected phosphate linkages); h) cleaving the oligonucleotide from the solid support; i) eluting the oligonucleotide from the solid support; j) purifying the oligonucleotide eluate using an ion exchange chromatography column; and 1) loading the oligonucleotide eluate from eluting step i) onto the ion exchange chromatography column; 2) deprotecting the protected hydroxyl group from the terminal nucleoside; and 3) eluting the oligonucleotide from the ion exchange chromatography column using a salt gradient; and k) concentrating the solution of the oligonucleotide compound from the ion exchange column, such as concentrating with thin film evaporation. In certain embodiments, the purifying step j) of the method of preparing an oligonucleotide may comprise 1) loading the oligonucleotide eluate from eluting step i) onto the ion exchange chromatography column; 2) deprotecting the protected hydroxyl group from the terminal nucleoside; 3) eluting the oligonucleotide from the ion exchange chromatography column using a salt gradient; and 4) desalting the oligonucleotide eluate from the ion exchange column via ultrafiltration and/or diafiltration. In certain embodiments, the method of preparing an oligonucleotide includes the step of independently capping the unreacted deprotected hydroxyl groups and/or the step of independently deprotecting the protected hydroxyl group of the nucleoside. In certain embodiments, the optional step of capping the unreacted deprotected hydroxyl groups and the optional step of deprotecting the protected hydroxyl group of the nucleoside are performed in each iteration of the reaction cycle, exclusive of the last iteration (i.e., not performed in the last iteration), according to the method of preparing an oligonucleotide described herein.

In certain embodiments, the method of preparing an oligonucleotide may further comprise a preswelling step between the capping step f) and the deprotecting step g) in at least one of said iterations. In certain embodiments, the method of preparing an oligonucleotide may further comprise optionally washing the support with an aprotic solvent, such as acetonitrile, between one or more of the steps in at least one of said iterations.

5.1 SMAD7 Antisense Oligonucleotides

In certain embodiments, the methods described herein are used to synthesize a SMAD7 antisense oligonucleotide (sometimes referred to as an anti-SMAD7 ODN or SMAD7 AON). In certain embodiments, the anti-SMAD7 ODN is a chemically modified SMAD7 ODN.

Antisense oligonucleotides are short synthetic oligonucleotide sequences complementary to the messenger RNA (mRNA), which encodes for the target protein (e.g., SMAD7). Without being bound by theory, antisense oligonucleotide sequences can hybridize to a complementary region in an mRNA molecule thereby producing a double-strand hybrid that can lead to the activation of ubiquitous catalytic enzymes, such as RNaseH, which degrades DNA/RNA hybrid strands, thus preventing protein translation. Without being bound by theory, an antisense oligonucleotide provided herein can hybridize to its target sequence as RNA or DNA. Thus, even if a DNA sequence is provided as target, the corresponding RNA sequence (including uracil instead of thymine) is included.

For example, anti-SMAD7 ODNs, when introduced into or taken up by a cell, can reduce SMAD7 expression in the cell by reducing the level of a SMAD7 mRNA in the cell or by reducing the level of SMAD7 protein in the cell. Specifically, an anti-SMAD7 ODN can reduce SMAD7 expression in vitro, e.g., in a cultured cell, or in vivo, e.g., in a subject (such as a human patient or in an animal model organism).

In certain embodiments, an anti-SMAD7 oligonucleotide can be chemically modified. In certain embodiments, the oligonucleotide, such as an anti-SMAD7 oligonucleotide or a chemically modified anti-SMAD7 oligonucleotide, may be in an anionic form, such as in an anionic form with a sodium counterion (“Na⁺”), or may be protontated to form an acidic form. In certain embodiments, the oligonucleotide, such as an anti-SMAD7 oligonucleotide or a chemically modified anti-SMAD7 oligonucleotide, may comprise a phosphorothioate backbone, which can be fully or partially protonated to form an acidic form.

In certain embodiments, the oligonucleotide, such as an anti-SMAD7 oligonucleotide or a chemically modified anti-SMAD7 oligonucleotide, may comprise at least one internucleoside linkage, which is a phosphate linkage, e.g., a monophosphate linkage. In certain embodiments, the oligonucleotide, such as an anti-SMAD7 oligonucleotide or a chemically modified anti-SMAD7 oligonucleotide, may comprise at least one internucleoside linkage, which is a phosphorothioate linkage. In certain embodiments, the oligonucleotide, such as an anti-SMAD7 oligonucleotide or a chemically modified anti-SMAD7 oligonucleotide, may comprise at least 2, 3, 4, 5, 6, 7, 8, 9, 10, 11, 12, 13, 14, 15, 16, 17, 18, 19, 20, 21, 22, 23, 24, 25, or more phosphorothioate linkages. In certain embodiments, at least 5%, 10%, 20%, 25%, 30%, 35%, 40%, 45%, 50%, 55%, 60%, 65%, 70%, 75%, 80%, 85%, 90%, 95%, or 100% of internucleoside linkages in the oligonucleotide, such as an anti-SMAD7 oligonucleotide or a chemically modified anti-SMAD7 oligonucleotide, are phosphorothioate linkages. In certain embodiments, all internucleoside linkages are phosphorothioate linkages.

5.2 Oligonucleotide Modifications

In certain embodiments an oligonucleotide (e.g., an anti-SMAD7 ODN) synthesized by the methods described herein is chemically modified. In certain specific embodiments, a SMAD7 ODN as described herein can have a sequence that is complementary to the nucleotide sequence of SMAD7 mRNA (i.e., the SMAD7 ODN can be an antisense oligonucleotide).

The oligonucleotides, such as an anti-SMAD7 oligonucleotides or a chemically modified anti-SMAD7 oligonucleotides, prepared according to the synthesis methods as described herein, can include, e.g., non-naturally occurring nucleobases, modified internucleoside (backbone) linkages, sugar modifications or modified 5′- or 3′-ends. In certain embodiments, the oligonucleotide, such as an anti-SMAD7 oligonucleotide or a chemically modified anti-SMAD7 oligonucleotide, may comprise a non-naturally-occurring sequence tag.

In certain embodiments, the oligonucleotides, anti-SMAD7 oligonucleotides, or chemically modified anti-SMAD7 oligonucleotides, prepared according to the synthesis methods as described herein, can include naturally occurring nucleobases, sugars, and covalent internucleoside (backbone) linkages, as well as non-naturally occurring portions. For example, the oligonucleotide, such as an anti-SMAD7 oligonucleotide or a chemically modified anti-SMAD7 oligonucleotide, may include a mixed-backbone, e.g., including one or more phosphorothioate linkages.

In certain embodiments, the oligonucleotides, anti-SMAD7 oligonucleotides, or chemically modified anti-SMAD7 oligonucleotides, prepared according to the synthesis methods as described herein, can also include nucleobase modifications or substitutions. As used herein, “unmodified” or “natural” nucleobases include the purine bases adenine (A) and guanine (G), and the pyrimidine bases thymine (T), cytosine (C) and uracil (U). The chemically modified SMAD7 ODNs can include, e.g., synthetic and natural nucleobases such as 5-methylcytosine (5-Me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine, or 3-deazaguanine and 3-deazaadenine. The oligonucleotide, such as an anti-SMAD7 oligonucleotide or a chemically modified anti-SMAD7 oligonucleotide, can further include nucleobases such as those disclosed in U.S. Pat. No. 3,687,808, those disclosed in “The Concise Encyclopedia of Polymer Science And Engineering,” pages 858-859, Kroschwitz, J. I., ed. John Wiley & Sons, 1990, those disclosed by Englisch et al., Angewandte Chemie, International Edition, 1991, 30, 613, or those disclosed by Sanghvi, Y. S., Chapter 15, Antisense Research and Applications, pages 289-302, Crooke, S. T. and Lebleu, B., ed., CRC Press, 1993, each of which are herein incorporated by reference in their entirety. In certain embodiments, the oligonucleotide can include nucleobases that can increase the binding affinity of the chemically modified SMAD7 ODN. Such nucleobases can include, e.g., 5-substituted pyrimidines, 6-azapyrimidines and N-2, N-6 and 0-6 substituted purines, including 2-aminopropyladenine, 5-propynyluracil and 5-propynylcytosine 5-methylcytosine substitutions. In certain embodiments, the oligonucleotide can include one or more of the above-mentioned modified nucleobases in combination with 2′-O-methoxyethyl sugar modifications.

In certain embodiments, the oligonucleotide, such as an anti-SMAD7 oligonucleotide or a chemically modified anti-SMAD7 oligonucleotide, may have one or more cytosine residues replaced by 5-methylcytosine. In certain embodiments the one or more cytosine residues form part of a CpG pair.

In certain embodiments, the oligonucleotide, such as an anti-SMAD7 oligonucleotide or a chemically modified anti-SMAD7 oligonucleotide, may include an artificial nucleoside, such as deoxycytidine and/or 5-methyl 2′-deoxycytidine, or may include an artificial nucleotide, such as 5-methyl-2′-deoxycytidine 5′-monophosphate and/or 5-methyl-2′-deoxycytidine 5′-monophosphorothioate. In certain embodiments, the oligonucleotide, such as an anti-SMAD7 oligonucleotide or a chemically modified anti-SMAD7 oligonucleotide, may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 artificial nucleosides. In certain embodiments, the oligonucleotide, such as an anti-SMAD7 oligonucleotide or a chemically modified anti-SMAD7 oligonucleotide, may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 artificial nucleotides.

In certain embodiments, the oligonucleotide, such as an anti-SMAD7 oligonucleotide or a chemically modified anti-SMAD7 oligonucleotide, comprises a CG dinucleotide sequence. In certain embodiments, the oligonucleotide, such as an anti-SMAD7 oligonucleotide or a chemically modified anti-SMAD7 oligonucleotide, comprises a GC dinucleotide sequence. In certain embodiments, the CG or the GC dinucleotide sequence is a plurality of CG dinucleotide sequences and/or a plurality of GC dinucleotide sequences. In certain embodiments, the plurality of CG or GC dinucleotide sequences is 2 or more, 3 or more, 4 or more, 5 or more, or 6 or more CG or GC dinucleotide sequences. In certain embodiments, the plurality of CG or GC dinucleotide sequences comprises one or more CG dinucleotide sequences and one or more GC dinucleotide sequences. In certain embodiments, the plurality of CG or GC dinucleotide sequences comprises only CG dinucleotide sequences or only GC dinucleotide sequences.

In certain embodiments, the oligonucleotide, such as an anti-SMAD7 oligonucleotide or a chemically modified anti-SMAD7 oligonucleotide, comprises at least one CG or GC dinucleotide sequence comprising a methylated base (e.g., 5-methyl-cytosine, 6-O-methyl-guanine, or 7-methyl-guanine). In certain embodiments, the cytosine in a CG or GC dinucleotide sequence is methylated (e.g., 5-methyl-cytosine). In certain embodiments, the guanine in the CG or GC dinucleotide sequence is methylated (e.g., 6-O-methyl-guanine, or 7-methyl-guanine). In certain embodiments, the cytosine and the guanine in the CG or GC dinucleotide sequence is methylated. In certain embodiments, the oligonucleotide, such as an anti-SMAD7 oligonucleotide or a chemically modified anti-SMAD7 oligonucleotide, comprises a plurality of CG or GC dinucleotide sequences comprising a methylated base (e.g., 5-methyl-cytosine, 6-O-methyl-guanine, or 7-methyl-guanine). In certain embodiments, the plurality of CG or GC dinucleotide sequences comprising a methylated base (e.g., 5-methyl-cytosine, 6-O-methyl-guanine, or 7-methyl-guanine) is 2 or more, 3 or more, 4 or more, 5 or more, or 6 or more CG or GC dinucleotide sequences.

In certain embodiments, the CG or GC dinucleotide sequence in the oligonucleotide, such as an anti-SMAD7 oligonucleotide or a chemically modified anti-SMAD7 oligonucleotide, is a CG or GC phosphate dinucleotide sequence. In certain embodiments, one or more CG or GC dinucleotide sequences in the oligonucleotide comprise a non-natural internucleoside linkage (e.g., a phosphorothioate linkage). In certain embodiments, the CG or GC dinucleotide is a CG or GC phosphorothioate dinucleotide sequence. In certain embodiments, a two or more CG or GC dinucleotide sequences in the oligonucleotide are phosphorothioate dinucleotide sequences. In certain embodiments, all CG or GC dinucleotide sequences in the oligonucleotide are phosphorothioate dinucleotide sequences. In certain embodiments, one or more of the CG or GC phosphorothioate dinucleotide sequences in the oligonucleotide comprise one or two methylated bases (e.g., 5-methyl-cytosine, 6-O-methyl-guanine, or 7-methyl-guanine). In certain embodiments, one or more CG or GC dinucleotide sequences in the oligonucleotide comprising a methylated base are phosphorothioate dinucleotide sequences. In certain embodiments, all CG or GC dinucleotide sequences in the oligonucleotide comprising a methylated base are phosphorothioate dinucleotide sequences.

In certain embodiments, the anti-SMAD7 ODN is a chemically modified anti-SMAD7 ODN. In certain embodiments, the chemically modified anti-SMAD7 ODN comprises, e.g., a non-naturally occurring internucleoside linkage, a non-naturally occurring sugar residue, a non-naturally occurring base, a label (e.g., a fluorescence label or isotope label, such as a deuterium or tritium label), or another modification.

In certain embodiments, the oligonucleotide prepared according to the synthesis methods as described herein, can comprise a nucleotide sequence complementary to a region of a nucleotide sequence from any mammalian organism, for example, and without limitation, a primate (e.g., human, monkey, chimpanzee, orangutan, or gorilla), a cat, a dog, a rabbit, a farm animal (e.g., cow, horse, goat, sheep, pig), or a rodent (e.g., mouse, rat, hamster, or guinea pig). For example, in certain embodiments, the anti-SMAD7 ODN prepared according to the synthesis methods as described herein, can comprise a nucleotide sequence complementary to a region of a SMAD7 nucleotide sequence from any mammalian organism, for example, and without limitation, a primate (e.g., human, monkey, chimpanzee, orangutan, or gorilla), a cat, a dog, a rabbit, a farm animal (e.g., cow, horse, goat, sheep, pig), or a rodent (e.g., mouse, rat, hamster, or guinea pig).

In certain embodiments, the anti-SMAD7 ODN comprises a nucleotide sequence complementary to a region in human SMAD7. In certain embodiments, the anti-SMAD7 ODN comprises a nucleotide sequence complementary to a region of 8 or more, 10 or more, 12 or more, 14 or more, 16 or more, 18 or more, or 20 or more nucleotides of human SMAD7. In certain embodiments, the anti-SMAD7 ODN comprises a nucleotide sequence complementary to a human SMAD7 sequence comprising the nucleotide sequence of SEQ ID NO: 1, or the corresponding RNA sequence.

SEQ ID NO: 1 (Coding Sequence: CDS (288-1568) of NM 005904.3; Homo sapiens SMAD family member 7 (SMAD7), transcript variant 1, mRNA) (region 108-128 underlined):

```
              ATG TTCAGGACCA AACGATCTGC GCTCGTCCGG
CGTCTCTGGA GGAGCCGTGC GCCCGGCGGC GAGGACGAGG
AGGAGGGCGC AGGGGGAGGT GGAGGAGGAG GCGA
  GGACA GCCGAGCGCA TGGGGCCGGT
GGCGGCGGCC CGGGCAGGGC TGGATGCTGC CTGGGCAAGG
CGGTGCGAGG TGCCAAAGGT CACCACCATC CCCACCCGCC
AGCCGCGGGC GCCGGCGCGG CCGGGGGCGC CGAGGCGGAT
CTGAAGGCGC TCACGCACTC GGTGCTCAAG AAACTGAAGG
AGCGGCAGCT GGAGCTGCTG CTCCAGGCCG TGGAGTCCCG
CGGCGGGACG CGCACCGCGT GCCTCCTGCT GCCCGGCCGC
CTGGACTGCA GGCTGGGCCC GGGGGCGCCC GCCGGCGCGC
AGCCTGCGCA GCCGCCCTCG TCCTACTCGC TCCCCCTCCT
GCTGTGCAAA GTGTTCAGGT GGCCGGATCT CAGGCATTCC
TCGGAAGTCA AGAGGCTGTG TTGCTGTGAA TCTTACGGGA
AGATCAACCC CGAGCTGGTG TGCTGCAACC CCCATCACCT
TAGCCGACTC TGCGAACTAG AGTCTCCCCC CCCTCCTTAC
TCCAGATACC CGATGGATTT TCTCAAACCA ACTGCAGACT
GTCCAGATGC TGTGCCTTCC TCCGCTGAAA CAGGGGGAAC
GAATTATCTG GCCCCTGGGG GGCTTTCAGA TTCCCAACTT
CTTCTGGAGC CTGGGGATCG GTCACACTGG TGCGTGGTGG
CATACTGGGA GGAGAAGACG AGAGTGGGGA GGCTCTACTG
TGTCCAGGAG CCCTCTCTGG ATATCTTCTA TGATCTACCT
CAGGGGAATG GCTTTTGCCT CGGACAGCTC AATTCGGACA
ACAAGAGTCA GCTGGTGCAG AAGGTGCGGA GCAAAATCGG
CTGCGGCATC CAGCTGACGC GGGAGGTGGA TGGTGTGTGG
GTGTACAACC GCAGCAGTTA CCCCATCTTC ATCAAGTCCG
CCACACTGGA CAACCCGGAC TCCAGGACGC TGTTGGTACA
CAAGGTGTTC CCCGGTTTCT CCATCAAGGC TTTCGACTAC
GAGAAGGCGT ACAGCCTGCA GCGGCCCAAT GACCACGAGT
TTATGCAGCA GCCGTGGACG GGCTTTACCG TGCAGATCAG
CTTTGTGAAG GGCTGGGGCC AGTGCTACAC CCGCCAGTTC
ATCAGCAGCT GCCCGTGCTG GCTAGAGGTC ATCTTCAACA
GCCGGTAG
```

In certain embodiments, the oligonucleotide, such as an anti-SMAD7 oligonucleotide or a chemically modified anti-SMAD7 oligonucleotide, that can be synthesized using the methods as described herein, comprises a nucleotide sequence complementary to a portion or a region of a human SMAD7 sequence of SEQ ID NO: 1, or the corresponding RNA sequence.

In certain embodiments, the methods provided herein can be used to synthesize an anti-SMAD7 oligonucleotide that hybridizes to a polymorphism containing region of SMAD7 mRNA. See, e.g., International Application No. PCT/EP2015/074066, which is herein incorporated by reference in its entirety.

In certain embodiments, the oligonucleotide, such as an anti-SMAD7 oligonucleotide or a chemically modified anti-SMAD7 oligonucleotide, that can be synthesized using the methods as described herein, comprises a nucleotide sequence complementary to region 108-128 of the human SMAD7 nucleotide sequence of SEQ ID NO: 1, or the corresponding RNA sequence.

In certain embodiments, the oligonucleotide, such as an anti-SMAD7 oligonucleotide or a chemically modified anti-SMAD7 oligonucleotide, that can be synthesized using the methods as described herein, comprises a nucleotide sequence complementary to a region comprising nucleotide 403, 233, 294, 295, 296, 298, 299 or 533 of the human SMAD7 nucleotide sequence of SEQ ID NO: 1, or the corresponding RNA sequence.

In certain embodiments, the oligonucleotide, such as an anti-SMAD7 oligonucleotide or a chemically modified anti-SMAD7 oligonucleotide, that can be synthesized using the methods as described herein, comprises a portion of at least 10 nucleotides (e.g., 11 or more, 12 or more, 13 or more, 14 or more, 15, or more, 16 or more, 17 or more, 18 or more, 19 or more, or 20 or more nucleotides) of, consists of, or includes up to 21 nucleotides in length of, the following nucleic acid sequence (SEQ ID NO: 2):

5′-GTXYCCCCTTCTCCCXYCAGC-3′

wherein:
X independently represents a nucleotide comprising a nitrogenous base selected from the group consisting of cytosine, 5-methylcytosine and 2′-O-methylcytosine, and
Y independently represents a nucleotide comprising a nitrogenous base selected from the group consisting of guanine, 5-methylguanine, or 2′-O-methylguanine
provided at least one of the nucleotides X or Y comprises a methylated nitrogenous base;
   or the complementary sequence thereto.

In certain embodiments, the oligonucleotide, such as an anti-SMAD7 oligonucleotide or a chemically modified anti-SMAD7 oligonucleotide, having a sequence comprising or consisting of SEQ ID NO: 2, at least one of the internucleoside linkages is a phosphorothioate linkage (i.e., an O,O-linked phosphorothioate). In certain embodiments, the oligonucleotide, such as an anti-SMAD7 oligonucleotide or a chemically modified anti-SMAD7 oligonucleotide, having a sequence comprising or consisting of SEQ ID NO: 2, at least 2, 3, 4, 5, 6, 7, 8, 9, or 10, of the internucleoside linkages are O,O-linked phosphorothioates. In certain embodiments, the oligonucleotide, such as an anti-SMAD7 oligonucleotide or a chemically modified anti-SMAD7 oligonucleotide, having a sequence comprising or consisting of SEQ ID NO: 2, all of the internucleoside linkages are O,O-linked phosphorothioates.

In certain embodiments, the oligonucleotide, such as an anti-SMAD7 oligonucleotide or a chemically modified anti-SMAD7 oligonucleotide, has a sequence as described in U.S. Pat. No. 9,279,126, which is herein incorporated by reference in its entirety.

In certain embodiments, the oligonucleotide, such as an anti-SMAD7 oligonucleotide or a chemically modified anti-SMAD7 oligonucleotide, comprises or consists of the following sequence (SEQ ID NO: 3):

5′-GTXGCCCCTTCTCCCXGCAGC-3′

or the complementary sequence thereto;
wherein X represents 5-methyl-2′-deoxycytidine (“5-Me-dC”), and wherein at least one internucleoside linkage, for example, at least 2, 3, 4, 5, 6, 7, 8, 9, 10, or all of the internucleoside linkage are O,O-linked phosphorothioate linkages. For example, in certain embodiments, the oligonucleotide comprises or consists of a SEQ ID NO: 3, or the complementary sequence thereto; wherein X represents 5-methyl-2′-deoxycytidine (“5-Me-dC”), and wherein each of the 20 internucleotide linkages are O,O-linked phosphorothioate linkages.

In certain embodiments, the oligonucleotide, such as an anti-SMAD7 oligonucleotide or a chemically modified anti-SMAD7 oligonucleotide, comprises or consists of the following sequence (SEQ ID NO: 4):

5′-GTCGCCCCTTCTCCCCGCAG-3′

or the complementary sequence thereto;
wherein at least one internucleoside linkage, for example, at least 2, 3, 4, 5, 6, 7, 8, 9, 10, or all of the internucleoside linkage are O,O-linked phosphorothioate linkages.

In certain embodiments, the oligonucleotide, such as an anti-SMAD7 oligonucleotide or a chemically modified anti-SMAD7 oligonucleotide, comprises or consists of the following sequence (SEQ ID NO: 5):

5′-GTXGCCCCTTCTCCCXGCAG-3′

or the complementary sequence thereto;
wherein X represents 5-methyl-2′-deoxycytidine (“5-Me-dC”); and wherein at least one internucleoside linkage, for example, at least 2, 3, 4, 5, 6, 7, 8, 9, 10, or all of the internucleoside linkage are O,O-linked phosphorothioate linkages.

In certain embodiments, the oligonucleotide, such as an anti-SMAD7 oligonucleotide or a chemically modified anti-SMAD7 oligonucleotide, comprises or consists of the following sequence (SEQ ID NO: 6):

5′-GTTTGGTCCTGAACATGC-3′

or the complementary sequence thereto;
wherein at least one internucleoside linkage, for example, at least 2, 3, 4, 5, 6, 7, 8, 9, 10, or all of the internucleoside linkage are O,O-linked phosphorothioate linkages.

In certain embodiments, the oligonucleotide, such as an anti-SMAD7 oligonucleotide or a chemically modified anti-SMAD7 oligonucleotide, comprises or consists of the following sequence (SEQ ID NO: 7):

5′-GTTTGGTCCTGAACAT-3′

or the complementary sequence thereto;
wherein at least one internucleoside linkage, for example, at least 2, 3, 4, 5, 6, 7, 8, 9, 10, or all of the internucleoside linkage are O,O-linked phosphorothioate linkages.

In certain embodiments, the oligonucleotide, such as an anti-SMAD7 oligonucleotide or a chemically modified anti-SMAD7 oligonucleotide, comprises or consists of the following sequence (SEQ ID NO: 8):

5′-GTTTGGTCCTGAACATG-3′

or the complementary sequence thereto;
wherein at least one internucleoside linkage, for example, at least 2, 3, 4, 5, 6, 7, 8, 9, 10, or all of the internucleoside linkage are O,O-linked phosphorothioate linkages.

In certain embodiments, the oligonucleotide, such as an anti-SMAD7 oligonucleotide or a chemically modified anti-SMAD7 oligonucleotide, comprises or consists of the following sequence (SEQ ID NO: 9):

5′-AGCACCGAGTGCGTGAGC-3′

or the complementary sequence thereto;
wherein at least one internucleoside linkage, for example, at least 2, 3, 4, 5, 6, 7, 8, 9, 10, or all of the internucleoside linkage are O,O-linked phosphorothioate linkages.

In certain embodiments, the oligonucleotide, such as an anti-SMAD7 oligonucleotide or a chemically modified anti-SMAD7 oligonucleotide, comprises or consists of the following sequence (SEQ ID NO: 10):

5′-CGAACATGACCTCCGCAC-3′

or the complementary sequence thereto;
wherein at least one internucleoside linkage, for example, at least 2, 3, 4, 5, 6, 7, 8, 9, 10, or all of the internucleoside linkage are O,O-linked phosphorothioate linkages.

In certain embodiments, the oligonucleotide, such as an anti-SMAD7 oligonucleotide or a chemically modified anti-SMAD7 oligonucleotide, comprises or consists of the following sequence (SEQ ID NO: 11):

5′-GATCGTTTGGTCCTGAA-3′

or the complementary sequence thereto;
wherein at least one internucleoside linkage, for example, at least 2, 3, 4, 5, 6, 7, 8, 9, 10, or all of the internucleoside linkage are O,O-linked phosphorothioate linkages.

In certain embodiments, the oligonucleotide, such as an anti-SMAD7 oligonucleotide or a chemically modified anti-SMAD7 oligonucleotide, comprises or consists of the following sequence (SEQ ID NO: 12):

5′-ATCGTTTGGTCCTGAAC-3′

or the complementary sequence thereto;
wherein at least one internucleoside linkage, for example, at least 2, 3, 4, 5, 6, 7, 8, 9, 10, or all of the internucleoside linkage are O,O-linked phosphorothioate linkages.

The sequence listing of each of SEQ ID NO. 1-12 is also provided in Table 7.

Methods described herein can be used to synthesize oligonucleotides as described in U.S. Pat. No. 9,279,126.

In certain embodiments, the oligonucleotide, such as an anti-SMAD7 oligonucleotide or a chemically modified anti-SMAD7 oligonucleotide, may include at least one unnatural nucleoside, e.g., deoxycytidine and/or 5-methyl-2′-deoxycytidine. In certain embodiments, the oligonucleotide, such as an anti-SMAD7 oligonucleotide or a chemically modified anti-SMAD7 oligonucleotide, may include at least one unnatural nucleotide, e.g., 5-methyl-2′-deoxycytidine-5′-monophosphate or 5-methyl-2′-deoxycytidine-5′-monophosphorothioate. In certain embodiments, the oligonucleotide may include 2, 3, 4, 5, 6, 7, 8, 9, 10, 11, 12, 13, 14, 15, 16, 17, 18, 19, 20, 21, 22, 23, 24, 25, or more, deoxycytidine and/or 5-methyl 2′-deoxycytidines. In certain embodiments, at least 5%, 10%, 20%, 25%, 30%, 35%, 40%, 45%, 50%, 55%, 60%, 65%, 70%, 75%, 80%, 85%, 90%, 95%, or 100% of nucleotides in the oligonucleotide may include deoxycytidine and/or 5-methyl-2′-deoxycytidine. In certain embodiments, the oligonucleotide may include at least 2, 3, 4, 5, 6, 7, 8, 9, 10, 11, 12, 13, 14, 15, 16, 17, 18, 19, 20, 21, 22, 23, 24, 25, or more, deoxycytidine and/or 5-methyl 2′-deoxycytidine. In certain embodiments, the oligonucleotide may include one or more deoxycytidines and no 5-methyl 2′-deoxycytidine. In certain embodiments, the oligonucleotide may include one or more 5-methyl 2′-deoxycytidine and no deoxycytidine.

In certain embodiments, the oligonucleotide, such as an anti-SMAD7 oligonucleotide or a chemically modified anti-SMAD7 oligonucleotide, synthesized using the methods as described herein, may include methylphosphonate linkages that are placed at the 5′- and/or 3′-ends of the oligonucleotide.

In certain embodiments, the oligonucleotide, such as an anti-SMAD7 oligonucleotide or a chemically modified anti-SMAD7 oligonucleotide, may include pharmaceutically acceptable salts or solvates. In certain embodiments, the solvates are hydrates. In certain embodiments, the oligonucleotide, such as an anti-SMAD7 oligonucleotide or a chemically modified anti-SMAD7 oligonucleotide, comprises or is an alkali metal salt (e.g. a sodium salt) or an alkaline earth metal salt (e.g., a magnesium salt), such as the oligonucleotide comprising the nucleic acid sequence of SEQ ID NO: 3, that optionally can include 1 to 20 O,O-linked phosphorothioate internucleotide linkages. Contemplated salts of the oligonucleotide, such as an anti-SMAD7 oligonucleotide or a chemically modified anti-SMAD7 oligonucleotide, synthesized using the methods as described herein, may include those that are fully neutralized, e.g., each phosphorothioate linkage is associated with an alkali metal ion, such as Na⁺. In certain embodiments the oligonucleotide is only partially neutralized, e.g., less than all phosphorothioate linkages are associated with an alkali metal ion or an alkaline earth metal ion (e.g., less than 99%, less than 95%, less than 90%, less than 85%, less than 80%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 3%, or less than 1% are neutralized).

5.3 Solid Support and Linker

In certain embodiments, a solid support suitable for the method of preparing an oligonucleotide disclosed herein may be a crosslinked polystyrene or a controlled pore glass. For example, in certain embodiments, the solid support is a crosslinked polystyrene with a linker attached thereto or a porous, polydispersed divinyl benzene crosslinked polystyrene with a linker attached thereto, for example a linker comprising a protected hydroxyl group, such as a UNYLINKER. In certain embodiments, for example, the crosslinked polystyrene solid support is NITTOPHASE-HL, such as NITTOPHASE UNYLINKER 350 or high loaded NITTOPHASE UNYLINKER 350, or a NITTOPHASE solid support pre-loaded with a nucleotide, such as 2′-deoxycytidine. In certain embodiments, for example, the porous, polydispersed divinyl benzene crosslinked polystyrene solid support is PRIMER SUPPORT 5G, where the linker may be a succinyl group based linker. The loading capacity (or synthesis scale) of a solid support, such as a solid support having a linker attached thereto, refers to the molar amount of the solid support, such as the solid support having a linker attached thereto, that is available to react to prepare an oligonucleotide, e.g., an equivalent molar amount of the oligonucleotide. For example, the amount of the oligonucleotide prepared that is equivalent to the loading capacity of the solid support having a linker attached thereto is considered the theoretical amount. The actual amount of the oligonucleotide prepared according to the methods disclosed herein utilizing the solid support having a linker attached thereto may be identical to the theoretical amount, or may be less than the theoretical amount. For example, in certain embodiments, the actual amount of the oligonucleotide prepared according to the methods disclosed herein utilizing the solid support having a linker attached thereto is less than the theoretical amount, for example is 95% of the theoretical amount, such as 90%, 85%, 80%, 75%, 70%, 65%, 60%, 55%, or 50% of the theoretical amount, or for example, is between 40-98%, 40-95%, 40-90%, 40-85%, 40-80%, 40-75%, 40-70%, 50-98%, 50-95%, 50-90%, 50-85%, 50-80%, 50-75%, 50-70%, 60-98%, 60-95%, 60-90%, 60-85%, 60-80%, 60-75%, or 60-70% of the theoretical amount. In certain embodiments, the solid support having a linker attached thereto that is utilized in the methods disclosed herein has a loading capacity sufficient to prepare the oligonucleotide in an amount in the range of 300-5,400 mmol, for example, prepare the oligonucleotide in an amount in the range of 300-4,500 mmol, 300-4,000 mmol, 300-3,600 mmol, 300-3,000 mmol, 600-3,000 mmol, 700-3,600 mmol, 700-2,700 mmol, 700-2,500 mmol, 700-2,400 mmol, 700-2,000 mmol, 700-1,900 mmol, 700-1,800 mmol, 700-1,700 mmol, 700-1,600 mmol, 700-1,500 mmol, 700-1,400 mmol, 700-1,300 mmol, 700-1,200 mmol, 700-1,100 mmol, 700-1,000 mmol, 700-900 mmol, 800-1,200 mmol, 800-1,000 mmol, 900-1,600 mmol, 900-1,800 mmol, 900-2,400 mmol, 900-2,700 mmol, 900-3,600 mmol, 1,800-2,700 mmol, 1,800-3,600 mmol, 1,000-3,000 mmol, 1,000-2,500 mmol, 1,000-2,000 mmol, 1,000-1,500 mmol, 1,250-1,750 mmol, 1,500-3,000 mmol, 1,500-2,500 mmol, 1,500-2,000 mmol, 1,600-2,400 mmol, 2,000-3,000 mmol, 2,500-3,000 mmol, 2,700-3,600 mmol, 2,700-4,500 mmol, 3,000-4,000 mmol, 3,600-4,500 mmol, or 3,600-5,400 mmol. In certain embodiments, the solid support having a linker attached thereto that is utilized in the methods disclosed herein has a loading capacity sufficient to prepare the oligonucleotide in an amount of at least 300 mmol, for example, prepare the oligonucleotide in an amount at least 300 mmol, such as at least 400 mmol, at least 500 mmol, at least 600 mmol, at least 700 mmol, at least 800 mmol, at least 900 mmol, at least 1,000 mmol, at least 1,100 mmol, at least 1,200 mmol, at least 1,300 mmol, at least 1,400 mmol, at least 1,600 mmol, at least 1,800 mmol, at least 2,400 mmol, at least 2,700 mmol, at least 3,000 mmol, at least 3,600 mmol, or at least 4,500 mmol. In certain embodiments, the solid support having a linker attached thereto that is utilized in the methods disclosed herein has a loading capacity sufficient to prepare the oligonucleotide in an amount of 300 mmol, 400 mmol, 500 mmol, 600 mmol, 700 mmol, 800 mmol, 900 mmol, 1,000 mmol, 1,100 mmol, 1,200 mmol, 1,300 mmol, 1,400 mmol, 1,500 mmol, 1,600 mmol, 1,700 mmol, 1,800 mmol, 1,900 mmol, 2,000 mmol, 2,100 mmol, 2,200 mmol, 2,300 mmol, 2,400 mmol, 2,500 mmol, 2,600 mmol, 2,700 mmol, 2,800 mmol, 2,900 mmol, 3,000 mmol, 3,400 mmol, 3,600 mmol, 4,000 mmol, 4,200 mmol, 4,500 mmol, 5,000 mmol, or 5,400 mmol.

In certain embodiments, the loading density of the amount of linker attached to the solid support relative to the amount of solid support that is utilized during the synthesis of the oligonucleotide compound prepared according to the methods disclosed herein is in the range of 300-400 micromole of linker/gram of solid support, for example, a loading density in the range of 300-375 micromole of linker/gram of solid support, such as in the range of 300-350, 300-350, 300-325, 325-400, 325-375, 350-400, or 350-375 micromole of linker/gram of solid support. In certain embodiments, the loading density of the amount of linker attached to the solid support relative to the amount of solid support that is utilized during the synthesis of the oligonucleotide compound prepared according to the methods disclosed herein is at least 300 micromole linker/gram of solid support, for example, at least 320, at least 325, at least 330, at least 340, at least 350, or at least 360 micromole linker/gram of solid support.

In certain embodiments, the column housing the solid support having a linker attached thereto that is utilized in the methods disclosed herein has a column inner diameter in the range of between 35-100 cm, for example, an inner diameter in the range of between 50-100 cm, 50-75 cm, 60-80 cm, 55-75 cm, or 75-100 cm, such as an inner diameter of 35 cm, 40 cm, 50 cm, 60 cm, 70 cm, 80 cm, 90 cm, or 100 cm. In certain embodiments, the solid support having a linker attached thereto that is utilized in the methods disclosed herein has a bed height in the range of between 4-20 cm, for example, a bed height in the range of between 7-20 cm, 7-15 cm, 7-10 cm, 7.5-8.5, 8-20 cm, 8-15 cm, 8-10 cm, 8-9 cm, 8-8.5 cm, or 8.5-9 cm, such as a bed height of 4 cm, 5 cm, 6 cm, 7 cm, 7.25 cm, 7.34 cm, 7.5 cm, 7.66 cm, 7.75 cm, 8 cm, 8.25 cm, 8.34 cm, 8.5 cm, 8.66 cm, 8.75 cm, 9 cm, 9.25 cm, 9.34 cm, 9.5 cm, 9.66 cm, 9.75 cm, 10 cm, 10.25 cm, 10.34 cm, 10.5 cm, 10.66 cm, 10.75 cm, or 11 cm. In certain embodiments, the column housing the solid support having a linker attached thereto that is utilized in the methods disclosed herein has an inner diameter in the range of between 35-100 cm, for example, an inner diameter of 35 cm, 40 cm, 50 cm, 60 cm, 70 cm, 80 cm, 90 cm, or 100 cm, and a bed height in the range of between 4-20 cm, for example, the bed height in the range of between 7-20 cm, between 7-15 cm, between 7-10 cm, between 7.5-8.5, between 8-20 cm, between 8-15 cm, between 8-10 cm, between 8-9 cm, between 8-8.5 cm, or between 8.5-9 cm. For example, in certain embodiments, the column housing the solid support having a linker attached thereto that is utilized in the methods disclosed herein has an inner diameter of 60 cm, and the solid support has a bed height of 7 cm, 7.34 cm, 7.5 cm, 7.66 cm, 8 cm, 8.34 cm, 8.5 cm, 8.66 cm, 9 cm, 9.34 cm, 9.5 cm, 9.66 cm, or 10 cm; or the column housing the solid support has an inner diameter of 70 cm, and the solid support has a bed height of 7 cm, 7.34 cm, 7.5 cm, 7.66 cm, 8 cm, 8.34 cm, 8.5 cm, 8.66 cm, 9 cm, 9.34 cm, 9.5 cm, 9.66 cm, or 10 cm; or the column housing the solid support has an inner diameter of 80 cm, and the solid support has a bed height of 7 cm, 7.34 cm, 7.5 cm, 7.66 cm, 8 cm, 8.34 cm, 8.5 cm, 8.66 cm, 9 cm, 9.34 cm, 9.5 cm, 9.66 cm, or 10 cm; or the column housing the solid support has an inner diameter of 90 cm, and the solid support has a bed height of 7 cm, 7.34 cm, 7.5 cm, 7.66 cm, 8 cm, 8.34 cm, 8.5 cm, 8.66 cm, 9 cm, 9.34 cm, 9.5 cm, 9.66 cm, or 10 cm.

In certain embodiments, the column housing the solid support having a linker attached thereto that is utilized in the methods disclosed herein has an inner diameter of 60 cm, the solid support has a bed height of 7 cm, 7.34 cm, 7.5 cm, 7.66 cm, 8 cm, 8.34 cm, 8.5 cm, 8.66 cm, 9 cm, 9.34 cm, 9.5 cm, 9.66 cm, or 10 cm, and the solid support has a loading capacity sufficient to prepare the oligonucleotide in an amount of at least 600 mmol, such as at least 700 mmol, at least 900 mmol, at least 1,600 mmol, at least 2,400 mmol, at least 2,700 mmol, at least 3,000 mmol, at least 3,600 mmol, or at least 4,500 mmol. In certain embodiments, the column housing the solid support having a linker attached thereto that is utilized in the methods disclosed herein has an inner diameter of 80 cm, the solid support has a bed height of 7 cm, 7.34 cm, 7.5 cm, 7.66 cm, 8 cm, 8.34 cm, 8.5 cm, 8.66 cm, 9 cm, 9.34 cm, 9.5 cm, 9.66 cm, or 10 cm, and the solid support has a loading capacity sufficient to prepare the oligonucleotide in an amount of at least 600 mmol, such as at least 700 mmol, at least 900 mmol, at least 1,600 mmol, at least 2,400 mmol, at least 2,700 mmol, at least 3,000 mmol, at least 3,600 mmol, or at least 4,500 mmol. In certain embodiments, the column housing the solid support having a linker attached thereto that is utilized in the methods disclosed herein has an inner diameter in the range of between 50-100 cm, for example, an inner diameter of 50 cm, 60 cm, 70 cm, 80 cm, 90 cm, or 100 cm, and a column volume in the range of between 20-35 L, for example, a column volume in the range of between 20-25 L, between 23.5-30 L, between 25-30 L, between 30-35 L, or between 20-30 L, such as a column volume of 21, 22, 23, 24, 25, 26, 27, 28, 29, 30, 31, 32, 33, 34, or 35 L.

In certain embodiments, the hydroxyl protecting group of the hydroxyl group on the linker attached to the solid support described herein can include protecting groups such as acetyl, i-butyryl, t-butyl, t-butoxymethyl, methoxymethyl, tetrahydropyranyl, 1-ethoxyethyl, 1-(2-chloroethoxyl)ethyl, p-chlorophenyl, 2,4-dinitrophenyl, benzyl, 2,6-dichlorobenzyl, diphenylmethyl, p-nitrobenzyl, bis(2-acetoxyethoxy)methyl (ACE), 2-trimethylsilylethyl, trimethylsilyl, triethylsilyl, t-butyldimethylsilyl, t-butyldiphenylsilyl, triphenylsilyl, [(triisopropylsilyl)oxy]methyl (TOM), benzoylformate, chloroacetyl, trichloroacetyl, trifluoroacetyl, pivaloyl, benzoyl, p-phenylbenzoyl, 9-fluorenylmethyl carbonate, mesylate, tosylate, triphenylmethyl (trityl), monomethoxytrityl, dimethoxytrityl (DMT), trimethoxytrityl, 1(2-fluorophenyl)-4-methoxypiperidin-4-yl (FPMP), 9-phenylxanthine-9-yl (Pixyl) and 9-(p-methoxyphenyl)xanthine-9-yl (MOX). In certain embodiments, the hydroxyl protecting group on the linker attached to the solid support can include trityl and dimethoxytrityl (DMT), for example the hydroxyl protecting group is dimethoxytrityl (DMT).

5.4 Deprotecting Linker

In certain embodiments, the protected hydroxyl group of the linker is deprotected according to the method of preparing an oligonucleotide disclosed herein, thereby creating a deprotected hydroxyl group. For example, in certain embodiments, the protected hydroxyl group of the linker is deprotected with a protic acid, for example dichloroacetic acid, such as dichloroacetic acid in an aromatic solvent, for example, dichloroacetic acid in toluene. In certain embodiments, the protected hydroxyl group of the linker is deprotected with 3-15 wt. % of dichloroacetic acid in an aromatic solvent (v/v), for example, 5 wt. % or 10 wt. % of dichloroacetic acid in an aromatic solvent (v/v), such as 3-15 wt. % of dichloroacetic acid in toluene (v/v), for example, 5 wt. % or 10 wt. % of dichloroacetic acid in toluene (v/v). In certain embodiments, the volume of the protic acid utilized to deprotect the protected hydroxyl group of the linker, such as 5 wt. % or 10 wt. % of dichloroacetic acid in toluene (v/v), is in the range of between 1-10 column volumes, for example, in the range of between 1-7 column volumes, 1-6 column volumes, 1-5 column volumes, 1-4 column volumes, 1-3 column volumes, 2-5 column volumes, 2-4 column volumes, 2-3 column volumes, 3-5 column volumes, or 3-4 column volumes. In certain embodiments, following deprotection of the protected hydroxyl group of the linker, the support-bound deprotected material is washed with solvent, such as acetonitrile, in preparation for the next step of the method, such as the next reaction on the solid support, for example the support may be washed with between 1-7 column volumes, 1-5 column volumes, 1-4 column volumes, 1-3 column volumes, or 2-4 column volumes, in preparation for the next step of the method.

5.5 Nucleoside Phosphoramidite Coupling

In certain embodiments, the deprotected hydroxyl group of the linker, or the deprotected hydroxyl group of the nucleoside from the previous iteration of the reaction cycle, is coupled to a nucleoside phosphoramidite according to the method of preparing an oligonucleotide disclosed herein, thereby creating a phosphite triester linked nucleoside, such as a protected phosphite triester linked nucleoside. In certain embodiments, the coupling involves providing the nucleoside phosphoramidite to the solid support whereby the deprotected 5′-hydroxyl group of the linker or the deprotected 5′-hydroxyl group of the nucleoside from the previous iteration of the reaction cycle reacts with, or couples to, the nucleoside phosphoramidite. In certain embodiments, the coupling step of the method described herein results in the formation of a phosphite triester linked nucleoside, for example, a protected phosphite triester linked nucleoside, such as a phosphite triester linked nucleoside comprising a 2-cyanoethoxy-protecting group, such as a 2-cyanoethoxy-protected phosphite triester linked nucleoside. In certain embodiments, prior to providing the nucleoside phosphoramidite, the method further comprises optionally pre-swelling the support, such as, optionally pre-swelling the support with a polar aprotic solvent, such as dimethylformamide (DMF). In certain embodiments, the support is not pre-swelled prior to providing the nucleoside phosphoramidite. In certain embodiments, the method may further comprise optionally washing the support with an aprotic solvent, such as acetonitrile, upon completion of the coupling step and prior to the thiolation (or oxidation) step. For example, in certain embodiments, the support may be washed with between 1-10 column volumes, such as between 1-7 column volumes, 1-6 column volumes, 1-5 column volumes, 1-4 column volumes, 1-3 column volumes, or 2-4 column volumes, upon completion of the coupling step and prior to the thiolation (or oxidation) step.

In certain embodiments, the coupling comprises providing the nucleoside phosphoramidite with an activator, such as providing the nucleoside phosphoramidite and activator as a pre-mixed solution, such as a pre-mixed solution of the nucleoside phosphoramidite with the activator, or for example separately providing a solution of the nucleoside phosphoramidite and a solution of the activator. In certain embodiments, the activator is dicyanoimidazole (DCI). In certain embodiments, the solvent utilized to prepare the pre-mixed solution of the nucleoside phosphoramidite with the activator, or the each of the separate solutions of the nucleoside phosphoramidite and the activator, is an aprotic solvent, such as acetonitrile. In certain embodiments, the amount of the activator provided during the coupling step of the method is in the range of 2-8 equivalents, relative to the equivalents of the solid support, for example, in the range of 3-8 equivalents, 4-8 equivalents, 5-8 equivalents, 6-8 equivalents, 6-7 equivalents, or 4-7 equivalents, relative to the equivalents of the solid support.

In certain embodiments, the coupling step comprises providing an excess amount of the nucleoside phosphoramidite. In certain embodiments, the nucleoside phosphoramidite is a protected nucleoside phosphoramidite, such as a protected nucleoside phosphoramidite comprising a 5′-hydroxyl protected group and 3′-hydroxyl protected group.

In certain embodiments, the 5′-hydroxyl protected group of the provided nucleoside phosphoramidite can include protecting groups such as acetyl, i-butyryl, t-butyl, t-butoxymethyl, methoxymethyl, tetrahydropyranyl, 1-ethoxyethyl, 1-(2-chloroethoxyl)ethyl, p-chlorophenyl, 2,4-dinitrophenyl, benzyl, 2,6-dichlorobenzyl, diphenylmethyl, p-nitrobenzyl, bis(2-acetoxyethoxy)methyl (ACE), 2-trimethylsilylethyl, trimethylsilyl, triethylsilyl, t-butyldimethylsilyl, t-butyldiphenylsilyl, triphenylsilyl, [(triisopropylsilyl)oxy]methyl (TOM), benzoylformate, chloroacetyl, trichloroacetyl, trifluoroacetyl, pivaloyl, benzoyl, p-phenylbenzoyl, 9-fluorenylmethyl carbonate, mesylate, tosylate, triphenylmethyl (trityl), monomethoxytrityl, dimethoxytrityl (DMT), trimethoxytrityl, 1(2-fluorophenyl)-4-methoxypiperidin-4-yl (FPMP), 9-phenylxanthine-9-yl (Pixyl) and 9-(p-methoxyphenyl)xanthine-9-yl (MOX). In certain embodiments, the hydroxyl protecting group can include benzyl, 2,6-dichlorobenzyl, t-butyldimethylsilyl, t-butyldiphenylsilyl, benzoyl, mesylate, tosylate, dimethoxytrityl (DMT), 9-phenylxanthine-9-yl (Pixyl) and 9-(p-methoxyphenyl)xanthine-9-yl (MOX). In certain embodiments, the hydroxyl protecting group can include acetyl, i-butyryl, and dimethoxytrityl (DMT). In certain embodiments, the hydroxyl protecting group is i-butyryl. In certain embodiments, the hydroxyl protecting group is dimethoxytrityl (DMT).

In certain embodiments, the 3′-hydroxyl protected group of the provided nucleoside phosphoramidite is a phosphoramidite group, such as a (2-cyanoethyl)-N,N-diisopropyl-phosphoramidite group.

For example, in certain embodiments, the nucleoside phosphoramidite provided in the coupling step may be selected from the group consisting of:
3′-O—[(N,N-diisopropylamino)-2-cyanoethoxyphosphinyl]-5′-O-(4,4′-dimethoxytrityl)-N⁶-benzoyldeoxyadenosine (dA-(Bz) or Bz-dA-phosphoramidite);
3′-O—[(N,N-diisopropylamino)-2-cyanoethoxyphosphinyl]-5′-O-(4,4′-dimethoxytrityl)-N⁴-benzoyldeoxycytidine (dC-(Bz) or Bz-dC-phosphoramidite);
3′-O—[(N,N-diisopropylamino)-2-cyanoethoxyphosphinyl]-5′-O-(4,4′-dimethoxytrityl)-N²-isobutyryldeoxyguanosine (dG-(iBu) or i-Bu-dG-phosphoramidite);
3′-O—[(N,N-diisopropylamino)-2-cyanoethoxyphosphinyl]-5′-O-(4,4′-dimethoxytrityl)-thymidine (dT or dT-phosphoramidite); and
3′-O—[(N,N-diisopropylamino)-2-cyanoethoxyphosphinyl]-5′-O-(4,4′-dimethoxytrityl)-N⁴-benzoyl-5-methyldeoxycytidine (d5MeC—(Bz) or 5-Me-dC-phosphoramidite).

In certain embodiments, the amount of the nucleoside phosphoramidite provided during the coupling step of the method is an excess amount of equivalents, relative to the equivalents of the solid support. In certain embodiments, the amount of the nucleoside phosphoramidite provided during the coupling step of the method is in the range of 1-8 equivalents, relative to the equivalents of the solid support, for example, the amount of the nucleoside phosphoramidite provided during the coupling step of the method is in the range of 1-7 equivalents, relative to the equivalents of the solid support, such as in the range of 1-6 equivalents, 1-5 equivalents, 1-4 equivalents, 1-3 equivalents, 1-2 equivalents, 1.1-2 equivalents, 1.1-1.75 equivalents, 1.2-1.75 equivalents, 1.5-1.75 equivalents, 1.5-2 equivalents, 1.5-2.5 equivalents, 2-3 equivalents, 2-4 equivalents, or 2-5 equivalents, relative to the equivalents of the solid support. In certain embodiments, the amount of the nucleoside phosphoramidite provided during the coupling step of the method is in the range of 1-4 equivalents, relative to the equivalents of the solid support, for example, the amount of the nucleoside phosphoramidite provided during the coupling step of the method is 1 equivalent, relative to the equivalents of the solid support, such as 1.1, 1.2, 1.25, 1.5, 1.75, 2, 2.25, 2.5, 2.75, 3, 3.5, or 4 equivalents, relative to the equivalents of the solid support. In certain embodiments, the amount of the nucleoside phosphoramidite provided during the coupling step of the method is in the range of 0.1-1 equivalents, relative to the equivalents of activator provided, for example, in the range of 0.1-0.9 equivalents, 0.1-0.8 equivalents, 0.1-0.7 equivalents, 0.1-0.6 equivalents, 0.1-0.5 equivalents, 0.1-0.4 equivalents, 0.1-0.3 equivalents, 0.1-0.2 equivalents, 0.25-0.9 equivalents, 0.25-0.75 equivalents, 0.25-0.5 equivalents, 0.3-0.8 equivalents, 0.3-0.75 equivalents, or 0.5-1 equivalents, relative to the equivalents of activator provided. In certain embodiments, the amount of the nucleoside phosphoramidite provided during the coupling step of the method is in the range of 0.05-1 equivalents, relative to the equivalents of activator provided, for example, the amount of the nucleoside phosphoramidite provided during the coupling step of the method is 0.1 equivalents, relative to the equivalents of activator provided, such as 0.1, 0.2, 0.25, 0.3, 0.4, 0.5, 0.6, 0.7, 0.75, 0.8, 0.9, or 1 equivalents, relative to the equivalents of activator provided. In certain embodiments, the nucleoside phosphoramidite is provided as an aprotic solution, such as an acetonitrile solution, wherein the nucleoside phosphoramidite is selected from the group consisting of: dA-(Bz), dC-(Bz), dG-(iBu), dT, and d5MeC—(Bz).

In certain embodiments, the coupling comprises providing the activator and the nucleoside phosphoramidite in a molar ratio of 1-5:1, for example, in a molar ratio of 2-5:1, such as 3-5:1, 4:1, or 3.5:1.

5.6 Thiolation or Oxidation

In certain embodiments, the protected phosphite triester linkage, such as the protected phosphite triester linkage from the previous iteration of the reaction cycle, is thiolated according to the method of preparing an oligonucleotide disclosed herein, thereby creating a protected phosphorothioate linkage. In certain embodiments, the protected phosphite triester linkage, such as the 2-cyanoethoxy-protected phosphite triester linked nucleoside, is thiolated with a thiolating agent, thereby forming a protected phosphorothioate linkage, such as a 2-cyanoethoxy-protected phosphorothioate linkage. In certain embodiments, the thiolating agent is provided in an amount in the range of 1-8 equivalents, relative to the equivalents of the solid support, for example, in the range of 2-8 equivalents, 3-8 equivalents, 4-8 equivalents, 5-8 equivalents, 5-7 equivalents, 5-6 equivalents, 1-5 equivalents, or 3-6 equivalents, relative to the equivalents of the solid support. In certain embodiments, the thiolating agent may be xanthane hydride (XH), such as xanthane hydride in pyridine, a disulfide, such as phenylacetyl disulfide (or di(phenylacetyl) disulfide) or a disulfide of a sulfonic acid, tetraethylhiuram disulfide, dibenzoyl tetrasulfide, 1,2,4-dithivazoline-5-one (DtsNH), 3-ethoxy-1,2,4-dithivazoline-5-one (EDITH), thiophosphorous compounds, Beaucage reagent, or a 3-aryl-1,2,4-dithiazoline-5-one (as disclosed in U.S. Pat. No. 6,500,944, herein incorporated by reference in its entirety). In certain embodiments, the method may further comprise optionally washing the support with an aprotic solvent, such as acetonitrile, upon completion of the thiolation step. For example, in certain embodiments, the support may be washed with between 1-10 column volumes, such as between 1-7 column volumes, 1-6 column volumes, 1-5 column volumes, 1-4 column volumes, 1-3 column volumes, or 2-4 column volumes, upon completion of the thiolation step.

In certain embodiments, the protected phosphite triester linkage, such as the protected phosphite triester linkage from the previous iteration of the reaction cycle, is oxidized according to the method of preparing an oligonucleotide disclosed herein, thereby creating a protected phosphate linkage. In certain embodiments, the protected phosphite triester linkage, such as the 2-cyanoethoxy-protected phosphite triester linked nucleoside, is oxidized with an oxidizing agent, thereby forming a protected phosphate linkage, such as a 2-cyanoethoxy-protected phosphate linkage. In certain embodiments, the oxidizing agent is provided in an amount in the range of 1-8 equivalents, relative to the equivalents of the solid support, for example, provided in an amount in the range of 2-8 equivalents, such as in the range of 3-8 equivalents, 4-8 equivalents, 5-8 equivalents, 5-7 equivalents, 5-6 equivalents, 1-5 equivalents, or 3-6 equivalents, relative to the equivalents of the solid support. In certain embodiments, the oxidizing agent may be iodine or t-butyl hydroperoxide. In certain embodiments, the method may further comprise optionally washing the support with an aprotic solvent, such as acetonitrile, upon completion of the oxidation step. For example, in certain embodiments, the support may be washed with between 1-10 column volumes, such as between 1-7 column volumes, 1-6 column volumes, 1-5 column volumes, 1-4 column volumes, 1-3 column volumes, or 2-4 column volumes, upon completion of the oxidation step.

5.7 Capping

In certain embodiments, the unreacted deprotected hydroxyl group(s) from the previous iteration of the reaction cycle is capped (protected) according to the method of preparing an oligonucleotide disclosed herein, thereby creating capped hydroxyl group(s). In certain embodiments, the unreacted deprotected hydroxyl group(s) is capped with an acyl group, such as capped with an alkyl acyl group. In certain embodiments, the alkyl acyl group is an isobutyl acyl group. In certain embodiments, the capping of the unreacted deprotected hydroxyl group(s) from the previous iteration of the reaction cycle comprises providing a first capping solution (Cap A), comprising N-methylimidazole (NMI), pyridine, and acetonitrile, and providing a second capping solution (Cap B), comprising capping agent and acetonitrile. In certain embodiments, the capping of the unreacted deprotected hydroxyl group(s) from the previous iteration of the reaction cycle comprises providing a first capping solution (Cap A), comprising 10-30% N-methylimidazole (NMI), 20-40% pyridine, and 40-60% acetonitrile (v/v/v), for example, a first capping solution (Cap A), comprising 20% N-methylimidazole (NMI), 30% pyridine, and 50% acetonitrile (v/v/v), and providing a second capping solution (Cap B), comprising 10-30% capping agent in acetonitrile (v/v), for example, a second capping solution (Cap B), comprising 20% capping agent in acetonitrile (v/v). In certain embodiments, the first capping solution (Cap A) and the second capping solution (Cap B) are premixed before introducing to the solid support to cap the unreacted deprotected hydroxyl group(s) from the previous iteration of the reaction cycle. In certain embodiments, a single capping solution is introduced to the solid support to cap the unreacted deprotected hydroxyl group(s) from the previous iteration of the reaction cycle, comprising N-methylimidazole (NMI), pyridine, capping agent, and acetonitrile. In certain embodiments, the capping agent is an alkyl ester or an alkyl anhydride. In certain embodiments, the capping agent is an alkyl ester, for example, the capping agent is an alkyl methyl ester, such as isopropyl methyl ester. In certain embodiments, the capping agent is an alkyl anhydride, for example, the capping agent is isobutyric anhydride. In certain embodiments, prior to capping of the unreacted deprotected hydroxyl group(s) from the previous iteration of the reaction cycle, the method further comprises optionally pre-swelling the support, such as, optionally pre-swelling the support with a polar aprotic solvent, such as dimethylformamide (DMF). In certain embodiments, the support is not pre-swelled prior to capping of the unreacted deprotected hydroxyl group(s) from the previous iteration of the reaction cycle. In certain embodiments, the method may further comprise optionally washing the support with an aprotic solvent, such as acetonitrile, upon completion of the capping step. For example, in certain embodiments, the support may be washed with between 1-10 column volumes, such as between 1-7 column volumes, 1-6 column volumes, 1-5 column volumes, 1-4 column volumes, 1-3 column volumes, or 2-4 column volumes, upon completion of the capping step.

5.8 Deprotecting 5′-Terminal Nucleoside

In certain embodiments, the protected hydroxyl group of the nucleoside, such as the protected hydroxyl group of the nucleoside from the previous iteration of the reaction cycle, is deprotected according to the method of preparing an oligonucleotide disclosed herein, thereby creating a deprotected hydroxyl group. For example, in certain embodiments, the protected hydroxyl group of the nucleoside, such as the protected 5′-hydroxyl group of the 5′-terminal nucleoside of the oligonucleotide, is deprotected with a protic acid, for example dichloroacetic acid, such as dichloroacetic acid in an aromatic solvent, for example, dichloroacetic acid in toluene, or in a halogenated solvent, such as dichloromethane. In certain embodiments, the protected 5′-hydroxyl group of the 5′-terminal nucleoside of the oligonucleotide is deprotected with 2-15 wt. % of a protic acid, for example, deprotected with 2-10 wt. % 3-10 wt. % of a protic acid, such as 3-10 wt. %, 5-10 wt. %, 3-15 wt. %, 10-15 wt. %, 3-5 wt. %, 5-8 wt. %, or 3-7 wt. % of a protic acid. In certain embodiments, the protected 5′-hydroxyl group of the 5′-terminal nucleoside of the oligonucleotide is deprotected with 2-15 wt. % of dichloroacetic acid in an aromatic solvent (v/v), such as 3-10 wt. %, for example, 3 wt. % of dichloroacetic acid in an aromatic solvent (v/v). For example, in certain embodiments, the protected 5′-hydroxyl group of the 5′-terminal nucleoside of the oligonucleotide is deprotected with 2-15 wt. % of dichloroacetic acid in toluene (v/v), such as 3-10 wt. %, for example, 3 wt. %, 5 wt. %, 7 wt. %, or 10 wt. %, of dichloroacetic acid in toluene (v/v). In certain embodiments, the volume of the protic acid utilized to deprotect the protected 5′-hydroxyl group of the 5′-terminal nucleoside of the oligonucleotide, for example 2-15 wt. % of dichloroacetic acid in toluene (v/v), such as 3-10 wt. %, for example, 5 wt. % or 10 wt. % of dichloroacetic acid in toluene (v/v), is in the range of between 1-10 column volumes, for example, in the range of between 1-7 column volumes, 1-6 column volumes, 1-5 column volumes, 1-4 column volumes, 1-3 column volumes, 2-5 column volumes, 2-4 column volumes, 2-3 column volumes, 3-5 column volumes, or 3-4 column volumes. In certain embodiments, the volume of the protic acid utilized to deprotect the protected 5′-hydroxyl group of the 5′-terminal nucleoside of the oligonucleotide may vary from synthesis cycle to synthesis cycle in preparing the target oligonucleotide, such as increasing volumes or decreasing volumes over the course of preparing the oligonucleotide. For example, as an illustration, in preparing a 21-mer oligonucleotide, the volume of the protic acid utilized to deprotect the protected 5′-hydroxyl group of the 5′-terminal nucleoside of the oligonucleotide, for example 2-15 wt. % of dichloroacetic acid in toluene (v/v), such as 3-10 wt. %, for example, 5 wt. % or 10 wt. % of dichloroacetic acid in toluene (v/v), may be in the range of between 3-4 column volumes during the first cycle, in the range of between 2-3 column volumes during cycles 2-16, and in the range of between 2-3 column volumes during cycles 17-21. In certain embodiments, following deprotection of the protected 5′-hydroxyl group of the 5′-terminal nucleoside of the oligonucleotide, the support-bound deprotected material is washed with solvent, such as acetonitrile, in preparation for the next step of the method, such as the next reaction on the solid support, for example, the support may be washed with between 1-10 column volumes, such as between 1-7 column volumes, 1-6 column volumes, 1-5 column volumes, 1-4 column volumes, 1-3 column volumes, or 2-4 column volumes, in preparation for the next step of the method.

5.9 Reaction Cycle Iterations

In certain embodiments, the steps of providing and coupling of a nucleoside phosphoramidite, thiolating (or oxidizing) of the formed phosphite triester linkage, optionally capping of unreacted deprotected hydroxyl group(s), and optionally deprotecting of the protected 5′-hydroxyl group of the 5′-terminal nucleoside of the oligonucleotide, are repeated a predetermined number of times (reaction cycle interations) according to the method of preparing an oligonucleotide disclosed herein, to provide a solid support-bound oligonucleotide. In certain embodiments, the coupling of a nucleoside phosphoramidite, thiolating (or oxidizing) of the formed phosphite triester linkage, optionally capping of unreacted deprotected hydroxyl group(s), and optionally deprotecting of the protected 5′-hydroxyl group of the 5′-terminal nucleoside of the oligonucleotide, steps are repeated a predetermined number of times (reaction cycle interations) according to the method of preparing an oligonucleotide disclosed herein, to provide a solid support-bound oligonucleotide. In certain embodiments, the predetermined number of times (reaction cycle interations), according to the method of preparing an oligonucleotide disclosed herein, is exclusive of performing the optional step of capping of unreacted deprotected hydroxyl group(s) and the optional step of deprotecting of the protected 5′-hydroxyl group of the 5′-terminal nucleoside of the oligonucleotide during the last reaction cycle iteration. In certain embodiments, according to the method of preparing an oligonucleotide disclosed herein, one or more of the cycles of the predetermined number of times (reaction cycle interations) performs the step of capping the unreacted deprotected hydroxyl group(s) and deprotecting of the protected 5′-hydroxyl group of the 5′-terminal nucleoside of the oligonucleotide, and is exclusive of performing the capping step and the deprotecting step during the last reaction cycle iteration. In certain embodiments, following at least one of the repeated predetermined steps, the support-bound material is washed with solvent, such as acetonitrile, in preparation for the next reaction. In certain embodiments, following each of the repeated predetermined steps during the initial reaction cycle iterations, for example up to the first 3-10 reaction cycle iterations, such as up to the first 7 reaction cycle iterations, the support-bound material is washed with solvent, such as acetonitrile, in preparation for the next reaction. In certain embodiments, following each of the repeated predetermined steps, the support-bound material is washed with solvent, such as acetonitrile, in preparation for the next reaction.

In certain embodiments, the reaction cycle of performing the steps of providing and coupling of a nucleoside phosphoramidite, thiolating (or oxidizing) of the formed phosphite triester linkage, optionally capping of unreacted deprotected hydroxyl group(s), and optionally deprotecting of the protected 5′-hydroxyl group of the 5′-terminal nucleoside of the oligonucleotide, are repeated 9-99 times to provide the solid support-bound oligonucleotide, for example, are repeated 9-89 times, such as 9-79 times, 9-69 times, 9-59 times, 9-49 times, 9-39 times, 9-29 times, 9-24 times, 9-20 times, 10-30 times, 15-25 times, 20-30 times, 14-24 times, 14-20 times, or 10-24 times. In certain embodiments, the reaction cycle of performing the steps of providing/coupling of a nucleoside phosphoramidite, thiolating (or oxidizing) of the formed phosphite triester linkage, optionally capping of unreacted deprotected hydroxyl group(s), and optionally deprotecting of the protected 5′-hydroxyl group of the 5′-terminal nucleoside of the oligonucleotide, are repeated 9-99 times to provide the solid support-bound oligonucleotide, wherein the optional step of capping the unreacted deprotected hydroxyl group(s) and the optional step of deprotecting the protected 5′-hydroxyl group of the 5′-terminal nucleoside of the oligonucleotide are excluded during the last reaction cycle iteration, for example, are repeated 9-89 times, such as 9-79 times, 9-69 times, 9-59 times, 9-49 times, 9-39 times, 9-29 times, 9-24 times, 9-20 times, 14-24 times, 14-20 times, or 10-24 times, wherein the optional step of capping the unreacted deprotected hydroxyl group(s) and the optional step of deprotecting the protected 5′-hydroxyl group of the 5′-terminal nucleoside of the oligonucleotide are excluded during the last reaction cycle iteration. In certain embodiments, the reaction cycle of performing the steps of providing/coupling of a nucleoside phosphoramidite, thiolating (or oxidizing) of the formed phosphite triester linkage, optionally capping of unreacted deprotected hydroxyl group(s), and optionally deprotecting of the protected 5′-hydroxyl group of the 5′-terminal nucleoside of the oligonucleotide, are repeated a predetermined number of times to provide the solid support-bound oligonucleotide having 10-100 monomer subunits, for example, having 10-90 monomer subunits, 10-80 monomer subunits, 10-70 monomer subunits, 10-60 monomer subunits, 10-50 monomer subunits, 10-40 monomer subunits, 10-30 monomer subunits, 10-25 monomer subunits, 15-25 monomer subunits, 20-30 monomer subunits, 20-25 monomer subunits, 20-90 monomer subunits, 30-90 monomer subunits, or 40-70 monomer subunits, wherein the optional step of capping the unreacted deprotected hydroxyl group(s) and the optional step of deprotecting the protected 5′-hydroxyl group of the 5′-terminal nucleoside of the oligonucleotide are excluded during the last reaction cycle iteration. In certain embodiments, the reaction cycle of performing the steps of providing/coupling of a nucleoside phosphoramidite, thiolating (or oxidizing) of the formed phosphite triester linkage, optionally capping of unreacted deprotected hydroxyl group(s), and optionally deprotecting of the protected 5′-hydroxyl group of the 5′-terminal nucleoside of the oligonucleotide, are repeated 14 times to provide the solid support-bound oligonucleotide, for example, are repeated 15 times, such as 16, 17, 18, 19, 20, 21, 22, 23, 24, 25, 26, 27, 28, 29, or 30 times, wherein the optional step of capping the unreacted deprotected hydroxyl group(s) and the optional step of deprotecting the protected 5′-hydroxyl group of the 5′-terminal nucleoside of the oligonucleotide are excluded during the last reaction cycle iteration. In certain embodiments, the reaction cycle of performing the steps of providing/coupling of a nucleoside phosphoramidite, thiolating (or oxidizing) of the formed phosphite triester linkage, optionally capping of unreacted deprotected hydroxyl group(s), and optionally deprotecting of the protected 5′-hydroxyl group of the 5′-terminal nucleoside of the oligonucleotide, are repeated a predetermined number of times to provide the solid support-bound oligonucleotide having 15 monomer subunits, for example, having 16, 17, 18, 19, 20, 21, 22, 23, 24, 25, 26, 27, 28, 29, or 30 monomer subunits, wherein the optional step of capping the unreacted deprotected hydroxyl group(s) and the optional step of deprotecting the protected 5′-hydroxyl group of the 5′-terminal nucleoside of the oligonucleotide are excluded during the last reaction cycle iteration.

In certain embodiments, the reaction cycle of performing the steps of providing/coupling of a nucleoside phosphoramidite, thiolating (or oxidizing) of the formed phosphite triester linkage, optionally capping of unreacted deprotected hydroxyl group(s), and optionally deprotecting of the protected 5′-hydroxyl group of the 5′-terminal nucleoside of the oligonucleotide, are repeated a predetermined number of times to provide the solid support-bound oligonucleotide in an amount (such as a theoretical amount based on the synthesis scale or loading capacity of the solid support having a linker attached thereto) of 300 mmol or greater, for example, in an amount of 600 mmol or greater, 700 mmol or greater, 800 mmol or greater, 900 mmol or greater, 1,000 mmol or greater, 1,100 mmol or greater, 1,200 mmol or greater, 1,300 mmol or greater, 1,400 mmol or greater, 1,500 mmol or greater, 1,600 mmol or greater, 1,700 mmol or greater, 1,800 mmol or greater, 1,900 mmol or greater, 2,000 mmol or greater, 2,100 mmol or greater, 2,200 mmol or greater, 2,300 mmol or greater, 2,400 mmol or greater, 2,500 mmol or greater, 2,600 mmol or greater, 2,700 mmol or greater, 2,800 mmol or greater, 2,900 mmol or greater, 3,000 mmol or greater, 3,200 mmol or greater, 3,400 mmol or greater, 3,600 mmol or greater, 4,000 mmol or greater, 4,200 mmol or greater, 4,500 mmol or greater, 5,000 mmol or greater, or 5,400 mmol or greater, or for example, in the range of between 300-5,400 mmol, for example, between 300-4,500 mmol, 300-4,000 mmol, 300-3,600 mmol, 300-3,000 mmol, 600-3,000 mmol, 700-3,600 mmol, 700-2,700 mmol, 700-2,500 mmol, 700-2,400 mmol, 700-2,000 mmol, 700-1,900 mmol, 700-1,800 mmol, 700-1,700 mmol, 700-1,600 mmol, 700-1,500 mmol, 700-900 mmol, 800-1,200 mmol, 800-1,000 mmol, 900-1,600 mmol, 900-1,800 mmol, 900-2,400 mmol, 900-2,700 mmol, 900-3,600 mmol, 1,800-2,700 mmol, 1,800-3,600 mmol, 1,000-3,000 mmol, 1,000-2,500 mmol, 1,000-2,000 mmol, 1,000-1,500 mmol, 1,250-1,750 mmol, 1,500-3,000 mmol, 1,500-2,500 mmol, 1,500-2,000 mmol, 1,600-2,400 mmol, 2,000-3,000 mmol, 2,500-3,000 mmol, 2,700-3,600 mmol, 2,700-4,500 mmol, 3,000-4,000 mmol, 3,600-4,500 mmol, or 3,600-5,400 mmol, wherein the optional step of capping the unreacted deprotected hydroxyl group(s) and the optional step of deprotecting the protected 5′-hydroxyl group of the 5′-terminal nucleoside of the oligonucleotide are excluded during the last reaction cycle iteration. In certain embodiments, the reaction cycle of performing the steps of providing/coupling of a nucleoside phosphoramidite, thiolating (or oxidizing) of the formed phosphite triester linkage, optionally capping of unreacted deprotected hydroxyl group(s), and optionally deprotecting of the protected 5′-hydroxyl group of the 5′-terminal nucleoside of the oligonucleotide, are repeated a predetermined number of times to provide the solid support-bound oligonucleotide in an amount (such as a theoretical amount based on the synthesis scale or loading capacity of the solid support having a linker attached thereto) of 300 mmol, 400 mmol, 500 mmol, 600 mmol, 700 mmol, 800 mmol, 900 mmol, 1,000 mmol, 1,100 mmol, 1,200 mmol, 1,300 mmol, 1,400 mmol, 1,500 mmol, 1,600 mmol, 1,700 mmol, 1,800 mmol, 1,900 mmol, 2,000 mmol, 2,100 mmol, 2,200 mmol, 2,300 mmol, 2,400 mmol, 2,500 mmol, 2,600 mmol, 2,700 mmol, 2,800 mmol, 2,900 mmol, 3,000 mmol, 3,400 mmol, 3,600 mmol, 4,000 mmol, 4,200 mmol, 4,500 mmol, 5,000 mmol, or 5,400 mmol, wherein the optional step of capping the unreacted deprotected hydroxyl group(s) and the optional step of deprotecting the protected 5′-hydroxyl group of the 5′-terminal nucleoside of the oligonucleotide are excluded during the last reaction cycle iteration.

5.10 Deprotecting Phosphorothioate Linkages

In certain embodiments, the protected phosphorothioate linkages of the solid support-bound oligonucleotide are deprotected according to the method of preparing an oligonucleotide disclosed herein. For example, in certain embodiments, the 2-cyanoethoxy-protected phosphorothioate linkages of the solid support-bound oligonucleotide are deprotected. In certain embodiments, the protected phosphorothioate linkages, such as the 2-cyanoethoxy-protected phosphorothioate linkages, of the solid support-bound oligonucleotide are deprotected with an amine. In certain embodiments, the protected phosphorothioate linkages are deprotected with an amine, e.g., triethylamine, for example, 10-50% triethylamine in an aprotic solvent (v/v), for example, with 10-30% triethylamine in an aprotic solvent (v/v), such as with 20% triethylamine in acetonitrile (v/v). In certain embodiments, the step of deprotecting the protected phosphorothioate linkages forms phosphorothioate linkages, for example, unprotected phosphorothioate linkages or an amine salt form of the phosphorothioate linkages, such as a triethylamine salt form of the phosphorothioate linkages. In certain embodiments, the step of deprotecting the protected phosphorothioate linkages forms a solid support-bound oligonucleotide having phosphorothioate linkages, such as a solid support-bound oligonucleotide having unprotected phosphorothioate linkages. In certain embodiments, following deprotection step, the support-bound deprotected material is washed with solvent, such as acetonitrile, in preparation for the next reaction, for example, the support may be washed with between 1-10 column volumes, such as between 1-7 column volumes, 1-6 column volumes, 1-5 column volumes, 1-4 column volumes, 1-3 column volumes, or 2-4 column volumes, in preparation for the next step of the method.

5.11 Cleavage and Elution from Solid Support

In certain embodiments, the solid support-bound oligonucleotide having unprotected phosphorothioate linkages is cleaved from the solid support according to the method of preparing an oligonucleotide disclosed herein. In certain embodiments, the cleaving step of the method disclosed herein comprises providing a solution of ammonium hydroxide, for example, a heated solution of ammonium hydroxide, such as a heated solution having a temperature of 40-70° C. or 40-60° C., such as a temperature of 40° C., 50° C., 60° C., or 65° C., for example, providing the heated ammonium hydroxide solution and recirculating the heated ammonium hydroxide solution through the column housing to cleave said deprotected solid support-bound oligonucleotide from the solid support. In certain embodiments, the cleaving step of the method disclosed herein comprises providing a 28-30% ammonia aqueous solution (w/w), for example, a heated 28-30% ammonia aqueous solution (w/w), such as a heated solution having a temperature of 40-70° C. or 40-60° C., such as a temperature of 40° C., 50° C., 60° C., or 65° C., for example, providing the heated 28-30% ammonia aqueous solution (w/w) and recirculating the heated ammonia aqueous solution through the column housing to cleave said deprotected solid support-bound oligonucleotide from the solid support. In certain embodiments, the heated solution is recirculated through the support for 8-36 hours, such as for 24 hours. In certain embodiments, the heated solution is recirculated through the support and in contact with said support for 8-36 hours, for example, 12-36 hours, such as for 24 hours.

In certain embodiments, the cleaving step of the method further deprotects exocyclic amino protecting groups of the solid support-bound oligonucleotide having unprotected phosphorothioate linkages, of the cleaved oligonucleotide having unprotected phosphorothioate linkages, or both. In certain embodiments, the exocyclic amino protecting groups comprises benzoyl and isobutyryl groups. In certain embodiments, the cleaving step further deprotects the benzoyl- and isobutyryl-amino protecting groups of the solid support-bound oligonucleotide having unprotected phosphorothioate linkages, of the cleaved oligonucleotide having unprotected phosphorothioate linkages, or both.

In certain embodiments, the cleaved oligonucleotide comprises a 5′-hydroxyl protected group, for example the terminal nucleoside of the cleaved oligonucleotide comprises a 5′-hydroxyl protected group. In certain embodiments, the 5′-hydroxyl protected group of the cleaved oligonucleotide can include protecting groups such as acetyl, i-butyryl, t-butyl, t-butoxymethyl, methoxymethyl, tetrahydropyranyl, 1-ethoxyethyl, 1-(2-chloroethoxyl)ethyl, p-chlorophenyl, 2,4-dinitrophenyl, benzyl, 2,6-dichlorobenzyl, diphenylmethyl, p-nitrobenzyl, bis(2-acetoxyethoxy)methyl (ACE), 2-trimethylsilylethyl, trimethylsilyl, triethylsilyl, t-butyldimethylsilyl, t-butyldiphenylsilyl, triphenylsilyl, [(triisopropylsilyl)oxy]methyl (TOM), benzoylformate, chloroacetyl, trichloroacetyl, trifluoroacetyl, pivaloyl, benzoyl, p-phenylbenzoyl, 9-fluorenylmethyl carbonate, mesylate, tosylate, triphenylmethyl (trityl), monomethoxytrityl, dimethoxytrityl (DMT), trimethoxytrityl, 1(2-fluorophenyl)-4-methoxypiperidin-4-yl (FPMP), 9-phenylxanthine-9-yl (Pixyl) and 9-(p-methoxyphenyl)xanthine-9-yl (MOX). In certain embodiments, the hydroxyl protecting group of the cleaved oligonucleotide can include trityl or dimethoxytrityl (DMT). In certain embodiments, the hydroxyl protecting group of the cleaved oligonucleotide is dimethoxytrityl (DMT).

In certain embodiments, the cleaving of the solid support-bound oligonucleotide provides the cleaved oligonucleotide, such as the cleaved oligonucleotide comprising a 5′-hydroxyl protected group on the terminal nucleoside, in an amount (such as a theoretical amount based on the synthesis scale or loading capacity of the solid support having a linker attached thereto) of 300 mmol or greater, for example, in an amount of 600 mmol or greater, 700 mmol or greater, 800 mmol or greater, 900 mmol or greater, 1,000 mmol or greater, 1,100 mmol or greater, 1,200 mmol or greater, 1,300 mmol or greater, 1,400 mmol or greater, 1,500 mmol or greater, 1,600 mmol or greater, 1,700 mmol or greater, 1,800 mmol or greater, 1,900 mmol or greater, 2,000 mmol or greater, 2,100 mmol or greater, 2,200 mmol or greater, 2,300 mmol or greater, 2,400 mmol or greater, 2,500 mmol or greater, 2,600 mmol or greater, 2,700 mmol or greater, 2,800 mmol or greater, 2,900 mmol or greater, 3,000 mmol or greater, 3,200 mmol or greater, 3,400 mmol or greater, 3,600 mmol or greater, 4,000 mmol or greater, 4,200 mmol or greater, 4,500 mmol or greater, 5,000 mmol or greater, or 5,400 mmol or greater, or for example, in the range of between 300-5,400 mmol, for example, between 300-4,500 mmol, 300-4,000 mmol, 300-3,600 mmol, 300-3,000 mmol, 600-3,000 mmol, 700-3,600 mmol, 700-2,700 mmol, 700-2,500 mmol, 700-2,400 mmol, 700-2,000 mmol, 700-1,900 mmol, 700-1,800 mmol, 700-1,700 mmol, 700-1,600 mmol, 700-1,500 mmol, 700-900 mmol, 800-1,200 mmol, 800-1,000 mmol, 900-1,600 mmol, 900-1,800 mmol, 900-2,400 mmol, 900-2,700 mmol, 900-3,600 mmol, 1,800-2,700 mmol, 1,800-3,600 mmol, 1,000-3,000 mmol, 1,000-2,500 mmol, 1,000-2,000 mmol, 1,000-1,500 mmol, 1,250-1,750 mmol, 1,500-3,000 mmol, 1,500-2,500 mmol, 1,500-2,000 mmol, 1,600-2,400 mmol, 2,000-3,000 mmol, 2,500-3,000 mmol, 2,700-3,600 mmol, 2,700-4,500 mmol, 3,000-4,000 mmol, 3,600-4,500 mmol, or 3,600-5,400 mmol. In certain embodiments, the cleaving of the solid support-bound oligonucleotide provides the cleaved oligonucleotide, such as the cleaved oligonucleotide comprising a 5′-hydroxyl protected group on the terminal nucleoside, in an amount (such as a theoretical amount based on the synthesis scale or loading capacity of the solid support having a linker attached thereto) of 300 mmol, 400 mmol, 500 mmol, 600 mmol, 700 mmol, 800 mmol, 900 mmol, 1,000 mmol, 1,100 mmol, 1,200 mmol, 1,300 mmol, 1,400 mmol, 1,500 mmol, 1,600 mmol, 1,700 mmol, 1,800 mmol, 1,900 mmol, 2,000 mmol, 2,100 mmol, 2,200 mmol, 2,300 mmol, 2,400 mmol, 2,500 mmol, 2,600 mmol, 2,700 mmol, 2,800 mmol, 2,900 mmol, 3,000 mmol, 3,400 mmol, 3,600 mmol, 4,000 mmol, 4,200 mmol, 4,500 mmol, 5,000 mmol, or 5,400 mmol.

In certain embodiments, the cleaved oligonucleotide is eluted from the solid support according to the method of preparing an oligonucleotide disclosed herein. In certain embodiments, the eluting of the cleaved oligonucleotide, for example, the cleaved oligonucleotide comprising a 5′-hydroxyl protected group on the terminal nucleoside, from the solid support comprises washing the support with water, for example with an aqueous solution or a buffered aqueous solution.

In certain embodiments, the eluting of the cleaved oligonucleotide from the solid support provides the oligonucleotide, such as the cleaved oligonucleotide comprising a 5′-hydroxyl protected group on the terminal nucleoside, in an amount (such as a theoretical amount based on the synthesis scale or loading capacity of the solid support having a linker attached thereto) in the range of between 300-5,400 mmol, for example, between 300-4,500 mmol, 300-4,000 mmol, 300-3,600 mmol, 300-3,000 mmol, 600-3,000 mmol, 700-3,600 mmol, 700-2,700 mmol, 700-2,500 mmol, 700-2,400 mmol, 700-2,000 mmol, 700-1,900 mmol, 700-1,800 mmol, 700-1,700 mmol, 700-1,600 mmol, 700-1,500 mmol, 700-900 mmol, 800-1,200 mmol, 800-1,000 mmol, 900-1,600 mmol, 900-1,800 mmol, 900-2,400 mmol, 900-2,700 mmol, 900-3,600 mmol, 1,800-2,700 mmol, 1,800-3,600 mmol, 1,000-3,000 mmol, 1,000-2,500 mmol, 1,000-2,000 mmol, 1,000-1,500 mmol, 1,250-1,750 mmol, 1,500-3,000 mmol, 1,500-2,500 mmol, 1,500-2,000 mmol, 1,600-2,400 mmol, 2,000-3,000 mmol, 2,500-3,000 mmol, 2,700-3,600 mmol, 2,700-4,500 mmol, 3,000-4,000 mmol, 3,600-4,500 mmol, or 3,600-5,400 mmol. In certain embodiments, the eluting of the cleaved oligonucleotide from the solid support provides the oligonucleotide, such as the cleaved oligonucleotide comprising a 5′-hydroxyl protected group on the terminal nucleoside, in an amount (such as a theoretical amount based on the synthesis scale or loading capacity of the solid support having a linker attached thereto) of 300 mmol, 400 mmol, 500 mmol, 600 mmol, 700 mmol, 800 mmol, 900 mmol, 1,000 mmol, 1,100 mmol, 1,200 mmol, 1,300 mmol, 1,400 mmol, 1,500 mmol, 1,600 mmol, 1,700 mmol, 1,800 mmol, 1,900 mmol, 2,000 mmol, 2,100 mmol, 2,200 mmol, 2,300 mmol, 2,400 mmol, 2,500 mmol, 2,600 mmol, 2,700 mmol, 2,800 mmol, 2,900 mmol, 3,000 mmol, 3,400 mmol, 3,600 mmol, 4,000 mmol, 4,200 mmol, 4,500 mmol, 5,000 mmol, or 5,400 mmol.

5.12 Ion Exchange Purification

In certain embodiments, the oligonucleotide eluate comprising the cleaved oligonucleotide from the solid support is purified using ion exchange chromatography according to the method of preparing an oligonucleotide disclosed herein. The capacity or purification scale of an ion-exchange column utilized to purify an oligonucleotide by ion-exchange chromatography according to the methods disclosed herein, refers to the molar amount of ion-exchange capacity available in the ion-exchange column to purify the oligonucleotide. Accordingly, the amount of the oligonucleotide purified that is equivalent to the capacity or purification scale of the ion-exchange column utilized is considered the theoretical amount. The actual amount of the oligonucleotide purified according to the methods disclosed herein utilizing the ion-exchange column may be identical to the theoretical amount, or may be less than the theoretical amount. For example, in certain embodiments, the actual amount of the oligonucleotide purified according to the methods disclosed herein utilizing the ion-exchange column is less than the theoretical amount, for example is 95% of the theoretical amount, such as 90%, 85%, 80%, 75%, 70%, 65%, 60%, 55%, or 50% of the theoretical amount, or for example, is between 40-98%, 40-95%, 40-90%, 40-85%, 40-80%, 40-75%, 40-70%, 50-98%, 50-95%, 50-90%, 50-85%, 50-80%, 50-75%, 50-70%, 60-98%, 60-95%, 60-90%, 60-85%, 60-80%, 60-75%, or 60-70% of the theoretical amount.

In certain embodiments, the purifying of the cleaved oligonucleotide eluate, for example, the cleaved oligonucleotide eluate comprising a 5′-hydroxyl protected group on the terminal nucleoside, using ion exchange chromatography, for example anion exchange chromatography, comprises utilizing a salt gradient, for example, a sodium chloride gradient, or utilizing a basic salt gradient, for example, a basic sodium chloride gradient. In certain embodiments, the purifying step via ion exchange chromatography comprises loading the cleaved oligonucleotide eluate directly onto the ion exchange chromatography column, or alternatively, diluting the cleaved oligonucleotide eluate with an aqueous buffer prior to loading onto the ion exchange chromatography column. In certain embodiments, the 5′-hydroxyl group of the terminal nucleoside of the cleaved oligonucleotide contained within the cleaved oligonucleotide eluate to be purified remains protected during the loading step onto the ion exchange column. In certain embodiments, the 5′-hydroxyl group of the terminal nucleoside of the cleaved oligonucleotide contained within the cleaved oligonucleotide eluate loaded onto the ion exchange column is the only group protected on said loaded oligonucleotide eluate. For example, in certain embodiments, the 5′-hydroxyl protecting group of the cleaved oligonucleotide, such as the cleaved oligonucleotide loaded onto the ion exchange column, can include trityl or dimethoxytrityl (DMT). In certain embodiments, the 5′-hydroxyl protecting group of the cleaved oligonucleotide or the loaded, cleaved oligonucleotide is dimethoxytrityl (DMT).

In certain embodiments, the amount of the cleaved oligonucleotide contained within the cleaved oligonucleotide eluate that is loaded onto the ion exchange column, such as the cleaved oligonucleotide comprising a 5′-hydroxyl protected group on the terminal nucleoside, may be of 300 mmol or greater, for example, in an amount of 600 mmol or greater, 700 mmol or greater, 800 mmol or greater, 900 mmol or greater, 1,000 mmol or greater, 1,100 mmol or greater, 1,200 mmol or greater, 1,300 mmol or greater, 1,400 mmol or greater, 1,500 mmol or greater, 1,600 mmol or greater, 1,700 mmol or greater, 1,800 mmol or greater, 1,900 mmol or greater, 2,000 mmol or greater, 2,100 mmol or greater, 2,200 mmol or greater, 2,300 mmol or greater, 2,400 mmol or greater, 2,500 mmol or greater, 2,600 mmol or greater, 2,700 mmol or greater, 2,800 mmol or greater, 2,900 mmol or greater, 3,000 mmol or greater, 3,200 mmol or greater, 3,400 mmol or greater, 3,600 mmol or greater, 4,000 mmol or greater, 4,200 mmol or greater, 4,500 mmol or greater, 5,000 mmol or greater, or 5,400 mmol or greater, or for example, may be in the range of between 300-5,400 mmol (e.g., in terms of purification scale), for example, between 300-4,500 mmol, 300-4,000 mmol, 300-3,600 mmol, 300-3,000 mmol, 600-3,000 mmol, 700-3,600 mmol, 700-2,700 mmol, 700-2,500 mmol, 700-2,400 mmol, 700-2,000 mmol, 700-1,900 mmol, 700-1,800 mmol, 700-1,700 mmol, 700-1,600 mmol, 700-1,500 mmol, 700-900 mmol, 800-1,200 mmol, 800-1,000 mmol, 900-1,600 mmol, 900-1,800 mmol, 900-2,400 mmol, 900-2,700 mmol, 900-3,600 mmol, 1,800-2,700 mmol, 1,800-3,600 mmol, 1,000-3,000 mmol, 1,000-2,500 mmol, 1,000-2,000 mmol, 1,000-1,500 mmol, 1,250-1,750 mmol, 1,500-3,000 mmol, 1,500-2,500 mmol, 1,500-2,000 mmol, 1,600-2,400 mmol, 2,000-3,000 mmol, 2,500-3,000 mmol, 2,700-3,600 mmol, 2,700-4,500 mmol, 3,000-4,000 mmol, 3,600-4,500 mmol, or 3,600-5,400 mmol. In certain embodiments, the amount of the cleaved oligonucleotide contained within the cleaved oligonucleotide eluate that is loaded onto the ion exchange column, such as the cleaved oligonucleotide comprising a 5′-hydroxyl protected group on the terminal nucleoside, may be in an amount of 300 mmol (e.g., in terms of purification scale), such as 400 mmol, 500 mmol, 600 mmol, 700 mmol, 800 mmol, 900 mmol, 1,000 mmol, 1,100 mmol, 1,200 mmol, 1,300 mmol, 1,400 mmol, 1,500 mmol, 1,600 mmol, 1,700 mmol, 1,800 mmol, 1,900 mmol, 2,000 mmol, 2,100 mmol, 2,200 mmol, 2,300 mmol, 2,400 mmol, 2,500 mmol, 2,600 mmol, 2,700 mmol, 2,800 mmol, 2,900 mmol, 3,000 mmol, 3,400 mmol, 3,600 mmol, 4,000 mmol, 4,200 mmol, 4,500 mmol, 5,000 mmol, or 5,400 mmol.

In certain embodiments, the amount of the cleaved oligonucleotide contained within the cleaved oligonucleotide eluate that is loaded onto the ion exchange column, such as the cleaved oligonucleotide comprising a 5′-hydroxyl protected group on the terminal nucleoside, is an amount that is pooled from at least 1, 2, 3, 4, 5, 6, 7, 8, 9, or 10, oligonucleotide synthesis columns (or synthesis runs utilizing one or more synthesis columns), or for example, is an amount that is pooled from between 1-10 oligonucleotide synthesis columns (or synthesis runs utilizing one or more synthesis columns), such as between 1-8, between 2-10, between 3-9, between 4-7, between 4-6, between 6-10, or between 8-10, oligonucleotide synthesis columns (or synthesis runs utilizing one or more synthesis columns). In certain embodiments, the independent amount of the cleaved oligonucleotide resulting from a single, a plurality, or each, of the oligonucleotide synthesis column(s) (or synthesis run(s) utilizing one or more synthesis column(s)), that is to be pooled prior to loading onto or purifying via an ion exchange column, may be in an amount of 300 mmol or greater of the cleaved oligonucleotide, for example, may be in an amount of 600 mmol or greater, 700 mmol or greater, 800 mmol or greater, 900 mmol or greater, 1,000 mmol or greater, 1,100 mmol or greater, 1,200 mmol or greater, 1,300 mmol or greater, 1,400 mmol or greater, 1,500 mmol or greater, 1,600 mmol or greater, 1,700 mmol or greater, 1,800 mmol or greater, 1,900 mmol or greater, 2,000 mmol or greater, 2,100 mmol or greater, 2,200 mmol or greater, 2,300 mmol or greater, 2,400 mmol or greater, 2,500 mmol or greater, 2,600 mmol or greater, 2,700 mmol or greater, 2,800 mmol or greater, 2,900 mmol or greater, 3,000 mmol or greater, 3,200 mmol or greater, 3,400 mmol or greater, 3,600 mmol or greater, 4,000 mmol or greater, 4,200 mmol or greater, 4,500 mmol or greater, 5,000 mmol or greater, or 5,400 mmol or greater, of the cleaved oligonucleotide, or for example, may be in the range of between 300-5,400 mmol, for example, in the range between 300-4,500 mmol, 300-4,000 mmol, 600-3,000 mmol, 700-3,600 mmol, 700-3,000 mmol, 700-2,700 mmol, 700-2,400 mmol, 700-2,000 mmol, 700-1,500 mmol, 700-1,000 mmol, 700-900 mmol, 800-1,000 mmol, 900-1,600 mmol, 900-2,400 mmol, 1,000-3,000 mmol, 1,000-2,000 mmol, 1,500-2,000 mmol, 1,600-2,400 mmol, 1,500-2,500 mmol, 1,500-3,000 mmol, 2,000-3,000 mmol, 2,000-2,500 mmol, 2,500-3,000 mmol, 2,600-2,800 mmol, 2,700-3,000 mmol, 900-2,000 mmol, 900-2,500 mmol, 900-2,700 mmol, 900-3,600 mmol, 1,000-4,000 mmol, 1,500-3,500 mmol, 1,800-3,600 mmol, 2,000-4,000 mmol, 2,500-3,500 mmol, 2,700-3,600 mmol, 2,700-4,500 mmol, 3,000-4,000 mmol, 3,600-4,500 mmol, or 3,600-5,400 mmol, of the cleaved oligonucleotide.

In certain embodiments, a single, a plurality, or each, of the ion exchange column(s) utilized in the methods disclosed herein has a loading capacity (or ion-exchange capacity) sufficient to provide the purified and fully deprotected oligonucleotide in an amount in the range of 300-5,400 mmol, for example, provide the purified and fully deprotected oligonucleotide in an amount in the range of 300-4,500 mmol, 300-4,000 mmol, 300-3,600 mmol, 300-3,000 mmol, 600-3,000 mmol, 700-2,500 mmol, 700-2,400 mmol, 700-2,000 mmol, 700-1,900 mmol, 700-1,800 mmol, 700-1,700 mmol, 700-1,600 mmol, 700-1,500 mmol, 700-1,400 mmol, 700-1,300 mmol, 700-1,200 mmol, 700-1,100 mmol, 700-1,000 mmol, 700-900 mmol, 800-1,200 mmol, 800-1,000 mmol, 900-1,600 mmol, 900-1,800 mmol, 900-2,400 mmol, 900-2,700 mmol, 900-3,600 mmol, 1,800-2,700 mmol, 1,800-3,600 mmol, 1,000-3,000 mmol, 1,000-2,500 mmol, 1,000-2,000 mmol, 1,000-1,500 mmol, 1,250-1,750 mmol, 1,500-3,000 mmol, 1,500-2,500 mmol, 1,500-2,000 mmol, 1,600-2,400 mmol, 2,000-3,000 mmol, 2,500-3,000 mmol, 2,700-3,600 mmol, 2,700-4,500 mmol, 3,000-4,000 mmol, 3,600-4,500 mmol, or 3,600-5,400 mmol. In certain embodiments, a single, a plurality, or each, of the ion exchange column(s) utilized in the methods disclosed herein has a loading capacity sufficient to provide the purified and fully deprotected oligonucleotide in an amount of at least 300 mmol, for example, provide the purified and fully deprotected oligonucleotide in an amount at least 300 mmol, such as at least 400 mmol, at least 500 mmol, at least 600 mmol, at least 700 mmol, at least 800 mmol, at least 900 mmol, at least 1,000 mmol, at least 1,100 mmol, at least 1,200 mmol, at least 1,300 mmol, at least 1,400 mmol, at least 1,600 mmol, at least 1,800 mmol, at least 2,400 mmol, at least 2,700 mmol, at least 3,000 mmol, at least 3,600 mmol, or at least 4,500 mmol. In certain embodiments, a single, a plurality, or each, of the ion exchange column(s) utilized in the methods disclosed herein has a loading capacity (or ion-exchange capacity) sufficient to provide the purified and fully deprotected oligonucleotide in an amount of 300 mmol, such as 400 mmol, 500 mmol, 600 mmol, 700 mmol, 800 mmol, 900 mmol, 1,000 mmol, 1,100 mmol, 1,200 mmol, 1,300 mmol, 1,400 mmol, 1,500 mmol, 1,600 mmol, 1,700 mmol, 1,800 mmol, 1,900 mmol, 2,000 mmol, 2,100 mmol, 2,200 mmol, 2,300 mmol, 2,400 mmol, 2,500 mmol, 2,600 mmol, 2,700 mmol, 2,800 mmol, 2,900 mmol, 3,000 mmol, 3,100 mmol, 3,200 mmol, 3,300 mmol, 3,400 mmol, 3,500 mmol, 3,600 mmol, 4,000 mmol, 4,200 mmol, 4,500 mmol, 5,000 mmol, or 5,400 mmol.

In certain embodiments, the purification step using ion exchange chromatography according to the method of preparing an oligonucleotide disclosed herein, is anion exchange chromatography and comprises utilizing a salt gradient, such a sodium chloride gradient. In certain embodiments, the ion exchange chromatography column comprises using Q Sepharose FF as the support media.

In certain embodiments, the cleaved oligonucleotide eluate is loaded onto the ion exchange chromatography column and then initially washed with a basic solution, for example, initially washed with a sodium hydroxide solution, to complete the loading step onto the ion exchange column.

In certain embodiments, the cleaved oligonucleotide eluate is loaded onto the ion exchange chromatography column, followed by washing the cleaved oligonucleotide-loaded column, for example, washing the cleaved oligonucleotide-loaded column with a basic solution, such as, followed by initially washing the cleaved oligonucleotide-loaded column with a sodium hydroxide solution, and then subsequently washing the cleaved oligonucleotide-loaded column with a salt gradient, for example, a basic salt gradient, such as a gradient comprising varying amounts of a sodium chloride solution and sodium hydroxide solution, to complete the loading step onto the ion exchange column.

In certain embodiments, the cleaved oligonucleotide eluate is loaded onto the ion exchange chromatography column, followed by washing the cleaved oligonucleotide-loaded column, for example, washing the cleaved oligonucleotide-loaded column with a basic solution, such as, followed by initially washing the cleaved oligonucleotide-loaded column with a sodium hydroxide solution, subsequently washing the cleaved oligonucleotide-loaded column with a salt gradient, for example, a basic salt gradient, such as a gradient comprising varying amounts of a sodium chloride solution and sodium hydroxide solution, and then further washing the cleaved oligonucleotide-loaded column with a basic solution, for example, further washing the cleaved oligonucleotide-loaded column with a sodium hydroxide solution, to complete the loading step onto the ion exchange column.

In certain embodiments, the protecting hydroxyl group on the terminal nucleoside of the cleaved oligonucleotide loaded onto the ion exchange column is deprotected, for example, the 5′-hydroxyl protected group on the terminal nucleoside of the cleaved oligonucleotide loaded onto the anion exchange column is deprotected and then subsequently washed, according to the method of preparing an oligonucleotide disclosed herein. For example, in certain embodiments, deprotection of the 5′-hydroxyl protected group from the terminal nucleoside of the cleaved oligonucleotide loaded onto the ion exchange column may be effected by introducing a protic acid, such as an acetic acid solution, for example an 80% aqueous acetic acid solution, to the ion exchange column. In certain embodiments, deprotection of the 5′-hydroxyl protected group from the terminal nucleoside of the cleaved oligonucleotide loaded onto the ion exchange column may be effected by providing a protic acid solution, such as an acetic acid solution, for example an 80% aqueous acetic acid solution, to the ion exchange column, holding the flow of the protic acid solution for a period of time, and then reinitiating the flow of the solution through the ion exchange column to complete the deprotection of the 5′-hydroxyl protected group from the terminal nucleoside of the cleaved oligonucleotide loaded onto the ion exchange column. In certain embodiments, the volume of the protic acid utilized to deprotect the 5′-hydroxyl protected group from the terminal nucleoside of the cleaved oligonucleotide loaded onto the ion exchange column may be at least 10 L, for example, in the range of between 10-40 L, such as between 20-40 L, 10-30 L, 25-35 L, or 15-25 L. In certain embodiments, the volume of the protic acid utilized to deprotect the 5′-hydroxyl protected group from the terminal nucleoside of the cleaved oligonucleotide loaded onto the ion exchange column may be at least 1 column volume, for example, in the range of between 1-5 column volumnes, such as between 1-3, 2-4, 3-5, or 1-4 column volumes. In certain embodiments, the 5′-hydroxyl protecting group of the cleaved oligonucleotide loaded onto the ion exchange column that is deprotected is trityl or dimethoxytrityl (DMT). In certain embodiments, the resulting deprotected oligonucleotide remaining on the ion exchange column is devoid of protecting groups.

In certain embodiments, the loaded and fully deprotected oligonucleotide on the ion exchange column is first neutralized with a basic solution, for example, with a 25-200 mM sodium hydroxide solution, such as with a 0.1 M sodium hydroxide solution, prior to elution from the ion exchange column with a salt gradient.

In certain embodiments, the resulting deprotected oligonucleotide remaining on the ion exchange column, such as an anion exchange column, is eluted from the column using a salt gradient, according to the method of preparing an oligonucleotide disclosed herein. For example, in certain embodiments, the salt gradient utilized to elute the resulting deprotected oligonucleotide from the ion exchange column may be a sodium chloride gradient, for example a basic salt gradient, such as a gradient comprising varying amounts of a sodium chloride solution and sodium hydroxide solution. In certain embodiments, the resulting deprotected oligonucleotide remaining on the ion exchange column, is first washed with water, then eluted from the ion exchange chromatography column using a salt gradient, according to the method of preparing an oligonucleotide disclosed herein. In certain embodiments, the resulting deprotected oligonucleotide remaining on the ion exchange column, is first washed with water, secondly, washed with a basic solution, such as an aqueous sodium hydroxide solution, to adjust the pH of the column, and thirdly, eluted from the ion exchange chromatography column using a salt gradient, such a sodium chloride gradient or a basic salt gradient, such as a gradient comprising varying amounts of a sodium chloride solution and sodium hydroxide solution, thereby completing the elution of the resulting deprotected oligonucleotide from the ion exchange column. In certain embodiments, the salt gradient utilized to elute the resulting deprotected oligonucleotide from the ion exchange column ranges from 0.1 to 2 M of an aqueous salt solution, for example, a basic aqueous salt solution, such as a salt gradient ranging from 0.1 to 2 M of an aqueous basic solution comprising sodium chloride, such as a salt gradient ranging from 0.2 to 1.8 M sodium chloride in an aqueous basic solution. In certain embodiments, the salt gradient utilized to elute the resulting deprotected oligonucleotide from the ion exchange column ranges from 0.1 to 2 M sodium chloride in a 10-50 nM aqueous sodium hydroxide solution, such as a salt gradient from 0.2 to 1.8 M sodium chloride in an 10-50 nM aqueous sodium hydroxide solution. In certain embodiments, the resulting oligonucleotide eluted from the ion exchange column is a fully deprotected oligonucleotide.

In certain embodiments, the amount of the fully deprotected oligonucleotide eluted from the ion exchange column may be in the range of between 300-5,400 mmol, for example, in the range of between 300-4,500 mmol, 300-4,000 mmol, 300-3,600 mmol, 300-3,000 mmol, 600-3,000 mmol, 700-3,600 mmol, 700-2,700 mmol, 700-2,500 mmol, 700-2,400 mmol, 700-2,000 mmol, 700-1,900 mmol, 700-1,800 mmol, 700-1,700 mmol, 700-1,600 mmol, 700-1,500 mmol, 700-900 mmol, 800-1,200 mmol, 800-1,000 mmol, 900-1,600 mmol, 900-1,800 mmol, 900-2,400 mmol, 900-2,700 mmol, 900-3,600 mmol, 1,800-2,700 mmol, 1,800-3,600 mmol, 1,000-3,000 mmol, 1,000-2,500 mmol, 1,000-2,000 mmol, 1,000-1,500 mmol, 1,250-1,750 mmol, 1,500-3,000 mmol, 1,500-2,500 mmol, 1,500-2,000 mmol, 1,600-2,400 mmol, 2,000-3,000 mmol, 2,500-3,000 mmol, 2,700-3,600 mmol, 2,700-4,500 mmol, 3,000-4,000 mmol, 3,600-4,500 mmol, or 3,600-5,400 mmol. In certain embodiments, the amount of the fully deprotected oligonucleotide eluted from the ion exchange column may be in an amount of 300 mmol, such as 400 mmol, 500 mmol, 600 mmol, 700 mmol, 800 mmol, 900 mmol, 1,000 mmol, 1,100 mmol, 1,200 mmol, 1,300 mmol, 1,400 mmol, 1,500 mmol, 1,600 mmol, 1,700 mmol, 1,800 mmol, 1,900 mmol, 2,000 mmol, 2,100 mmol, 2,200 mmol, 2,300 mmol, 2,400 mmol, 2,500 mmol, 2,600 mmol, 2,700 mmol, 2,800 mmol, 2,900 mmol, 3,000 mmol, 3,400 mmol, 3,600 mmol, 4,000 mmol, 4,200 mmol, 4,500 mmol, 5,000 mmol, or 5,400 mmol.

In certain embodiments, the overall yield of preparing an oligonucleotide according to the method disclosed herein, beginning from synthesis through collected oligonucleotide from the purification step using ion exchange chromatography, is at least 50%, as determined by optical density/mL (OD/mL) at a wavelength at 260 nm, for example, in the range of between 55-100%, such as between 55-95%, 55-90%, 55-85%, 55-80%, 55-75%, 60-100%, 60-90%, 60-80%, 50-75%, 70-100%, 80-90%, 85-95%, 90-100%, or 95-100%, as determined by OD/mL at a wavelength at 260 nm. In certain embodiments, the purity the oligonucleotide prepared according to the method disclosed herein, beginning from synthesis through collected oligonucleotide from the purification step using ion exchange chromatography, is at least 50%, for example, purity in the range of between 50-100%, such as between 50-95%, 50-85%, 50-75%, 60-90%, 60-80%, 80-100%, 90-100%, 85-95%, or 95-100%, as determined by RP-HPLC.

5.13 Ultrafiltration and/or Diafiltration (UFDF) Process

In certain embodiments, the eluate comprising the fully deprotected oligonucleotide from the ion exchange column is desalted via an ultrafiltration and/or diafiltration process, according to the method of preparing an oligonucleotide disclosed herein. In certain embodiments, the eluate comprising the fully deprotected oligonucleotide resulting from the ion exchange column is optionally neutralized with a dilute basic solution, such as a dilute sodium hydroxide solution, or with a dilute acid solution, such as a dilute hydrochloric acid solution, or with combination of each to adjust the pH to neutrality (e.g., a pH in the range of 6-8, such as 6.5-7.5), prior to desalting via the ultrafiltration and/or diafiltration processes, according to the method of preparing an oligonucleotide disclosed herein. For example, the eluate comprising the fully deprotected oligonucleotide resulting from the ion exchange column may be optionally neutralized with a dilute basic solution, such as a dilute sodium hydroxide solution (e.g., 0.1-1 M sodium hydroxide), or with a dilute acid solution, such as a dilute hydrochloric acid solution (e.g., 0.1-1 M hydrochloric acid), or with combination of each to adjust the pH towards neutrality (e.g., a pH in the range of 6-8, such as 6.5-7.5). In certain embodiments, the eluate comprising the fully deprotected oligonucleotide resulting from the ion exchange column (which may be optionally neutralized) is desalted via an ultrafiltration and/or diafiltration process, followed by concentrating the resulting desalted fully deprotected oligonucleotide retentate solution, such as concentrating by thin film evaporation, or concentrated by thin film evaporation followed by freeze drying, according to the method of preparing an oligonucleotide disclosed herein.

In certain embodiments, the process of desalting the fully deprotected oligonucleotide via an ultrafiltration and/or diafiltration process, for example, desalting prior to concentrating (e.g., such as concentrating by thin film evaporation), utilizes a water solution at a pH in the range of 5-8 to provide a desalted fully deprotected oligonucleotide retentate solution, for example, utilizes a water solution at a pH in the range of 5.5-8, such as 6-8, 6.5-8, 6.8-8, 5.5-7.5, 6-7.5, 6.5-7.5, 6.8-7.5, 5.5-7, 6-7, 6.5-7, 6.8-8, 6.8-7.5, or 6.8-7.3, to provide a desalted fully deprotected oligonucleotide retentate solution. In certain embodiments, the desalting step begins by utilizing a water solution at a pH in the range of 6.8-7.3, and ends the desalting step by utilizing a water solution at a pH in the range of 6.5-7.5, to provide a desalted fully deprotected oligonucleotide retentate solution. In certain embodiments, the process of desalting the fully deprotected oligonucleotide via an ultrafiltration and/or diafiltration process utilizes distilled water, or distilled, de-ionized water, to provide a desalted fully deprotected oligonucleotide retentate solution.

In certain embodiments, the process of desalting the eluate comprising the fully deprotected oligonucleotide via an ultrafiltration and/or diafiltration process, for example, desalting prior to concentrating (e.g., such as concentrating by thin film evaporation), utilizes a regenerated cellulose membrane to accomplish the desalting process, such as a regenerated cellulose membrane having a 1,000-3,000 Da molecular weight cutoff.

In certain embodiments, the effectiveness of desalting the fully deprotected oligonucleotide eluate via an ultrafiltration and/or diafiltration process is determined by conductivity measurements of the resulting permeate (i.e., the filtrate from the ultrafiltration and/or diafiltration process) as a means of determining the salt concentration of the resulting desalted fully deprotected oligonucleotide retentate solution. For example, in certain embodiments, the conductivity of the resulting permeate (sometimes referred to as diafiltrate) from desalting the fully deprotected oligonucleotide eluate via an ultrafiltration and/or diafiltration process is less than 900 uS/cm, such as less than 800 uS/cm, less than 700 uS/cm, less than 600 uS/cm, less than 500 uS/cm, less than 400 uS/cm, less than 300 uS/cm, less than 200 uS/cm, less than 100 uS/cm, or less than 75 uS/cm. In certain embodiments, the conductivity of the resulting permeate from desalting the fully deprotected oligonucleotide eluate via an ultrafiltration and/or diafiltration process is in the range of between 40-900 uS/cm, for example, between 40-850 uS/cm, 40-750 uS/cm, 40-650 uS/cm, 40-550 uS/cm, 40-450 uS/cm, 40-350 uS/cm, 40-250 uS/cm, 40-150 uS/cm, 40-100 uS/cm, or 40-75 uS/cm. In certain embodiments, the effectiveness of desalting the fully deprotected oligonucleotide eluate via an ultrafiltration and/or diafiltration process results in bringing the sodium content of the resulting desalted fully deprotected oligonucleotide retentate solution to a level in the range of between 6-8 wt. %, for example, between 6-7 wt. %, 7-8 wt. %, or 6.5-7.5 wt. %.

In certain embodiments, the amount of the fully deprotected oligonucleotide in the eluate from the ion exchange column that is desalted via an ultrafiltration and/or diafiltration process, for example, that is desalted prior to concentrating (e.g., such as concentrating by thin film evaporation), may be in the range of between 300-5,400 mmol, for example, in the range of between 300-4,500 mmol, 300-4,000 mmol, 300-3,600 mmol, 300-3,000 mmol, 600-3,000 mmol, 700-3,600 mmol, 700-2,700 mmol, 700-2,500 mmol, 700-2,400 mmol, 700-2,000 mmol, 700-1,900 mmol, 700-1,800 mmol, 700-1,700 mmol, 700-1,600 mmol, 700-1,500 mmol, 700-900 mmol, 800-1,200 mmol, 800-1,000 mmol, 900-1,600 mmol, 900-1,800 mmol, 900-2,400 mmol, 900-2,700 mmol, 900-3,600 mmol, 1,800-2,700 mmol, 1,800-3,600 mmol, 1,000-3,000 mmol, 1,000-2,500 mmol, 1,000-2,000 mmol, 1,000-1,500 mmol, 1,250-1,750 mmol, 1,500-3,000 mmol, 1,500-2,500 mmol, 1,600-2,400 mmol, 1,500-2,000 mmol, 2,000-3,000 mmol, 2,500-3,000 mmol, 2,700-3,600 mmol, 2,700-4,500 mmol, 3,000-4,000 mmol, 3,600-4,500 mmol, or 3,600-5,400 mmol. In certain embodiments, the amount of the fully deprotected oligonucleotide in the eluate from the ion exchange column that is desalted via an ultrafiltration and/or diafiltration process, for example, that is desalted prior to concentrating (e.g., such as concentrating by thin film evaporation), may be in an amount of 300 mmol, 400 mmol, 500 mmol, 600 mmol, 700 mmol, 800 mmol, 900 mmol, 1,000 mmol, 1,100 mmol, 1,200 mmol, 1,300 mmol, 1,400 mmol, 1,500 mmol, 1,600 mmol, 1,700 mmol, 1,800 mmol, 1,900 mmol, 2,000 mmol, 2,100 mmol, 2,200 mmol, 2,300 mmol, 2,400 mmol, 2,500 mmol, 2,600 mmol, 2,700 mmol, 2,800 mmol, 2,900 mmol, 3,000 mmol, 3,400 mmol, 3,600 mmol, 4,000 mmol, 4,200 mmol, 4,500 mmol, 5,000 mmol, or 5,400 mmol.

In certain embodiments, the amount of the fully deprotected oligonucleotide following purification via ion exchange chromatography that is subjected to desalting via an ultrafiltration and/or diafiltration process, for example, that is subjected to desalting prior to concentrating (e.g., such as concentrating by thin film evaporation), is an amount that is pooled from at least 1, 2, 3, 4, 5, 6, 7, 8, 9, or 10, ion exchange chromatography purification columns (or purification runs utilizing one or more ion exchange chromatography purification columns), or for example, is an amount that is pooled from between 1-10 ion exchange chromatography purification columns (or purification runs utilizing one or more ion exchange chromatography purification columns), such as between 1-8, between 2-10, between 3-9, between 4-7, between 4-6, between 6-10, or between 8-10, ion exchange chromatography purification columns (or purification runs utilizing one or more ion exchange chromatography purification columns). In certain embodiments, the independent amount of the fully deprotected and purified oligonucleotide resulting from a single, a plurality, or each, of the ion exchange chromatography purification column(s) (or purification run(s) utilizing one or more ion exchange chromatography purification column(s)), that is to be pooled prior to desalting via an ultrafiltration and/or diafiltration process, may be in an amount of 700 mmol or greater of the fully deprotected and purified oligonucleotide, for example, may be in an amount of 800 mmol or greater, 900 mmol or greater, 1,000 mmol or greater, 1,100 mmol or greater, 1,200 mmol or greater, 1,300 mmol or greater, 1,400 mmol or greater, 1,500 mmol or greater, 1,600 mmol or greater, 1,700 mmol or greater, 1,800 mmol or greater, 1,900 mmol or greater, 2,000 mmol or greater, 2,100 mmol or greater, 2,200 mmol or greater, 2,300 mmol or greater, 2,400 mmol or greater, 2,500 mmol or greater, 2,600 mmol or greater, 2,700 mmol or greater, 2,800 mmol or greater, 2,900 mmol or greater, 3,000 mmol or greater, 3,200 mmol or greater, 3,400 mmol or greater, 3,600 mmol or greater, 4,000 mmol or greater, 4,200 mmol or greater, 4,500 mmol or greater, 5,000 mmol or greater, or 5,400 mmol or greater, of the fully deprotected and purified oligonucleotide, or for example, may be an amount in the range of between 700-5,400 mmol, such as between 700-4,500 mmol, 700-4,000 mmol, 800-4,000 mmol, 900-4,000 mmol, 900-3,000 mmol, 900-3,600 mmol, 900-3,000 mmol, 900-2,700 mmol, 900-2,500 mmol, 900-2,400 mmol, 900-2,000 mmol, 900-1,600 mmol, 900-1,500 mmol, 900-1,000 mmol, 700-1,000 mmol, 1,000-3,000 mmol, 1,000-2,000 mmol, 1,500-2,000 mmol, 1,600-2,400 mmol, 1,500-2,700 mmol, 1,500-3,000 mmol, 2,000-3,000 mmol, 2,000-2,500 mmol, 2,500-3,000 mmol, 2,600-2,800 mmol, 2,700-3,000 mmol, 2,700-3,600 mmol, 1,000-4,000 mmol, 1,500-3,500 mmol, 2,000-4,000 mmol, 2,500-3,500 mmol, 2,700-4,500 mmol, 3,000-4,000 mmol, 3,600-4,500 mmol, or 3,600-5,400 mmol, of the fully deprotected and purified oligonucleotide.

In certain embodiments, the ultrafiltration and/or diafiltration process utilized in the methods disclosed herein has a loading capacity sufficient to provide the desalted fully deprotected oligonucleotide in an amount in the range of 300-5,400 mmol, for example, provide the desalted fully deprotected oligonucleotide in an amount in the range of 300-4,500 mmol, 300-4,000 mmol, 300-3,600 mmol, 300-3,000 mmol, 600-3,000 mmol, 700-3,600 mmol, 700-2,700 mmol, 700-2,500 mmol, 700-2,400 mmol, 700-2,000 mmol, 700-1,900 mmol, 700-1,800 mmol, 700-1,700 mmol, 700-1,600 mmol, 700-1,500 mmol, 700-1,400 mmol, 700-1,300 mmol, 700-1,200 mmol, 700-1,100 mmol, 700-1,000 mmol, 700-900 mmol, 800-1,200 mmol, 800-1,000 mmol, 900-1,600 mmol, 900-1,800 mmol, 900-2,400 mmol, 900-2,700 mmol, 900-3,600 mmol, 1,800-2,700 mmol, 1,800-3,600 mmol, 1,000-3,000 mmol, 1,000-2,500 mmol, 1,000-2,000 mmol, 1,000-1,500 mmol, 1,250-1,750 mmol, 1,500-3,000 mmol, 1,500-2,500 mmol, 1,500-2,000 mmol, 1,600-2,400 mmol, 2,000-3,000 mmol, 2,500-3,000 mmol, 2,700-3,600 mmol, 2,700-4,500 mmol, 3,000-4,000 mmol, 3,600-4,500 mmol, or 3,600-5,400 mmol. In certain embodiments, the ultrafiltration and/or diafiltration process utilized in the methods disclosed herein has a loading capacity sufficient to provide the desalted fully deprotected oligonucleotide in an amount of at least 300 mmol, for example, provide the desalted fully deprotected oligonucleotide in an amount at least 300 mmol, such as at least 400 mmol, at least 500 mmol, at least 600 mmol, at least 700 mmol, at least 800 mmol, at least 900 mmol, at least 1,000 mmol, at least 1,100 mmol, at least 1,200 mmol, at least 1,300 mmol, at least 1,400 mmol, at least 1,600 mmol, at least 1,800 mmol, at least 2,400 mmol, at least 2,700 mmol, at least 3,000 mmol, at least 3,600 mmol, or at least 4,500 mmol. In certain embodiments, the ultrafiltration and/or diafiltration process utilized in the methods disclosed herein has a loading capacity sufficient to provide the desalted fully deprotected oligonucleotide in an amount of 300 mmol, 400 mmol, 500 mmol, 600 mmol, 700 mmol, 800 mmol, 900 mmol, 1,000 mmol, 1,100 mmol, 1,200 mmol, 1,300 mmol, 1,400 mmol, 1,500 mmol, 1,600 mmol, 1,700 mmol, 1,800 mmol, 1,900 mmol, 2,000 mmol, 2,100 mmol, 2,200 mmol, 2,300 mmol, 2,400 mmol, 2,500 mmol, 2,600 mmol, 2,700 mmol, 2,800 mmol, 2,900 mmol, 3,000 mmol, 3,100 mmol, 3,200 mmol, 3,300 mmol, 3,400 mmol, 3,500 mmol, 3,600 mmol, 4,000 mmol, 4,200 mmol, 4,500 mmol, 5,000 mmol, or 5,400 mmol.

5.14 Liquid Composition Concentration Process

In certain embodiments, the fully deprotected oligonucleotide eluate from the ion exchange column is concentrated, such as concentrated with a Thin Film Evaporation (TFE) process, according to the method of preparing an oligonucleotide disclosed herein. In certain embodiments, the desalted fully deprotected oligonucleotide retentate solution from the ultrafiltration and/or diafiltration process is concentrated, such as concentrated with a thin film evaporation process, according to the method of preparing an oligonucleotide disclosed herein.

In certain embodiments, for example, the concentrating of the fully deprotected oligonucleotide eluate, or concentrating the desalted fully deprotected oligonucleotide retentate solution from an ultrafiltration and/or diafiltration process, is by thin film evaporation utilizing a thin film evaporation jacket temperature of 30° or greater, for example, in the range of 30−95° C., for example, in the range of 30−90° C., 60-90° C., 60-85° C., 60-80° C., 60-75° C., 65-90° C., 65-85° C., 65-80° C., 65-75° C., 70-90° C., 70-85° C., 70-80° C., or 85-95° C., such as at a temperature of 30° C., 40° C., 50° C., 60° C., 65° C., 70° C., 75° C., 80° C., 85° C., 90°, or 95°. In certain embodiments, the concentrating of the fully deprotected oligonucleotide eluate, or concentrating the desalted fully deprotected oligonucleotide retentate solution from an ultrafiltration and/or diafiltration process, is by thin film evaporation at a pressure in the range of 5-100 Torr, for example, in the range of 20-80 Torr, 20-75 Torr, 20-70 Torr, 20-60 Torr, 20-50 Torr, 30-100 Torr, 30-80 Torr, 30-75 Torr, 30-65 Torr, 30-55 Torr, 30-50 Torr, or 40-50 Torr, such as at a pressure of 5 Torr, 10 Torr, 15 Torr, 20 Torr, 25 Torr, 30 Torr, 35 Torr, 40 Torr, 45 Torr, 50 Torr, 55 Torr, 60 Torr, 65 Torr, 70 Torr, 75 Torr, 80 Torr, 85 Torr, 90 Torr, 95 Torr, or 100 Torr. In certain embodiments, the concentrating of the fully deprotected oligonucleotide eluate, or concentrating the desalted fully deprotected oligonucleotide retentate solution from an ultrafiltration and/or diafiltration process, is by thin film evaporation utilizing a thin film evaporation jacket temperature in the range of 60-90° C. and at a pressure in the range of 20-80 Torr.

In certain embodiments, for example, the amount of the fully deprotected oligonucleotide contained in the eluate from an ion exchange purification, or the amount of the desalted fully deprotected oligonucleotide contained in the retentate solution from an ultrafiltration and/or diafiltration process, that is concentrated, such as concentrated by thin film evaporation, may be of 300 mmol or greater, for example, in an amount of 600 mmol or greater, 700 mmol or greater, 800 mmol or greater, 900 mmol or greater, 1,000 mmol or greater, 1,100 mmol or greater, 1,200 mmol or greater, 1,300 mmol or greater, 1,400 mmol or greater, 1,500 mmol or greater, 1,600 mmol or greater, 1,700 mmol or greater, 1,800 mmol or greater, 1,900 mmol or greater, 2,000 mmol or greater, 2,100 mmol or greater, 2,200 mmol or greater, 2,300 mmol or greater, 2,400 mmol or greater, 2,500 mmol or greater, 2,600 mmol or greater, 2,700 mmol or greater, 2,800 mmol or greater, 2,900 mmol or greater, 3,000 mmol or greater, 3,200 mmol or greater, 3,400 mmol or greater, 3,600 mmol or greater, 4,000 mmol or greater, 4,200 mmol or greater, 4,500 mmol or greater, 5,000 mmol or greater, or 5,400 mmol or greater, or for example, may be in the range of between 300-5,400 mmol, for example, in the range of 300-4,500 mmol, 300-4,000 mmol, 300-3,600 mmol, 300-3,000 mmol, 600-3,000 mmol, 700-3,600 mmol, 700-2,700 mmol, 700-2,500 mmol, 700-2,400 mmol, 700-2,000 mmol, 700-1,900 mmol, 700-1,800 mmol, 700-1,700 mmol, 700-1,600 mmol, 700-1,500 mmol, 700-900 mmol, 800-1,200 mmol, 800-1,000 mmol, 900-1,600 mmol, 900-1,800 mmol, 900-2,400 mmol, 900-2,700 mmol, 900-3,600 mmol, 1,800-2,700 mmol, 1,800-3,600 mmol, 1,000-3,000 mmol, 1,000-2,500 mmol, 1,000-2,000 mmol, 1,000-1,500 mmol, 1,250-1,750 mmol, 1,500-3,000 mmol, 1,500-2,500 mmol, 1,500-2,000 mmol, 1,600-2,400 mmol, 2,000-3,000 mmol, 2,500-3,000 mmol, 2,700-3,600 mmol, 2,700-4,500 mmol, 3,000-4,000 mmol, 3,600-4,500 mmol, or 3,600-5,400 mmol. In certain embodiments, the amount of the fully deprotected oligonucleotide contained in the eluate from an ion exchange purification, or the amount of the desalted fully deprotected oligonucleotide contained in the retentate solution from an ultrafiltration and/or diafiltration process, that is concentrated, such as concentrated by thin film evaporation, may be in an amount of 300 mmol, 400 mmol, 500 mmol, 600 mmol, 700 mmol, 800 mmol, 900 mmol, 1,000 mmol, 1,100 mmol, 1,200 mmol, 1,300 mmol, 1,400 mmol, 1,500 mmol, 1,600 mmol, 1,700 mmol, 1,800 mmol, 1,900 mmol, 2,000 mmol, 2,100 mmol, 2,200 mmol, 2,300 mmol, 2,400 mmol, 2,500 mmol, 2,600 mmol, 2,700 mmol, 2,800 mmol, 2,900 mmol, 3,000 mmol, 3,400 mmol, 3,600 mmol, 4,000 mmol, 4,200 mmol, 4,500 mmol, 5,000 mmol, or 5,400 mmol.

In certain embodiments, for example, the amount of the fully deprotected oligonucleotide contained in the eluate from an ion exchange purification, or the amount of the desalted fully deprotected oligonucleotide contained in the retentate solution from an ultrafiltration and/or diafiltration process, that is concentrated, such as concentrated by thin film evaporation, may be in the range of between 800-7,000 OD/mL at a wavelength of 260 nm, for example, between 800-6,500 OD/mL, 800-6,000 OD/mL, 800-6,500 OD/mL, 800-6,000 OD/mL, 800-5,500 OD/mL, 800-5,000 OD/mL, 800-4,500 OD/mL, 800-4,000 OD/mL, 800-3,500 OD/mL, 800-3,000 OD/mL, 800-2,500 OD/mL, 800-2,000 OD/mL, 800-1,500 OD/mL, 800-1,000 OD/mL, 1,000-7,000 OD/mL, 1,000-6,500 OD/mL, 1,000-6,000 OD/mL, 1,000-5,500 OD/mL, 1,000-5,000 OD/mL, 1,000-4,500 OD/mL, 1,000-4,000 OD/mL, 1,000-3,500 OD/mL, 1,000-3,000 OD/mL, 2,000-6,000 OD/mL, 3,000-7,000 OD/mL, 4,000-6,500 OD/mL, or 5,000-7,000 OD/mL, at a wavelength of 260 nm. In certain embodiments, the amount of the fully deprotected oligonucleotide contained in the eluate from an ion exchange purification, or the amount of the desalted fully deprotected oligonucleotide contained in the retentate solution from an ultrafiltration and/or diafiltration process, that is concentrated, such as concentrated by thin film evaporation, may be in an amount of 800 OD/mL at a wavelength of 260 nm, for example, in an amount of 900 OD/mL, 1,000 OD/mL, 1,500 OD/mL, 2,000 OD/mL, 2,500 OD/mL, 3,000 OD/mL, 3,500 OD/mL, 4,000 OD/mL, 4,500 OD/mL, 5,000 OD/mL, 5,500 OD/mL, 6,000 OD/mL, 6,500 OD/mL, or 7,000 OD/mL, at a wavelength of 260 nm.

In certain embodiments, for example, the resulting desalted fully deprotected oligonucleotide solution from a ultrafiltration and/or diafiltration process that is concentrated, such as concentrated by thin film evaporation, may have a water content of at most 25 wt. % water, for example, a water content of at most 20 wt. % water, such as at most 15 wt. % water, at most 10 wt. % water, or at most 5 wt. % water, or for example, a water content in the range of between 25-5 wt. % water, such as between 25-10 wt. % water, between 25-15 wt. % water, between 25-20 wt. % water, between 20-15 wt. % water, or between 20-10 wt. % water.

5.15 Freeze Drying Process

In certain embodiments, the concentrated oligonucleotide, such as the concentrated oligonucleotide resulting from thin film evaporation process, is further subjected to a freeze drying process, according to the method of preparing an oligonucleotide disclosed herein.

In certain embodiments, the concentrated oligonucleotide, such as the concentrated oligonucleotide resulting from thin film evaporation process, is further subjected to a freeze drying process, wherein the freeze drying process utilizes a vacuum in the range of between 1-500 millitorr, such as in the range of between 1-450 millitorr, 1-400 millitorr, 1-350 millitorr, 1-300 millitorr, 1-250 millitorr, 1-200 millitorr, 1-150 millitorr, 1-100 millitorr, 1-50 millitorr, 100-500 millitorr, 100-400 millitorr, 50-300 millitorr, or 25-250 millitorr, and at a temperature in the range of between −50 to 35° C., such as in the range of −50 to −30° C., −45 to 30° C., −45 to 20° C., −45 to 15° C., −45 to 10° C., −45 to 5° C., −45 to 0° C., −45 to −5° C., −45 to −20° C., −40 to 20° C., −40 to 15° C., −40 to 10° C., −40 to 5° C., −40 to 0° C., −40 to −5° C., −35 to 20° C., −35 to 15° C., −35 to 10° C., −35 to 5° C.,-35 to 0° C., −10 to 20° C., 15-25° C., or −35 to −5° C.

In certain embodiments, the amount of the concentrated oligonucleotide, such as the concentrated oligonucleotide resulting from thin film evaporation process, that is further subjected to a freeze drying process may be in the range of between 300-5,400 mmol, for example, between 300-4,500 mmol, 300-4,000 mmol, 300-3,600 mmol, 300-3,000 mmol, 600-3,000 mmol, 700-3,600 mmol, 700-2,700 mmol, 700-2,500 mmol, 700-2,400 mmol, 700-2,000 mmol, 700-1,900 mmol, 700-1,800 mmol, 700-1,700 mmol, 700-1,600 mmol, 700-1,500 mmol, 700-900 mmol, 800-1,200 mmol, 800-1,000 mmol, 900-1,600 mmol, 900-1,800 mmol, 900-2,400 mmol, 900-2,700 mmol, 900-3,600 mmol, 1,800-2,700 mmol, 1,800-3,600 mmol, 1,000-3,000 mmol, 1,000-2,500 mmol, 1,000-2,000 mmol, 1,000-1,500 mmol, 1,250-1,750 mmol, 1,500-3,000 mmol, 1,500-2,500 mmol, 1,500-2,000 mmol, 1,600-2,400 mmol, 2,000-3,000 mmol, 2,500-3,000 mmol, 2,700-3,600 mmol, 2,700-4,500 mmol, 3,000-4,000 mmol, 3,600-4,500 mmol, or 3,600-5,400 mmol. In certain embodiments, the amount of the concentrated oligonucleotide, such as the concentrated oligonucleotide resulting from thin film evaporation process, that is further subjected to a freeze drying process may be 300 mmol, 400 mmol, 500 mmol, 600 mmol, 700 mmol, 800 mmol, 900 mmol, 1,000 mmol, 1,100 mmol, 1,200 mmol, 1,300 mmol, 1,400 mmol, 1,500 mmol, 1,600 mmol, 1,700 mmol, 1,800 mmol, 1,900 mmol, 2,000 mmol, 2,100 mmol, 2,200 mmol, 2,300 mmol, 2,400 mmol, 2,500 mmol, 2,600 mmol, 2,700 mmol, 2,800 mmol, 2,900 mmol, 3,000 mmol, 3,400 mmol, 3,600 mmol, 4,000 mmol, 4,200 mmol, 4,500 mmol, 5,000 mmol, or 5,400 mmol.

In certain embodiments, the amount of the concentrated oligonucleotide, such as the concentrated oligonucleotide resulting from thin film evaporation process, that is further subjected to a freeze drying process may be in the range of between 800-7,000 OD/mL at a wavelength of 260 nm, for example, between 800-6,500 OD/mL, 800-6,000 OD/mL, 800-6,500 OD/mL, 800-6,000 OD/mL, 800-5,500 OD/mL, 800-5,000 OD/mL, 800-4,500 OD/mL, 800-4,000 OD/mL, 800-3,500 OD/mL, 800-3,000 OD/mL, 800-2,500 OD/mL, 800-2,000 OD/mL, 800-1,500 OD/mL, 800-1,000 OD/mL, 1,000-7,000 OD/mL, 1,000-6,500 OD/mL, 1,000-6,000 OD/mL, 1,000-5,500 OD/mL, 1,000-5,000 OD/mL, 1,000-4,500 OD/mL, 1,000-4,000 OD/mL, 1,000-3,500 OD/mL, 1,000-3,000 OD/mL, 2,000-6,000 OD/mL, 3,000-7,000 OD/mL, 4,000-6,500 OD/mL, or 5,000-7,000 OD/mL, at a wavelength of 260 nm. In certain embodiments, the amount of the concentrated oligonucleotide, such as the concentrated oligonucleotide resulting from thin film evaporation process, that is further subjected to a freeze drying process may be in the range of 800-7,000 OD/mL at a wavelength of 260 nm, for example, in an amount of 800 OD/mL, 900 OD/mL, 1,000 OD/mL, 1,500 OD/mL, 2,000 OD/mL, 2,500 OD/mL, 3,000 OD/mL, 3,500 OD/mL, 4,000 OD/mL, 4,500 OD/mL, 5,000 OD/mL, 5,500 OD/mL, 6,000 OD/mL, 6,500 OD/mL, or 7,000 OD/mL, at a wavelength of 260 nm.

In certain embodiments, the amount of the concentrated oligonucleotide resulting from the freeze drying process may be in the range of between 300-5,400 mmol, for example, between 300-4,500 mmol, 300-4,000 mmol, 300-3,600 mmol, 300-3,000 mmol, 600-3,000 mmol, 700-3,600 mmol, 700-2,700 mmol, 700-2,500 mmol, 700-2,400 mmol, 700-2,000 mmol, 700-1,900 mmol, 700-1,800 mmol, 700-1,700 mmol, 700-1,600 mmol, 700-1,500 mmol, 700-900 mmol, 800-1,200 mmol, 800-1,000 mmol, 900-1,600 mmol, 900-1,800 mmol, 900-2,400 mmol, 900-2,700 mmol, 900-3,600 mmol, 1,800-2,700 mmol, 1,800-3,600 mmol, 1,000-3,000 mmol, 1,000-2,500 mmol, 1,000-2,000 mmol, 1,000-1,500 mmol, 1,250-1,750 mmol, 1,500-3,000 mmol, 1,500-2,500 mmol, 1,500-2,000 mmol, 1,600-2,400 mmol, 2,000-3,000 mmol, 2,500-3,000 mmol, 2,700-3,600 mmol, 2,700-4,500 mmol, 3,000-4,000 mmol, 3,600-4,500 mmol, or 3,600-5,400 mmol. In certain embodiments, the amount of the concentrated oligonucleotide resulting from the freeze drying process may be 300 mmol, 400 mmol, 500 mmol, 600 mmol, 700 mmol, 800 mmol, 900 mmol, 1,000 mmol, 1,100 mmol, 1,200 mmol, 1,300 mmol, 1,400 mmol, 1,500 mmol, 1,600 mmol, 1,700 mmol, 1,800 mmol, 1,900 mmol, 2,000 mmol, 2,100 mmol, 2,200 mmol, 2,300 mmol, 2,400 mmol, 2,500 mmol, 2,600 mmol, 2,700 mmol, 2,800 mmol, 2,900 mmol, 3,000 mmol, 3,400 mmol, 3,600 mmol, 4,000 mmol, 4,200 mmol, 4,500 mmol, 5,000 mmol, or 5,400 mmol.

In certain embodiments, the concentrated oligonucleotide resulting from the freeze drying process may have a water content of at most 25 wt. % water, for example, a water content of at most 20 wt. % water, such as at most 15 wt. % water, at most 10 wt. % water, or at most 5 wt. % water, or for example, a water content in the range of between 25-5 wt. % water, such as between 25-10 wt. % water, 25-15 wt. % water, 25-20 wt. % water, 20-15 wt. % water, 20-10 wt. % water, 10-5 wt. % water, or 6-8 wt. % water.

5.16 Synthesis Scale, Water Content, and Molecular Weight Features

In certain embodiments, an oligonucleotide composition, such as a single batch composition, a substantially pure oligonucleotide composition, or an oligonucleotide composition prepared according to the method disclosed herein, may comprise or consist of at least 300 mmol of an oligonucleotide (e.g., an anti-SMAD7 oligonucleotide or a chemically modified anti-SMAD7 oligonucleotide, synthesized using the methods as described herein; e.g., an oligonucleotide comprising a portion of at least 10 nucleotides of, consists of, or includes up to 21 nucleotides in length of, having a sequence of any one of SEQ ID NO: 2, 3, 4, 5, 6, 7, 8, 9, 10, 11, or 12) and at most 25 wt. % water. For example, in certain embodiments, the composition may comprise or consist of at least 400 mmol of the oligonucleotide, such as at least 500 mmol, at least 600 mmol, at least 700 mmol, at least 800 at least 900 mmol, at least 1,000 mmol, at least 1,100 mmol, at least 1,200 mmol, at least 1,300 mmol, at least 1,400 mmol, at least 1,500 mmol, at least 1,600 mmol, at least 1,700 mmol, at least 1,800 mmol, at least 1,900 mmol, at least 2,000 mmol, at least 2,100 mmol, at least 2,200 mmol, at least 2,300 mmol, at least 2,400 mmol, at least 2,500 mmol, at least 2,600 mmol, at least 2,700 mmol, at least 2,800 mmol, at least 2,900 mmol, at least 3,000 mmol, at least 3,600 mmol, or at least 4,500 mmol, of the oligonucleotide, for example, in the range of between 300-5,400 mmol of the oligonucleotide, such as in the range of between 300-4,500 mmol, 300-4,000 mmol, 300-3,600 mmol, 300-3,000 mmol, 600-3,000 mmol, 700-3,600 mmol, 700-3,000 mmol, 700-2,700 mmol, 700-2,500 mmol, 700-2,400 mmol, 700-2,000 mmol, 700-1,500 mmol, 700-1,000 mmol, 700-900 mmol, 800-1,000 mmol, 900-1,600 mmol, 900-2,400 mmol, 1,000-3,000 mmol, 900-3,600 mmol, 1,000-2,000 mmol, 1,500-2,000 mmol, 1,600-2,400 mmol, 1,500-2,500 mmol, 1,500-3,000 mmol, 1,800-3,600 mmol, 2,000-3,000 mmol, 2,000-2,500 mmol, 2,500-3,000 mmol, 2,600-2,800 mmol, 2,700-3,000 mmol, 900-2,000 mmol, 900-2,500 mmol, 900-2,700 mmol, 1,000-4,000 mmol, 1,500-3,500 mmol, 2,000-4,000 mmol, 2,500-3,500 mmol, 2,700-3,600 mmol, 2,700-4,500 mmol, 3,000-4,000 mmol, 3,600-4,500 mmol, or 3,600-5,400 mmol, of the oligonucleotide, and at most 25 wt. % water, such as at most 24 wt. %, at most 23 wt. %, at most 22 wt. %, at most 21 wt. %, at most 20 wt. %, at most 19 wt. %, at most 18 wt. %, at most 17 wt. %, at most 16 wt. %, at most 15 wt. %, at most 10 wt. %, at most 9 wt. %, at most 8 wt. %, at most 7 wt. %, at most 6 wt. %, at most 5 wt. % water, or in the range of between 25-5 wt. % water, for example, in the range of between 25-20 wt. % water, such as between 20-15 wt. %, 20-10 wt. %, 15-10 wt. %, 15-5 wt. %, or 10-5 wt. % water.

In certain embodiments, an oligonucleotide composition, such as a single batch composition, a substantially pure oligonucleotide composition, or an oligonucleotide composition prepared according to the method disclosed herein, may comprise or consist of at least 300 mmol, such as at least 700 mmol, of an oligonucleotide (e.g., an anti-SMAD7 oligonucleotide or a chemically modified anti-SMAD7 oligonucleotide, synthesized using the methods as described herein; e.g., an oligonucleotide comprising a portion of at least 10 nucleotides of, consists of, or includes up to 21 nucleotides in length of, having a sequence of any one of SEQ ID NO: 2, 3, 4, 5, 6, 7, 8, 9, 10, 11, or 12) and at most 25 wt. % water, wherein the oligonucleotide has a molecular weight of at least 3,000 Da, wherein the molecular weight is a protonated molecular weight, an alkai metal molecular weight, such as a sodium salt form molecular weight, or an alkaline metal molecular weight, such as a magnesium salt form molecular weight. For example, in certain embodiments, the composition of the oligonucleotide may comprise or consist of at least 300 mmol, such as at least 700 mmol, of the oligonucleotide and at most 25 wt. % water, wherein the oligonucleotide has a molecular weight of at least 3,500 Da, such as a molecular weight of at least 4,000 Da, at least 4,500 Da, at least 5,000 Da, at least 5,500 Da, at least 6,000 Da, at least 6,500 Da, at least 7,000 Da, at least 7,500 Da, at least 8,000 Da, at least 8,500 Da, at least 9,000 Da, at least 9,500 Da, or at least 10,000 Da, for example, a molecular weight in the range of between 3,000-20,000 Da, such as a molecular weight in the range of between 3,000-17,000 Da, 3,000-15,000 Da, 3,000-12,000 Da, 3,000-10,000 Da, 3,000-8,000 Da, 3,000-5,000 Da, 5,000-20,000 Da, 5,000-15,000 Da, 5,000-13,000 Da, 5,000-10,000 Da, 5,000-9,000 Da, 5,000-7,000 Da, 6,000-20,000 Da, 6,000-16,000 Da, 6,000-11,000 Da, 6,000-9,000 Da, 6,500-7,500 Da, 10,000-20,000 Da, 10,000-15,000 Da, 12,000-18,000 Da, or 15,000-20,000 Da, wherein the molecular weight is a protonated molecular weight, an alkai metal molecular weight, such as a sodium salt form molecular weight, or an alkaline metal molecular weight, such as a magnesium salt form molecular weight.

In certain embodiments, the method disclosed herein provides an oligonucleotide composition, such as a single batch composition, a substantially pure oligonucleotide composition, or an oligonucleotide composition, in a yield of at least 2 g/mmol of an at least 700 mmol synthesis scale (e.g., an oligonucleotide synthesis column having sufficient loading capacity of providing at least 700 mmol of the oligonucleotide being synthesized) of an oligonucleotide (e.g., an anti-SMAD7 oligonucleotide or a chemically modified anti-SMAD7 oligonucleotide, synthesized using the methods as described herein; e.g., an oligonucleotide comprising a portion of at least 10 nucleotides of, consists of, or includes up to 21 nucleotides in length of, having a sequence of any one of SEQ ID NO: 2, 3, 4, 5, 6, 7, 8, 9, 10, 11, or 12) and at most 25 wt. % water, wherein the yield may be determined by optical density/mL (OD/mL) at a wavelength at 260 nm or may be determined by dry weight of the oligonucleotide composition corrected for moisture content relative to the synthesis scale to prepare the oligonucleotide. For example, in certain embodiments, the yield of the composition prepared according to the method disclosed herein may be at least 2.25 g/mmol of an at least 700 mmol synthesis scale of the oligonucleotide, wherein the yield may be determined by optical density/mL (OD/mL) at a wavelength at 260 nm or may be determined by dry weight of the oligonucleotide composition corrected for moisture content relative to the synthesis scale to prepare the oligonucleotide, such as at least 2.5 g/mmol, at least 2.75 g/mmol, at least 3 g/mmol, at least 3.25 g/mmol, at least 3.3 g/mmol, at least 3.4 g/mmol, at least 3.5 g/mmol, at least 3.6 g/mmol, at least 3.7 g/mmol, at least 3.75 g/mmol, at least 3.8 g/mmol, at least 3.9 g/mmol, at least 4 g/mmol, at least 4.25 g/mmol, at least 4.5 g/mmol, at least 4.75 g/mmol, at least 5 g/mmol, at least 5.5 g/mmol, at least 6 g/mmol, at least 6.5 g/mmol, at least 7 g/mmol, at least 7.5 g/mmol, at least 8 g/mmol, at least 8.5 g/mmol, at least 9 g/mmol, at least 9.5 g/mmol, at least 10 g/mmol, at least 10.5 g/mmol, at least 11 g/mmol, at least 11.5 g/mmol, at least 12 g/mmol, at least 12.5 g/mmol, at least 13 g/mmol, at least 13.5 g/mmol, at least 14 g/mmol, at least 14.5 g/mmol, or at least 15 g/mmol of an at least 700 mmol synthesis scale of the oligonucleotide, for example, in the range of between 2-25 g/mmol synthesis scale of the oligonucleotide, such as in the range of between 2-20 g/mmol, between 2-15 g/mmol, between 2-12.5 g/mmol, 2-10 g/mmol, 2-8 g/mmol, 2-6 g/mmol, 2-5.5 g/mmol, 2-5 g/mmol, 2-4.5 g/mmol, 2-4.25 g/mmol, 2-4 g/mmol, 3.5-4.5 g/mmol, 3.5-4.25 g/mmol, 3.5-4 g/mmol, between 5-20 g/mmol, between 5-15 g/mmol, between 5-12.5 g/mmol, 5-10 g/mmol, 5-7.5 g/mmol, 6-8 g/mmol, 6-10 g/mmol, 6-15 g/mmol, 6-20 g/mmol, 7-10 g/mmol, 8-12 g/mmol, 10-15 g/mmol, 10-20 g/mmol, 20-30 g/mmol, or 15-25 g/mmol, of an at least 700 mmol synthesis scale of the oligonucleotide, wherein the yield may be determined by optical density/mL (OD/mL) at a wavelength at 260 nm or may be determined by dry weight of the oligonucleotide composition corrected for moisture content relative to the synthesis scale to prepare the oligonucleotide, and at most 25 wt. % water, such as at most 24 wt. %, at most 23 wt. %, at most 22 wt. %, at most 21 wt. %, at most 20 wt. %, at most 19 wt. %, at most 18 wt. %, at most 17 wt. %, at most 16 wt. %, at most 15 wt. %, at most 10 wt. %, at most 9 wt. %, at most 8 wt. %, at most 7 wt. %, at most 6 wt. %, at most 5 wt. % water, or in the range of between 25-5 wt. % water, for example, in the range of between 25-20 wt. % water, such as between 20-15 wt. %, 20-10 wt. %, 15-10 wt. %, 15-5 wt. %, or 10-5 wt. % water.

In certain embodiments, the method disclosed herein provides an oligonucleotide composition, such as a single batch composition, a substantially pure oligonucleotide composition, or an oligonucleotide composition, in a yield of at least 2 g/mmol of an at least 700 mmol synthesis scale (e.g., an oligonucleotide synthesis column having sufficient loading capacity of providing at least 700 mmol of the oligonucleotide being synthesized) of an oligonucleotide (e.g., an anti-SMAD7 oligonucleotide or a chemically modified anti-SMAD7 oligonucleotide, synthesized using the methods as described herein; e.g., an oligonucleotide comprising a portion of at least 10 nucleotides of, consists of, or includes up to 21 nucleotides in length of, having a sequence of any one of SEQ ID NO: 2, 3, 4, 5, 6, 7, 8, 9, 10, 11, or 12) and at most 25 wt. % water, wherein the oligonucleotide has a molecular weight of at least 3,000 Da, wherein the molecular weight is a protonated molecular weight, an alkai metal molecular weight, such as a sodium salt form molecular weight, or an alkaline metal molecular weight, such as a magnesium salt form molecular weight, wherein the yield may be determined by optical density/mL (OD/mL) at a wavelength at 260 nm or may be determined by dry weight of the oligonucleotide composition corrected for moisture content relative to the synthesis scale to prepare the oligonucleotide. For example, in certain embodiments, the yield of the composition prepared according to the method disclosed herein may be at least 2 g/mmol of an at least 700 mmol synthesis scale of the oligonucleotide and at most 25 wt. % water, wherein the yield may be determined by optical density/mL (OD/mL) at a wavelength at 260 nm or may be determined by dry weight of the oligonucleotide composition corrected for moisture content relative to the synthesis scale to prepare the oligonucleotide, wherein the oligonucleotide has a molecular weight of at least 3,500 Da, such as a molecular weight of at least 4,000 Da, at least 4,500 Da, at least 5,000 Da, at least 5,500 Da, at least 6,000 Da, at least 6,500 Da, at least 7,000 Da, at least 7,500 Da, at least 8,000 Da, at least 8,500 Da, at least 9,000 Da, at least 9,500 Da, or at least 10,000 Da, for example, a molecular weight in the range of between 3,000-20,000 Da, such as a molecular weight in the range of between 3,000-17,000 Da, 3,000-15,000 Da, 3,000-12,000 Da, 3,000-10,000 Da, 3,000-8,000 Da, 3,000-5,000 Da, 5,000-20,000 Da, 5,000-15,000 Da, 5,000-13,000 Da, 5,000-10,000 Da, 5,000-9,000 Da, 5,000-7,000 Da, 6,000-20,000 Da, 6,000-16,000 Da, 6,000-11,000 Da, 6,000-9,000 Da, 6,500-7,500 Da, 10,000-20,000 Da, 10,000-15,000 Da, 12,000-18,000 Da, or 15,000-20,000 Da, and wherein the molecular weight is a protonated molecular weight, an alkai metal molecular weight, such as a sodium salt form molecular weight, or an alkaline metal molecular weight, such as a magnesium salt form molecular weight.

In certain embodiments, the method disclosed herein provides an oligonucleotide composition, such as a single batch composition, a substantially pure oligonucleotide composition, or an oligonucleotide composition, in a yield of at least 2.0 g/mmol of an at least 700 mmol synthesis scale (e.g., an oligonucleotide synthesis column having sufficient loading capacity of providing at least 700 mmol of the oligonucleotide being synthesized), or of two or more pooled at least 700 mmol synthesis scale preparations, of an oligonucleotide (e.g., an anti-SMAD7 oligonucleotide or a chemically modified anti-SMAD7 oligonucleotide, synthesized using the methods as described herein; e.g., an oligonucleotide comprising a portion of at least 10 nucleotides of, consists of, or includes up to 21 nucleotides in length of, having a sequence of any one of SEQ ID NO: 2, 3, 4, 5, 6, 7, 8, 9, 10, 11, or 12) and at most 25 wt. % water, wherein the yield may be determined by optical density/mL (OD/mL) at a wavelength at 260 nm or may be determined by dry weight of the oligonucleotide composition corrected for moisture content relative to the synthesis scale to prepare the oligonucleotide. For example, in certain embodiments, the yield of the composition prepared according to the method disclosed herein may be at least 2.0 g/mmol, such as at least 2.25, 2.5, 2.75, 3.0, 3.25, 3.3, 3.4, 3.5, 3.6, 3.7, 3.8, 3.9, or at least 4.0 g/mmol, of an at least 700 mmol synthesis scale, or of two or more pooled at least 700 mmol synthesis scale preparations, such as 3, 4, 5, 6, 7, 8, 9, or 10 or more pooled at least 700 mmol synthesis scale preparations, of the oligonucleotide, wherein the yield may be determined by optical density/mL (OD/mL) at a wavelength at 260 nm or may be determined by dry weight of the oligonucleotide composition corrected for moisture content relative to the synthesis scale to prepare the oligonucleotide, for example, in the range of between 2.0-25 g/mmol synthesis scale of the oligonucleotide, such as in the range of between 2-25, 2-20, 2-15, 2-12.5, 2-10, 2-8, 2-6, 2-5.5, 2-5, 2-4.5, 2-4.25, 2-4, 3.5-4.5, 3.5-4.25, 3.5-4, 5-20, 5-15, 5-12.5, 5-10, 5-7.5, 6-8, 6-10, 6-15, 6-20, 7-10, 8-12, 10-15, 10-20, 20-30, or 15-25 g/mmol, synthesis scale of the oligonucleotide, wherein the yield may be determined by optical density/mL (OD/mL) at a wavelength at 260 nm or may be determined by dry weight of the oligonucleotide composition corrected for moisture content relative to the synthesis scale to prepare the oligonucleotide, and at most 25 wt. % water, such as at most 20 wt. %, at most 15 wt. %, at most 10 wt. %, at most 9 wt. %, at most 8 wt. %, at most 7 wt. %, at most 6 wt. %, or at most 5 wt. % water, or in the range of between 25-5 wt. % water, for example, in the range of between 20-5 wt. % water, between 15-5 wt. % water, between 10-5 wt. % water, between 9-5 wt. % water, such as between 8-5 wt. %, 7-5 wt. %, 6-5 wt. %, 9-6 wt. %, or 8-7 wt. % water. In certain embodiments, the yield of the composition prepared according to the method disclosed herein may be at least 2.0 g/mmol, such as at least 2.25, 2.5, 2.75, 3.0, 3.25, 3.3, 3.4, 3.5, 3.6, 3.7, 3.8, 3.9, or at least 4.0 g/mmol, of an at least 700 mmol synthesis scale, or of 2, 3, 4, 5, 6, 7, 8, 9, or 10 least 700 mmol oligonucleotide synthesis scale preparations pooled together, for example, between 1-10 at least 700 mmol oligonucleotide synthesis scale preparations pooled together, such as between 1-8, 2-10, 3-9, 4-7, 4-6, 6-10, or 8-10, at least 700 mmol oligonucleotide synthesis scale preparations pooled together, such as oligonucleotide synthesis scale preparations of at least 800 mmol, at least 900 mmol, at least 1,000 mmol, at least 1,100 mmol, at least 1,200 mmol, at least 1,300 mmol, at least 1,400 mmol, at least 1,600 mmol, at least 1,800 mmol, at least 2,400 mmol, at least 2,700 mmol, at least 3,000 mmol, at least 3,600 mmol, or at least 4,500 mmol, or oligonucleotide synthesis scale preparations in the range of between 300-5,400 mmol, such as in the range of between 300-4,500 mmol, 300-4,000 mmol, 300-3,600 mmol, 300-3,000 mmol, 600-3,000 mmol, 700-3,600 mmol, 700-3,000 mmol, 700-2,700 mmol, 700-2,500 mmol, 700-2,400 mmol, 700-2,000 mmol, 700-1,500 mmol, 700-1,000 mmol, 700-900 mmol, 800-1,000 mmol, 900-1,600 mmol, 900-2,400 mmol, 900-3,600 mmol, 1,000-3,000 mmol, 1,000-2,000 mmol, 1,500-2,000 mmol, 1,600-2,400 mmol, 1,500-2,500 mmol, 1,500-3,000 mmol, 1,800-3,600 mmol, 2,000-3,000 mmol, 2,000-2,500 mmol, 2,500-3,000 mmol, 2,600-2,800 mmol, 2,700-3,000 mmol, 900-2,000 mmol, 900-2,500 mmol, 900-2,700 mmol, 1,000-4,000 mmol, 1,500-3,500 mmol, 2,000-4,000 mmol, 2,500-3,500 mmol, 2,700-3,600 mmol, 2,700-4,500 mmol, 3,000-4,000 mmol, 3,600-4,500 mmol, or 3,600-5,400 mmol.

In certain embodiments, the method disclosed herein provides an oligonucleotide composition, such as a single batch composition, a substantially pure oligonucleotide composition, or an oligonucleotide composition, in a yield of at least 2.0 g/mmol of an at least 700 mmol synthesis scale (e.g., an oligonucleotide synthesis column having sufficient loading capacity of providing at least 700 mmol of the oligonucleotide being synthesized), or of two or more pooled at least 700 mmol synthesis scale preparations, of an oligonucleotide (e.g., an anti-SMAD7 oligonucleotide or a chemically modified anti-SMAD7 oligonucleotide, synthesized using the methods as described herein; e.g., an oligonucleotide comprising a portion of at least 10 nucleotides of, consists of, or includes up to 21 nucleotides in length of, having a sequence of any one of SEQ ID NO: 2, 3, 4, 5, 6, 7, 8, 9, 10, 11, or 12) and at most 25 wt. % water, wherein the oligonucleotide has a molecular weight of at least 3,000 Da, wherein the molecular weight is a protonated molecular weight, an alkai metal molecular weight, such as a sodium salt form molecular weight, or an alkaline metal molecular weight, such as a magnesium salt form molecular weight, wherein the yield may be determined by optical density/mL (OD/mL) at a wavelength at 260 nm or may be determined by dry weight of the oligonucleotide composition corrected for moisture content relative to the synthesis scale to prepare the oligonucleotide. For example, in certain embodiments, the yield of the composition prepared according to the method disclosed herein may be at least 2.0 g/mmol, such as at least 2.25, 2.5, 2.75, 3.0, 3.25, 3.3, 3.4, 3.5, 3.6, 3.7, 3.8, 3.9, or at least 4.0 g/mmol, for example, in the range of between 2.0-25 g/mmol synthesis scale of the oligonucleotide, such as in the range of between 2-25, 2-20, 2-15, 2-12.5, 2-10, 2-8, 2-6, 2-5.5, 2-5, 2-4.5, 2-4.25, 2-4, 3.5-4.5, 3.5-4.25, 3.5-4, 5-20, 5-15, 5-12.5, 5-10, 5-7.5, 6-8, 6-10, 6-15, 6-20, 7-10, 8-12, 10-15, 10-20, 20-30, or 15-25 g/mmol, of an at least 700 mmol synthesis scale, or of two or more at least 700 mmol oligonucleotide synthesis scale preparations pooled together, such as 3, 4, 5, 6, 7, 8, 9, or 10 or more at least 700 mmol oligonucleotide synthesis scale preparations pooled together, for example oligonucleotide synthesis scale preparations of at least 800 mmol, at least 900 mmol, at least 1,000 mmol, at least 1,100 mmol, at least 1,200 mmol, at least 1,300 mmol, at least 1,400 mmol, at least 1,600 mmol, at least 1,800 mmol, at least 2,400 mmol, at least 2,700 mmol, at least 3,000 mmol, at least 3,600 mmol, or at least 4,500 mmol, or oligonucleotide synthesis scale preparations in the range of between 300-5,400 mmol, such as in the range of between 300-3,600 mmol, 300-3,000 mmol, 600-3,000 mmol, 700-3,600 mmol, 700-3,000 mmol, 700-2,700 mmol, 700-2,500 mmol, 700-2,400 mmol, 700-2,000 mmol, 700-1,500 mmol, 700-1,000 mmol, 700-900 mmol, 800-1,000 mmol, 900-1,600 mmol, 900-2,400 mmol, 900-3,600 mmol, 1,000-3,000 mmol, 1,000-2,000 mmol, 1,500-2,000 mmol, 1,600-2,400 mmol, 1,500-2,500 mmol, 1,500-3,000 mmol, 1,800-3,600 mmol, 2,000-3,000 mmol, 2,000-2,500 mmol, 2,500-3,000 mmol, 2,600-2,800 mmol, 2,700-3,000 mmol, 900-2,000 mmol, 900-2,500 mmol, 900-2,700 mmol, 1,000-4,000 mmol, 1,500-3,500 mmol, 2,000-4,000 mmol, 2,500-3,500 mmol, 2,700-3,600 mmol, 2,700-4,500 mmol, 3,000-4,000 mmol, 3,600-4,500 mmol, or 3,600-5,400 mmol, and at most 25 wt. % water, wherein the yield may be determined by optical density/mL (OD/mL) at a wavelength at 260 nm or may be determined by dry weight of the oligonucleotide composition corrected for moisture content relative to the synthesis scale to prepare the oligonucleotide, wherein the oligonucleotide has a molecular weight of at least 3,500 Da, such as a molecular weight of at least 4,000 Da, at least 4,500 Da, at least 5,000 Da, at least 5,500 Da, at least 6,000 Da, at least 6,500 Da, at least 7,000 Da, at least 7,500 Da, at least 8,000 Da, at least 8,500 Da, at least 9,000 Da, at least 9,500 Da, or at least 10,000 Da, for example, a molecular weight in the range of between 3,000-20,000 Da, such as a molecular weight in the range of between 3,000-17,000 Da, 3,000-15,000 Da, 3,000-12,000 Da, 3,000-10,000 Da, 3,000-8,000 Da, 3,000-5,000 Da, 5,000-20,000 Da, 5,000-15,000 Da, 5,000-13,000 Da, 5,000-10,000 Da, 5,000-9,000 Da, 5,000-7,000 Da, 6,000-20,000 Da, 6,000-16,000 Da, 6,000-11,000 Da, 6,000-9,000 Da, 6,500-7,500 Da, 10,000-20,000 Da, 10,000-15,000 Da, 12,000-18,000 Da, or 15,000-20,000 Da, and wherein the molecular weight is a protonated molecular weight, an alkai metal molecular weight, such as a sodium salt form molecular weight, or an alkaline metal molecular weight, such as a magnesium salt form molecular weight.

In certain embodiments, an oligonucleotide composition, such as a single batch composition, a substantially pure oligonucleotide composition, or an oligonucleotide composition prepared according to the method disclosed herein, may comprise or consist of at least 2 kg of an oligonucleotide (e.g., an anti-SMAD7 oligonucleotide or a chemically modified anti-SMAD7 oligonucleotide, synthesized using the methods as described herein; e.g., an oligonucleotide comprising a portion of at least 10 nucleotides of, consists of, or includes up to 21 nucleotides in length of, having a sequence of any one of SEQ ID NO: 2, 3, 4, 5, 6, 7, 8, 9, 10, 11, or 12) and at most 25 wt. % water. For example, in certain embodiments, the composition may comprise or consist of at least 2.5 kg of the oligonucleotide, such as at least 3 kg, at least 3.5 kg, at least 4 kg, at least 4.5 kg, at least 5 kg, at least 5.5 kg, at least 6 kg, at least 6.5 kg, at least 7 kg, at least 7.5 kg, at least 8 kg, at least 8.5 kg, at least 9 kg, at least 9.5 kg, at least 10 kg, at least 11 kg, at least 12 kg, at least 13 kg, at least 14 kg, or at least 15 kg, of the oligonucleotide, for example, in the range of between 2-15 kg of the oligonucleotide, such as in the range of between 2-12.5 kg, 2-10 kg, 2-7.5 kg, 2-5 kg, 3-15 kg, 3-12.5 kg, 3-10 kg, 3-8 kg, 3-6 kg, 5-15 kg, 5-13 kg, 5-10 kg, 5-7.5 kg, 6-15 kg, 6-10 kg, 8-15 kg, 8-12 kg, or 10-15 kg, of the oligonucleotide, and at most 25 wt. % water, such as at most 24 wt. %, at most 23 wt. %, at most 22 wt. %, at most 21 wt. %, at most 20 wt. %, at most 19 wt. %, at most 18 wt. %, at most 17 wt. %, at most 16 wt. %, at most 15 wt. %, at most 10 wt. %, at most 9 wt. %, at most 8 wt. %, at most 7 wt. %, at most 6 wt. %, at most 5 wt. % water, or in the range of between 25-5 wt. % water, for example, in the range of between 25-20 wt. % water, such as between 20-15 wt. %, 20-10 wt. %, 15-10 wt. %, 15-5 wt. %, or 10-5 wt. % water.

In certain embodiments, an oligonucleotide composition, such as a single batch composition, a substantially pure oligonucleotide composition, or an oligonucleotide composition prepared according to the method disclosed herein, may comprise or consist of at least 2 kg of an oligonucleotide (e.g., an anti-SMAD7 oligonucleotide or a chemically modified anti-SMAD7 oligonucleotide, synthesized using the methods as described herein; e.g., an oligonucleotide comprising a portion of at least 10 nucleotides of, consists of, or includes up to 21 nucleotides in length of, having a sequence of any one of SEQ ID NO: 2, 3, 4, 5, 6, 7, 8, 9, 10, 11, or 12) and at most 25 wt. % water, wherein the oligonucleotide has a molecular weight of at least 3,000 Da, wherein the molecular weight is a protonated molecular weight, an alkai metal molecular weight, such as a sodium salt form molecular weight, or an alkaline metal molecular weight, such as a magnesium salt form molecular weight. For example, in certain embodiments, the composition may comprise or consist of at least 2 kg of the oligonucleotide and at most 25 wt. % water, wherein the oligonucleotide has a molecular weight of at least 3,500 Da, such as a molecular weight of at least 4,000 Da, at least 4,500 Da, at least 5,000 Da, at least 5,500 Da, at least 6,000 Da, at least 6,500 Da, at least 7,000 Da, at least 7,500 Da, at least 8,000 Da, at least 8,500 Da, at least 9,000 Da, at least 9,500 Da, or at least 10,000 Da, for example, a molecular weight in the range of between 3,000-20,000 Da, such as a molecular weight in the range of between 3,000-17,000 Da, 3,000-15,000 Da, 3,000-12,000 Da, 3,000-10,000 Da, 3,000-8,000 Da, 3,000-5,000 Da, 5,000-20,000 Da, 5,000-15,000 Da, 5,000-13,000 Da, 5,000-10,000 Da, 5,000-9,000 Da, 5,000-7,000 Da, 6,000-20,000 Da, 6,000-16,000 Da, 6,000-11,000 Da, 6,000-9,000 Da, 6,500-7,500 Da, 10,000-20,000 Da, 10,000-15,000 Da, 12,000-18,000 Da, or 15,000-20,000 Da, wherein the molecular weight is a protonated molecular weight, an alkai metal molecular weight, such as a sodium salt form molecular weight, or an alkaline metal molecular weight, such as a magnesium salt form molecular weight.

In certain embodiments, an oligonucleotide composition, such as a single batch composition, a substantially pure oligonucleotide composition, or an oligonucleotide composition prepared according to the method disclosed herein, may comprise or consist of at least 50 mol % of an oligonucleotide output from at least one 300 mmol or greater oligonucleotide synthesis column (i.e., an oligonucleotide synthesis column having a loading capacity of providing at least one 300 mmol or greater of the oligonucleotide being synthesized), such as from at least one 700 mmol or greater oligonucleotide synthesis column, and at most 25 wt. % water, wherein the oligonucleotide is as disclosed herein, such as, an anti-SMAD7 oligonucleotide or a chemically modified anti-SMAD7 oligonucleotide, synthesized using the methods as described herein; e.g., an oligonucleotide comprising a portion of at least 10 nucleotides of, consists of, or includes up to 21 nucleotides in length of, having a sequence of any one of SEQ ID NO: 2, 3, 4, 5, 6, 7, 8, 9, 10, 11, or 12, wherein the output is determined by optical density/mL (OD/mL) or may be determined by dry weight of the oligonucleotide composition corrected for moisture content relative to the synthesis scale to prepare the oligonucleotide. In certain embodiments, the composition may comprise or consist of at least 50 mol % of the oligonucleotide output (as determined by optical density/mL (OD/mL) or determined by dry weight of the oligonucleotide composition corrected for moisture content relative to the synthesis scale to prepare the oligonucleotide), for example at least 55 mol %, at least 60 mol %, at least 65 mol %, at least 70 mol %, at least 75 mol %, at least 80 mol %, at least 85 mol %, at least 90 mol %, at least 95 mol %, at least 97 mol %, at least 98 mol %, or at least 99 mol %, of the oligonucleotide output, such as in the range of between 50-99 mol %, 60-99 mol %, 70-99 mol %, 80-99 mol %, 90-99 mol %, 95-99 mol %, 75-95 mol %, 80-97 mol %, 85-95 mol %, or 70-90 mol %, of the oligonucleotide output, from at least one 300 mmol or greater oligonucleotide synthesis column, such as at least one 700 mmol or greater oligonucleotide synthesis column, and at most 25 wt. % water. In certain embodiments, the oligonucleotide output is pooled from at least 1, 2, 3, 4, 5, 6, 7, 8, 9, or 10, oligonucleotide synthesis columns of 300 mmol or greater, for example, from between 1-10 oligonucleotide synthesis columns of 300 mmol or greater, such as between 1-8, 2-10, 3-9, 4-7, 4-6, 6-10, or 8-10, oligonucleotide synthesis columns of 300 mmol or greater, such as, from oligonucleotide synthesis columns of 600 mmol or greater, 700 mmol or greater, 800 mmol or greater, 900 mmol or greater, 1,000 mmol or greater, 1,100 mmol or greater, 1,200 mmol or greater, 1,300 mmol or greater, 1,400 mmol or greater, 1,500 mmol or greater, 1,600 mmol or greater, 1,700 mmol or greater, 1,800 mmol or greater, 1,900 mmol or greater, 2,000 mmol or greater, 2,100 mmol or greater, 2,200 mmol or greater, 2,300 mmol or greater, 2,400 mmol or greater, 2,500 mmol or greater, 2,600 mmol or greater, 2,700 mmol or greater, 2,800 mmol or greater, 2,900 mmol or greater, 3,000 mmol or greater, 3,200 mmol or greater, 3,400 mmol or greater, 3,600 mmol or greater, 4,000 mmol or greater, 4,200 mmol or greater, 4,500 mmol or greater, 5,000 mmol or greater, or 5,400 mmol or greater, or from oligonucleotide synthesis columns in the range of between 300-5,400 mmol, such as in the range of between 300-4,500 mmol, 300-4,000 mmol, 300-3,600 mmol, 300-3,000 mmol, 600-3,000 mmol, 700-3,600 mmol, 700-3,000 mmol, 700-2,700 mmol, 700-2,500 mmol, 700-2,400 mmol, 700-2,000 mmol, 700-1,500 mmol, 700-1,000 mmol, 700-900 mmol, 800-1,000 mmol, 900-1,600 mmol, 900-2,400 mmol, 900-3,600 mmol, 1,000-3,000 mmol, 1,000-2,000 mmol, 1,500-2,000 mmol, 1,600-2,400 mmol, 1,500-2,500 mmol, 1,500-3,000 mmol, 1,800-3,600 mmol, 2,000-3,000 mmol, 2,000-2,500 mmol, 2,500-3,000 mmol, 2,600-2,800 mmol, 2,700-3,000 mmol, 900-2,000 mmol, 900-2,500 mmol, 900-2,700 mmol, 1,000-4,000 mmol, 1,500-3,500 mmol, 2,000-4,000 mmol, 2,500-3,500 mmol, 2,700-3,600 mmol, 2,700-4,500 mmol, 3,000-4,000 mmol, 3,600-4,500 mmol, or 3,600-5,400 mmol, and at most 25 wt. % water. In certain embodiments, the composition may comprise or consist of at least 50 mol % of an oligonucleotide output from at least one oligonucleotide synthesis column of 300 mmol or greater (as determined by optical density/mL (OD/mL) or determined by dry weight of the oligonucleotide composition corrected for moisture content relative to the synthesis scale to prepare the oligonucleotide), such as at least one 700 mmol or greater oligonucleotide synthesis column, for example, at least 2, 3, 4, 5, 6, 7, 8, 9, or 10, or in the range of between 1-10, 1-8, 2-10, 3-9, 4-7, 4-6, 6-10, or 8-10, oligonucleotide synthesis columns of 300 mmol or greater, such as at least one 700 mmol or greater oligonucleotide synthesis column, and at most 25 wt. % water, such as and at most 25 wt. % water, such as at most 24 wt. %, at most 23 wt. %, at most 22 wt. %, at most 21 wt. %, at most 20 wt. %, at most 19 wt. %, at most 18 wt. %, at most 17 wt. %, at most 16 wt. %, at most 15 wt. %, at most 10 wt. %, at most 9 wt. %, at most 8 wt. %, at most 7 wt. %, at most 6 wt. %, at most 5 wt. % water, or in the range of between 25-5 wt. % water, for example, in the range of between 25-20 wt. % water, such as between 20-15 wt. %, 20-10 wt. %, 15-10 wt. %, 15-5 wt. %, or 10-5 wt. % water, wherein the oligonucleotide has a molecular weight of at least 3,000 Da, for example, a molecular weight of at least 3,500 Da, such as at least 4,000 Da, at least 4,500 Da, at least 5,000 Da, at least 5,500 Da, at least 6,000 Da, at least 6,500 Da, at least 7,000 Da, at least 7,500 Da, at least 8,000 Da, at least 8,500 Da, at least 9,000 Da, at least 9,500 Da, or at least 10,000 Da, for example, a molecular weight in the range of between 3,000-20,000 Da, such as a molecular weight of in the range of between 3,000-17,000 Da, 3,000-15,000 Da, 3,000-12,000 Da, 3,000-10,000 Da, 3,000-8,000 Da, 3,000-5,000 Da, 5,000-20,000 Da, 5,000-15,000 Da, 5,000-13,000 Da, 5,000-10,000 Da, 5,000-9,000 Da, 5,000-7,000 Da, 6,000-20,000 Da, 6,000-16,000 Da, 6,000-11,000 Da, 6,000-9,000 6,500-7,500 Da, Da, 10,000-20,000 Da, 10,000-15,000 Da, 12,000-18,000 Da, or 15,000-20,000 Da, wherein the molecular weight is a protonated molecular weight, an alkai metal molecular weight, such as a sodium salt form molecular weight, or an alkaline metal molecular weight, such as a magnesium salt form molecular weight.

In certain embodiments, the degree of purity of an oligonucleotide composition, such as a single batch composition, a substantially pure oligonucleotide composition, or an oligonucleotide composition prepared according to the method disclosed herein, comprising or consisting of an oligonucleotide (e.g., an anti-SMAD7 oligonucleotide or a chemically modified anti-SMAD7 oligonucleotide, synthesized using the methods as described herein; e.g., an oligonucleotide comprising a portion of at least 10 nucleotides of, consists of, or includes up to 21 nucleotides in length of, having a sequence of any one of SEQ ID NO: 2, 3, 4, 5, 6, 7, 8, 9, 10, 11, or 12) may be 60% or greater (as determined, for example, by RP-HPLC), such as a degree of purity of 65% or greater, 70% or greater, 75% or greater, 80% or greater, 85% or greater, 90% or greater, 95% or greater, 97% or greater, or 99% or greater, such as a degree of purity in the range of between 50-99%, for example, between 60-99%, 70-99%, 80-99%, 90-99%, 95-99%, 75-95%, 80-97%, 85-95%, or 70-90% (as determined, for example, by RP-HPLC), wherein the molecular weight of the oligonucleotide in said composition is at least 3,000 Da, such as a molecular weight of at least 3,500 Da, at least 4,000 Da, at least 4,500 Da, at least 5,000 Da, at least 5,500 Da, at least 6,000 Da, at least 6,500 Da, at least 7,000 Da, at least 7,500 Da, at least 8,000 Da, at least 8,500 Da, at least 9,000 Da, at least 9,500 Da, or at least 10,000 Da, for example, a molecular weight in the range of between 3,000-20,000 Da, such as a molecular weight of in the range of between 3,000-17,000 Da, 3,000-15,000 Da, 3,000-12,000 Da, 3,000-10,000 Da, 3,000-8,000 Da, 3,000-5,000 Da, 5,000-20,000 Da, 5,000-15,000 Da, 5,000-13,000 Da, 5,000-10,000 Da, 5,000-9,000 Da, 5,000-7,000 Da, 6,000-20,000 Da, 6,000-16,000 Da, 6,000-11,000 Da, 6,000-9,000 Da, 6,500-7,500 Da, 10,000-20,000 Da, 10,000-15,000 Da, 12,000-18,000 Da, or 15,000-20,000 Da, wherein the molecular weight is a protonated molecular weight, an alkai metal molecular weight, such as a sodium salt form molecular weight, or an alkaline metal molecular weight, such as a magnesium salt form molecular weight.

In certain embodiments, an oligonucleotide composition, such as a single batch composition, a substantially pure oligonucleotide composition, or an oligonucleotide composition prepared according to the method disclosed herein, of an oligonucleotide (e.g., an anti-SMAD7 oligonucleotide or a chemically modified anti-SMAD7 oligonucleotide, synthesized using the methods as described herein; e.g., an oligonucleotide comprising a portion of at least 10 nucleotides of, consists of, or includes up to 21 nucleotides in length of, having a sequence of any one of SEQ ID NO: 2, 3, 4, 5, 6, 7, 8, 9, 10, 11, or 12) designed to have a predetermined number of nucleotides (N) may contain an oligonucleotide having (N+1) number of nucleotides in an amount of no more than 4 wt. %, relative to the weight of the composition, for example, an oligonucleotide having (N+1) number of nucleotides in an amount of no more than 3.5 wt. %, relative to the weight of the composition, such as no more than 3.0 wt. %, no more than 2.5 wt. %, no more than 2.0 wt. %, no more than 1.5 wt. %, no more than 1.0 wt. %, or no more than 0.5 wt. %, relative to the weight of the single batch composition of an oligonucleotide designed to have a predetermined number of nucleotides (N). For example, in certain embodiments, the composition of an oligonucleotide designed to have a predetermined number of nucleotides (N=21) may contain an oligonucleotide having (N+1) number of nucleotides (i.e., N+1=22, or a 22-mer) in an amount of no more than 4 wt. %, relative to the weight of the composition of an oligonucleotide designed to have a predetermined number of nucleotides (N=21, or a 21-mer).

In certain embodiments, an oligonucleotide composition, such as a single batch composition, a substantially pure oligonucleotide composition, or an oligonucleotide composition prepared according to the method disclosed herein, may comprise or consist of an oligonucleotide wherein the 5′-hydroxyl group of the 5′-terminal nucleoside is protected (e.g., a protected anti-SMAD7 oligonucleotide or a chemically modified anti-SMAD7 oligonucleotide, synthesized using the methods as described herein; e.g., a protected oligonucleotide comprising a portion of at least 10 nucleotides of, consists of, or includes up to 21 nucleotides in length of, having a sequence of any one of SEQ ID NO: 2, 3, 4, 5, 6, 7, 8, 9, 10, 11, or 12). For example, in certain embodiments, the composition may comprise or consist of a protected oligonucleotide wherein only the 5′-hydroxyl group of the 5′-terminal nucleoside is protected. In certain embodiments, the composition may comprise or consist of a protected oligonucleotide obtained after cleavage and elution from a synthesis column, wherein the 5′-hydroxyl group of the 5′-terminal nucleoside is protected. In certain embodiments, the degree of purity of the composition comprising or consisting of the protected oligonucleotide having the 5′-hydroxyl group of the 5′-terminal nucleoside protected, is 60% or greater (as determined, for example, by RP-HPLC), such as a degree of purity of 65% or greater, 70% or greater, 75% or greater, 80% or greater, 85% or greater, 90% or greater, 95% or greater, 97% or greater, or 99% or greater, such as a degree of purity in the range of between 50-99%, 60-99%, 70-99%, 80-99%, 90-99%, 95-99%, 75-95%, 80-97%, 85-95%, or 70-90% (as determined, for example, by RP-HPLC), wherein, the molecular weight of the protected oligonucleotide having the 5′-hydroxyl group of the 5′-terminal nucleoside protected in the substantially pure oligonucleotide composition is at least 3,000 Da, such as a molecular weight of at least 3,500 Da, at least 4,000 Da, at least 4,500 Da, at least 5,000 Da, at least 5,500 Da, at least 6,000 Da, at least 6,500 Da, at least 7,000 Da, at least 7,500 Da, at least 8,000 Da, at least 8,500 Da, at least 9,000 Da, at least 9,500 Da, or at least 10,000 Da, for example, a molecular weight in the range of between 3,000-20,000 Da, such as a molecular weight of in the range of between 3,000-17,000 Da, 3,000-15,000 Da, 3,000-12,000 Da, 3,000-10,000 Da, 3,000-8,000 Da, 3,000-5,000 Da, 5,000-20,000 Da, 5,000-15,000 Da, 5,000-13,000 Da, 5,000-10,000 Da, 5,000-9,000 Da, 5,000-7,000 Da, 6,000-20,000 Da, 6,000-16,000 Da, 6,000-11,000 Da, 6,000-9,000 Da, 6,500-7,500 Da, 10,000-20,000 Da, 10,000-15,000 Da, 12,000-18,000 Da, or 15,000-20,000 Da, wherein the molecular weight is a protonated molecular weight, an alkai metal molecular weight, such as a sodium salt form molecular weight, or an alkaline metal molecular weight, such as a magnesium salt form molecular weight.

In certain embodiments, an oligonucleotide composition, such as a single batch composition, a substantially pure oligonucleotide composition, or an oligonucleotide composition prepared according to the method disclosed herein, may be derived from the oligonucleotide synthesis output from the at least one, or the plurality of, oligonucleotide synthesis column(s) (or synthesis run(s)) having the solid support with a loading capacity of the specified quantity, wherein said oligonucleotide synthesis output may: (1) provide the single batch composition (or single batch oligonucleotide composition) as a liquid oligonucleotide composition or a solid oligonucleotide composition; or (2) be pooled or combined (such that the resulting pooled/combined oligonucleotide composition has uniform character and quality, e.g., the physical and chemical properties are distributed throughout said resulting pooled/combined oligonucleotide composition) to provide the single batch composition (or single batch oligonucleotide composition) as a liquid oligonucleotide composition or a solid oligonucleotide composition. In certain embodiments, an oligonucleotide composition, such as a single batch composition, a substantially pure oligonucleotide composition, or an oligonucleotide composition prepared according to the method disclosed herein, may be derived from the oligonucleotide synthesis output from the at least one, or the plurality of, oligonucleotide synthesis column(s) (or synthesis run(s)) having the solid support with a loading capacity of the specified quantity, wherein said oligonucleotide synthesis output may: (1) be processed in a single iteration or in a plurality of parallel iterations (not necessarily conducted simultaneously) by at least one further process(s), comprising: an oligonucleotide chemical transformation process (e.g., an oligonucleotide deprotection or cleavage from solid support), an oligonucleotide purification process (e.g., ion-exchange chromatography), an oligonucleotide desalting process (e.g., ultrafiltration and/or diafiltration), a liquid composition concentration process (e.g., thin film evaporation), or a drying process (e.g., freeze drying), or combinations thereof; (2) be pooled or combined (such that the resulting pooled/combined oligonucleotide composition has uniform character and quality, e.g., the physical and chemical properties are distributed throughout said resulting pooled/combined oligonucleotide composition): (a) before being processed in a single iteration or in a plurality of parallel iterations (not necessarily conducted simultaneously) by the at least one further process(s) (or combinations thereof); or (b) followed by partitioning the resulting pooled/combined oligonucleotide composition into smaller portions before being processed in a plurality of parallel iterations (not necessarily conducted simultaneously) by the at least one further process(s) (or combinations thereof); or (3) be partitioned into smaller portions before being processed in a plurality of parallel iterations (not necessarily conducted simultaneously) by the at least one further process(s) (or combinations thereof). In certain embodiments, an oligonucleotide composition, such as a single batch composition, a substantially pure oligonucleotide composition, or an oligonucleotide composition prepared according to the method disclosed herein, may be derived from the oligonucleotide synthesis output from the at least one, or the plurality of, oligonucleotide synthesis column(s) (or synthesis run(s)) having the solid support with a loading capacity of the specified quantity, wherein said oligonucleotide synthesis output may be processed in a single iteration or in a plurality of parallel iterations (not necessarily conducted simultaneously) by at least one further process(s), comprising: an oligonucleotide chemical transformation process (e.g., an oligonucleotide deprotection or cleavage from solid support), an oligonucleotide purification process (e.g., ion-exchange chromatography), an oligonucleotide desalting process (e.g., ultrafiltration and/or diafiltration), a liquid composition concentration process (e.g., thin film evaporation), or a drying process (e.g., freeze drying), or combinations thereof; wherein: (1) the oligonucleotide synthesis output may: (a) be pooled or combined (such that the resulting pooled/combined oligonucleotide composition has uniform character and quality, e.g., the physical and chemical properties are distributed throughout said resulting pooled/combined oligonucleotide composition): (i) before being processed in a single iteration or in a plurality of parallel iterations (not necessarily conducted simultaneously) by the at least one further process(s) (or combinations thereof); or (ii) followed by partitioning the resulting pooled/combined oligonucleotide composition into smaller portions before being processed in a plurality of parallel iterations (not necessarily conducted simultaneously) by the at least one further process(s) (or combinations thereof); or (b) be partitioned into smaller portions before being processed in a plurality of parallel iterations (not necessarily conducted simultaneously) by the at least one further process(s) (or combinations thereof); and/or (2) the output resulting from any one, multiple, or each, of the at least one further process(s) may: (a) provide the single batch composition (or single batch oligonucleotide composition) as a liquid oligonucleotide composition or a solid oligonucleotide composition; (b) be pooled or combined (such that the resulting pooled/combined oligonucleotide composition has uniform character and quality, e.g., the physical and chemical properties are distributed throughout said resulting pooled/combined oligonucleotide composition): (i) to provide the single batch composition (or single batch oligonucleotide composition) as a liquid oligonucleotide composition or a solid oligonucleotide composition; (ii) before being processed in a single iteration or in a plurality of parallel iterations (not necessarily conducted simultaneously) by a second, next, or downstream (see, e.g., FIG. 1) at least one further process(s) (or combinations thereof); or (iii) followed by partitioning the resulting pooled/combined oligonucleotide composition into smaller portions before being processed in a plurality of parallel iterations (not necessarily conducted simultaneously) by a second, next, or downstream (see, e.g., FIG. 1) at least one further process(s) (or combinations thereof); or (c) be partitioned into smaller portions before being processed in a plurality of parallel iterations (not necessarily conducted simultaneously) by a second, next, or downstream (see, e.g., FIG. 1) at least one further process(s) (or combinations thereof).

In certain embodiments, an oligonucleotide composition, such as a single batch composition, a substantially pure oligonucleotide composition, or an oligonucleotide composition prepared according to the method disclosed herein, may be derived from (or prepared by) subjecting the oligonucleotide synthesis output (such as the at least one, the plurality, the partitioned, or the pooled (or combined), oligonucleotide synthesis output) to an oligonucleotide chemical transformation process (e.g., an oligonucleotide deprotection or cleavage from solid support) in a single iteration or in a plurality of parallel iterations (not necessarily conducted simultaneously), and wherein the chemically transformed oligonucleotide output from the single or plurality of parallel iterations may be: (1) pooled or combined (such that the resulting pooled/combined oligonucleotide composition has uniform character and quality, e.g., the physical and chemical properties are distributed throughout said resulting pooled/combined oligonucleotide composition): (a) to provide the single batch composition (or single batch oligonucleotide composition) as a liquid oligonucleotide composition; (b) before being processed in a single iteration or in a plurality of parallel iterations (not necessarily conducted simultaneously) by at least one further process(s), comprising: an oligonucleotide purification, an oligonucleotide desalting process, a liquid composition concentration process, or a drying process, or combinations thereof; or (c) followed by partitioning the resulting pooled/combined oligonucleotide composition into smaller portions before being processed in a plurality of parallel iterations (not necessarily conducted simultaneously) by the at least one further process(s) (or combinations thereof); (2) further processed in a single iteration or in a plurality of parallel iterations (not necessarily conducted simultaneously) by the at least one further process(s) (or combinations thereof); or (3) partitioned into smaller portions before being processed in a plurality of parallel iterations (not necessarily conducted simultaneously) by the at least one further process(s) (or combinations thereof).

In certain embodiments, an oligonucleotide composition, such as a single batch composition, a substantially pure oligonucleotide composition, or an oligonucleotide composition prepared according to the method disclosed herein, may be derived from (or prepared by) subjecting the chemically transformed oligonucleotide output (such as the at least one, the plurality, the partitioned, or the pooled (or combined), chemically transformed oligonucleotide output) to an oligonucleotide purification process (e.g., ion-exchange chromatography) in a single iteration or in a plurality of parallel iterations (not necessarily conducted simultaneously), and wherein the purified oligonucleotide output from the single or plurality of parallel iterations may be: (1) pooled or combined (such that the resulting pooled/combined oligonucleotide composition has uniform character and quality, e.g., the physical and chemical properties are distributed throughout said resulting pooled/combined oligonucleotide composition): (a) to provide the single batch composition (or single batch oligonucleotide composition) as a liquid oligonucleotide composition; (b) before being processed in a single iteration or in a plurality of parallel iterations (not necessarily conducted simultaneously) by at least one further process(s), comprising: an oligonucleotide desalting process, a liquid composition concentration process, or a drying process, or combinations thereof; or (c) followed by partitioning the resulting pooled/combined oligonucleotide composition into smaller portions before being processed in a plurality of parallel iterations (not necessarily conducted simultaneously) by the at least one further process(s) (or combinations thereof); (2) further processed in a single iteration or in a plurality of parallel iterations (not necessarily conducted simultaneously) by the at least one further process(s) (or combinations thereof); or (3) partitioned into smaller portions before being processed in a plurality of parallel iterations (not necessarily conducted simultaneously) by the at least one further process(s) (or combinations thereof). In certain embodiments, the oligonucleotide purification process (e.g., ion-exchange chromatography) may be conducted in a single iteration, such as utilizing a single ion-exchange column having an ion exchange capacity sufficient to purify a specified quantity, or may be conducted in a plurality of parallel iterations, such as utilizing a plurality of ion-exchange columns or conducting a plurality of runs on one or more parallel columns or subsequent to each other (not necessarily conducted simultaneously) that have an ion exchange capacity sufficient to purify a specified quantity, for example, may be conducted utilizing 1, 2, 3, 4, 5, 6, 7, 8, 9, 10, 15, 20, 25, 30, 35, 40, 45, 50, 55, or 60 ion-exchange column(s) having an ion exchange capacity sufficient to purify a specified quantity or purification runs on an ion-exchange column(s) (e.g., run on parallel columns or subsequent to each other) with an ion exchange capacity sufficient to purify a specified quantity, for example, no more than between 1-60, 1-55, 1-50, 1-45, 1-40, 1-35, 1-30, 1-25, 1-20, 1-15, 1-10, 1-9, 1-8, 1-7, 1-6, 1-5, 1-4, 1-3, 1-2, 3-10, 3-8, 3-5, 4-6, 5-10, 10-60, 10-50, 10-40, 10-30, 10-25, 10-20, 10-15, 20-50, 30-60, or 40-55 ion-exchange column(s) having an ion exchange capacity sufficient to purify a specified quantity or purification runs on an ion-exchange column(s) (e.g., run on parallel columns or subsequent to each other) with an ion exchange capacity sufficient to purify a specified quantity.

In certain embodiments, an oligonucleotide composition, such as a single batch composition, a substantially pure oligonucleotide composition, or an oligonucleotide composition prepared according to the method disclosed herein, may be derived from (or prepared by) subjecting the purified oligonucleotide output (such as the at least one, the plurality, the partitioned, or the pooled (or combined), purified oligonucleotide output) to an oligonucleotide desalting process (e.g., ultrafiltration and/or diafiltration) in a single iteration or in a plurality of parallel iterations (not necessarily conducted simultaneously), and wherein the desalted oligonucleotide output from the single or plurality of parallel iterations may be: (1) pooled or combined (such that the resulting pooled/combined oligonucleotide composition has uniform character and quality, e.g., the physical and chemical properties are distributed throughout said resulting pooled/combined oligonucleotide composition): (a) to provide the single batch composition (or single batch oligonucleotide composition) as a liquid oligonucleotide composition; (b) before being processed in a single iteration or in a plurality of parallel iterations (not necessarily conducted simultaneously) by at least one further process(s), comprising: a liquid composition concentration process, or a drying process, or combinations thereof; or (c) followed by partitioning the resulting pooled/combined oligonucleotide composition into smaller portions before being processed in a plurality of parallel iterations (not necessarily conducted simultaneously) by the at least one further process(s) (or combinations thereof); (2) further processed in a single iteration or in a plurality of parallel iterations (not necessarily conducted simultaneously) by the at least one further process(s) (or combinations thereof); or (3) partitioned into smaller portions before being processed in a plurality of parallel iterations (not necessarily conducted simultaneously) by the at least one further process(s) (or combinations thereof).

In certain embodiments, an oligonucleotide composition, such as a single batch composition, a substantially pure oligonucleotide composition, or an oligonucleotide composition prepared according to the method disclosed herein, may be derived from (or prepared by) subjecting the desalted oligonucleotide output (such as the at least one, the plurality, the partitioned, or the pooled (or combined), desalted oligonucleotide output) to a liquid composition concentration process (e.g., thin film evaporation) in a single iteration or in a plurality of parallel iterations (not necessarily conducted simultaneously), and wherein the concentrated oligonucleotide liquid composition output from the single or plurality of parallel iterations may be: (1) pooled or combined (such that the resulting pooled/combined oligonucleotide composition has uniform character and quality, e.g., the physical and chemical properties are distributed throughout said resulting pooled/combined oligonucleotide composition): (a) to provide the single batch composition (or single batch oligonucleotide composition) as a liquid oligonucleotide composition; (b) before being processed in a single iteration or in a plurality of parallel iterations (not necessarily conducted simultaneously) by a drying process; or (c) followed by partitioning the resulting pooled/combined oligonucleotide composition into smaller portions before being processed in a plurality of parallel iterations (not necessarily conducted simultaneously) by the drying process; (2) further processed in a single iteration or in a plurality of parallel iterations (not necessarily conducted simultaneously) by the drying process; or (3) partitioned into smaller portions before being processed in a plurality of parallel iterations (not necessarily conducted simultaneously) by the drying process.

In certain embodiments, an oligonucleotide composition, such as a single batch composition, a substantially pure oligonucleotide composition, or an oligonucleotide composition prepared according to the method disclosed herein, may be derived from (or prepared by) subjecting the concentrated oligonucleotide liquid composition output (such as the at least one, the plurality, the partitioned, or the pooled (or combined), concentrated oligonucleotide liquid composition output) to a drying process (e.g., freeze drying) in a single iteration or in a plurality of parallel iterations (not necessarily conducted simultaneously), and wherein the dried oligonucleotide solid composition output from the single or plurality of parallel iterations may be pooled (or combined) such that the resulting pooled/combined oligonucleotide composition has uniform character and quality, e.g., the physical and chemical properties are distributed throughout said resulting pooled/combined oligonucleotide composition to provide the single batch composition (or single batch oligonucleotide composition) as a solid oligonucleotide composition.

In certain embodiments, the amount of oligonucleotide contained within an output oligonucleotide composition, for example, the oligonucleotide synthesis output or the output from one or more of the further processes (such as the chemically transformed oligonucleotide output, the purified oligonucleotide output, the desalted oligonucleotide output, the concentrated oligonucleotide liquid composition output, and/or the dried oligonucleotide composition output), may be a specified quantity. In certain embodiments, the output oligonucleotide composition (such as the oligonucleotide synthesis output or the output from one or more of the further processes) may be an at least one output, a pooled or combined output, or a partitioned output. In certain embodiments, the specified quantity may be at least 100 mmol, such as, at least 150 mmol, at least 200 mmol, at least 250 mmol, at least 300 mmol, at least 500 mmol, at least 600 mmol, at least 700 mmol, at least 800 mmol, at least 900 mmol, at least 1,000 mmol, at least 1,200 mmol, at least 1,400 mmol, at least 1,600 mmol, at least 1,800 mmol, at least 2,000 mmol, at least 2,500 mmol, at least 3,000 mmol, at least 3,500 mmol, at least 4,000 mmol, or at least 5,000 mmol, for example, the specified quantity may be in the range of between 300-5,400 mmol, such as in the range of between 300-4,500 mmol, 300-4,000 mmol, 300-3,600 mmol, 300-3,000 mmol, 600-3,000 mmol, 700-3,600 mmol, 700-2,700 mmol, 700-2,500 mmol, 700-2,400 mmol, 700-2,000 mmol, 700-1,900 mmol, 700-1,800 mmol, 700-1,700 mmol, 700-1,600 mmol, 700-1,500 mmol, 700-1,400 mmol, 700-1,300 mmol, 700-1,200 mmol, 700-1,100 mmol, 700-1,000 mmol, 700-900 mmol, 800-1,200 mmol, 800-1,000 mmol, 900-1,600 mmol, 900-1,800 mmol, 900-2,400 mmol, 900-2,700 mmol, 900-3,600 mmol, 1,800-2,700 mmol, 1,800-3,600 mmol, 1,000-3,000 mmol, 1,000-2,500 mmol, 1,000-2,000 mmol, 1,000-1,500 mmol, 1,250-1,750 mmol, 1,500-3,000 mmol, 1,500-2,500 mmol, 1,500-2,000 mmol, 1,600-2,400 mmol, 2,000-3,000 mmol, 2,500-3,000 mmol, 2,700-3,600 mmol, 2,700-4,500 mmol, 3,000-4,000 mmol, 3,600-4,500 mmol, or 3,600-5,400 mmol.

In certain embodiments, the plurality of parallel iterations (not necessarily conducted simultaneously), such as the plurality of parallel iterations involving the oligonucleotide chemical transformation process (e.g., an oligonucleotide deprotection or cleavage from solid support), the oligonucleotide purification process (e.g., ion-exchange chromatography), the oligonucleotide desalting process (e.g., ultrafiltration and/or diafiltration), the liquid composition concentration process (e.g., thin film evaporation), or the drying process (e.g., freeze drying), may be no more than 2, 3, 4, 5, 6, 7, 8, 9, 10, 15, 20, 25, 30, 35, 40, 45, 50, 55, or at most 60 parallel iterations, for example, no more than between 2-60, 2-55, 2-50, 2-45, 2-40, 2-35, 2-30, 2-25, 2-20, 2-15, 2-10, 2-9, 2-8, 2-7, 2-6, 2-5, 2-4, 2-3, 3-10, 3-8, 3-5, 4-6, 5-10, 10-60, 10-50, 10-40, 10-30, 10-25, 10-20, 10-15, 20-50, 30-60, or 40-55 parallel iterations. In certain embodiments, the further process(s) may be conducted downstream from the synthesis process in the following sequence: an oligonucleotide chemical transformation process (e.g., an oligonucleotide deprotection or cleavage from solid support), an oligonucleotide purification process (e.g., ion-exchange chromatography), an oligonucleotide desalting process (e.g., ultrafiltration and/or diafiltration), a liquid composition concentration process (e.g., thin film evaporation), and a drying process (e.g., freeze drying). In certain embodiments, the further process(s) may be conducted downstream from the synthesis process in the following sequence: an oligonucleotide chemical transformation process (e.g., an oligonucleotide deprotection or cleavage from solid support), an oligonucleotide purification process (e.g., ion-exchange chromatography), and an oligonucleotide desalting process (e.g., ultrafiltration and/or diafiltration); wherein the sequence may be followed by a liquid composition concentration process (e.g., thin film evaporation) and/or a drying process (e.g., freeze drying); or wherein the sequence may be exclusive of a liquid composition concentration process (e.g., thin film evaporation) and/or a drying process (e.g., freeze drying).

In certain embodiments, a single batch composition disclosed herein, may be derived from the following sequence of processes (each performed in a single iteration, a plurality of iterations done in parallel (not necessarily simultaneously), or combinations thereof): an oligonucleotide synthesis process, a purification process of the synthesized oligonucleotide, a desalting process of the purified oligonucleotide, a liquid concentration process of the desalted oligonucleotide, and a drying process of the concentrated oligonucleotide, to provide the single batch composition as a solid composition. In certain embodiments, a single batch composition disclosed herein, may involve pooling the output of a plurality of iterations (or runs) of a particular process step before proceeding to the next process step (if any), for example, pooling the output of a plurality of oligonucleotide synthesis column(s) (or synthesis run(s)), of a plurality of ion-exchange purification column(s) (or purification run(s)) of the synthesized oligonucleotide, of a plurality of desalting the purified oligonucleotide via ultrafiltration and/or diafiltration process(s), and/or of a plurality of liquid concentrating the desalted oligonucleotide (such as via a plurality of thin film evaporation process(s)), before proceeding to the next process step. In certain embodiments, a single batch composition disclosed herein, may be a solid composition derived from pooling the output of a plurality of drying the liquid concentrated, desalted, oligonucleotide (such as via a plurality of freeze drying process(s)). In certain embodiments, a single batch composition disclosed herein, may be a liquid composition, such as a concentrated liquid composition, in which case the single batch composition is derived exclusive of a drying process (e.g., exclusive of a freeze drying process).

In certain embodiments, a single batch composition disclosed herein, may involve partitioning the output of a particular process step before proceeding to the next process step (if any) which is to be conducted in a plurality of parallel iterations (or runs, not necessarily simultaneously), for example, partitioning the output of an oligonucleotide synthesis column(s) (or synthesis run(s)), of an ion-exchange purification column(s) (or purification run(s)) of the synthesized oligonucleotide, of a desalting the purified oligonucleotide via ultrafiltration and/or diafiltration process(s), and/or of a liquid concentrating the desalted oligonucleotide (such as via a plurality of thin film evaporation process(s)), before proceeding to the next process step (if any) which is to be conducted in a plurality of parallel iterations (or runs, not necessarily simultaneously). In certain embodiments, a single batch composition disclosed herein, may be a solid composition derived from partitioning the output of a plurality of drying the liquid concentrated, desalted, oligonucleotide (such as via a plurality of freeze drying process(s)). In certain embodiments, a single batch composition disclosed herein, may be a liquid composition, such as a concentrated liquid composition, in which case the single batch composition is derived exclusive of a drying process (e.g., exclusive of a freeze drying process).

In certain embodiments, a single batch composition disclosed herein, may involve directly proceeding to process the output of a particular process step in the next process step (if any), exclusive of a pooling or partitioning of the output from the particular process step, wherein the next process step may involve processing only a single output from the particular process step or may involve processing a plurality of outputs from the particular step in a series of parallel iterations (or runs, not necessarily simultaneously), for example, directly proceeding to process the output of an oligonucleotide synthesis column(s) (or synthesis run(s)), of an ion-exchange purification column(s) (or purification run(s)) of the synthesized oligonucleotide, of a desalting the purified oligonucleotide via ultrafiltration and/or diafiltration process(s), and/or of a liquid concentrating the desalted oligonucleotide (such as via a plurality of thin film evaporation process(s)), in the next process step (if any) which may be conducted in a single or in a plurality of parallel iterations (or runs, not necessarily simultaneously). In certain embodiments, a single batch composition disclosed herein, may be a solid composition derived from directly proceeding to dry a single output or a plurality of outputs of the liquid concentrated, desalted, oligonucleotide (such as via a single or plurality of freeze drying process(s)).

In certain embodiments, a single batch composition disclosed herein, may be derived from the following sequence of processes (each performed in a single iteration, a plurality of iterations done in parallel (not necessarily simultaneously), or combinations thereof): an oligonucleotide synthesis process, a purification process of the synthesized oligonucleotide, a desalting process of the purified oligonucleotide, a liquid concentration process of the desalted oligonucleotide, and a drying process of the concentrated oligonucleotide, to provide the single batch composition as a solid composition; wherein the output of a particular step in the sequence may be pooled before proceeding to the next process step (if any), may be partitioned before proceeding to the next process step (if any), or may be processed directly in the next process step (if any) exclusive of pooling or partitioning the output from the particular process step, or combinations thereof (e.g., some process steps may involve pooling while other process steps directly process the output of the prior step exclusive of pooling or partitioning).

A schematic represention of certain embodiments in preparing a single batch oligonucleotide composition, as used herein, is provided in FIG. 1. For example, the column of boxes on the left side of FIG. 1, illustrates a series of general process steps utilized to prepare a single batch composition as a solid composition, and a particular sequence in which the series of general process steps are conducted: an oligonucleotide synthesis process, a purification process of the synthesized oligonucleotide, a desalting process of the purified oligonucleotide, a liquid concentration process of the desalted oligonucleotide, and a drying process of the concentrated oligonucleotide. The second column of boxes from the left side of FIG. 1, illustrates one particular type of process that may be utilized to achieve a particular general process step (e.g., the oligonucleotide synthesis process may be achieved utilizing an oligonucleotide synthesis column having a solid support with a loading capacity of a specified quantity; the purification process may be achieved utilizing an ion-exchange chromatography column; the desalting process may be achieved utilizing an ultrafiltration and/or diafiltration process; the liquid concentration process may be achieved utilizing a thin film evaporation process; and the drying process may be achieved utilizing a freeze drying process). The remaining columns of boxes on the right side of FIG. 1 illustrate a 3×900 mmol, 4×900 mmol, and a 5×900 mmol scale preparation of a single batch oligonucleotide composition (e.g., for the particular embodiments illustrated in FIG. 1: synthesis step: an oligonucleotide synthesis column(s) is utilized having a solid support with a 900 mmol loading capacity, wherein 3, 4, or 5, oligonucleotide synthesis columns may be run in parallel or as a single column ran in series 3, 4, or 5 times, or combinations thereof, to achieve the 3×900 mmol, 4×900 mmol, and a 5×900 mmol scale synthesis; purification step: an ion-exchange chromatography column(s) is utilized having an ion-exchange capacity to purify an oligonucleotide composition containing 900 mmol of the oligonucleotide, wherein the oligonucleotide synthesis output from the oligonucleotide synthesis column(s) (e.g., from 3, 4, or 5 columns) proceeded directly to the purification step (exclusive of a pooling or partitioning of the synthesis output), and wherein 3, 4, or 5, ion-exchange chromatography columns may be run in parallel or as a single column ran in series 3, 4, or 5 times, or combinations thereof, to achieve the 3×900 mmol, 4×900 mmol, and a 5×900 mmol scale purification; desalting step: an ultrafiltration and/or diafiltration process is utilized having a capacity to desalt a pooled oligonucleotide composition containing 2,700 mmol, 3,600 mmol, or 4,500 mmol, of the oligonucleotide to achieve the 3×900 mmol, 4×900 mmol, and a 5×900 mmol scale desalting, wherein the pooled oligonucleotide composition was provided by pooling the output from 3, 4, or 5, ion-exchange chromatography column(s) (run in parallel or sequentially), respectively, before proceeding to the desalting process; liquid concentration step: an thin film evaporation process is utilized having a capacity to concentrate a desalted, liquid oligonucleotide composition containing 2,700 mmol, 3,600 mmol, or 4,500 mmol of the oligonucleotide to achieve the 3×900 mmol, 4×900 mmol, and a 5×900 mmol scale liquid concentration, wherein the desalting output from the UF/DF process proceeded directly to the liquid concentration step (exclusive of a pooling or partitioning of the desalting output); and drying step: a freeze drying process is utilized having a capacity to dry a concentrated liquid composition containing 2,700 mmol, 3,600 mmol, or 4,500 mmol of the oligonucleotide to achieve the 3×900 mmol, 4×900 mmol, and a 5×900 mmol scale drying, wherein the liquid concentration output from the TFE process proceeded directly to the drying step (exclusive of a pooling or partitioning of the liquid concentrated output).

In certain embodiments, an oligonucleotide composition, such as a single batch composition, a substantially pure oligonucleotide composition, or an oligonucleotide composition prepared according to the method disclosed herein, may be derived from a series of processes including an oligonucleotide synthesis process, an oligonucleotide purification process, an oligonucleotide desalting process, and an oligonucleotide liquid composition concentration process, and optionally an oligonucleotide drying process, wherein at least one process in the series is conducted in no more than one single iteration and all remaining downstream processes in the series (if any) are conducted in no more than one single iteration so as to create a single output from said at least one process and thereby provide a purified, desalted, and concentrated (and optionally dried) oligonucleotide composition.

In certain embodiments, an oligonucleotide composition, such as a single batch composition, a substantially pure oligonucleotide composition, or an oligonucleotide composition prepared according to the method disclosed herein, may be derived from the series of processes wherein one or more of the processes in the series may be conducted in a plurality of iterations and at least one process in said series that is downstream from the process conducted in a plurality of iterations is conducted in no more than one single iteration and combines (or pools) all the output of the prior process conducted in a plurality of iterations so as to create a single output from the at least one downstream process and thereby provide a purified, desalted, and concentrated (and optionally dried) oligonucleotide composition.

In certain embodiments, an oligonucleotide composition, such as a single batch composition, a substantially pure oligonucleotide composition, or an oligonucleotide composition prepared according to the method disclosed herein, may be derived from the series of processes that includes a single iteration of an oligonucleotide synthesis process, a single iteration of an oligonucleotide purification process, a single iteration of an oligonucleotide desalting process, and a single iteration of an oligonucleotide liquid composition concentration process, and optionally a single iteration of an oligonucleotide drying process, and thereby providing a purified, desalted, and concentrated (and optionally dried) oligonucleotide composition.

In certain embodiments, an oligonucleotide composition, such as a single batch composition, a substantially pure oligonucleotide composition, or an oligonucleotide composition prepared according to the method disclosed herein, may be derived from the series of processes that includes a plurality of iterations of an oligonucleotide synthesis process, a plurality of iterations of an oligonucleotide purification process, a single iteration of an oligonucleotide desalting process conducted by desalting the combined or pooled output from the plurality of iterations of the oligonucleotide purification process, and a single iteration of an oligonucleotide liquid composition concentration process, and optionally a single iteration of an oligonucleotide drying process, and thereby providing a purified, desalted, and concentrated (and optionally dried) oligonucleotide composition.

In certain embodiments, an oligonucleotide composition, such as a single batch composition, a substantially pure oligonucleotide composition, or an oligonucleotide composition prepared according to the method disclosed herein, may be derived from the series of processes that includes a plurality of iterations of a single oligonucleotide synthesis process followed a single oligonucleotide purification process, a single iteration of an oligonucleotide desalting process conducted by desalting the combined or pooled output from the plurality of iterations of the single oligonucleotide synthesis process followed the single oligonucleotide purification process, and a single iteration of an oligonucleotide liquid composition concentration process, and optionally a single iteration of an oligonucleotide drying process thereby, and thereby providing a purified, desalted, and concentrated (and optionally dried) oligonucleotide composition.

In certain embodiments, an oligonucleotide composition, such as a single batch composition, a substantially pure oligonucleotide composition, or an oligonucleotide composition prepared according to the method disclosed herein, may be derived from the series of processes that is exclusive of the optional oligonucleotide drying process, to provide a liquid oligonucleotide composition. In certain embodiments, an oligonucleotide composition, such as a single batch composition, a substantially pure oligonucleotide composition, or an oligonucleotide composition prepared according to the method disclosed herein, may be derived from the series of processes that is inclusive of the optional oligonucleotide drying process, to provide a solid oligonucleotide composition.

In certain embodiments, an oligonucleotide composition, such as a single batch composition, a substantially pure oligonucleotide composition, or an oligonucleotide composition prepared according to the method disclosed herein, may comprise or consist of an oligonucleotide at a degree of homogeneity of at least 65%, such as at least 70%, at least 75%, at least 80%, at least 85%, at least 90%, at least 91%, at least 92%, at least 93%, at least 94%, at least 95%, at least 96%, at least 97%, at least 98%, or at least 99%, such as a degree of homogeneity in the range of between 65-99%, 70-99%, 75-99%, 80-99%, 85-99%, 90-99%, 95-99%, 65-95%, 70-95%, 75-95%, 80-97%, or 85-95%, wherein the oligonucleotide may be an anti-SMAD7 oligonucleotide, for example, an oligonucleotide having the nucleic acid sequence of SEQ ID NO: 2, 3, 4, 5, 6, 7, 8, 9, 10, 11, or 12, or the complementary sequence thereto, such as Mongersen (formerly GED-0301). In certain embodiments, the degree of homogeneity may be determined by RP-HPLC. For example, in certain embodiments, an oligonucleotide composition, such as a single batch composition, a substantially pure oligonucleotide composition, or an oligonucleotide composition prepared according to the method disclosed herein, may comprise or consist of at least 700 mmol, or at least 2 kg, of an oligonucleotide at a degree of homogeneity of at least 65%, such as at least 70%, at least 75%, at least 80%, at least 85%, at least 90%, at least 91%, at least 92%, at least 93%, at least 94%, at least 95%, at least 96%, at least 97%, at least 98%, or at least 99%, such as a degree of homogeneity in the range of between 65-99%, 70-99%, 75-99%, 80-99%, 85-99%, 90-99%, 95-99%, 65-95%, 70-95%, 75-95%, 80-97%, or 85-95%, wherein the oligonucleotide may be an anti-SMAD7 oligonucleotide, for example, an oligonucleotide having the nucleic acid sequence of SEQ ID NO: 2, 3, 4, 5, 6, 7, 8, 9, 10, 11, or 12, or the complementary sequence thereto, such as Mongersen (formerly GED-0301).

5.17 Formulations and Uses of the Oligonucleotides

An oligonucleotide synthesized according to the methods provided herein, such as an oligonucleotide obtained from a single batch composition, a substantially pure oligonucleotide composition, or an oligonucleotide composition prepared according to the method disclosed herein, see, e.g., as described in Sections 5-5.16, can be formulated into a pharmaceutical composition, see, e.g., as described in Section 5.18, and published Internation Publication WO 2016/105516, herein incorporated by reference in its entirety. For example, the oligonucleotide that can be formulated into a pharmaceutical composition may be a SMAD7 antisense oligonucleotide or a chemically modified SMAD7 antisense oligonucleotide, e.g., an oligonucleotide comprising a portion of at least 10 nucleotides of, consists of, or includes up to 21 nucleotides in length of, having a nucleic acid sequence of any one of SEQ ID NO: 2, 3, 4, 5, 6, 7, 8, 9, 10, 11, or 12, preferably having a nucleic acid sequence: SEQ ID NO: 3: (5′-GTX GCC CCT TCT CCC XGC AGC-3′), wherein X represents 5-methyl-2′-deoxycytidine, or an oligonucleotide obtained from a single batch composition, a substantially pure oligonucleotide composition, or an oligonucleotide composition prepared according to the method disclosed herein. These oligonucleotides and pharmaceutical compositions can be used in the treatment of indications, such as inflammatory indications, for example, inflammatory bowel diseases (IBD) (such as Crohn's disease (CD) or ulcerative colitis (UC)), in a patient in need thereof, see, e.g., as described in Sections 5.18-5.19, and published Internation Publication WO 2016/105516.

5.18 Pharmaceutical Compositions

The pharmaceutical compositions described in this section can be used in the methods of use provided herein. See, e.g., as described in Section 5.19. In certain embodiments, the pharmaceutical composition, such as an oral dosage form, comprises an oligonucleotide synthesized according to the methods provided herein, such as an oligonucleotide obtained from a single batch composition, a substantially pure oligonucleotide composition, or an oligonucleotide composition prepared according to the method disclosed herein, and a pharmaceutically acceptable adjuvant and/or excipient. In certain embodiments, the pharmaceutical composition is an oral pharmaceutical composition. In certain embodiments, the pharmaceutical composition comprises an enteric coating to topically deliver the oligonucleotide of the single batch composition, the substantially pure oligonucleotide composition, or the oligonucleotide composition prepared according to the method disclosed herein, to the terminal ileum and/or right colon of an IBD patient. In certain embodiments, the pharmaceutical composition is a gastro-resistant granules formulation.

Contemplated SMAD7 antisense oligonucleotides comprise oligonucleotides that act against SMAD7 and can be administered orally. Disclosed therapies can, when administered orally to a subject suffering from inflammatory bowel disease (IBD), deliver an effective amount of a SMAD7 antisense oligonucleotide to the intestinal system of a patient, e.g. deliver an effective amount of a SMAD7 antisense oligonucleotide to the terminal ileum and/or right colon of a patient.

In certain embodiments of the methods of treating IBD provided herein, the anti-SMAD7 therapy (e.g., a therapy comprising a SMAD7 antisense oligonucleotide) can be suitable for oral delivery of a SMAD7 antisense oligonucleotide, e.g., tablets, that comprise an enteric coating, e.g., a gastro-resistant coating, such that the compositions can deliver the antisense compound to, e.g., the terminal ileum and right colon of a patient. For example, such administration can result in a topical effect, substantially topically applying the antisense compound directly to an affected portion of the intestine of a subject. Such administration, can, in certain embodiments, substantially avoid unwanted systemic absorption of the antisense compound.

For example, a tablet for oral administration can comprise granules (e.g., is at least partially formed from granules) that comprise a disclosed SMAD7 antisense oligonucleotide and pharmaceutically acceptable excipients. Such a tablet can be coated with an enteric coating. Contemplated tablets can comprise pharmaceutically acceptable excipients such as fillers, binders, disintegrants, and/or lubricants, as well as coloring agents, release agents, coating agents, sweetening, flavoring such as wintergreen, orange, xylitol, sorbitol, fructose, and maltodextrin, and perfuming agents, preservatives and/or antioxidants. In certain embodiments, the pharmaceutical composition, such as an oral dosage form (e.g., tablet or coated tablet, as disclosed herein), comprises formulating (such as combining, mixing, or blending) a portion of a single batch composition comprising a disclosed SMAD7 antisense oligonucleotide, such as an oligonucleotide synthesized according to the methods provided herein, with a pharmaceutically acceptable adjuvant and/or excipient. For example, in certain embodiments, the SMAD7 antisense oligonucleotide has a nucleic acid sequence of any one of SEQ ID NO: 2, 3, 4, 5, 6, 7, 8, 9, 10, 11, or 12, preferably having a nucleic acid sequence: SEQ ID NO: 3: (5′-GTX GCC CCT TCT CCC XGC AGC-3′), wherein X represents 5-methyl-2′-deoxycytidine.

In certain embodiments, contemplated pharmaceutical formulations comprise an intra-granular phase that comprises a contemplated SMAD7 antisense oligonucleotide or a pharmaceutically acceptable salt and a pharmaceutically acceptable filler. For example, the oligonucleotide is an oligonucleotide synthesized according to the methods provided herein, such as an oligonucleotide obtained from a single batch composition, a substantially pure oligonucleotide composition, or an oligonucleotide composition prepared according to the method disclosed herein, of a SMAD7 antisense oligonucleotide or a chemically modified SMAD7 antisense oligonucleotide; e.g., an oligonucleotide comprising a portion of at least 10 nucleotides of, consists of, or includes up to 21 nucleotides in length of, having a nucleic acid sequence of any one of SEQ ID NO: 2, 3, 4, 5, 6, 7, 8, 9, 10, 11, or 12, preferably having a nucleic acid sequence: SEQ ID NO: 3: (5′-GTX GCC CCT TCT CCC XGC AGC-3′), wherein X represents 5-methyl-2′-deoxycytidine, and a filler can be blended together, with optionally other excipients, and formed into granules. In certain embodiments, the intragranular phase can be formed using wet granulation, e.g. a liquid (e.g., water) is added to the blended antisense compound and filler, and then the combination is dried, milled and/or sieved to produce granules. One of skill in the art would understand that other processes can be used to achieve an intragranular phase.

In certain embodiments, contemplated formulations comprise an extra-granular phase, which can comprise one or more pharmaceutically acceptable excipients, and which can be blended with the intragranular phase to form a disclosed formulation.

An anti-SMAD7 therapy formulation can comprise an intragranular phase that comprises a filler. Exemplary fillers comprise, but are not limited to, cellulose, gelatin, calcium phosphate, lactose, sucrose, glucose, mannitol, sorbitol, microcrystalline cellulose, pectin, polyacrylates, dextrose, cellulose acetate, hydroxypropylmethyl cellulose, partially pregelatinized starch, calcium carbonate, and others including combinations thereof.

In certain embodiments, an anti-SMAD7 therapy formulation can comprise an intragranular phase and/or an extragranular phase that comprises a binder, which can generally function to hold the ingredients of the pharmaceutical formulation together. Exemplary binders comprise, for example, the following: starches, sugars, cellulose or modified cellulose such as hydroxypropyl cellulose, lactose, pregelatinized maize starch, polyvinyl pyrrolidone, hydroxypropyl cellulose, hydroxypropylmethyl cellulose, low substituted hydroxypropyl cellulose, sodium carboxymethyl cellulose, methyl cellulose, ethyl cellulose, sugar alcohols and others, including combinations thereof.

Contemplated anti-SMAD7 therapy formulations, e.g., that comprise an intragranular phase and/or an extragranular phase, can comprise a disintegrant, such as, but not limited to, starch, cellulose, crosslinked polyvinyl pyrrolidone, sodium starch glycolate, sodium carboxymethyl cellulose, alginates, corn starch, crosmellose sodium, crosslinked carboxymethyl cellulose, low substituted hydroxypropyl cellulose, acacia, and others including combinations thereof. For example, an intragranular phase and/or an extragranular phase can comprise a disintegrant.

In certain embodiments, a contemplated anti-SMAD7 therapy formulation comprises an intra-granular phase comprising a disclosed antisense compound and excipients chosen from: mannitol, microcrystalline cellulose, hydroxypropylmethyl cellulose, and sodium starch glycolate, or combinations thereof, and an extra-granular phase comprising one or more of: microcrystalline cellulose, sodium starch glycolate, and magnesium stearate, or mixtures thereof.

In certain embodiments, a contemplated anti-SMAD7 therapy formulation can comprise a lubricant, e.g., an extra-granular phase can contain a lubricant. Lubricants comprise but are not limited to talc, silica, fats, stearin, magnesium stearate, calcium phosphate, silicone dioxide, calcium silicate, calcium phosphate, colloidal silicon dioxide, metallic stearates, hydrogenated vegetable oil, corn starch, sodium benzoate, polyethylene glycols, sodium acetate, calcium stearate, sodium lauryl sulfate, sodium chloride, magnesium lauryl sulfate, talc, and stearic acid.

In certain embodiments, the pharmaceutical formulation comprises an enteric coating. Generally, enteric coatings create a barrier for the oral medication that controls the location at which the drug is absorbed along the digestive track. Enteric coatings can comprise a polymer that disintegrates at different rates according to pH. Enteric coatings can comprise, for example, cellulose acetate phthalate, methyl acrylate-methacrylic acid copolymers, cellulose acetate succinate, hydroxylpropylmethyl cellulose phthalate, methyl methacrylate-methacrylic acid copolymers, ethylacrylate-methacrylic acid copolymers, methacrylic acid copolymer type C, polyvinyl acetate-phthalate, and cellulose acetate phthalate.

In certain embodiments, the enteric coating comprises an anionic, cationic, or neutral copolymer based on methacrylic acid, methacrylic/acrylic esters or their derivatives. In certain embodiments, the enteric coating comprises an ethylacrylate-methacrylic acid copolymer. Commercially available enteric coatings comprise Opadry® AMB, Acryl-EZE®, Eudragit®. In certain embodiments, the enteric coating makes up about 5% to about 10%, about 5% to about 20%, about 8 to about 15%, about 8% to about 18%, about 10% to about 12%, or about 12% to about 16%, of a contemplated tablet by weight.

For example, an anti-SMAD7 therapy in the form of a tablet is provided that comprises or consists essentially of about 0.5% to about 70%, e.g., about 0.5% to about 10%, or about 1% to about 20%, by weight of a SMAD7 antisense oligonucleotide or a pharmaceutically acceptable salt thereof. Such a tablet can comprise for example, about 0.5% to about 60% by weight of mannitol, e.g., about 30% to about 50% by weight mannitol, e.g., about 40% by weight mannitol; and/or about 20% to about 40% by weight of microcrystalline cellulose, or about 10% to about 30% by weight of microcrystalline cellulose. For example, a contemplated tablet can comprise an intragranular phase that comprises about 30% to about 60%, e.g., about 45% to about 65% by weight, or alternatively, about 5 to about 10% by weight of an oligonucleotide that is synthesized according to the methods provided herein, such as an oligonucleotide obtained from a single batch composition, a substantially pure oligonucleotide composition, or an oligonucleotide composition prepared according to the method disclosed herein, wherein the oligonucleotide has a nucleic acid sequence of any one of SEQ ID NO: 2, 3, 4, 5, 6, 7, 8, 9, 10, 11, or 12, preferably having a nucleic acid sequence: SEQ ID NO: 3: (5′-GTX GCC CCT TCT CCC XGC AGC-3′), wherein X represents 5-methyl-2′-deoxycytidine, about 30% to about 50%, or alternatively, about 5% to about 15% by weight mannitol, about 5% to about 15% microcrystalline cellulose, about 0% to about 4%, or about 1% to about 7% hydroxypropylmethyl cellulose, and about 0% to about 4%, e.g., about 2% to about 4% sodium starch glycolate by weight.

Exemplary anti-SMAD7 therapy formulations comprise dosage forms that comprise or consist essentially of about 10 mg to about 500 mg of an SMAD7 antisense oligonucleotide that is synthesized according to the methods provided herein, such as obtained from a single batch composition, a substantially pure oligonucleotide composition, or an oligonucleotide composition prepared according to the method disclosed herein, wherein the oligonucleotide has a nucleic acid sequence of SEQ ID NO: 3: (5′-GTX GCC CCT TCT CCC XGC AGC-3′), wherein X represents 5-methyl-2′-deoxycytidine, for example, tablets that comprise between about 30 mg and about 310 mg, between about 50 mg and about 290 mg, between about 70 mg and about 270 mg, between about 70 mg and about 250 mg, between about 90 mg and about 230 mg, between about 110 mg and about 210 mg, or between 130 mg and about 190 mg, or between 150 mg and about 170 mg of the SMAD7 antisense oligonucleotide, are contemplated herein. In certain embodiments, the tablets comprise between about 5 mg and about 90 mg, between about 10 mg and about 70 mg, or between about 30 mg and about 50 mg of an SMAD7 antisense oligonucleotide that is synthesized according to the methods provided herein, such as obtained from a single batch composition, a substantially pure oligonucleotide composition, or an oligonucleotide composition prepared according to the method disclosed herein, wherein the oligonucleotide has a nucleic acid sequence of SEQ ID NO: 3: (5′-GTX GCC CCT TCT CCC XGC AGC-3′), wherein X represents 5-methyl-2′-deoxycytidine. In certain embodiments, the tablets comprise about 20 mg, about 40 mg, about 60 mg, about 80 mg, about 100 mg, about 120 mg, about 140 mg, about 160 mg, about 180 mg, about 200 mg, about 220 mg, about 240 mg, about 260 mg, about 280 mg, or about 300 mg of an SMAD7 antisense oligonucleotide that is synthesized according to the methods provided herein, such as obtained from a single batch composition, a substantially pure oligonucleotide composition, or an oligonucleotide composition prepared according to the method disclosed herein, wherein the oligonucleotide has a nucleic acid sequence of SEQ ID NO: 3: (5′-GTX GCC CCT TCT CCC XGC AGC-3′), wherein X represents 5-methyl-2′-deoxycytidine.

In one embodiment, the anti-SMAD7 therapy can be a tablet for oral use comprising: about 0.5% to about 10% by weight of an SMAD7 antisense oligonucleotide that is synthesized according to the methods provided herein, such as obtained from a single batch composition, a substantially pure oligonucleotide composition, or an oligonucleotide composition prepared according to the method disclosed herein, wherein the oligonucleotide has a nucleic acid sequence of SEQ ID NO: 3: (5′-GTX GCC CCT TCT CCC XGC AGC-3′), wherein X represents 5-methyl-2′-deoxycytidine, or a pharmaceutically acceptable salt thereof; about 30% to about 50% by weight mannitol; and about 10% to about 30% by weight microcrystalline cellulose.

In an exemplary embodiment of the invention, a pharmaceutically acceptable tablet for oral administration is provided that comprises an intra-granular phase that can comprise about 50% by weight of an SMAD7 antisense oligonucleotide that is synthesized according to the methods provided herein, such as obtained from a single batch composition, a substantially pure oligonucleotide composition, or an oligonucleotide composition prepared according to the method disclosed herein, wherein the oligonucleotide has a nucleic acid sequence of SEQ ID NO: 3: (5′-GTX GCC CCT TCT CCC XGC AGC-3′), wherein X represents 5-methyl-2′-deoxycytidine, (or salt thereof), about 11.5% by weight mannitol, about 10% by weight microcrystalline cellulose, about 3% by weight hydroxypropylmethyl cellulose, and about 2.5% by weight sodium starch glycolate; and an extra-granular phase that can comprise about 20% by weight microcrystalline cellulose, about 2.5% by weight sodium starch glycolate, and about 0.5% by weight magnesium stearate. The tablet can also comprise an enteric coating.

In another exemplary embodiment, a pharmaceutically acceptable tablet for oral administration is provided that comprises or consists essentially of: an intra-granular phase that can comprise or consist essentially of about 5% to about 10%, e.g., about 8% by weight of an SMAD7 antisense oligonucleotide that is synthesized according to the methods provided herein, such as obtained from a single batch composition, a substantially pure oligonucleotide composition, or an oligonucleotide composition prepared according to the method disclosed herein, wherein the oligonucleotide has a nucleic acid sequence of SEQ ID NO: 3: (5′-GTX GCC CCT TCT CCC XGC AGC-3′), wherein X represents 5-methyl-2′-deoxycytidine, about 40% by weight mannitol, about 8% by weight microcrystalline cellulose, about 5% by weight hydroxypropylmethyl cellulose, and about 2% by weight sodium starch glycolate; and an extra-granular phase that can comprise about 17% by weight microcrystalline cellulose, about 2% by weight sodium starch glycolate, and about 0.4% by weight magnesium stearate.

Contemplated tablets can also comprise an enteric coating, e.g., a disclosed tablet can comprise about 10%, about 11%, about 12%, about 13%, about 14%, about 15%, about 16%, about 17%, or about 18% by weight of an enteric coating, e.g., ethylacrylate-methacrylic acid copolymers (e.g., AcyrlEZE®).

In certain embodiments, the tablet is a coated tablet, and the coated tablet comprises about 11% by weight of an enteric coating, e.g., ethylacrylate-methacrylic acid copolymers (e.g., AcyrlEZE®) and about 40 mg or 160 mg of an SMAD7 antisense oligonucleotide that is synthesized according to the methods provided herein, such as obtained from a single batch composition, a substantially pure oligonucleotide composition, or an oligonucleotide composition prepared according to the method disclosed herein, wherein the oligonucleotide has a nucleic acid sequence of SEQ ID NO: 3: (5′-GTX GCC CCT TCT CCC XGC AGC-3′), wherein X represents 5-methyl-2′-deoxycytidine. For example, the anti-SMAD7 therapy can be in the form of a pharmaceutically acceptable tablet for oral use comprising an intra-granular phase and extra-granular phase, wherein for example, the intra-granular phase comprises about 5% to about 10%, by weight (for example about 8% by weight) of an SMAD7 antisense oligonucleotide that is synthesized according to the methods provided herein, such as obtained from a single batch composition, a substantially pure oligonucleotide composition, or an oligonucleotide composition prepared according to the method disclosed herein, wherein the oligonucleotide has a nucleic acid sequence of SEQ ID NO: 3: (5′-GTX GCC CCT TCT CCC XGC AGC-3′), wherein X represents 5-methyl-2′-deoxycytidine, or a pharmaceutically acceptable salt thereof, about 40% by weight mannitol, about 8% by weight microcrystalline cellulose, about 5% by weight hydroxypropylmethyl cellulose, and about 2% by weight sodium starch glycolate, and, e.g., the extra-granular phase comprises about 17% by weight microcrystalline cellulose, about 2% by weight sodium starch glycolate, and about 0.4% by weight magnesium stearate, where the tablet can further comprise an enteric coating.

In certain embodiments, an oligonucleotide synthesized according to the methods provided herein, such as an oligonucleotide obtained from a single batch composition, a substantially pure oligonucleotide composition, or an oligonucleotide composition prepared according to the method disclosed herein, see, e.g., as described in Sections 5-5.16, can be formulated into a series of pharmaceutical compositions, for example, oral dosage forms (e.g., tablets or coated tablets as described herein), according to the methods disclosed herein. For example, in certain embodiments, a method of preparing a series of tablets (e.g., coated tablets), comprises partitioning a single batch composition of an oligonucleotide comprising at least 700 mmol of the oligonucleotide, e.g., at least 700 mmol of the oligonucleotide and at most 25 wt. % water, into a portion suitable for oral dosage, and combining the portion with a pharmaceutically acceptable adjuvant and/or excipient; wherein the oligonucleotide is an SMAD7 antisense oligonucleotide, for example, wherein the oligonucleotide has a nucleic acid sequence of any one of SEQ ID NO: 2, 3, 4, 5, 6, 7, 8, 9, 10, 11, or 12, preferably having a nucleic acid sequence: SEQ ID NO: 3: (5′-GTX GCC CCT TCT CCC XGC AGC-3′), wherein X represents 5-methyl-2′-deoxycytidine. For example, in certain embodiments, a method of preparing a series of tablets (e.g., coated tablets), comprises partitioning a single batch composition of an oligonucleotide comprising at least 2 g/mmol of an at least 700 mmol synthesis scale of the oligonucleotide, e.g., at least 2 g/mmol of an at least 700 mmol synthesis scale of the oligonucleotide and at most 25 wt. % water, into one or more portions suitable for oral dosage, and combining at least one, or each, of the one or more portions with a pharmaceutically acceptable adjuvant and/or excipient; wherein the oligonucleotide is an SMAD7 antisense oligonucleotide, for example, wherein the oligonucleotide has a nucleic acid sequence of any one of SEQ ID NO: 2, 3, 4, 5, 6, 7, 8, 9, 10, 11, or 12, preferably having a nucleic acid sequence: SEQ ID NO: 3: (5′-GTX GCC CCT TCT CCC XGC AGC-3′), wherein X represents 5-methyl-2′-deoxycytidine. For example, in certain embodiments, a method of preparing a series of tablets (e.g., coated tablets), comprises partitioning a single batch composition of an oligonucleotide comprising at least 2 kg of the oligonucleotide, e.g., comprising at least 2 kg of the oligonucleotide and at most 25 wt. % water, into one or more portions suitable for oral dosage, and combining at least one, or each, of the one or more portions with a pharmaceutically acceptable adjuvant and/or excipient; wherein the oligonucleotide is an SMAD7 antisense oligonucleotide, for example, wherein the oligonucleotide has a nucleic acid sequence of any one of SEQ ID NO: 2, 3, 4, 5, 6, 7, 8, 9, 10, 11, or 12, preferably having a nucleic acid sequence: SEQ ID NO: 3: (5′-GTX GCC CCT TCT CCC XGC AGC-3′), wherein X represents 5-methyl-2′-deoxycytidine. For example, in certain embodiments, a method of preparing a series of tablets (e.g., coated tablets), comprises partitioning a single batch composition of an oligonucleotide comprising at least 50 mol % of the oligonucleotide output from at least one 700 mmol or greater oligonucleotide synthesis column, e.g., comprising at least 50 mol % of the oligonucleotide output from at least one 700 mmol or greater oligonucleotide synthesis column and at most 25 wt. % water, into one or more portions suitable for oral dosage, and combining at least one, or each, of the portions with a pharmaceutically acceptable adjuvant and/or excipient; wherein the oligonucleotide is an SMAD7 antisense oligonucleotide, for example, wherein the oligonucleotide has a nucleic acid sequence of any one of SEQ ID NO: 2, 3, 4, 5, 6, 7, 8, 9, 10, 11, or 12, preferably having a nucleic acid sequence: SEQ ID NO: 3: (5′-GTX GCC CCT TCT CCC XGC AGC-3′), wherein X represents 5-methyl-2′-deoxycytidine. In certain embodiments, the series of tablets (e.g., coated tablets) is at least 100 tablets (or coated tablets), for example, at least 500, such as at least 1,000, at least 2,000, at least 5,000, at least 10,000, at least 20,000, at least 50,000, at least 100,000, or at least 200,000 tablets (or coated tablets), for example, between 100-1,000,000 tablets (or coated tablets), such as between 1,000-1,000,000, between 10,000-1,000,000, between 50,000-1,000,000, between 100,000-1,000,000, or between 500-1,000,000 tablets (or coated tablets). In certain embodiments, the single batch composition utilized in the method of preparing the series of tablets complies with the terms batch or lot as defined under 21 CFR 210.3(2) and 21 CFR 210.3(10), respectively. In certain embodiments, one or more, such as a plurality, of the single batch compositions prepared according to the methods disclosed herein, for example, 2, 3, 4, 5, 6, 7, 8, 9, or 10, single batch compositions, are utilized in the method of preparing the series of tablets.

In certain embodiments, a pharmaceutical composition batch is provided, comprising at least a portion of a single batch composition or a substantially pure oligonucleotide composition as disclosed herein, or an oligonucleotide composition prepared according to the methods of preparing as disclosed herein, see, e.g., as described in Sections 5-5.16, and a pharmaceutically acceptable adjuvant and/or excipient. For example, in certain embodiments, the pharmaceutical composition batch comprises at least 10 wt. %, at least 20 wt. %, at least 30 wt. %, at least 40 wt. %, at least 50 wt. %, at least 60 wt. %, at least 70 wt. %, at least 80 wt. %, at least 90 wt. %, at least 95 wt. %, or 100 wt. %, of the single batch composition, the substantially pure oligonucleotide composition, or the oligonucleotide composition prepared according to the methods of preparing as disclosed herein. In certain embodiments, for example, the pharmaceutical composition batch comprises at least one oral dosage form (e.g., a tablet or coated tablet as described herein), for example, a series of oral dosage forms, such as a series of tablets or a series of coated tablets. In certain embodiments, the pharmaceutical composition batch is prepared by formulating (or combining) at least a portion of a single batch composition or a substantially pure oligonucleotide composition as disclosed herein, or an oligonucleotide composition prepared according to the methods of preparing as disclosed herein, see, e.g., as described in Sections 5-5.16, with a pharmaceutically acceptable adjuvant and/or excipient. In certain embodiments, for example, the pharmaceutical composition batch comprises at least a portion of a single batch composition of an oligonucleotide comprising at least 700 mmol of the oligonucleotide, e.g., at least 700 mmol of the oligonucleotide and at most 25 wt. % water, and a pharmaceutically acceptable adjuvant and/or excipient; wherein the oligonucleotide is an SMAD7 antisense oligonucleotide, for example, wherein the oligonucleotide has a nucleic acid sequence of any one of SEQ ID NO: 2, 3, 4, 5, 6, 7, 8, 9, 10, 11, or 12, preferably having a nucleic acid sequence: SEQ ID NO: 3: (5′-GTX GCC CCT TCT CCC XGC AGC-3′), wherein X represents 5-methyl-2′-deoxycytidine. For example, in certain embodiments, the pharmaceutical composition batch comprises at least a portion of a single batch composition of an oligonucleotide comprising at least 2 g/mmol of an at least 700 mmol synthesis scale of the oligonucleotide, e.g., at least 2 g/mmol of an at least 700 mmol synthesis scale of the oligonucleotide and at most 25 wt. % water, and a pharmaceutically acceptable adjuvant and/or excipient; wherein the oligonucleotide is an SMAD7 antisense oligonucleotide, for example, wherein the oligonucleotide has a nucleic acid sequence of any one of SEQ ID NO: 2, 3, 4, 5, 6, 7, 8, 9, 10, 11, or 12, preferably having a nucleic acid sequence: SEQ ID NO: 3: (5′-GTX GCC CCT TCT CCC XGC AGC-3′), wherein X represents 5-methyl-2′-deoxycytidine. For example, in certain embodiments, the pharmaceutical composition batch comprises at least a portion of a single batch composition of an oligonucleotide comprising at least 2 kg of the oligonucleotide, e.g., comprising at least 2 kg of the oligonucleotide and at most 25 wt. % water, and a pharmaceutically acceptable adjuvant and/or excipient; wherein the oligonucleotide is an SMAD7 antisense oligonucleotide, for example, wherein the oligonucleotide has a nucleic acid sequence of any one of SEQ ID NO: 2, 3, 4, 5, 6, 7, 8, 9, 10, 11, or 12, preferably having a nucleic acid sequence: SEQ ID NO: 3: (5′-GTX GCC CCT TCT CCC XGC AGC-3′), wherein X represents 5-methyl-2′-deoxycytidine. For example, in certain embodiments, the pharmaceutical composition batch comprises at least a portion of a single batch composition of an oligonucleotide comprising at least 50 mol % of the oligonucleotide output from at least one 700 mmol or greater oligonucleotide synthesis column, e.g., comprising at least 50 mol % of the oligonucleotide output from at least one 700 mmol or greater oligonucleotide synthesis column and at most 25 wt. % water, and a pharmaceutically acceptable adjuvant and/or excipient; wherein the oligonucleotide is an SMAD7 antisense oligonucleotide, for example, wherein the oligonucleotide has a nucleic acid sequence of any one of SEQ ID NO: 2, 3, 4, 5, 6, 7, 8, 9, 10, 11, or 12, preferably having a nucleic acid sequence: SEQ ID NO: 3: (5′-GTX GCC CCT TCT CCC XGC AGC-3′), wherein X represents 5-methyl-2′-deoxycytidine. In certain embodiments, the pharmaceutical composition batch comprises, or is, a series of oral dosage forms (e.g., tablets or coated tablets). For example, in certain embodiments, the pharmaceutical composition batch comprises, or is, a series of at least 100 tablets (or coated tablets), for example, at least 500, such as at least 1,000, at least 2,000, at least 5,000, at least 10,000, at least 20,000, at least 50,000, at least 100,000, or at least 200,000 tablets (or coated tablets), for example, between 100-1,000,000 tablets (or coated tablets), such as between 1,000-1,000,000, between 10,000-1,000,000, between 50,000-1,000,000, between 100,000-1,000,000, or between 500-1,000,000 tablets (or coated tablets). In certain embodiments, the pharmaceutical composition batch, or the single batch composition contained in the pharmaceutical composition batch or utilized in preparing the pharmaceutical composition batch, complies with the terms batch or lot as defined under 21 CFR 210.3(2) and 21 CFR 210.3(10), respectively. In certain embodiments, one or more, such as a plurality, of the single batch compositions prepared according to the methods disclosed herein, for example, 2, 3, 4, 5, 6, 7, 8, 9, or 10, single batch compositions, are contained within a pharmaceutical composition batch, or utilized in preparing the pharmaceutical composition batch.

Contemplated formulations, e.g., tablets or coated tablets, in certain embodiments, when orally administered to the patient can result in minimal plasma concentration of the oligonucleotide in the patient. In another embodiment, contemplated formulations, when orally administered to a patient, topically deliver to the terminal ileum and/or right colon of a patient, e.g., to an affected or diseased intestinal site of a patient.

5.19 Treatment Regimen

5.19.1 Administration Regimen

In one aspect, provided herein is a method for treating or managing inflammatory bowel disease (IBD) in a patient having IBD, wherein the method comprises (a) administering to the patient a SMAD7 antisense-oligonucleotide during a first treatment period at a first dose; and (b) administering to the patient the SMAD7 antisense-oligonucleotide during a second treatment period at a second dose.

In certain embodiments, the dose of the anti-SMAD7 therapy administered during the first treatment period is higher than the dose administered during the second treatment period.

The first and second treatment periods each can have a duration of weeks, months, or years. The length of the first and second period can be adjusted depending, e.g., on whether an IBD patient responds to the anti-SMAD7 therapy, on how strongly the patient responds (e.g., the degree of the clinical response or the occurrence of remission), or on whether a patient, who has previously responded to the anti-SMAD7 therapy, relapses.

In certain embodiments, the inflammatory bowel diseases (IBD) is Crohn's disease (CD). In certain embodiments, the inflammatory bowel diseases (IBD) is ulcerative colitis (UC).

5.19.2 IBD Treatment and Management

The methods recited in this section are useful to treat or manage IBD in a patient or subject having IBD, including, e.g., mild, medium, or severe forms of IBD (e.g., mild, medium, or severe forms of Crohn's disease (CD) or ulcerative colitis (UC)), e.g., as determined by a clinical activity parameter or a biomarker level. In certain embodiments, the methods are useful to prevent IBD, e.g., in a patient at risk of developing IBD, e.g., as determined by the presence of certain risk factors in the patient, which are known in the art (e.g., genetic, environmental, or lifestyle factors). In certain embodiments, the methods provided herein are useful to prevent the reoccurrence of IBD in a patient who has previously received an IBD treatment (e.g., an aminosalicylate treatment or a steroid treatment), which is failing, or in a treatment naive patient who is experiencing a chronic disease with few or no clinical symptoms.

In certain embodiments, treating or managing IBD comprises reducing one or more clinical symptoms of IBD. In certain embodiments, treating or managing IBD comprises reducing a symptom of CD, such as belly pain (including, e.g., cramping, soreness to touch, constant ache), diarrhea (including, e.g., blood in stool), loss of appetite, fever, weight loss, anemia, intestinal inflammation, or an infection (e.g., an abscess), an anal fissure, joint pain, eye problems, a skin rash, or liver disease. In certain embodiments, treating or managing IBD comprises reducing one or more symptoms of UC, such as intestinal swelling, intestinal inflammation, sores in the lining of the large intestine (colon), diarrhea, belly pain, or bleeding from the rectum. In certain embodiments, treating or managing IBD comprises reducing one or more IBD symptoms during a chronic phase of the disease. In certain embodiments, treating or managing IBD comprises reducing one or more IBD symptoms during an acute phase of the disease (e.g., during a disease “flare up”). In certain embodiments, treating or managing IBD comprises increasing the time to relapse in an IBD patient who has responded to an IBD treatment such as an anti-SMAD7 therapy (e.g., an SMAD7 antisense-oligonucleotide).

In certain embodiments, treating or managing IBD comprises reducing the intensity of a disease flare up. In certain embodiments, treating or managing IBD comprises reducing the frequency with which flare ups occur the IBD patient.

In certain embodiments, treating of managing IBD comprises improving the quality of life of an IBD patient (e.g., as determined by a patient survey), e.g., by reducing pain in the IBD patient, improving appetite in the IBD patient, or improving the IBD patient's sleep (e.g., length of uninterrupted sleep).

5.19.3 Prevention

In certain embodiments, preventing IBD or preventing the reoccurrence of IBD comprises preventing the occurrence or reoccurrence of one or more clinical symptoms of IBD, either partially or completely. In certain embodiments, preventing IBD or preventing the reoccurrence of IBD comprises preventing the occurrence or reoccurrence of a symptom of CD, such as belly pain (including, e.g., cramping, soreness to touch, constant ache), diarrhea (including, e.g., blood in stool), loss of appetite, fever, weight loss, anemia, intestinal inflammation, or an infection (e.g., an abscess), an anal fissure, joint pain, eye problems, a skin rash, or liver disease. In certain embodiments, preventing IBD or preventing the reoccurrence of IBD comprises preventing the occurrence or the reoccurrence of one or more symptoms of UC, such as intestinal swelling, intestinal inflammation, sores in the lining of the large intestine (colon), diarrhea, belly pain, or bleeding from the rectum. In certain embodiments, preventing IBD or preventing the reoccurrence of IBD comprises preventing one or more IBD symptoms during a chronic phase of the disease. In certain embodiments, preventing IBD or preventing the reoccurrence of IBD comprises reducing one or more IBD symptoms during an acute phase of the disease (e.g., during a disease “flare up”). In certain embodiments, preventing the reoccurrence of IBD comprises increasing the time to relapse in an IBD patient who has responded to an IBD treatment such as an anti-SMAD7 therapy (e.g., an SMAD7 antisense-oligonucleotide).

In certain embodiments, preventing IBD or preventing the reoccurrence of IBD comprises preventing the occurrence or reoccurrence of a disease flare up or of a disease flare up of a certain intensity. In certain embodiments, preventing IBD or preventing the reoccurrence of IBD comprises preventing the occurrence or reoccurrence of flare ups at a certain frequency (e.g., at a frequency observed in the patient having IBD directly prior to the administration of an IBD treatment regiment or at a (e.g., median, average or mean) frequency observed in a control group of untreated IBD patients).

In certain embodiments, preventing IBD or preventing the reoccurrence of IBD comprises preventing a (further) deterioration in the quality of life of an IBD patient (e.g., as determined by a patient survey), e.g., by preventing the increase in pain in the IBD patient, preventing (further) loss of appetite in the IBD patient, or preventing a worsening of sleeplessness in the IBD patient.

5.19.4 Patient Population

In certain embodiments, an IBD patient to be treated with a method provided herein is a UC patient or a CD patient. In certain embodiments, the patient having IBD was diagnosed with CD or UC at least 3 months prior to the initial screening period or the first treatment period. In certain embodiments, the patient having IBD was diagnosed with ileitis, or ileocolitis, e.g., as determined by endoscopic, radiographic or another imaging method (e.g., magnetic resonance imaging [MM], computed tomography [CT] scan), within 2 years prior to the screening period or to the first treatment period. In certain embodiments, the patient has IBD involving the distal to mid transverse colon. In certain embodiments, the patient has extensive colitis.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference in their entirety to the same extent as if each independent patent and publication was specifically and individually indicated to be incorporated by reference in its entirety.

The following examples are provided by way of illustration, not limitation.

### EXAMPLES

### Example 1—Preparing 21-Mer Oligonucleotide Having Nucleic Acid Sequence SEQ ID NO: 3

Oligonucleotide prepared: a deoxyribonucleotide having a nucleic acid sequence:

SEQ ID NO: 3:
(5′-GTX GCC CCT TCT CCC XGC AGC-3′),

wherein X represents 5-methyl-2′-deoxycytidine, and wherein all of the internucleotide linkages are O,O-linked phosphorothioates.

Scale: A 3×900 mmol scale and a 5×900 mmol scale preparation of the targeted 21-mer oligonucleotide were conducted. Specifically, the synthesis and ion-exchange purification of the oligonucleotide were each conducted at a scale to produce 900 mmol of the targeted 21-mer oligonucleotide, and the synthesis and ion-exchange purification processes, in their entirety, were repeated another 2-4 times at a 900 mmol scale (i.e., the loading capacity of each synthesis column, and separately, the ion-exchange capacity of each ion-exchange purification column, were each 900 mmol, thereby capable of producing the targeted 21-mer oligonucleotide is a theoretical amount of 2,700-4,500 mmol, respectively). The resulting oligonucleotide products from the ion-exchange purification processes were then pooled before desalting via the ultrafiltration/diafiltration process, which was conducted at a 2,700-4,500 mmol scale, respectively. The Thin Film Evaporation process for the removal of water, thereby concentrating the solution of the final oligonucleotide product, was conducted at a 2,700-4,500 mmol scale, respectively. Finally, the Freeze-Drying process for removing water and concentrating the final 21-mer oligonucleotide product to form a solid product, was conducted at a 2,700-4,500 mmol scale. The 3×900 mmol scale and a 5×900 mmol scale preparations, as detailed below, are represented schematically in FIG. 1, wherein the optional drying step was conducted to provide a solid single batch composition of the targeted 21-mer oligonucleotide.

Synthesizer and Column: Synthesis of the oligonucleotide having the nucleic acid sequence of SEQ ID NO: 3 was conducted with a 1454 Synthesizer using a synthesis column having an inner diameter of 50-80 cm that was loaded with 900 mmol of a crosslinked polystyrene solid support (e.g., NITTOPHASE-HL or PRIMER SUPPORT 5G) having attached thereto a linker containing hydroxyl groups protected with 4,4′-dimethoxytrityl group (DMT), that when deprotected were available to initiate assembly of the targeted oligonucleotide (e.g., UNYLINKER). To achieve a loading capacity of 900 mmol of the linker-bound support into the synthesis column, between 2000-3000 g of the linker-bound support (having 330-370 micromoles/g of solid linker-bound support available to react with protected nucleoside phosphoramidites to assemble the targeted oligonucleotide) was loaded into the synthesis column to a packing density of 8-11 mL/g (0.08-0.15 g/ml), a bed height of 7-10 cm, and a column volume of 22-25 L.

Synthesis Procedure: The following synthesis procedure utilized phosphoramidite chemistry for preparing the 21-mer oligonucleotide having the sequence SEQ ID NO: 3:

A) Detritylation: In the beginning of the synthesis, the high loaded NITTOPHASE UNYLINKER 350 was subjected to an acid catalyzed removal of the 4,4′-dimethoxytrityl (DMT) protecting group from the 5′-hydroxyl group of the UNYLINKER, by introducing a solution of 10% DCA in toluene to the solid linker-bound support (monitoring by UV at 440 nm). The detritylated solid linker-bound support was washed with 1-2 column volumes of acetonitrile. For the remaining portion of the synthesis, after each reaction cycle iteration, a new reaction cycle iteration began with removal of the corresponding DMT protecting group from the 5′-oxygen atom of the support-bound oligonucleotide utilizing 3% DCA in toluene (utilizing about 6-7 column volumes and UV monitoring at 440 nm). The detritylated support-bound oligonucleotide was washed with 1-2 column volumes of acetonitrile.

B) Coupling: The oligonucleotide was assembled from the 3′ to the 5′ terminus by extending the oligonucleotide chain via reacting the detritylated solid support (for the first coupling) or the 5′-hydroxyl group of the detritylated support-bound oligonucleotide (for post-first couplings) with an excess of a solution of a protected nucleoside phosphoramidite, in the presence of the activator.

Specifically, to the detritylated solid linker-bound support (for the first coupling) or the 5′-hydroxyl group of the detritylated support-bound oligonucleotide (for post-first couplings) was introduced an excess of acetonitrile solution of a protected nucleoside phosphoramidite (1.1-2 equivalents of one of the following: 0.2 M dA-(Bz), 0.2 M dC-(Bz), 0.2 M dG-(iBu), 0.2 M dT, or 0.2 M d5MeC—(Bz)), in the presence 5-8 equivalents of the activator (0.5-1 M dicyanoimidazole (DCI) in acetonitrile) relative to the 900 mmol of the linker-bound support, thereby providing an activator:amidite molar ratio of 3-5:1. The total volume of the coupling solutions provided was between 14-17 L. The specific protected nucleoside phosphoramidite required for each step was determined by the oligonucleotide sequence SEQ ID NO: 3. After allowing sufficient time for the coupling reaction to complete, thereby forming a phosphite triester internucleotide linkage, the excess phosphoramidite and activator were rinsed from the column with 1-2 column volumes of acetonitrile.

C) Thiolation: Deliver an oxidizing or sulfurizing solution to the solid support; The phosphite triester internucleotide linkage formed during the coupling step was then thiolated with the introduction of 0.2M xanthane hydride in pyridine (about 16-20 L, providing 2-6 equivalents XH relative to the 900 mmol of the linker-bound support) to form the corresponding phosphorothioate linkage. After the thiolation was complete, the excess reagent was removed from the column by rinsing the support with 1-2 column volumes of acetonitrile.

D) Capping: To prevent any 5′-hydroxyl groups that failed to couple with the activated phosphoramidite during any given reaction cycle iteration from being available to react in any subsequent reaction cycle, these unreacted 5′-hydroxyl groups were blocked (or capped) with an acyl group as follows: introducing a 1:1 mixture of capping reagents Cap A and Cap B (Cap A: 10-30% N-methylimidazole, 20-40% pyridine, 40-60% acetonitrile (v/v/v); and Cap B: 10-30% isobutyric anhydride in acetonitrile (v/v)) to the support-bound oligonucleotide having phosphorothioate linkages to form 5′-O-isobutyrated (“capped”) support-bound oligonucleotide sequences. The total volume of the mixture of capping reagents Cap A and Cap B was between 0.5-2 column volumes. After the capping was complete, the excess reagents were removed from the column by rinsing the support with 1-2 column volumes of acetonitrile.

E) Deprotecting Protected Phosphorothioate Linkages: Upon completion of the coupling, thiolation, capping, and detritylation for the predetermined number of reaction cycle iterations to form the 21-mer oligonucleotide having the sequence SEQ ID NO: 3 (with the exception that the capping and detritylation steps were not performed during the last reaction cycle iteration), the crude oligonucleotide bound to the solid support was washed with 10-30% triethylamine in acetonitrile (about 3-5 column volumes) to remove the cyanoethyl protecting groups on the phosphorothioate linkages. After the deprotection was complete, the excess reagents were removed from the column by rinsing the support with 3-5 column volumes of acetonitrile.

F) Cleavage of Crude Oligonucleotide from Solid Support: To the crude oligonucleotide bound to the solid support was introduced (and recirculated through) a heated ammonium hydroxide solution (28-30% ammonia, heated to about 40-60° C., about 2-5 column volumes) for about 16-40 hours. This resulted in cleaving the crude oligonucleotide from the solid support and simultaneously deprotecting the exocyclic amino groups (benzoyl and isobutyryl) on the crude oligonucleotide. The final reaction mixture was removed from the synthesis column by filtration and the solid support was washed with water, to provide the crude oligonucleotide having only the 5′-hydroxyl group of the terminal nucleoside protected with a 4,4′-dimethoxytrityl (DMT) protecting group.

The results for a 3×900 mmol (1A, 1B, and 1C) and a 5×900 mmol (2A, 2B, 2C, 2D, and 2E) scale synthesis of the target 21-mer oligonucleotide having the sequence SEQ ID NO: 3 are provided in Table 1 and Table 2, respectively.

The amount of crude product cleaved from the synthesis column and collected in the filtrate (“Total Crude OD”) was measured via optical density (OD) at wavelength 260 nm.

The Crude Yield of the product cleaved and collected from the synthesis column (“Crude Yield (OD/umol)”) was determined as follows: [Total Crude OD/900 mmol scale]/(1000 umol/mmol).

The Crude Yield of the product cleaved and collected from the synthesis column (“Crude Yield (g/mmol)”) was determined using a conversion factor for the target 21-mer oligonucleotide having the sequence SEQ ID NO: 3 (i.e., 21856.3 OD/g), as follows: [Total Crude OD/(Conversion Factor (OD/g))]/(900 mmol scale).

The Conversion Factor is specific for the target 21-mer oligonucleotide having the sequence SEQ ID NO: 3 (i.e., 21856.3 OD/g). Accordingly, use of this specific coversion factor at the synthesis stage of the process is as a means to estimate or approximate the amount of the DMT-protected target oligonucleotide cleaved and collected from the synthesis column. The Conversion Factor was provided as follows: [(extinction coefficient (L*mol⁻¹*cm⁻¹) of target oligonucleotide at wavelength 260 nm)/(molecular weight (g/mol) of sodium salt of the target 21-mer oligonucleotide having the sequence SEQ ID NO: 3)]×1000; wherein the target 21-mer oligonucleotide having the sequence SEQ ID NO: 3 has a molecular weight of 7044.05 g/mol (as sodium salt) and an extinction coefficient of 153957 L*mol⁻¹*cm⁻¹ at wavelength 260 nm.

The Step Yield (mol. %) for all recovered oligonucleotide product(s), i.e., the crude yield for the synthesis portion of this process, was determined as follows: [(Total Crude OD)/[(900 mmol scale)×(extinction coefficient (L*mol⁻¹*cm⁻¹) of target oligonucleotide at wavelength 260 nm)]]×100; wherein the extinction coefficient is 153957 L*mol⁻¹*cm⁻¹ at wavelength 260 nm.

The mole percent value of the specific DMT-protected target oligonucleotide contained in the crude product recovered from the synthesis column, referred to as the percent Full Length Product (“% FLP (mol. %)”), was determined via reverse phase ion-pair HPLC (“RPIP-HPLC”).

The percent yield of the specific DMT-protected target oligonucleotide produced from the synthesis column, referred to as Full Length Product Yield (“FLP Yield (mol. %)”), was determined as follows: [(step yield mol. %)×(% FLP mol. %)]/100.

TABLE 1

| Synthesis | 1A | 1B | 1C |
|---|---|---|---|
| Total Crude OD | 124162435 | 127914818 | 124751301 |
| Crude Yield | 138 | 142 | 139 |
| (OD/umol) | | | |
| Crude Yield | 6.31 | 6.50 | 6.34 |
| (g/mmol) | | | |
| Step Yield | 90 | 92 | 90 |
| (mol. %) | | | |
| % FLP (mol. %) | 77.1 | 70.2 | 73.9 |
| FLP Yield | 69 | 65 | 67 |
| (mol. %) | | | |

TABLE 2

| Synthesis | 2A | 2B | 2C | 2D | 2E |
|---|---|---|---|---|---|
| Total Crude OD | 128134943 | 128697940 | 127897440 | 130172903 | 127055151 |
| Crude Yield | 142 | 143 | 142 | 145 | 141 |
| (OD/umol) | | | | | |
| Crude Yield | 6.51 | 6.54 | 6.50 | 6.62 | 6.46 |
| (g/mmol) | | | | | |
| Step Yield | 92 | 93 | 92 | 94 | 92 |
| (mol. %) | | | | | |
| % FLP | 74.7 | 75.4 | 73.5 | 74.8 | 77.5 |
| (mol. %) | | | | | |
| FLP Yield | 69 | 70 | 68 | 70 | 71 |
| (mol. %) | | | | | |

Purification Procedure: The crude yield of the crude, cleaved oligonucleotide, having only the 5′-hydroxyl group of the terminal nucleoside protected with a 4,4′-dimethoxytrityl (DMT) protecting group, was loaded onto an anion exchange column (having Q Sepharose FF column media, an inner diameter of 60-100 cm, a bed height of about 17-25 cm, and a column volume of about 100-120 L) using 1-4 column volumes of a 25 mM sodium hydroxide solution to achieve a column loading of about 1000-1200 OD/mL. The purifier utilized was an AktaProcess. The crude oligonucleotide, once loaded onto the anion exchange column, was washed with 1-3 column volumes of a mixture of a 25 mM sodium hydroxide solution and a 2 M sodium chloride solution, and then washed with 1-3 column volumes of a 25 mM sodium hydroxide solution.

To remove the 4,4′-dimethoxytrityl (DMT) protecting group from the 5′-hydroxyl of the terminal nucleoside, the loaded, crude oligonucleotide was detritylated by introducing 80% aqueous acetic acid (about 1 column volume at 1,000-1,200 L/hr) until the resulting eluent had a pH of about 2 or less, thereby forming a fully deprotected, crude oligonucleotide that was now loaded onto the anion exchange column. The pH of the anion exchange column was readjusted to a pH of about 10 or more with a 0.025-0.20 N sodium hydroxide solution (at 1,000-1,200 L/hr) before purifying with a basic salt gradient was initiated (using a gradient of 5%-95% of an aqueous solution containing 20-30 mM sodium hydroxide and 1-3 M sodium chloride; with a gradient slope of about 10-20 column volumes, and using a flow rate of no more than 1,200 L/hr; a salt gradient from 0.2 to 1.8 M sodium chloride in aqueous 25 nM sodium hydroxide solution). The final wash to retrieve the purified oligonucleotide product was completed with at least 3 column volumes of an aqueous solution containing 20-50 mM sodium hydroxide and 2-4M sodium chloride.

The purification results for a 3×900 mmol (1A, 1B, and 1C) and a 5×900 mmol (2A, 2B, 2C, 2D, and 2E) scale synthesis of the target 21-mer oligonucleotide having the sequence SEQ ID NO: 3 are provided in Table 3 and Table 4, respectively.

The amount of crude, cleaved DMT-protected target oligonucleotide loaded onto the anion exchange column, reported as (“OD Loaded onto Column”), was measured via optical density (OD) at wavelength 260 nm.

The amount of purified target oligonucleotide product recovered from the anion exchange column, reported as (“Total OD Recovered from Column”), was measured via optical density (OD) at wavelength 260 nm.

The percentage of purified target oligonucleotide product recovered from the anion exchange column, referred to as “% OD Recovery (Step Yield mol. %)”, was determined as follows: [(Total OD Recovered from Column)/(OD Loaded onto Column)]×100.

The mass of purified target oligonucleotide product recovered from the anion exchange column, referred to as “Projected Mass of Target Oligonucleotide(g)”, was determined as follows: [(Total OD Recovered from Column)/(Conversion Factor (OD/g))]; wherein the Conversion Factor is specific for the target 21-mer oligonucleotide having the sequence SEQ ID NO: 3 (i.e., 21856.3 OD/g).

The mole percent value of the purified target oligonucleotide product recovered from the anion exchange column, referred to as the percent Full Length Product (“% FLP (mol. %)”), was determined via reverse phase ion-pair HPLC (“RPIP-HPLC”).

The percent yield of the purified target oligonucleotide product recovered from the anion exchange column, referred to as Full Length Product Yield (“FLP Yield (mol. %)”), was determined as follows: [(% OD Recovery (Step Yield mol. %))×(% FLP mol. %)]/100.

TABLE 3

| Purification | 1A | 1B | 1C |
|---|---|---|---|
| OD Loaded onto Column | 124162435 | 127914818 | 124751301 |
| Total OD Recovered from Column | 84304000 | 95404000 | 91176000 |
| % OD Recovery (Step Yield mol. %) | 68 | 75 | 73 |
| Projected Mass of Target | 3857.19 | 4365.05 | 4171.61 |
| Oligonucleotide (g) | | | |
| % FLP (mol. %) | 91.4 | 91.3 | 91.1 |
| FLP Yield (mol. %) | 62 | 68 | 67 |

TABLE 4

| Purification | 2A | 2B | 2C | 2D | 2E |
|---|---|---|---|---|---|
| OD Loaded onto Column | 128134943 | 128697940 | 127897440 | 130172903 | 127055151 |
| Total OD Recovered from Column | 84235000 | 85312000 | 79541000 | 88176000 | 89445000 |
| % OD Recovery (Step Yield mol. %) | 66 | 66 | 62 | 68 | 70 |
| Projected Mass of Target | 3854.03 | 3903.31 | 3639.27 | 4034.35 | 4092.41 |
| Oligonucleotide (g) | | | | | |
| % FLP (mol. %) | 91.4 | 91.1 | 90.8 | 91.3 | 91.9 |
| FLP Yield (mol. %) | 60 | 60 | 56 | 62 | 65 |

Ultrafiltration/Diafiltration Procedure: The purified oligonucleotide eluates from 3-4 synthesis/purification runs (each at 900 mmol scales) were pooled together and were desalted via an ultrafiltration/diafiltration process using a CUF-1 system having a cassette of Regenerated Cellulose (3,000 DA molecular weight cut-off; with a cassette area 20-25 m² (i.e., 8-10×2.5 m²). The pooled, purified oligonucleotide eluates, which were loaded at an initial concentration of about 650-750 OD/mL and had a cross-flow velocity of about 5-6 L/min/m², were desalted by neutralizing to a pH of about 6.5-7.5 (using 0.5-2 M HCl and 0.05-0.25 M HCl to achieve pH 6.5-7.5; adjusting with 0.05-0.25 N NaOH solution if necessary), and then diafiltrated with water for at least 7 exchanges until the conductivity of the permeate (diafiltrate) was less than 50 μS/cm, and the resulting concentration of the desalted oligonucleotide in the retentate solution was at least 1,000 OD/mL.

The ultrafiltration/diafiltration process results for a 3×900 mmol (“1 (A-C)”, i.e., ultrafiltration/diafiltration of pooled 1A, 1B, and 1C) and a 5×900 mmol (“2 (A-E)”, i.e., ultrafiltration/diafiltration of pooled 2A, 2B, 2C, 2D, and 2E) scale synthesis of the target 21-mer oligonucleotide having the sequence SEQ ID NO: 3 are provided in Table 5.

The amount of purified target oligonucleotide product that was desalted via an ultrafiltration/diafiltration process, reported as (“Total OD Loaded”), was measured via optical density (OD) at wavelength 260 nm.

The amount of desalted, purified target oligonucleotide product contained in the retentate solution, reported as (“Total OD in Final Retentate”), was measured via optical density (OD) at wavelength 260 nm.

The percentage of desalted, purified target oligonucleotide product contained in the retentate via the ultrafiltration/diafiltration process, referred to as “% OD Recovery (Step Yield mol. %)”, was determined as follows: [(Total OD in Final Retentate)/(Total OD Loaded)]×100.

The conductivity of the permeate (diafiltrate) at the end of the ultrafiltration/diafiltration process (“Final Permeate Conductivity (uS/cm)”), was measured directly from the final permeate filtrate.

TABLE 5

| Ultrafiltration/Diafdtration | 1 (A-C) | 2 (A-E) |
|---|---|---|
| Total OD loaded | 270884000 | 426709000 |
| Total OD in Final Retentate | 240841600 | 388870000 |
| OD Recovery (Step Yield; mol. %) | 89 | 91 |
| Final Permeate Conductivity (uS/cm) | 45 | 48 |

Concentration via Thin Film Evaporation and Freeze-Drying Processes: The resulting desalted oligonucleotide retentate solution was initially concentrated via Thin Film Evaporation on a TFE-280 system, using a jacket temperature at about 60-80° C., a vacuum pressure at about 30-100 Torr, to achieve a final concentration of at least 3000 OD/mL for 2700 mmol scale and at least 4000 OD/mL for 3600 mmol scale.

The concentrated, desalted oligonucleotide resulting from Thin Film Evaporation was further concentrated and dried via a freeze drying process on a Boc-Edwards Freeze-Dryer or KTS Freeze-Dryer, loading at a temperature of 15-25° C., freezing at a temperature of −50 to −30° C., evacuating at −50 to −30° C. and 100-500 millitorr, and drying at a temperature of −10 to 20° C., and a pressure of 100-500 to 1 millitorr, to achieve a solid final product of the target oligonucleotide having the nucleic acid sequence of SEQ ID NO: 3.

The thin film evaporation and freeze drying process results for a 3×900 mmol (“1 (A-C)”) and a 5×900 mmol (“2 (A-E)”) scale synthesis of the target 21-mer oligonucleotide having the sequence SEQ ID NO: 3 are provided in Table 6.

The amount of desalted target oligonucleotide product that was concentrated via a thin film evaporation process, reported as (“Total Starting OD”), was measured via optical density (OD) at wavelength 260 nm.

The amount of solid, concentrated target oligonucleotide product recovered from the freeze drying process, reported as (“Total Amount: Harvested Solid (g)”), is a weight measurement.

The amount of solid, concentrated target oligonucleotide product recovered from the freeze drying process that is corrected for moisture content, reported as (“Total Amount: Harvested Solid Corrected for Moisture (g)”), was determined by a water content analysis, such as a Karl Fischer titration.

The amount of solid, concentrated target oligonucleotide product recovered from the freeze drying process that is corrected for moisture content, reported in terms of optical density (“Total Amount: Equivalent OD Value (OD)”), was determined as follows: [(Total Amount: Harvested Solid Corrected for Moisture (g))×(Conversion Factor (OD/g))]; wherein the Conversion Factor is specific for the target 21-mer oligonucleotide having the sequence SEQ ID NO: 3 (i.e., 21856.3 OD/g).

The amount of solid, concentrated target oligonucleotide product recovered from the freeze drying process that is corrected for moisture content, reported in terms of mmol (“Total Amount: mmol”), was determined as follows: [(Total Amount: Harvested Solid Corrected for Moisture (g))/(molecular weight (g/mol) of sodium salt of the target 21-mer oligonucleotide having the sequence SEQ ID NO: 3)]×1000; wherein the target 21-mer oligonucleotide having the sequence SEQ ID NO: 3 has a molecular weight of 7044.05 g/mol (as sodium salt).

The Step Yield Corrected for Moisture (mol. %) for the thin film evaporation and freeze drying process to provide the solid, concentrated target oligonucleotide product recovered from the freeze drying process that is corrected for moisture content, was determined as follows: [(Total Amount: Equivalent OD Value (OD))/(Total Starting OD)]×100.

The Overall Yield Corrected for Moisture (g/mmol) for the entire process (i.e., from synthesis, purification, desalting, and through concentrating process (thin film evaporation and freeze drying processes)) to provide the solid, concentrated target oligonucleotide product recovered from the freeze drying process that is corrected for moisture content, reported as “Overall Yield Corrected for Moisture (g isolated/starting mmol)”, was determined as follows: [(Total Amount: Harvested Solid Corrected for Moisture (g))/[(900 mmol)×(number of iterations pooled)]]; wherein the “number of iterations pooled” is 3 for “1 (A-C)” and 5 for “2 (A-E)”.

The Overall Yield Corrected for Moisture (mol. %) for the entire process (i.e., from synthesis, purification, desalting, and through concentrating process (thin film evaporation and freeze drying processes)) to provide the solid, concentrated target oligonucleotide product recovered from the freeze drying process that is corrected for moisture content, reported as “Overall Yield (mol. %)”, was determined as follows: [(Total Amount: mmol)/[(900 mmol)×(number of iterations pooled)]]×100; wherein the “number of iterations pooled” is 3 for “1 (A-C)” and 5 for “2 (A-E)”.

The amount of concentrated, purified target oligonucleotide product recovered from the entire process that is corrected for moisture content, referred to as “Target Oligonucleotide (dry wt. %)”, was measured via reverse phase ion-pair HPLC (“RPIP-HPLC”).

The sodium content of the concentrated, purified target oligonucleotide product recovered from the entire process, referred to as “Sodium Content (mol. %)”, was measured via inductively coupled plasma (ICP).

TABLE 6

| Thin Film Evaporation/Freeze Drying Process | 1 (A-C) | 2 (A-E) |
|---|---|---|
| Total Starting OD | 240841600 | 388870000 |
| Total Amount: Harvested Solid (g) | 10602 | 17770 |
| Total Amount: Harvested Solid Corrected for Moisture (g) | 9966 | 16348 |
| Total Amount: Equivalent OD Value (OD) | 217817447 | 357315837 |
| Total Amount: mmol | 1415 | 2321 |
| Step Yield Corrected for Moisture (%) | 90 | 92 |
| Overall Yield Corrected for Moisture (g isolated/starting mmol) | 3.7 | 3.6 |
| Overall Yield (mol. %) | 52 | 52 |
| Target Oligonucleotide (dry wt. %) | 92.4 | 94.1 |
| Sodium Content (mol. %) | 6.5 | 6.9 |

### Exemplary Embodiments

In an embodiment, a single batch composition of an oligonucleotide comprises at least 700 mmol of the oligonucleotide and at most 25 wt. % water.

In an embodiment, a single batch composition of an oligonucleotide comprises at least 2 kg of the oligonucleotide and at most 25 wt. % water.

In an embodiment, a single batch composition of an oligonucleotide comprises at least 50 mol % of the oligonucleotide output from at least one 700 mmol or greater oligonucleotide synthesis column and at most 25 wt. % water.

In an embodiment, a single batch composition of an oligonucleotide comprises at least 700 mmol of the oligonucleotide, wherein the single batch composition is a liquid composition.

In an embodiment, a single batch composition of an oligonucleotide comprises at least 2 kg of the oligonucleotide, wherein the single batch composition is a liquid composition.

In an embodiment, a single batch composition of an oligonucleotide comprises at least 50 mol % of the oligonucleotide output from at least one 700 mmol or greater oligonucleotide synthesis column, wherein the single batch composition is a liquid composition.

In an embodiment, an oligonucleotide composition comprises a plurality of at least 700 mmol scale single batch synthetic preparations of an oligonucleotide, wherein the oligonucleotide composition has at most 25 wt. % water.

In an embodiment, an oligonucleotide composition comprises a plurality of at least 700 mmol scale single batch synthetic preparations of an oligonucleotide, wherein the single batch composition is a liquid composition.

In an embodiment, a substantially pure oligonucleotide composition of an oligonucleotide wherein the 5′-hydroxyl group of the 5′-terminal nucleoside is protected.

In an embodiment, a method of preparing an oligonucleotide, wherein the method comprises:
a) providing a linker attached to a solid support wherein the linker comprises a protected hydroxyl group;
b) deprotecting the protected hydroxyl group of the linker thereby creating a deprotected hydroxyl group;
c) independently providing a nucleoside phosphoramidite, wherein the nucleoside phosphoramidite comprises a protected hydroxyl group and a protected phosphoramidite;
d) independently coupling a nucleoside phosphoramidite to the deprotected hydroxyl group of the linker, or to the deprotected hydroxyl group of the nucleoside from the previous iteration of the reaction cycle, thereby creating a phosphite triester linked nucleoside;
e) independently thiolating the protected phosphite triester linkage thereby creating a protected phosphorothioate linkage;
f) optionally, independently capping unreacted deprotected hydroxyl groups;
g) optionally, independently deprotecting the protected hydroxyl group of the nucleoside;
h) repeating the providing, coupling, thiolating, capping, and deprotecting steps (steps c) through g)) a predetermined number of times to provide a solid support-bound oligonucleotide;
i) deprotecting the protected phosphorothioate linkages;
j) cleaving the oligonucleotide from the solid support;
k) eluting the oligonucleotide from the solid support;
l) purifying the oligonucleotide eluate using an ion exchange chromatography column; and
m) concentrating the solution of the oligonucleotide compound, such as concentrating with thin film evaporation.

In an embodiment, an oligonucleotide composition comprising at least 700 mmol of an oligonucleotide having at most 25 wt. % water and a nucleic acid sequence:

SEQ ID NO: 3:
(5′-GTX GCC CCT TCT CCC XGC AGC-3′),

wherein:
1) X represents 5-methyl-2′-deoxycytidine; and
2) the oligonucleotide is prepared according to a method comprising:
   a) providing a linker attached to a solid support wherein the linker comprises a protected hydroxyl group;
   b) deprotecting the protected hydroxyl group of the linker thereby creating a deprotected hydroxyl group;
   c) independently providing a nucleoside phosphoramidite, wherein the nucleoside phosphoramidite comprises a protected hydroxyl group and a protected phosphoramidite;
   d) independently coupling the nucleoside phosphoramidite to the deprotected hydroxyl group of the linker, or to the deprotected hydroxyl group of the nucleoside from the previous iteration of the reaction cycle, thereby creating a phosphite triester linked nucleoside;
   e) independently thiolating the protected phosphite triester linkage thereby creating a protected phosphorothioate linkage;
   f) optionally, independently capping unreacted deprotected hydroxyl groups;
   g) optionally, independently deprotecting the protected hydroxyl group of the nucleoside;
   h) repeating the providing, coupling, thiolating, capping, and deprotecting steps (steps c) through g) a predetermined number of times to provide a solid support-bound oligonucleotide;
   i) deprotecting the protected phosphorothioate linkages;
   j) cleaving the oligonucleotide from the solid support;
   k) eluting the oligonucleotide from the solid support;
   l) purifying the oligonucleotide eluate using an ion exchange chromatography column; and
   m) concentrating the solution of the oligonucleotide compound, such as concentrating with thin film evaporation.

In certain embodiments, one or more than one (including for instance all) of the following further embodiments may comprise each of the other embodiments or parts thereof.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the purifying step 1) comprises:
1) loading the oligonucleotide eluate from eluting step k) onto the ion exchange chromatography column;
2) deprotecting the protected hydroxyl group from the terminal nucleoside; and
3) eluting the oligonucleotide from the ion exchange chromatography column using a salt gradient.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the purifying step 1) comprises:
1) loading the oligonucleotide eluate from eluting step k) onto the ion exchange chromatography column;
2) deprotecting the protected hydroxyl group from the terminal nucleoside;
3) eluting the oligonucleotide from the ion exchange chromatography column using a salt gradient; and
4) desalting the oligonucleotide eluate from the ion exchange column via ultrafiltration and/or diafiltration.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the concentrating step m) comprises concentrating the desalted solution of the oligonucleotide compound, such as concentrating with thin film evaporation.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the concentrating step m) comprises concentrating the desalted solution of the oligonucleotide compound, such as concentrating with thin film evaporation, and further concentrating the solution with a freeze drying process.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the method further comprises independently washing the support with an aprotic solvent, such as acetonitrile, between one or more steps of a reaction cycle iteration, for example washing the support with between 1-10 column volumes, for example, between 1-7 column volumes or between 1-5 column volumes, such as between 2-4 column volumes, between one or more steps of a reaction cycle iteration.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the composition comprises at least 900 mmol of the oligonucleotide and at most 25 wt. % water.

In a further embodiment, the single batch composition, the substantially pure oligonucleotide composition, or the oligonucleotide composition, of any one of the above embodiments and any one or more of the further embodiments herein, is provided by the method disclosed herein at a yield of at least 2 g/mmol of an at least 700 mmol synthesis scale of the oligonucleotide, or of a pooled plurality of serial combinations of an oligonucleotide synthesis of an at least 700 mmol synthesis scale via a single oligonucleotide synthesis column (or synthesis run) and a purification of the synthesized oligonucleotide of an at least 700 mmol purification scale via a single ion-exchange chromatography purification column (or purification run) of the oligonucleotide, and at most 25 wt. % water, wherein the yield may be determined by optical density/mL (OD/mL) at a wavelength at 260 nm or may be determined by dry weight of the oligonucleotide composition corrected for moisture content relative to the synthesis scale to prepare the oligonucleotide.

In a further embodiment, the single batch composition, the substantially pure oligonucleotide composition, or the oligonucleotide composition, of any one of the above embodiments and any one or more of the further embodiments herein, is provided by the method disclosed herein at a yield of at least 3 g/mmol of an at least 700 mmol synthesis scale of the oligonucleotide, or of a pooled plurality of serial combinations of an oligonucleotide synthesis of an at least 700 mmol synthesis scale via a single oligonucleotide synthesis column (or synthesis run) and a purification of the synthesized oligonucleotide of an at least 700 mmol purification scale via a single ion-exchange chromatography purification column (or purification run) of the oligonucleotide, and at most 25 wt. % water, wherein the yield may be determined by optical density/mL (OD/mL) at a wavelength at 260 nm or may be determined by dry weight of the oligonucleotide composition corrected for moisture content relative to the synthesis scale to prepare the oligonucleotide.

In a further embodiment, the single batch composition, the substantially pure oligonucleotide composition, or the oligonucleotide composition, of any one of the above embodiments and any one or more of the further embodiments herein, is provided by the method disclosed herein at a yield of at least 3.5 g/mmol of an at least 900 mmol synthesis scale of the oligonucleotide, or of a pooled plurality of serial combinations of an oligonucleotide synthesis of an at least 900 mmol synthesis scale via a single oligonucleotide synthesis column (or synthesis run) and a purification of the synthesized oligonucleotide of an at least 900 mmol purification scale via a single ion-exchange chromatography purification column (or purification run) of the oligonucleotide, and at most 25 wt. % water, wherein the yield may be determined by optical density/mL (OD/mL) at a wavelength at 260 nm or may be determined by dry weight of the oligonucleotide composition corrected for moisture content relative to the synthesis scale to prepare the oligonucleotide.

In a further embodiment, the single batch composition, the substantially pure oligonucleotide composition, or the oligonucleotide composition, of any one of the above embodiments and any one or more of the further embodiments herein, is provided by the method disclosed herein at a yield of at least 3.5 g/mmol of an at least 900 mmol synthesis scale of the oligonucleotide, or of a pooled plurality of serial combinations of an oligonucleotide synthesis of an at least 900 mmol synthesis scale via a single oligonucleotide synthesis column (or synthesis run) and a purification of the synthesized oligonucleotide of an at least 900 mmol purification scale via a single ion-exchange chromatography purification column (or purification run) of the oligonucleotide, and at most 10 wt. % water, wherein the yield may be determined by optical density/mL (OD/mL) at a wavelength at 260 nm or may be determined by dry weight of the oligonucleotide composition corrected for moisture content relative to the synthesis scale to prepare the oligonucleotide.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the composition comprises at least 3 kg of the oligonucleotide and at most 25 wt. % water.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the composition comprises at least 50 mol %, such as between 70-99 mol %, of the oligonucleotide output from at least one oligonucleotide synthesis column having a capacity of providing at least 300 mmol or greater, such as at least 700 mmol, at least 800 mmol or at least 900 mmol or greater, of the oligonucleotide being synthesized, and at most 25 wt. % water, wherein the output is determined by optical density/mL (OD/mL).

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the composition comprises at least 50 mol %, such as between 70-99 mol %, of the oligonucleotide output (as determined by optical density/mL (OD/mL)) from between 4-10 oligonucleotide synthesis columns having a capacity of providing at least 300 mmol or greater, such as at least 700 mmol, at least 800 mmol or at least 900 mmol or greater, of the oligonucleotide being synthesized, and at most 25 wt. % water.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the oligonucleotide has a molecular weight of at least 3,000 Da, such as a molecular weight in the range of between 3,000-20,000 Da.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the oligonucleotide has 10-100 monomer subunits, for example, 15-25 monomer subunits, such as 20, 21, or 22, monomer subunits.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the oligonucleotide is an anti-SMAD7 oligonucleotide.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the nucleotide sequence has the nucleic acid sequence of SEQ ID NO: 2, 3, 4, 5, 6, 7, 8, 9, 10, 11, or 12, or the complementary sequence thereto.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the nucleotide sequence has the nucleic acid sequence:

SEQ ID NO: 3:
(5′-GTX GCC CCT TCT CCC XGC AGC-3′);

wherein X represents 5-methyl-2′-deoxycytidine.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein at least one of the internucleotide linkages of the oligonucleotide is an O,O-linked phosphorothioate.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein all of the internucleotide linkages of the oligonucleotide are O,O-linked phosphorothioates.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the molecular weight is a protonated molecular weight.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the molecular weight is an alkai metal molecular weight, such as a sodium salt form molecular weight.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the molecular weight is an alkaline metal molecular weight, such as a magnesium salt form molecular weight.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the oligonucleotide composition comprises at most 20 wt. % water, at most 15 wt. % water, or at most 10 wt. % water, such as comprises in the range of between 5-10 wt. % water, for example, in the range of between 6-8 wt. % water.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein only the 5′-hydroxyl group of the 5′-terminal nucleoside is protected.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the protected oligonucleotide is obtained after cleavage and elution from a synthesis column.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the degree of purity of the composition comprising the oligonucleotide is 60% or greater (as determined, for example, by RP-HPLC).

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the degree of purity of the composition comprising the protected oligonucleotide is 60% or greater (as determined, for example, by RP-HPLC).

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the protected oligonucleotide has a molecular weight of at least 3,000 Da, such as a molecular weight in the range of between 3,000-20,000 Da.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the solid support having a linker attached thereto is a controlled pore glass.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the solid support having a linker attached thereto is a crosslinked polystyrene.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the solid support has a loading capacity sufficient to prepare the oligonucleotide in an amount in the range of 300-3,000 mmol, for example, prepare the oligonucleotide in an amount in the range of 700-3,000 mmol, such as prepare the oligonucleotide in an amount of at least 600 mmol, at least 700 mmol, at least 900 mmol, at least 1,600 mmol, or at least 2,400 mmol.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the loading density of the solid support is at least 300 micromole linker/gram of solid support, such as 325-375 micromole linker/gram of solid support.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the column housing the solid support has a column inner diameter in the range of between 35-100 cm, for example between 50-100 cm, such as an inner diameter of 35 cm, 40 cm, 50 cm, 60 cm, 70 cm, 80 cm, 90 cm, or 100 cm.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the solid support has a bed height in the range of between 4-20 cm, such as between 7-20 cm.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the solid support has a column volume of at least 20 L, such as a column volume in the range of between 20-35 L.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the solid support having a linker attached thereto is NITTOPHASE UNYLINKER 350.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the linker comprises a terminal hydroxyl group.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the linker comprises a terminal hydroxyl group that is reactive with a nucleoside phosphoramidite.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the linker comprises a protected terminal hydroxyl group that, when deprotected, is reactive with a nucleoside phosphoramidite.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the linker is a UNYLINKER.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the hydroxyl protecting group on the linker is a dimethoxytrityl (DMT) group.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the protected hydroxyl group of the linker is deprotected with a protic acid, for example, 3-15 wt. % of dichloroacetic acid in toluene (v/v), such as 2-10 wt. % of dichloroacetic acid in toluene (v/v), for example, 10 wt. % of dichloroacetic acid in toluene (v/v).

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the coupling comprises providing the nucleoside phosphoramidite, for example, providing an excess amount of the nucleoside phosphoramidite, such as 1-8 equivalents of the nucleoside phosphoramidite relative to the equivalents of the solid support.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the coupling comprises providing the nucleoside phosphoramidite with an activator, for example, dicyanoimidazole (DCI).

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the coupling comprises providing 0.1-1 equivalents of the nucleoside phosphoramidite relative to the equivalents of the activator provided.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the coupling comprises providing the activator and the nucleoside phosphoramidite in a molar ratio of 2-5:1.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the nucleoside phosphoramidite is a protected nucleoside phosphoramidite, such as a protected nucleoside phosphoramidite comprising a 5′-hydroxyl protected group and 3′-hydroxyl protected group.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the 5′-hydroxyl protecting group of the protected nucleoside phosphoramidite is a dimethoxytrityl (DMT) group

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the 3′-hydroxyl protecting group of the protected nucleoside phosphoramidite is a phosphoramidite group, such as a (2-cyanoethyl)-N,N-diisopropyl-phosphoramidite group.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the thiolation of the protected phosphite triester linkage with a thiolating agent forms a protected phosphorothioate linkage, such as a 2-cyanoethoxy-protected phosphorothioate linkage.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the thiolating agent is xanthane hydride (XH), such as xanthane hydride (XH) in pyridine.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein 1-8 equivalents, such as 5-8 equivalents, of the xanthane hydride (XH) is provided, relative to the equivalents of the solid support.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the unreacted deprotected hydroxyl groups are capped with an acyl group, such as an alkyl acyl group.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the capping of the unreacted deprotected hydroxyl groups comprises adding:
a) a first capping solution (Cap A), comprising N-methylimidazole (NMI), pyridine, and acetonitrile; and
b) a second capping solution (Cap B), comprising capping agent and acetonitrile.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the capping of the unreacted deprotected hydroxyl groups comprises adding a premixed mixture of:
a) a first capping solution (Cap A), comprising N-methylimidazole (NMI), pyridine, and acetonitrile; and
b) a second capping solution (Cap B), comprising capping agent and acetonitrile.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the capping of the unreacted deprotected hydroxyl groups comprises adding a capping solution, comprising N-methylimidazole (NMI), pyridine, capping agent, and acetonitrile.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the capping agent is isobutyric anhydride.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the protected 5′-hydroxyl group of the 5′-terminal nucleoside of the oligonucleotide is deprotected with a protic acid, for example, dichloroacetic acid, such as 3-10 wt. % of dichloroacetic acid in toluene (v/v).

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the steps of providing/coupling of a nucleoside phosphoramidite, thiolating (or oxidizing) the formed phosphite triester linkage, optionally capping the unreacted deprotected hydroxyl group(s), and optionally deprotecting the protected 5′-hydroxyl group of the 5′-terminal nucleoside of the oligonucleotide, are repeated a predetermined number of times, for example, repeated 9-99 times, repeated 14-24 times, such as 19, 20, or 21 times, to provide the solid support-bound oligonucleotide having a predetermined number of monomer subunits, for example, having 10-100 monomer subunits, having 15-25 monomer subunits, such as 20, 21, or 22 monomer subunits, respectively.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the steps of providing/coupling of a nucleoside phosphoramidite, thiolating (or oxidizing) the formed phosphite triester linkage, optionally capping the unreacted deprotected hydroxyl group(s), and optionally deprotecting the protected 5′-hydroxyl group of the 5′-terminal nucleoside of the oligonucleotide, are repeated a predetermined number of times, for example, repeated 9-99 times, repeated 14-24 times, such as 19, 20, or 21 times, to provide the solid support-bound oligonucleotide in an amount in the range of between 300-3,000 mmol, for example, between 700-3,000 mmol.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the protected phosphorothioate linkages, such as the 2-cyanoethoxy-protecting groups, of the solid support-bound oligonucleotide are deprotected with an amine, such as triethylamine.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the deprotection of the protected phosphorothioate linkages, such as the 2-cyanoethoxy-protected phosphorothioate linkages, forms unprotected phosphorothioate linkages.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the deprotection of the protected phosphorothioate linkages, such as the 2-cyanoethoxy-protected phosphorothioate linkages, forms an amine salt form of the phosphorothioate linkages, such as forms a triethylamine salt form of the phosphorothioate linkages.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the cleaving of the deprotected solid support-bound oligonucleotide comprises providing a solution of ammonium hydroxide, for example, providing a heated solution of ammonium hydroxide, such as providing a heated ammonium hydroxide solution and recirculating the heated ammonium hydroxide solution through the column housing said deprotected solid support-bound oligonucleotide.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the exocyclic amino protecting groups comprises benzoyl and isobutyryl groups.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the providing and/or recirculating of the heated ammonium hydroxide solution further deprotects the benzoyl- and isobutyryl-amino protecting groups of the solid support-bound oligonucleotide having unprotected phosphorothioate linkages.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the provided and/or recirculated heated ammonium hydroxide solution is a 28-30% ammonia aqueous solution (w/w).

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the temperature of the provided and/or recirculated heated ammonium hydroxide solution is 40-70° C. or 40-60° C., such as a temperature of 40° C., 50° C., 60° C., or 65° C.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the heated ammonium hydroxide solution is recirculated through the support for 8-36 hours, for example, 12-36 hours, such as 24 hours.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the cleaved oligonucleotide comprises a 5′-hydroxyl protected group, such as a dimethoxytrityl (DMT) group

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the eluting of the cleaved and deprotected oligonucleotide from the support comprises washing the support with an aqueous solution.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the amount of the cleaved oligonucleotide comprising a 5′-hydroxyl protected group on the terminal nucleoside eluted from the solid support is in the range of between 300-3,000 mmol, for example, between 600-3,000 mmol, 700-3,000 mmol, or 1,000-3,000 mmol.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the amount of the oligonucleotide eluate loaded onto the ion exchange chromatography column is in the range of between 300-3,000 mmol, for example, between 600-3,000 mmol, 700-3,000 mmol, or 1,000-3,000 mmol.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein prior to the loading of the oligonucleotide eluate onto the ion exchange chromatography column, the oligonucleotide eluate is diluted with an aqueous buffer.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the 5′-hydroxyl group of the terminal nucleoside of the loaded oligonucleotide eluate remains protected during the loading step.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the 5′-hydroxyl group of the terminal nucleoside of the loaded oligonucleotide eluate, such as a dimethoxytrityl (DMT) group, is the only group protected on said loaded oligonucleotide eluate.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the ion exchange chromatography is an anion exchange chromatography.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the ion exchange chromatography column comprises Q Sepharose FF as the support media.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the oligonucleotide eluate is loaded onto the ion exchange chromatography column with a basic aqueous solution, for example, loaded onto the ion exchange chromatography column and washed with a basic aqueous solution.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the oligonucleotide eluate is loaded onto the ion exchange chromatography column and washed with a mixture of a basic aqueous solution and an aqueous salt solution.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the protected 5′-hydroxyl group of the terminal nucleoside of the loaded oligonucleotide eluate, such as 5′-hydroxyl protecting group is a dimethoxytrityl (DMT) group, is deprotected with a protic acid, for example, with acetic acid, such as with 80% aqueous acetic acid.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the oligonucleotide loaded and deprotected onto the ion exchange chromatography column is devoid of protecting groups, i.e., the oligonucleotide is a fully deprotected oligonucleotide.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the fully deprotected oligonucleotide is eluted from the ion exchange chromatography column with a salt gradient, for example, a basic salt gradient, such as a basic sodium chloride gradient.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the basic salt gradient is formed by mixing varying amounts of a basic aqueous solution and an aqueous salt solution.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the basic aqueous solution is a sodium hydroxide solution.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the aqueous salt solution is a sodium chloride solution.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the salt gradient ranges from 5% to 95% of an aqueous basic solution comprising sodium chloride.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the oligonucleotide eluted from the ion exchange chromatography column is a fully deprotected oligonucleotide.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the overall yield of the oligonucleotide prepared according to the method, beginning from synthesis through collected oligonucleotide from the purification step using ion exchange chromatography, is at least 50%, as determined by OD/mL at a wavelength at 260 nm, for example, in the range of between 55-100%, as determined by OD/mL at a wavelength at 260 nm.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the overall yield of the oligonucleotide prepared according to the method, beginning from synthesis through collected oligonucleotide from the purification step using ion exchange chromatography, is at least 50%, as determined by optical density/mL (OD/mL) at a wavelength at 260 nm, for example, in the range of between 50-95%, as determined by optical density/mL (OD/mL) at a wavelength at 260 nm.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the purity of the oligonucleotide prepared according to the method, beginning from synthesis through collected oligonucleotide from the purification step using ion exchange chromatography, is at least 50%, as determined by RP-HPLC, for example, in the range of between 50-95%, as determined by RP-HPLC.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the fully deprotected oligonucleotide is desalted via an ultrafiltration and/or diafiltration process.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the ultrafiltration and/or diafiltration process utilizes regenerated cellulose, for example, a regenerated cellulose having a 1,000-3,000 Da molecular weight cutoff.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the ultrafiltration and/or diafiltration process utilizes water at a pH in the range of 5-8, for example, water at a pH in the range of 6.5-7.5, such as water at a pH in the range of 6.8-7.3.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the amount of the fully deprotected oligonucleotide eluate desalted via an ultrafiltration and/or diafiltration process is an amount pooled from the output of at least 1, 2, 3, 4, 5, 6, 7, 8, 9, or 10, ion exchange chromatography purification columns (or purification runs utilizing one or more ion exchange chromatography purification columns), for example, is an amount pooled from the output of between 1-10 ion exchange chromatography purification columns (or purification runs utilizing one or more ion exchange chromatography purification columns), such as between 1-8, between 2-10, between 3-9, between 4-7, between 4-6, between 6-10, or between 8-10, ion exchange chromatography purification columns (or purification runs utilizing one or more ion exchange chromatography purification columns).

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the fully deprotected oligonucleotide eluate desalted via an ultrafiltration and/or diafiltration process is pooled from the output of at least 1, 2, 3, 4, 5, 6, 7, 8, 9, or 10, ion exchange chromatography purification columns (or purification runs utilizing one or more ion exchange chromatography purification columns), for example from the output of between 1-10 ion exchange chromatography purification columns (or purification runs utilizing one or more ion exchange chromatography purification columns), and wherein a single, a plurality, or each, of the ion exchange purification column(s) have a loading capacity sufficient to provide 700 mmol or greater of the purified and fully deprotected oligonucleotide, for example, have a loading capacity sufficient to provide in the range of between 700-5,400 mmol of the purified oligonucleotide being synthesized.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the amount of the fully deprotected oligonucleotide eluate desalted via an ultrafiltration and/or diafiltration process is an amount pooled from the output of a plurality of serial combinations of an oligonucleotide synthesis via a single oligonucleotide synthesis column (or synthesis run) and a purification of the synthesized oligonucleotide via a single ion-exchange chromatography purification column (or purification run), such as the pooling of the output of 2, 3, 4, 5, 6, 7, 8, 9, or 10 serial combinations of oligonucleotide synthesis via a single oligonucleotide synthesis column (or synthesis run) and purification of the synthesized oligonucleotide via a single ion-exchange chromatography purification column (or purification run), such as between 1-8, between 2-10, between 3-9, between 4-7, between 4-6, between 6-10, or between 8-10 serial combinations.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the amount of the fully deprotected oligonucleotide eluate desalted via an ultrafiltration and/or diafiltration process is an amount pooled from the output of a plurality of serial combinations of an oligonucleotide synthesis via a single oligonucleotide synthesis column (or synthesis run) and a purification of the synthesized oligonucleotide via a single ion-exchange chromatography purification column (or purification run), such as the pooling of the output of 2, 3, 4, 5, 6, 7, 8, 9, or 10 serial combinations of oligonucleotide synthesis via a single oligonucleotide synthesis column (or synthesis run) and purification of the synthesized oligonucleotide via a single ion-exchange chromatography purification column (or purification run), and wherein a single, a plurality, or each, of the oligonucleotide synthesis column(s) and/or ion exchange purification column(s) have a loading capacity sufficient to provide 700 mmol or greater of the purified and fully deprotected oligonucleotide, for example, have a loading capacity sufficient to provide in the range of between 700-5,400 mmol of the purified oligonucleotide being synthesized.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the fully deprotected and purified oligonucleotide resulting from a single, a plurality, or each, of the ion exchange chromatography purification column(s) (or purification run(s) utilizing one or more ion exchange chromatography purification column(s)), that is to be pooled prior to desalting via an ultrafiltration and/or diafiltration process, is an independent amount of 700 mmol or greater of the fully deprotected and purified oligonucleotide, for example, 900 mmol or greater, 1,600 mmol or greater, 2,700 mmol or greater, 3,600 mmol or greater, 4,000 mmol or greater, 4,500 mmol or greater, 5,000 mmol or greater, or 5,400 mmol or greater, or may be an independent amount in the range of between 700-5,400 mmol, for example, between 700-4,500 mmol, 700-3,600 mmol, 900-1,600 mmol, 900-3,000 mmol, 900-2,700 mmol, 1,600-2,700 mmol, 2,700-3,600 mmol, 2,700-4,500 mmol, 3,000-4,000 mmol, 3,600-4,500 mmol, or 3,600-5,400 mmol, of the fully deprotected and purified oligonucleotide.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the amount of the fully deprotected and purified oligonucleotide eluate desalted via an ultrafiltration and/or diafiltration process is at least 900 mmol, for example, at least 1,600 mmol, at least 2,400 mmol, at least 2,700 mmol, at least 3,000 mmol, at least 3,600 mmol, or at least 4,500 mmol, or is in the range of between 900-5,400 mmol, for example between 900-4,500 mmol, 900-3,600 mmol, 900-3,000 mmol, 900-2,700 mmol, 1,600-2,700 mmol, 1,800-3,600 mmol, 1,600-2,700 mmol, 2,700-3,600 mmol, 2,700-4,500 mmol, 3,000-4,000 mmol, 3,600-4,500 mmol, or 3,600-5,400 mmol.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the amount of the fully deprotected oligonucleotide eluate desalted via an ultrafiltration and/or diafiltration process is a pooled amount of at least 700 mmol from at least 1, 2, 3, 4, 5, 6, 7, 8, 9, or 10, ion exchange chromatography purification columns (or purification runs utilizing one or more ion exchange chromatography purification columns), for example, a pooled amount of at least 900 mmol, at least 1,600 mmol, at least 2,400 mmol, at least 2,700 mmol, at least 3,600 mmol, or at least 4,500 mmol, or a pooled amount in the range of between 700-4,000 mmol, for example, between 900-1,600 mmol, 900-3,000 mmol, 900-2,700 mmol, 1,600-2,700 mmol, 2,700-3,600 mmol, 2,700-4,500 mmol, 3,000-4,000 mmol, 3,600-4,500 mmol, or 3,600-5,400 mmol, from at least 1, 2, 3, 4, 5, 6, 7, 8, 9, or 10, ion exchange chromatography purification columns (or purification runs utilizing one or more ion exchange chromatography purification columns).

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the ultrafiltration and/or diafiltration process effectively desalts the fully deprotected and purified oligonucleotide eluate such that the resulting permeate (diafiltrate) has a conductivity of less than 900 uS/cm, for example, has a conductivity in the range of between 40-900 uS/cm, such as between 40-150 uS/cm, between 40-100 uS/cm, or between 40-75 uS/cm.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the ultrafiltration and/or diafiltration process effectively desalts the fully deprotected and purified oligonucleotide eluate such that the resulting desalted fully deprotected oligonucleotide retentate solution has a sodium content in the range of between 6-8 wt. %, for example, between 6-7 wt. %, between 7-8 wt. %, or between 6.5-7.5 wt. %.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the desalted fully deprotected oligonucleotide retentate solution is concentrated.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the desalted fully deprotected oligonucleotide retentate solution is concentrated, such as concentrated with thin film evaporation.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the thin film evaporation jacket temperature is 30° or greater, for example, in the range of 30−95° C. or 60-90° C.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the thin film evaporation pressure is 5-100 Torr, for example, 30-100 Torr or 20-80 Torr.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the amount of the desalted fully deprotected oligonucleotide retentate solution from the ultrafiltration and/or diafiltration process that is concentrated, such as concentrated by thin film evaporation, is at least 900 mmol, for example, at least 1,600 mmol, at least 2,400 mmol, at least 2,700 mmol, at least 3,600 mmol, or at least 4,500 mmol, or is in the range of between 900-5,400 mmol, for example between 900-4,500 mmol, 900-4,000 mmol, 900-3,600 mmol, 900-3,000 mmol, 900-2,700 mmol, 1,600-2,700 mmol, 1,800-3,600 mmol, 2,700-3,600 mmol, 2,700-4,500 mmol, 3,000-4,000 mmol, 3,600-4,500 mmol, or 3,600-5,400 mmol.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the amount of the desalted fully deprotected oligonucleotide retentate solution from the ultrafiltration and/or diafiltration process that is concentrated, such as concentrated by thin film evaporation, is at least 800 OD/mL at a wavelength of 260 nm, for example, between in the range of between 800-7,000 OD/mL at a wavelength of 260 nm.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the single batch composition, the substantially pure oligonucleotide composition, or the oligonucleotide composition, is a liquid composition, or wherein the method of preparing results in preparing a liquid composition.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the single batch composition, the substantially pure oligonucleotide composition, or the oligonucleotide composition, is derived exclusive of a freeze drying process, or wherein the method of preparing is exclusive of a freeze drying process.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the concentrated oligonucleotide, such as the thin film evaporated oligonucleotide, is subjected to a freeze drying process.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the single batch composition, the substantially pure oligonucleotide composition, or the oligonucleotide composition, is a solid composition, or wherein the method of preparing results in preparing a solid composition.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the freeze drying process utilizes a vacuum in the range of 1-500 millitorr, for example, 1-300 millitorr or 100-500 millitorr.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the freeze drying process utilizes a temperature in the range of −50 to 35° C., for example, −50 to −30° C., 15-25° C., −10 to 20° C., or −45 to 20° C.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the amount of the concentrated oligonucleotide solution, such as the amount of the concentrated oligonucleotide solution from the thin film evaporation process, that is further concentrated and/or dried by a freeze drying process is at least 900 mmol, for example, at least 1,600 mmol, at least 2,400 mmol, at least 2,700 mmol, at least 3,600 mmol, or at least 4,500 mmol, or is in the range of between 900-5,400 mmol, for example between 900-4,500 mmol, 900-4,000 mmol, 900-3,600 mmol, 900-3,000 mmol, 900-2,700 mmol, 1,600-2,700 mmol, 1,800-3,600 mmol, 2,700-3,600 mmol, 2,700-4,500 mmol, 3,000-4,000 mmol, 3,600-4,500 mmol, or 3,600-5,400 mmol.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the amount of the concentrated oligonucleotide solution, such as the amount of the concentrated oligonucleotide solution from the thin film evaporation process, that is further concentrated and/or dried by a freeze drying process is at least 800 OD/mL at a wavelength of 260 nm, for example, between in the range of between 800-7,000 OD/mL at a wavelength of 260 nm.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the method further comprises independently pre-swelling the support prior to providing the nucleoside phosphoramidite during at least one reaction cycle iteration.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the concentrated oligonucleotide resulting from the freeze drying process may have a water content of at most 25 wt. % water, for example, a water content of at most 20 wt. % water, at most 15 wt. % water, at most 10 wt. % water, or at most 5 wt. % water, or for example, a water content in the range of between 25-5 wt. % water, such as between 25-10 wt. % water, between 15-5 wt. % water between 10-5 wt. % water, or between 6-8 wt. % water.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the oligonucleotide is a deoxyribonucleotide, for example, the anti-SMAD7 oligonucleotide, such as the oligonucleotide having the nucleic acid sequence of SEQ ID NO: 2, 3, 4, 5, 6, 7, 8, 9, 10, 11, or 12, or the complementary sequence thereto, is a deoxyribonucleotide.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the oligonucleotide is a ribonucleotide, for example, the anti-SMAD7 oligonucleotide, such as the oligonucleotide having the nucleic acid sequence of SEQ ID NO: 2, 3, 4, 5, 6, 7, 8, 9, 10, 11, or 12, or the complementary sequence thereto, is a deoxyribonucleotide.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the degree of homogeneity of the oligonucleotide is at least 90%, such as at least 95%, at least 96%, at least 97%, at least 98%, or at least 99%, and wherein the oligonucleotide may be an anti-SMAD7 oligonucleotide, for example, an oligonucleotide having the nucleic acid sequence of SEQ ID NO: 2, 3, 4, 5, 6, 7, 8, 9, 10, 11, or 12, or the complementary sequence thereto, such as Mongersen (formerly GED-0301).

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the method solid support is exclusive of pre-swelling the support prior to providing the nucleoside phosphoramidite during at least one reaction cycle iteration, for example exclusive of pre-swelling the support prior to providing the nucleoside phosphoramidite during each reaction cycle iteration.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the preswelling step comprising a preswelling agent, such as toluene or dimethylformamide.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the method steps are performed in the order in which they are recited.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the optional step of capping the unreacted deprotected hydroxyl group(s) and the optional step of deprotecting the protected 5′-hydroxyl group of the 5′-terminal nucleoside of the oligonucleotide are excluded during the last reaction cycle iteration.

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the optional step of capping the unreacted deprotected hydroxyl group(s) and the optional step of deprotecting the protected 5′-hydroxyl group of the 5′-terminal nucleoside of the oligonucleotide are performed in each iteration of the reaction cycle, exclusive of the last iteration (i.e., not performed in the last iteration).

In a further embodiment, the single batch composition, the substantially pure oligonucleotide composition, the oligonucleotide composition, the oligonucleotide or oligonucleotide composition prepared according to the methods of preparing, or the method of preparing, of any one of the above embodiments and any one or more of the further embodiments herein, wherein the oligonucleotide composition is obtained by a method as disclosed herein.

In an embodiment, an oligonucleotide obtained from the single batch composition, the substantially pure oligonucleotide composition, or the oligonucleotide composition prepared according to the methods of preparing as disclosed herein, of any one of the above embodiments and any one or more of the further embodiments herein, wherein the oligonucleotide is administered according to the methods described herein for treating or managing inflammatory bowel disease (IBD) in a patient having IBD.

In an embodiment, an oligonucleotide obtained from the single batch composition, the substantially pure oligonucleotide composition, or the oligonucleotide composition prepared according to the methods of preparing as disclosed herein, of any one of the above embodiments and any one or more of the further embodiments herein, wherein the oligonucleotide is formulated as a pharmaceutical composition described herein, in combination with a pharmaceutically acceptable adjuvant and/or excipient.

In an embodiment, a pharmaceutical composition comprises an oligonucleotide obtained from the single batch composition, the substantially pure oligonucleotide composition, or the oligonucleotide composition prepared according to the methods of preparing as disclosed herein, of any one of the above embodiments and any one or more of the further embodiments herein, with a pharmaceutically acceptable adjuvant and/or excipient.

In an embodiment, a pharmaceutical composition comprises at least a portion of the single batch composition, the substantially pure oligonucleotide composition, or the oligonucleotide composition prepared according to the methods of preparing as disclosed herein, of any one of the above embodiments and any one or more of the further embodiments herein, with a pharmaceutically acceptable adjuvant and/or excipient.

In a further embodiment, the pharmaceutical composition of any one of the above embodiments and any one or more of the further embodiments herein, wherein the pharmaceutical composition is an oral dosage form, such as a tablet or a coated tablet.

In a further embodiment, the pharmaceutical composition of any one of the above embodiments and any one or more of the further embodiments herein, wherein the at least a portion of the single batch composition, the substantially pure oligonucleotide composition, or the oligonucleotide composition prepared according to the methods of preparing as disclosed herein, comprises in the range of between 10-500 mg of the oligonucleotide having a nucleic acid sequence:

SEQ ID NO: 3:
(5′-GTX GCC CCT TCT CCC XGC AGC-3′),

wherein X represents 5-methyl-2′-deoxycytidine.

In a further embodiment, the pharmaceutical composition of any one of the above embodiments and any one or more of the further embodiments herein, wherein the at least a portion of the single batch composition, the substantially pure oligonucleotide composition, or the oligonucleotide composition prepared according to the methods preparing as disclosed herein, comprises about 40 mg or about 160 mg of the oligonucleotide having a nucleic acid sequence:

SEQ ID NO: 3:
(5′-GTX GCC CCT TCT CCC XGC AGC-3′),

wherein X represents 5-methyl-2′-deoxycytidine.

In an embodiment, a method of preparing the pharmaceutical composition of any one of the above embodiments and any one or more of the further embodiments herein, comprising formulating an oligonucleotide obtained from the single batch composition, the substantially pure oligonucleotide composition, or the oligonucleotide composition prepared according to the methods of preparing as disclosed herein, with the pharmaceutically acceptable adjuvant and/or excipient.

In an embodiment, a method of preparing the pharmaceutical composition of any one of the above embodiments and any one or more of the further embodiments herein, comprising formulating at least a portion of the single batch composition, the substantially pure oligonucleotide composition, or the oligonucleotide composition prepared according to the methods of preparing as disclosed herein, with the pharmaceutically acceptable adjuvant and/or excipient.

In an embodiment, a method of preparing a series of pharmaceutical compositions (for example, oral dosage forms, tablets, or coated tablets, as described herein), comprising partitioning the single batch composition, the substantially pure oligonucleotide composition, or the oligonucleotide composition prepared according to the methods of preparing as disclosed herein, into a series of portions, amounts, or doses, suitable for oral dosage, and combining each portion, amount, or dose, suitable for oral dosage, of the series of portions amounts, or doses, suitable for oral dosage, with a pharmaceutically acceptable adjuvant and/or excipient.

In a further embodiment, the method of preparing the series of pharmaceutical compositions of any one of the above embodiments and any one or more of the further embodiments herein, wherein the series of pharmaceutical compositions is at least 100 pharmaceutical compositions.

In a further embodiment, the method of preparing the series of tablets of any one of the above embodiments and any one or more of the further embodiments herein, wherein the series of pharmaceutical compositions is between 100-1,000,000 pharmaceutical compositions.

In a further embodiment, the method of preparing the series of pharmaceutical compositions of any one of the above embodiments and any one or more of the further embodiments herein, wherein each portion, amount, or dose, suitable for oral dosage, of the series of portions, amounts, or doses, suitable for oral dosage, comprises in the range of between 10-500 mg of the oligonucleotide having a nucleic acid sequence:

SEQ ID NO: 3:
(5′-GTX GCC CCT TCT CCC XGC AGC-3′),

wherein X represents 5-methyl-2′-deoxycytidine.

In a further embodiment, the method of preparing the series of pharmaceutical compositions of any one of the above embodiments and any one or more of the further embodiments herein, wherein each portion, amount, or dose, suitable for oral dosage, of the series of portions, amounts, or doses, suitable for oral dosage, comprises about 40 mg or about 160 mg of the oligonucleotide having a nucleic acid sequence:

SEQ ID NO: 3:
(5′-GTX GCC CCT TCT CCC XGC AGC-3′),

wherein X represents 5-methyl-2′-deoxycytidine.

In a further embodiment, the method of preparing the series of pharmaceutical compositions of any one of the above embodiments and any one or more of the further embodiments herein, wherein the series of pharmaceutical compositions is a series of tablets.

In a further embodiment, the method of preparing the series of pharmaceutical compositions of any one of the above embodiments and any one or more of the further embodiments herein, wherein the series of pharmaceutical compositions is a series of tablets, wherein the series of tablets is at least 100 tablets, at least 500 tablets, at least 1,000 tablets, at least 2,000 tablets, at least 5,000 tablets, at least 10,000 tablets, at least 20,000 tablets, at least 50,000 tablets, at least 100,000 tablets, or at least 200,000 tablets.

In a further embodiment, the method of preparing the series of pharmaceutical compositions of any one of the above embodiments and any one or more of the further embodiments herein, wherein the series of pharmaceutical compositions is a series of tablets, wherein the series of tablets is between 100-1,000,000 tablets, 1,000-1,000,000 tablets, 10,000-1,000,000 tablets, 50,000-1,000,000 tablets, 100,000-1,000,000 tablets, or 500-1,000,000 tablets.

In a further embodiment, the method of preparing the series of pharmaceutical compositions of any one of the above embodiments and any one or more of the further embodiments herein, wherein the series of pharmaceutical compositions is a series of coated tablets.

In an embodiment, a pharmaceutical composition batch, comprising at least a portion of the single batch composition, the substantially pure oligonucleotide composition, or the oligonucleotide composition prepared according to the methods of preparing as disclosed herein, and a pharmaceutically acceptable adjuvant and/or excipient.

In a further embodiment, the pharmaceutical composition batch of any one of the above embodiments and any one or more of the further embodiments herein, wherein the pharmaceutical composition batch comprises at least 10 wt. %, at least 20 wt. %, at least 30 wt. %, at least 40 wt. %, at least 50 wt. %, at least 60 wt. %, at least 70 wt. %, at least 80 wt. %, at least 90 wt. %, at least 95 wt. %, or 100 wt. %, of the single batch composition, the substantially pure oligonucleotide composition, or the oligonucleotide composition prepared according to the methods of preparing as disclosed herein.

In a further embodiment, the pharmaceutical composition batch of any one of the above embodiments and any one or more of the further embodiments herein, wherein the pharmaceutical composition batch comprises at least one oral dosage form.

In a further embodiment, the pharmaceutical composition batch of any one of the above embodiments and any one or more of the further embodiments herein, wherein the at least one oral dosage form is a tablet.

In a further embodiment, the pharmaceutical composition batch of any one of the above embodiments and any one or more of the further embodiments herein, wherein the pharmaceutical composition batch comprises a series of oral dosage forms.

In a further embodiment, the pharmaceutical composition batch of any one of the above embodiments and any one or more of the further embodiments herein, wherein the series of oral dosage forms is a series of tablets.

In a further embodiment, the pharmaceutical composition batch of any one of the above embodiments and any one or more of the further embodiments herein, wherein the series of tablets is at least 100 tablets.

In a further embodiment, the pharmaceutical composition batch of any one of the above embodiments and any one or more of the further embodiments herein, wherein the series of tablets is between 100-1,000,000 tablets.

In a further embodiment, the pharmaceutical composition batch of any one of the above embodiments and any one or more of the further embodiments herein, wherein the pharmaceutical composition batch comprises the tablet is a coated tablet.

In a further embodiment, the pharmaceutical composition batch of any one of the above embodiments and any one or more of the further embodiments herein, wherein the series of tablets is a series of coated tablets.

In a further embodiment, the pharmaceutical composition batch of any one of the above embodiments and any one or more of the further embodiments herein, wherein the at least one oral dosage form, the at least one tablet, the at least one coated tablet, each of the series of oral dosage forms, each of the series of tablets, or each of the series of coated tablets, comprises in the range of between 10-500 mg of the oligonucleotide having a nucleic acid sequence:

SEQ ID NO: 3:
(5′-GTX GCC CCT TCT CCC XGC AGC-3′),

wherein X represents 5-methyl-2′-deoxycytidine.

In a further embodiment, the pharmaceutical composition batch of any one of the above embodiments and any one or more of the further embodiments herein, wherein the at least one oral dosage form, the at least one tablet, the at least one coated tablet, each of the series of oral dosage forms, each of the series of tablets, or each of the series of coated tablets, comprises about 40 mg or 160 mg of the oligonucleotide having a nucleic acid sequence:

SEQ ID NO: 3:
(5′-GTX GCC CCT TCT CCC XGC AGC-3′),

wherein X represents 5-methyl-2′-deoxycytidine.

In an embodiment, the single batch composition, the pharmaceutical composition batch, the method of preparing the single batch composition, the method of preparing the pharmaceutical composition, or the method of preparing the series of pharmaceutical compositions, of any one of the above embodiments and any one or more of the further embodiments herein, complies with the terms batch or lot as defined under 21 CFR 210.3(2) and 21 CFR 210.3(10), respectively.

In a further embodiment, the single batch composition, the pharmaceutical composition batch, the pharmaceutical composition, the method of preparing the series of tablets, or the method of treating, of any one of the above embodiments and any one or more of the further embodiments herein, wherein the oligonucleotide has a nucleic acid sequence of any one of SEQ ID NO: 2, 3, 4, 5, 6, 7, 8, 9, 10, 11, or 12.

In a further embodiment, the single batch composition, the pharmaceutical composition batch, the pharmaceutical composition, the method of preparing the series of tablets, or the method of treating, of any one of the above embodiments and any one or more of the further embodiments herein, wherein the oligonucleotide has a nucleic acid sequence of SEQ ID NO: 3: (5′-GTX GCC CCT TCT CCC XGC AGC-3′), wherein X represents 5-methyl-2′-deoxycytidine.

In an embodiment, a method of treating or managing inflammatory bowel disease (IBD) in a patient having IBD, comprising administering the pharmaceutical composition of any one of the above embodiments and any one or more of the further embodiments herein.

In a further embodiment, the method of treating or managing inflammatory bowel disease (IBD) in a patient having IBD of any one of the above embodiments and any one or more of the further embodiments herein, wherein the inflammatory bowel disease (IBD) is Crohn's disease (CD).

In a further embodiment, the method of treating or managing inflammatory bowel disease (IBD) in a patient having IBD of any one of the above embodiments and any one or more of the further embodiments herein, wherein the inflammatory bowel disease (IBD) is ulcerative colitis (UC).

While preferred embodiments of the present invention have been shown and described herein, it will be obvious to those skilled in the art that such embodiments are provided by way of example only. It is intended that the following claims define the scope of the invention and that methods and structures within the scope of these claims and their equivalents be covered thereby.

TABLE 7
Sequence Listing

| | | Comments; |
|---|---|---|
| ID | SEQUENCE | Reference |
| SEQ | ATG TTCAGGACCA AACGATCTGC GCTCGTCCGG | Coding Sequence: |
| ID | CGTCTCTGGA GGAGCCGTGC GCCCGGCGGC GAGGACGAGG | CDS (288-1568) of |
| NO: 1 | AGGAGGGCGC AGGGGGAGGT GGAGGAGGAG GCGA | NM_005904.3; Homo |
| | GGACA GCCGAGCGCA TGGGGCCGGT | sapiens SMAD |
| | GGCGGCGGCC CGGGCAGGGC TGGATGCTGC CTGGGCAAGG | family member 7 |
| | CGGTGCGAGG TGCCAAAGGT CACCACCATC CCCACCCGCC | (SMAD7), |
| | AGCCGCGGGC GCCGGCGCGG CCGGGGGCGC CGAGGCGGAT | transcript variant |
| | CTGAAGGCGC TCACGCACTC GGTGCTCAAG AAACTGAAGG | 1, mRNA (region |
| | AGCGGCAGCT GGAGCTGCTG CTCCAGGCCG TGGAGTCCCG | 108-128 |
| | CGGCGGGACG CGCACCGCGT GCCTCCTGCT GCCCGGCCGC | underlined). |
| | CTGGACTGCA GGCTGGGCCC GGGGGCGCCC GCCGGCGCGC | |
| | AGCCTGCGCA GCCGCCCTCG TCCTACTCGC TCCCCCTCCT | |
| | GCTGTGCAAA GTGTTCAGGT GGCCGGATCT CAGGCATTCC | |
| | TCGGAAGTCA AGAGGCTGTG TTGCTGTGAA TCTTACGGGA | |
| | AGATCAACCC CGAGCTGGTG TGCTGCAACC CCCATCACCT | |
| | TAGCCGACTC TGCGAACTAG AGTCTCCCCC CCCTCCTTAC | |
| | TCCAGATACC CGATGGATTT TCTCAAACCA ACTGCAGACT | |
| | GTCCAGATGC TGTGCCTTCC TCCGCTGAAA CAGGGGGAAC | |
| | GAATTATCTG GCCCCTGGGG GGCTTTCAGA TTCCCAACTT | |
| | CTTCTGGAGC CTGGGGATCG GTCACACTGG TGCGTGGTGG | |
| | CATACTGGGA GGAGAAGACG AGAGTGGGGA GGCTCTACTG | |
| | TGTCCAGGAG CCCTCTCTGG ATATCTTCTA TGATCTACCT | |
| | CAGGGGAATG GCTTTTGCCT CGGACAGCTC AATTCGGACA | |
| | ACAAGAGTCA GCTGGTGCAG AAGGTGCGGA GCAAAATCGG | |
| | CTGCGGCATC CAGCTGACGC GGGAGGTGGA TGGTGTGTGG | |
| | GTGTACAACC GCAGCAGTTA CCCCATCTTC ATCAAGTCCG | |
| | CCACACTGGA CAACCCGGAC TCCAGGACGC TGTTGGTACA | |
| | CAAGGTGTTC CCCGGTTTCT CCATCAAGGC TTTCGACTAC | |
| | GAGAAGGCGT ACAGCCTGCA GCGGCCCAAT GACCACGAGT | |
| | TTATGCAGCA GCCGTGGACG GGCTTTACCG TGCAGATCAG | |
| | CTTTGTGAAG GGCTGGGGCC AGTGCTACAC CCGCCAGTTC | |
| | ATCAGCAGCT GCCCGTGCTG GCTAGAGGTC ATCTTCAACA | |
| | GCCGGTAG | |
| SEQ | 5′-GTXYCCCCTTCTCCCXYCAGC-3′, | |
| ID | or the complementary sequence thereto; | |
| NO: 2 | wherein X independently represents a | |
| | nucleotide comprising a nitrogenous base | |
| | selected from the group consisting of | |
| | cytosine, 5-methylcytosine and 2′-0- | |
| | methylcytosine, and Y independently | |
| | represents a nucleotide comprising a | |
| | nitrogenous base selected from the group | |
| | consisting of guanine, 5-methylguanine, or | |
| | 2′-O-methylguanine; provided at least one of | |
| | the nucleotides X or Y comprises a | |
| | methylated nitrogenous base. | |
| | In certain embodiments, at least one | |
| | internucleoside linkage, for example, at | |
| | least 2, 3, 4, 5, 6, 7, 8, 9, 10, or all of | |
| | the internucleoside linkage are O,O-linked | |
| | phosphorothioate linkages. | |
| | In certain embodiments, all of the | |
| | internucleoside linkages are O,O-linked | |
| | phosphorothioate linkages. | |
| SEQ | 5′-GTXGCCCCTTCTCCCXGCAGC-3′ | |
| ID | or the complementary sequence thereto; | |
| NO: 3 | wherein X represents 5-methyl-2′- | |
| | deoxycytidine (“5-Me-dC”), and wherein each | |
| | of the 20 internucleotide linkages are O,O- | |
| | linked phosphorothioate linkages. | |
| SEQ | 5′-GTCGCCCCTTCTCCCCGCAG-3′ | |
| ID | or the complementary sequence thereto; | |
| NO: 4 | wherein at least one internucleoside | |
| | linkage, for example, at least 2, 3, 4, 5, | |
| | 6, 7, 8, 9, 10, or all of the | |
| | internucleoside linkage are O,O-linked | |
| | phosphorothioate linkages. | |
| SEQ | 5′-GTXGCCCCTTCTCCCXGCAG-3′ | |
| ID | or the complementary sequence thereto; | |
| NO: 5 | wherein X represents 5-methyl-2′- | |
| | deoxycytidine (“5-Me-dC”); and wherein at | |
| | least one internucleoside linkage, for | |
| | example, at least 2, 3, 4, 5, 6, 7, 8, 9, | |
| | 10, or all of the internucleoside linkage | |
| | are O,O-linked phosphorothioate linkages. | |
| SEQ | 5′-GTTTGGTCCTGAACATGC- 3′ | |
| ID | or the complementary sequence thereto; | |
| NO: 6 | wherein at least one internucleoside | |
| | linkage, for example, at least 2, 3, 4, 5, | |
| | 6, 7, 8, 9, 10, or all of the | |
| | internucleoside linkage are O,O-linked | |
| | phosphorothioate linkages. | |
| SEQ | 5′-GTTTGGTCCTGAACAT-3′ | |
| ID | or the complementary sequence thereto; | |
| NO: 7 | wherein at least one internucleoside | |
| | linkage, for example, at least 2, 3, 4, 5, | |
| | 6, 7, 8, 9, 10, or all of the | |
| | internucleoside linkage are O,O-linked | |
| | phosphorothioate linkages. | |
| SEQ | 5′-GTTTGGTCCTGAACATG-3′ | |
| ID | or the complementary sequence thereto; | |
| NO: 8 | wherein at least one internucleoside | |
| | linkage, for example, at least 2, 3, 4, 5, | |
| | 6, 7, 8, 9, 10, or all of the | |
| | internucleoside linkage are O,O-linked | |
| | phosphorothioate linkages. | |
| SEQ | 5′-AGCACCGAGTGCGTGAGC-3′ | |
| ID | or the complementary sequence thereto; | |
| NO: 9 | wherein at least one internucleoside | |
| | linkage, for example, at least 2, 3, 4, 5, | |
| | 6, 7, 8, 9, 10, or all of the | |
| | internucleoside linkage are O,O-linked | |
| | phosphorothioate linkages. | |
| SEQ | 5′-CGAACATGACCTCCGCAC-3′ | |
| ID | or the complementary sequence thereto; | |
| NO: 10 | wherein at least one internucleoside | |
| | linkage, for example, at least 2, 3, 4, 5, | |
| | 6, 7, 8, 9, 10, or all of the | |
| | internucleoside linkage are O,O-linked | |
| | phosphorothioate linkages. | |
| SEQ | 5′-GATCGTTTGGTCCTGAA-3′ | |
| ID | or the complementary sequence thereto; | |
| NO: 11 | wherein at least one internucleoside | |
| | linkage, for example, at least 2, 3, 4, 5, | |
| | 6, 7, 8, 9, 10, or all of the | |
| | internucleoside linkage are O,O-linked | |
| | phosphorothioate linkages. | |
| SEQ | 5′-ATCGTTTGGTCCTGAAC-3′ | |
| ID | or the complementary sequence thereto; | |
| NO: 12 | wherein at least one internucleoside | |
| | linkage, for example, at least 2, 3, 4, 5, | |
| | 6, 7, 8, 9, 10, or all of the | |
| | internucleoside linkage are O,O-linked | |
| | phosphorothioate linkages. | |

What is claimed is:

1. A single batch composition of an oligonucleotide comprising at least 700 mmol of the oligonucleotide and at most 25 wt. % water; wherein the oligonucleotide has a nucleic acid sequence:
SEQ ID NO: 3:
(5′-GTX GCC CCT TCT CCC XGC AGC-3′),
wherein X represents 5-methyl-2′-deoxycytidine.

2. A single batch composition of an oligonucleotide comprising at least 2 g/mmol of an at least 700 mmol synthesis scale of the oligonucleotide and at most 25 wt. % water; wherein the oligonucleotide has a nucleic acid sequence:
SEQ ID NO: 3:
(5′-GTX GCC CCT TCT CCC XGC AGC-3′),
wherein X represents 5-methyl-2′-deoxycytidine.

3. A single batch composition of an oligonucleotide comprising at least 2 kg of the oligonucleotide and at most 25 wt. % water; wherein the oligonucleotide has a nucleic acid sequence:
SEQ ID NO: 3:
(5′-GTX GCC CCT TCT CCC XGC AGC-3′),
wherein X represents 5-methyl-2′-deoxycytidine.

4. A single batch composition of an oligonucleotide comprising at least 50 mol % of the oligonucleotide output from at least one 700 mmol or greater oligonucleotide synthesis column and at most 25 wt. % water; wherein the oligonucleotide has a nucleic acid sequence:
SEQ ID NO: 3:
(5′-GTX GCC CCT TCT CCC XGC AGC-3′),
wherein X represents 5-methyl-2′-deoxycytidine.

5. The single batch oligonucleotide composition of any one of claims 1-4, wherein at least one of the internucleotide linkages of the oligonucleotide is an O,O-linked phosphorothioate.

6. The single batch oligonucleotide composition of any one of claims 1-5, wherein all of the internucleotide linkages of the oligonucleotide are O,O-linked phosphorothioates.

7. A substantially pure oligonucleotide composition of an oligonucleotide wherein the 5′-hydroxyl group of the 5′-terminal nucleoside is protected; wherein the oligonucleotide has a nucleic acid sequence:
SEQ ID NO: 3:
(5′-GTX GCC CCT TCT CCC XGC AGC-3′),
wherein X represents 5-methyl-2′-deoxycytidine.

8. The substantially pure oligonucleotide composition of claim 7, wherein only the 5′-hydroxyl group of the 5′-terminal nucleoside is protected.

9. The substantially pure oligonucleotide composition of claim 8, wherein the protected oligonucleotide is obtained after cleavage and elution from a synthesis column.

10. The substantially pure oligonucleotide composition of any one of claims 7-9, wherein the degree of purity of the composition is 60% or greater.

11. The substantially pure oligonucleotide composition of any one of claims 7-10, wherein at least one of the internucleotide linkages of the oligonucleotide is an O,O-linked phosphorothioate.

12. The substantially pure oligonucleotide composition of any one of claims 7-11, wherein all of the internucleotide linkages of the oligonucleotide are O,O-linked phosphorothioates.

13. The single batch oligonucleotide composition of any one of claims 1-6 or the substantially pure oligonucleotide composition of any one of claims 7-12, wherein the oligonucleotide is prepared according to a method comprising:
a) providing a linker attached to a solid support wherein the linker comprises a protected hydroxyl group;
b) deprotecting the protected hydroxyl group of the linker thereby creating a deprotected hydroxyl group;
c) independently providing a nucleoside phosphoramidite, wherein the nucleoside phosphoramidite comprises a protected hydroxyl group and a protected phosphoramidite;
d) independently coupling the nucleoside phosphoramidite to the deprotected hydroxyl group of the linker, or to the deprotected hydroxyl group of the nucleoside from the previous iteration of the reaction cycle, thereby creating a phosphite triester linked nucleoside;
e) independently thiolating the protected phosphite triester linkage thereby creating a protected phosphorothioate linkage;
f) optionally, independently capping unreacted deprotected hydroxyl groups;
g) optionally, independently deprotecting the protected hydroxyl group of the nucleoside;
h) repeating the providing, coupling, thiolating, capping, and deprotecting steps (steps c) through g)) a predetermined number of times to provide a solid support-bound oligonucleotide;
i) deprotecting the protected phosphorothioate linkages;
j) cleaving the oligonucleotide from the solid support;
k) eluting the oligonucleotide from the solid support;
l) purifying the oligonucleotide eluate using an ion exchange chromatography column; and
m) concentrating the solution of the oligonucleotide compound with thin film evaporation.

14. The oligonucleotide composition of claim 13, wherein the purifying step 1) comprises:
1) loading the oligonucleotide eluate from eluting step k) onto the ion exchange chromatography column;
2) deprotecting the protected hydroxyl group from the terminal nucleoside; and
3) eluting the oligonucleotide from the ion exchange chromatography column using a salt gradient.

15. The oligonucleotide composition of claim 13, wherein the purifying step 1) comprises:
1) loading the oligonucleotide eluate from eluting step k) onto the ion exchange chromatography column;
2) deprotecting the protected hydroxyl group from the terminal nucleoside;
3) eluting the oligonucleotide from the ion exchange chromatography column using a salt gradient; and
4) desalting the oligonucleotide eluate from the ion exchange column via ultrafiltration and/or diafiltration.

16. The oligonucleotide composition of any one of claims 13-15, wherein the solid support having a linker attached thereto is a crosslinked polystyrene.

17. The oligonucleotide composition of any one of claims 13-16, wherein the unreacted deprotected hydroxyl groups are capped with an acyl group.

18. The oligonucleotide composition of any one of claims 13-17, wherein the capping of the unreacted deprotected hydroxyl groups comprises adding:
a) a first capping solution (Cap A), comprising N-methylimidazole (NMI), pyridine, and acetonitrile; and
b) a second capping solution (Cap B), comprising capping agent and acetonitrile.

19. The oligonucleotide composition of claim 18, wherein the capping agent is isobutyric anhydride.

20. The oligonucleotide composition of any one of claims 13-19, wherein the thiolating agent is xanthane hydride (XH).

21. The oligonucleotide composition of any one of claims 13-20, wherein the cleaving of the deprotected solid support-bound oligonucleotide comprises providing a solution of ammonium hydroxide.

22. The oligonucleotide composition of any one of claims 13-21, wherein the ion exchange chromatography is an anion exchange chromatography.

23. The oligonucleotide composition of any one of claims 13-22, wherein the oligonucleotide eluate is loaded onto the ion exchange chromatography column with a basic aqueous solution.

24. The oligonucleotide composition of any one of claims 13-23, wherein the 5′-hydroxyl protected group of the loaded oligonucleotide eluate on the ion exchange chromatography column is deprotected with 80% aqueous acetic acid.

25. The oligonucleotide composition of any one of claims 13-24, wherein the fully deprotected oligonucleotide is eluted from the ion exchange chromatography column with a basic salt gradient.

26. The oligonucleotide composition of any one of claims 13-25, wherein the fully deprotected oligonucleotide is desalted via an ultrafiltration and/or diafiltration process.

27. The oligonucleotide composition of any one of claims 13-26, wherein the ultrafiltration and/or diafiltration process utilizes water at a pH in the range of 5-8.

28. The oligonucleotide composition of any one of claims 13-27, wherein the fully deprotected oligonucleotide eluate is concentrated with thin film evaporation.

29. The oligonucleotide composition of any one of claims 13-28, wherein the method steps are performed in the order in which they are recited.

30. The oligonucleotide composition of any one of claims 13-29, wherein the optional step of capping the unreacted deprotected hydroxyl groups and the optional step of deprotecting the protected hydroxyl group of the nucleoside are performed in each iteration of the reaction cycle, exclusive of the last iteration.

31. A pharmaceutical composition, comprising at least a portion of the single batch oligonucleotide composition of any one of claims 1-6, the substantially pure oligonucleotide composition of any one of claims 7-12, or the oligonucleotide composition of any one of claims 13-30, with a pharmaceutically acceptable adjuvant and/or excipient.

32. The pharmaceutical composition of claim 31, wherein the pharmaceutical composition is an oral dosage form.

33. The pharmaceutical composition of claim 32, wherein the oral dosage form of the pharmaceutical composition is a tablet.

34. The pharmaceutical composition of claim 33, wherein the tablet is a coated tablet.

35. The pharmaceutical composition of any one of claims 31-34, wherein the at least a portion of the single batch oligonucleotide composition of any one of claims 1-6, the substantially pure oligonucleotide composition of any one of claims 7-12, or the oligonucleotide composition of any one of claims 13-30, comprises in the range of between 10-500 mg of the oligonucleotide having a nucleic acid sequence:
SEQ ID NO: 3:
(5′-GTX GCC CCT TCT CCC XGC AGC-3′),
wherein X represents 5-methyl-2′-deoxycytidine.

36. The pharmaceutical composition of any one of claims 31-35, wherein the at least a portion of the single batch oligonucleotide composition of any one of claims 1-6, the substantially pure oligonucleotide composition of any one of claims 7-12, or the oligonucleotide composition of any one of claims 13-30, comprises about 40 mg or about 160 mg of the oligonucleotide having a nucleic acid sequence:
SEQ ID NO: 3:
(5′-GTX GCC CCT TCT CCC XGC AGC-3′),
wherein X represents 5-methyl-2′-deoxycytidine.

37. A method of treating or managing inflammatory bowel disease (IBD) in a patient having IBD, comprising administering the pharmaceutical composition of any one of claims 31-36.

38. The method of treating of claim 37, wherein the inflammatory bowel disease (IBD) is Crohn's disease (CD).

39. The method of treating of claim 37, wherein the inflammatory bowel disease (IBD) is ulcerative colitis (UC).

40. A method of preparing the pharmaceutical composition of any one of claims 31-36, comprising formulating the at least a portion of the single batch oligonucleotide composition of any one of claims 1-6, the substantially pure oligonucleotide composition of any one of claims 7-12, or the oligonucleotide composition of any one of claims 13-30, with the pharmaceutically acceptable adjuvant and/or excipient.

41. A method of preparing a series of tablets, comprising partitioning the single batch oligonucleotide composition of any one of claims 1-6, the substantially pure oligonucleotide composition of any one of claims 7-12, or the oligonucleotide composition of any one of claims 13-30, into a series of portions, amounts, or doses, suitable for oral dosage, and combining each portion, amount, or dose, suitable for oral dosage, of the series of portions amounts, or doses, suitable for oral dosage, with a pharmaceutically acceptable adjuvant and/or excipient.

42. The method of preparing the series of tablets of claim 41, wherein the series of tablets is at least 100 tablets.

43. The method of preparing the series of tablets of claim 41, wherein the series of tablets is between 100-1,000,000 tablets.

44. The method of preparing the series of tablets of any one of claims 41-43, wherein the series of tablets is a series of coated tablets.

45. The method of preparing the series of tablets of any one of claims 41-44, wherein each portion, amount, or dose, suitable for oral dosage, of the series of portions, amounts, or doses, suitable for oral dosage, comprises in the range of between 10-500 mg of the oligonucleotide having a nucleic acid sequence:
SEQ ID NO: 3:
(5′-GTX GCC CCT TCT CCC XGC AGC-3′),
wherein X represents 5-methyl-2′-deoxycytidine.

46. The method of preparing the series of tablets of any one of claims 41-45, wherein each portion, amount, or dose, suitable for oral dosage, of the series of portions, amounts, or doses, suitable for oral dosage, comprises about 40 mg or about 160 mg of the oligonucleotide having a nucleic acid sequence:
SEQ ID NO: 3:
(5′-GTX GCC CCT TCT CCC XGC AGC-3′),
wherein X represents 5-methyl-2′-deoxycytidine.

47. A pharmaceutical composition batch, comprising at least a portion of the single batch oligonucleotide composition of any one of claims 1-6, the substantially pure oligonucleotide composition of any one of claims 7-12, or the oligonucleotide composition of any one of claims 13-30, and a pharmaceutically acceptable adjuvant and/or excipient.

48. The pharmaceutical batch composition of claim 47, wherein the pharmaceutical composition batch comprises at least 10 wt. %, at least 20 wt. %, at least 30 wt. %, at least 40 wt. %, at least 50 wt. %, at least 60 wt. %, at least 70 wt. %, at least 80 wt. %, at least 90 wt. %, at least 95 wt. %, or 100 wt. %, of the single batch oligonucleotide composition of any one of claims 1-6, the substantially pure oligonucleotide composition of any one of claims 7-12, or the oligonucleotide composition of any one of claims 13-30.

49. The pharmaceutical batch composition of claim 48 or 48, wherein the pharmaceutical composition batch comprises at least one tablet or at least one coated tablet.

50. The pharmaceutical batch composition of claim 48 or 48, wherein the pharmaceutical composition batch comprises a series of tablets.

51. The pharmaceutical batch composition of claim 50, wherein the series of tablets is at least 100 tablets.

52. The pharmaceutical batch composition of claim 50, wherein the series of tablets is between 100-1,000,000 tablets.

53. The pharmaceutical batch composition of any one of claims 50-52, wherein the series of tablets is a series of coated tablets.

54. The pharmaceutical batch composition of any one of claims 47-53, wherein the at least one tablet, the at least one coated tablet, each of the series of tablets, or each of the series of coated tablets, comprises in the range of between 10-500 mg of the oligonucleotide having a nucleic acid sequence:
SEQ ID NO: 3:
(5′-GTX GCC CCT TCT CCC XGC AGC-3′),
wherein X represents 5-methyl-2′-deoxycytidine.

55. The pharmaceutical batch composition of any one of claims 47-54, wherein the at least one tablet, the at least one coated tablet, each of the series of tablets, or each of the series of coated tablets, comprises about 40 mg or 160 mg of the oligonucleotide having a nucleic acid sequence:
SEQ ID NO: 3:
(5′-GTX GCC CCT TCT CCC XGC AGC-3′),
wherein X represents 5-methyl-2′-deoxycytidine.
